(19) United States
(12) Patent Application Publication
(10) Pub. No.: US 2007/0185063 A1
(43) Pub. Date: Aug. 9, 2007

(54) Seven-membered ring nucleosides

(75) Inventors: Richard Storer, Folkestone (GB); Gilles Gosselin, Montpellier (FR); David Dukhan, Montpellier (FR); Frederic Leroy, Jonquieres (FR); Jean-Christophe Meillon, Montpellier (FR); Thierry Convard, Sathonay-Camp (FR)

Correspondence Address: JONES DAY, 222 EAST 41ST ST, NEW YORK, NY, 10017, US

(73) Assignees: Idenix Pharmaceuticals, Inc.; Centre National De La Recherche Scientifique

(21) Appl. No.: 11/509,272

(22) Filed: Aug. 23, 2006

Related U.S. Application Data
(60) Provisional application No. 60/710,771, filed on Aug. 23, 2005.

(51) Int. Cl. A61K 31/675; C07F 9/6512; C07D 473/12; C07D 473/02; A61K 31/52; C07D 409/02; C07D 405/02; A61K 31/522; A61K 31/55; A61K 31/513

(52) U.S. Cl. 514/81; 514/217.06; 514/85; 514/263.23; 514/269; 514/275; 544/243; 544/244; 544/269; 544/276; 544/277; 544/310; 540/600; 540/601

(57) ABSTRACT

The present invention provides nucleoside analogue compounds that treat a host infected with a Flaviviridae virus infection, or other viruses that exhibit RNA-dependent RNA viral replication, compositions comprising these compounds and methods of using the compounds for the treatment and/or prophylaxis of viral infection, especially hepatitis C, in an infected host.

### CROSS-REFERENCE TO RELATED APPLICATIONS

This application claims the benefit of priority to U.S. Provisional Application No. 60/710,771, filed Aug. 23, 2005, the disclosure of which is incorporated by reference.

### FIELD OF THE INVENTION

This invention is in the area of pharmaceutical chemistry and, in particular, provides nucleoside analogues that include a seven membered ring that inhibit viral replication. Included in the invention are pharmaceutically acceptable salts, esters, derivatives and prodrugs of these nucleoside analogues, as well as syntheses and uses of these compounds as anti-Flaviviridae agents in the treatment of hosts, notably humans, infected with a Flaviviridae virus, and in particular, hepatitis C virus.

### BACKGROUND OF THE INVENTION

Infection of a human with hepatitis C (HCV) is a major health issue that an increasing percentage of the world's population. Left untreated, HCV infection can lead to chronic liver disease, cirrhosis, hepatocellular carcinoma, and death.

Flaviviridae Viruses

The Flaviviridae family of viruses comprises at least three distinct genera: pestiviruses, which cause disease in cattle and pigs; flaviviruses, which are the primary cause of diseases such as dengue fever and yellow fever; and hepaciviruses, whose sole member is HCV. The flavivirus genus includes more than 68 members separated into groups on the basis of serological relatedness (Calisher et al., J. Gen. Virol, 1993, 70, 37-43). Clinical symptoms of infection vary and include fever, encephalitis and hemorrhagic fever (Fields Virology, Editors: Fields, B. N., Knipe, D. M., and Howley, P. M., Lippincott-Raven Publishers, Philadelphia, Pa., 1996, Chapter 31, 931-959). Flaviviruses of global concern that are associated with human disease include the dengue hemorrhagic fever viruses (DHF), yellow fever virus, shock syndrome and Japanese encephalitis virus (Halstead, S. B., Rev. Infect. Dis., 1984, 6, 251-264; Halstead, S. B., Science, 239:476-481, 1988; Monath, T. P., New Eng. J. Med., 1988, 319, 641-643).

The pestivirus genus includes bovine viral diarrhea virus (BVDV), classical swine fever virus (CSFV, also called hog cholera virus) and border disease virus (BDV) of sheep (Moennig, V. et al. Adv. Vir. Res. 1992, 41, 53-98). Pestivirus infections of domesticated livestock (cattle, pigs and sheep) cause significant economic losses worldwide. BVDV causes mucosal disease in cattle and is of significant economic importance to the livestock industry (Meyers, G. and Thiel, H.-J., Advances in Virus Research, 1996, 47, 53-118; Moennig V., et al, Adv. Vir. Res. 1992, 41, 53-98). Human pestiviruses have not been as extensively characterized as the animal pestiviruses. However, serological surveys indicate considerable pestivirus exposure in humans.

Pestiviruses and hepaciviruses are closely related virus groups within the Flaviviridae family. Other closely related viruses in this family include the GB virus A, GB virus A-like agents, GB virus-B and GB virus-C (also called hepatitis G virus, HGV). The hepacivirus group (hepatitis C virus; HCV) consists of a number of closely related but genotypically distinguishable viruses that infect humans. There are approximately 6 HCV genotypes and more than 50 subtypes. Due to the similarities between pestiviruses and hepaciviruses, combined with the poor ability of hepaciviruses to grow efficiently in cell culture, bovine viral diarrhea virus (BVDV) is often used as a surrogate to study the HCV virus.

The genetic organization of pestiviruses and hepaciviruses is very similar. These positive stranded RNA viruses possess a single large open reading frame (ORF) encoding all the viral proteins necessary for virus replication. These proteins are expressed as a polyprotein, that is, co- and post-translationally processed by both cellular and virus-encoded proteinases to yield the mature viral proteins. The viral proteins responsible for the replication of the viral genome RNA are located within approximately the carboxy-terminal. Two-thirds of the ORF are termed nonstructural (NS) proteins. The genetic organization and polyprotein processing of the nonstructural protein portion of the ORF for pestiviruses and hepaciviruses is very similar. For both the pestiviruses and hepaciviruses, the mature nonstructural (NS) proteins, in sequential order from the amino-terminus of the nonstructural protein coding region to the carboxy-terminus of the ORF, consist of p7, NS2, NS3, NS4A, NS4B, NS5A, and NS5B.

The non-structural (NS) proteins of pestiviruses and hepaciviruses share sequence domains that are characteristic of specific protein functions. For example, the NS3 proteins of viruses in both groups possess amino acid sequence motifs characteristic of serine proteinases and of helicases (Gorbalenya et al. (1988) Nature 333:22; Bazan and Fletterick (1989) Virology 171:637-639; Gorbalenya et al. (1989) Nucleic Acid Res. 17.3889-3897). Similarly, the NS5B proteins of pestiviruses and hepaciviruses have the motifs characteristic of RNA-directed RNA polymerases (Koonin, E. V. and Dolja, V. V. (1993) Crit. Rev. Biochem. Molec. Biol. 28:375-430).

Hepatitis C Virus

The hepatitis C virus (HCV) is the leading cause of chronic liver disease worldwide. (Boyer, N. et al. J. Hepatol. 32:98-112, 2000). HCV causes a slow growing viral infection and is the major cause of cirrhosis and hepatocellular carcinoma (Di Besceglie, A. M. and Bacon, B. R., Scientific American, October: 80-85, (1999); Boyer, N. et al. J. Hepatol. 32:98-112, 2000). Cirrhosis caused by chronic hepatitis C infection accounts for 8,000-12,000 deaths per year in the United States, and HCV infection is the leading indication for liver transplantation.

HCV is known to cause at least 80% of post-transfusion hepatitis and a substantial proportion of sporadic acute hepatitis. Preliminary evidence also implicates HCV in many cases of “idiopathic” chronic hepatitis, “cryptogenic” cirrhosis, and probably hepatocellular carcinoma unrelated to other hepatitis viruses, such as hepatitis B virus (HBV). A small proportion of healthy persons appear to be chronic HCV carriers, varying with geography and other epidemiological factors. The numbers may substantially exceed those for HBV, though information is still preliminary. How many of these persons have subclinical chronic liver disease is unclear. (The Merck Manual, ch. 69, p. 901, 16th ed., (1992)).

A significant focus of current antiviral research is directed to the development of improved methods of treatment of chronic HCV infections in humans (Di Besceglie, A. M. and Bacon, B. R., Scientific American, October: 80-85, (1999)).

Treatment of HCV Infection with Interferon

Interferons (IFNs) have been commercially available for the treatment of chronic hepatitis for nearly a decade. IFNs are glycoproteins produced by immune cells in response to viral infection. IFNs inhibit replication of a number of viruses, including HCV, and when used as the sole treatment for hepatitis C infection, IFN can in certain cases suppress serum HCV-RNA to undetectable levels. Additionally, IFN can normalize serum amino transferase levels. Unfortunately, the effect of IFN is temporary and a sustained response occurs in only 8%-9% of patients chronically infected with HCV (Gary L. Davis. Gastroenterology 118:S104-S114, 2000). Most patients, however, have difficulty tolerating interferon treatment, which causes severe flu-like symptoms, weight loss, and lack of energy and stamina.

A number of patents disclose Flaviviridae, including HCV, treatments that use interferon-based therapies. For example, U.S. Pat. No. 5,980,884 to Blatt et al. discloses methods for retreatment of patients afflicted with HCV using consensus interferon. U.S. Pat. No. 5,942,223 to Bazer et al. discloses an anti-HCV therapy using ovine or bovine interferon-tau. U.S. Pat. No. 5,928,636 to Alber et al. discloses the combination therapy of interleukin-12 and interferon alpha for the treatment of infectious diseases including HCV. See also U.S. Pat. No. 5,849,696 to Chretien et al; U.S. Pat. No. 5,830,455 to Valtuena et al; and U.S. Pat. No. 5,738,845 to Imakawa. Other interferon-based treatments for HCV are disclosed in U.S. Pat. Nos. 5,676,942, 5,372,808, and 5,849,696. A number of patents also disclose pegylated forms of interferon, such as, U.S. Pat. Nos. 5,747,646, 5,792,834 and 5,834,594 to Hoffmann-La Roche Inc; PCT Publication No. WO 99/32139 and WO 99/32140 to Enzon; WO 95/13090 and U.S. Pat. Nos. 5,738,846 and 5,711,944 to Schering; and U.S. Pat. No. 5,908,621 to Glue et al.

Interferon alpha-2a and interferon alpha-2b are currently approved as monotherapy for the treatment of HCV. ROFERON®-A (Roche) is the recombinant form of interferon alpha-2a. PEGASYS® (Roche) is the pegylated (i.e. polyethylene glycol modified) form of interferon alpha-2a. INTRON®A (Schering Corporation) is the recombinant form of Interferon alpha-2b, and PEG-INTRON® (Schering Corporation) is the pegylated form of interferon alpha-2b.

Other forms of interferon alpha, as well as interferon beta, gamma, tau and omega are currently in clinical development for the treatment of HCV. For example, INFERGEN® (interferon alphacon-1) by InterMune, OMNIFERON® (natural interferon) by Viragen, ALBUFERON® by Human Genome Sciences, REBIF® (interferon beta-1a) by Ares-Serono, Omega Interferon by BioMedicine, Oral Interferon Alpha by Amarillo Biosciences, and interferon gamma, interferon tau, and interferon gamma-1b by InterMune are in development.

Ribivarin

Ribavirin (1-β-D-ribofuranosyl-1-1,2,4-triazole-3-carboxamide) is a synthetic, non-interferon-inducing, broad spectrum antiviral nucleoside analog sold under the trade name, Virazole® (The Merck Index, 11th edition, Editor: Budavari, S., Merck & Co., Inc., Rahway, N.J., pl 304, 1989). U.S. Pat. No. 3,798,209 and RE29,835 disclose and claim ribavirin. Ribavirin is structurally similar to guanosine, and has in vitro activity against several DNA and RNA viruses including Flaviviridae (Gary L. Davis. Gastroenterology 118:S104-S114, 2000).

Ribavirin reduces serum amino transferase levels to normal in 40% of patients, but it does not lower serum levels of HCV-RNA (Gary L. Davis. Gastroenterology 118:S104-S114, 2000). Thus, ribavirin alone is not effective in reducing viral RNA levels. Additionally, ribavirin has significant toxicity and is known to induce anemia.

Ribavirin is not approved for monotherapy against HCV. It has been approved in combination with interferon alpha-2a or interferon alpha-2b for the treatment of HCV.

Combination of Interferon and Ribavirin

The current standard of care for chronic hepatitis C is combination therapy with an alpha interferon and ribavirin. The combination of interferon and ribavirin for the treatment of HCV infection has been reported to be effective in the treatment of interferon naive patients (Battaglia, A. M. et al., Ann. Pharmacother. 34:487-494, 2000), as well as for treatment of patients when histological disease is present (Berenguer, M. et al. Antivir. Ther. 3(Suppl. 3):125-136, 1998). Studies have show that more patients with hepatitis C respond to pegylated interferon-alpha/ribavirin combination therapy than to combination therapy with unpegylated interferon alpha. However, as with monotherapy, significant side effects develop during combination therapy, including hemolysis, flu-like symptoms, anemia, and fatigue. (Gary L. Davis. Gastroenterology 118:S104-S114, 2000).

Combination therapy with PEG-INTRON® (peginterferon alpha-2b) and REBETOL® (Ribavirin, USP) Capsules is available from Schering Corporation. REBETOL® (Schering Corporation) has also been approved in combination with INTRON® A (Interferon alpha-2b, recombinant, Schering Corporation). Roche's PEGASYS® (pegylated interferon alpha-2a) and COPEGUS® (ribavirin) are also approved for the treatment of HCV.

PCT Publication Nos. WO 99/59621, WO 00/37110, WO 01/81359, WO 02/32414 and WO 03/024461 by Schering Corporation disclose the use of pegylated interferon alpha and ribavirin combination therapy for the treatment of HCV. PCT Publication Nos. WO 99/15194, WO 99/64016, and WO 00/24355 by Hoffinann-La Roche Inc also disclose the use of pegylated interferon alpha and ribavirin combination therapy for the treatment of HCV.

Additional Methods to Treat Flaviviridae Infections

The development of new antiviral agents for flaviviridae infections, especially hepatitis C, is currently underway. Specific inhibitors of HCV-derived enzymes such as protease, helicase, and polymerase inhibitors are being developed. Drugs that inhibit other steps in HCV replication are also in development, for example, drugs that block production of HCV antigens from the RNA (IRES inhibitors), drugs that prevent the normal processing of HCV proteins (inhibitors of glycosylation), drugs that block entry of HCV into cells (by blocking its receptor) and nonspecific cytoprotective agents that block cell injury caused by the virus infection. Further, molecular approaches are also being developed to treat hepatitis C, for example, ribozymes, which are enzymes that break down specific viral RNA molecules, and antisense oligonucleotides, which are small complementary segments of DNA that bind to viral RNA and inhibit viral replication, are under investigation. A number of HCV treatments are reviewed by Bymock et al. in Antiviral Chemistry & Chemotherapy, 11:2; 79-95 (2000) and De Francesco et al. in Antiviral Research, 58: 1-16 (2003).

In light of the fact that Flaviviridae virus infection, including HCV, has reached epidemic levels worldwide, and has tragic effects on the infected patient, there remains a strong need to provide new effective pharmaceutical agents to treat Flaviviridae virus infection that have low toxicity to the host.

Therefore, it is an object of the present invention to provide a compound, method, use, and composition for the treatment of a host infected with a Flaviviridae virus, and in particular hepatitis C virus.

It is another object of the present invention to provide a compound, method, use, and composition generally for the treatment of patients infected with pestiviruses, flaviviruses, or hepaciviruses.

### SUMMARY OF THE INVENTION

The present invention provides compounds, compositions and methods of use of certain nucleoside analogues for inhibiting replication of a Flaviviridae virus, including a pestivirus,flavivirus, or hepacivirus, and in particular HCV. The nucleoside analogues include a seven-membered ring as the sugar portion of the compound. Included within the invention are pharmaceutically acceptable salts, esters, prodrugs, and derivatives of the nucleoside analogues. In one embodiment, the present invention provides a pharmaceutically acceptable composition comprising the nucleoside analogue, optionally in a pharmaceutically acceptable carrier. In another embodiment, the invention provides methods of treatment of a host infected with a Flaviviridae virus infection, including a pestivirus, a flavivirus or HCV. In another embodiment, the invention provides the use of the nucleoside analogue, or its ester or salt in the manufacture of a medicament for the treatment of a Flaviviridae virus, including a pestivirus,flavivirus, or hepacivirus infection, and in particular HCV, in a host. The invention also includes processes for synthesis of the nucleoside analogue. The compounds of the present invention may be administered alone or in combination or alternation with one or more other anti-viral agents. The compounds can also be used prophylactically to prevent or retard the progression of clinical illness in individuals who carry an anti-Flaviviridae antibody, who are Flaviviridae-antigen positive, or who have been exposed to a Flaviviridae.

One embodiment of the present invention includes use of the compounds as inhibitors of positive-sense single-stranded RNA-dependent RNA viral replication and/or for the treatment of viral infection caused by positive-sense single-stranded RNA-dependent RNA viruses. In addition to the family of Flaviviridae, viruses in this category include the Picornaviridae as well, thereby embracing rhinovirus, poliovirus and hepatitis A virus. The Flaviviridae family of hepatitis C virus, dengue virus, yellow fever virus, West Nile virus, Japanese encephalitis virus, Banzi virus and bovine viral diarrhea virus (BVDV) likewise all are included here.

### BRIEF DESCRIPTION OF THE DRAWINGS

FIG. 1 is an illustration of 7-membered ring nucleoside structures of the present invention.

FIG. 2 is an illustration of branched 7-membered ring nucleoside structures of the present invention.

FIG. 3a is a depiction of a synthetic method to prepare an idoseptanoside.

FIG. 3b is a depiction of a synthetic method to prepare an altroseptanoside.

FIG. 3c is an illustration of a synthetic method to prepare a guloseptanoside.

FIG. 3d is a depiction of a synthetic method for preparing an alloseptanoside.

FIG. 4 is an illustration of an alternative scheme for preparing the target molecule 1 having an adenine nucleobase.

FIG. 5 is a depiction of yet another alternate route for preparing the target molecule 1 having an adenine nucleobase.

FIG. 6 is a depiction of a synthesis for preparing a 7-membered ring nucleoside having a pyrrolopyrimidine (7-deazapurine) nucleobase.

FIG. 7 is a depiction of a synthetic method for preparing a ribavirin analog having a 7-membered sugar ring.

FIG. 8 is a depiction of a synthetic method for preparing a 7-membered sugar ring having a 3-deazaadenine nucleobase.

FIG. 9 is an illustration of a synthesis for preparing a 7-membered sugar ring having an adenine nucleobase.

FIG. 10 is a depiction of yet another synthesis for preparing a 7-membered sugar ring having an adenine nucleobase.

FIG. 11 is an illustration of a synthesis from an isopropylidene sugar starting material for preparing a 7-membered sugar ring having an adenine base.

FIG. 12 is an illustration of a synthesis of the present invention for preparing an adenine nucleobase via a halogen substituent replacement reaction that includes an isopropylidene intermediate.

FIG. 13 is an illustration of another synthetic method of the present invention for preparing a 7-membered ring nucleoside having an adenine nucleobase.

FIG. 14 is an illustration of a synthetic method for preparing one addition methyl substituted 7-membered sugar ring nucleosides.

FIG. 15 is an illustration of still another synthesis of a 7-membered sugar ring having an adenine nucleobase.

FIG. 16 is a depiction of yet another synthesis of a 7-membered sugar ring having an adenine nucleobase with different 2′-position substituents.

FIG. 17 is a depiction of another synthesis of a 7-membered sugar ring having an adenine nucleobase with a 2′-position having an ethylene substituent.

FIG. 18 is a depiction of a synthesis for preparing a 7-membered sugar ring having a cytosine nucleobase.

FIG. 19 is a depiction of another synthesis for preparing a 7-membered sugar ring having a cytosine nucleobase.

FIG. 20 is an illustration of yet another synthesis for preparing a 7-membered sugar ring having a cytosine nucleobase.

FIG. 21 is a depiction of still another synthesis for preparing a 7-membered sugar ring having a cytosine nucleobase.

FIG. 22 is a depiction of still another synthesis for preparing a 2′-disubstituted fluorine 7-membered sugar ring and a cytosine nucleobase.

FIG. 23 is an illustration of a synthetic method for preparing a 7-membered sugar ring having either a cytosine or uracil/thymine nucleobase.

FIG. 24 is an illustration of another adaptive synthesis for preparing 7-membered sugar ring nucleosides having a natural nucleobase from the same sugar ring by the use of different reagents.

FIG. 25 is a depiction of still another adaptive synthesis for preparing 7-membered sugar ring nucleosides having a natural nucleobase from the same sugar ring by the use of different reagents.

FIG. 26 is a depiction of another adaptive synthesis for preparing 7-membered sugar ring nucleosides having a natural nucleobase from the same 3′-pseudo deoxy-sugar ring by the use of different reagents.

FIG. 27 is an illustration of another synthesis for preparing 7-membered sugar ring nucleosides having a natural nucleobase from the same sugar ring by the use of different reagents.

FIG. 28 is a depiction of a synthesis for preparing 7-membered sugar ring nucleosides from a single, fluorine-substituted sugar ring by the use of various reagents and reaction conditions.

FIG. 29 is a depiction of a synthetic method for preparing a 7-membered sugar ring nucleoside having a pyrrolo-pyrimidine nucleobase.

FIG. 30 is an illustration of yet another synthetic method for preparing a 7-membered sugar ring nucleoside having a pyrrolo-pyrimidine nucleobase.

FIG. 31 is a depiction of another synthetic method for preparing a 7-membered sugar ring nucleoside having a purine nucleobase and a 2′-Methyl-2′-fluoro-disubstituted sugar ring.

FIG. 32a is an illustration of nucleoside structures, T-1 to T-20, of the present invention.

FIG. 32b is an illustration of apionucleoside structures, T-21 to T-35 of the present invention.

FIG. 32c is a depiction of isonucleoside structures, T-36 to T47, of the present invention.

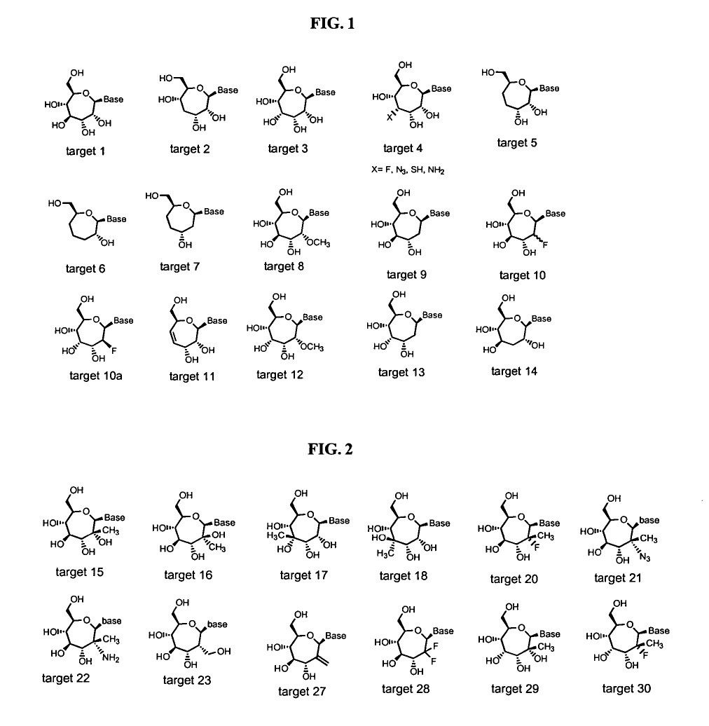

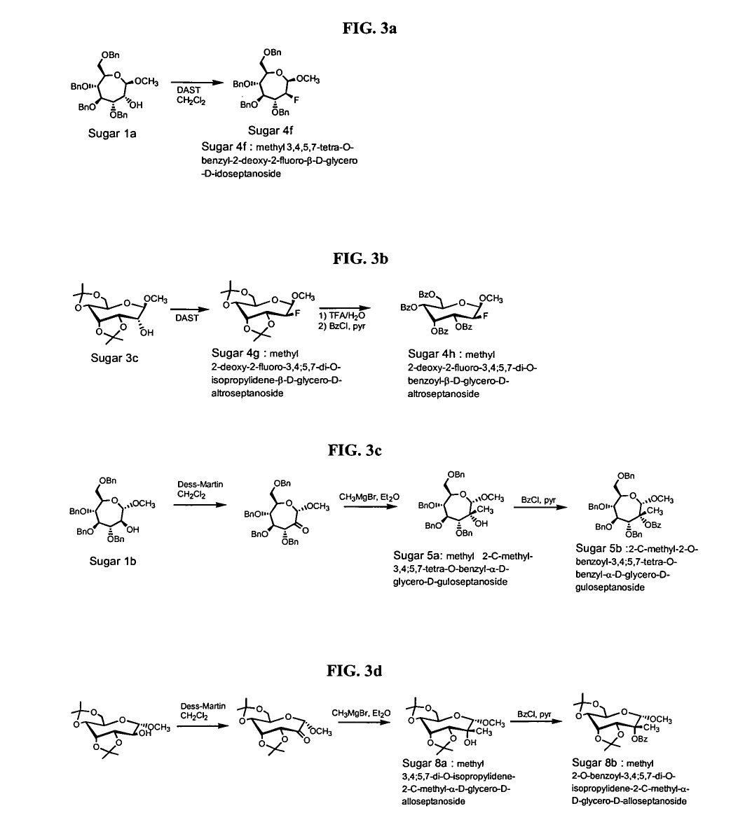

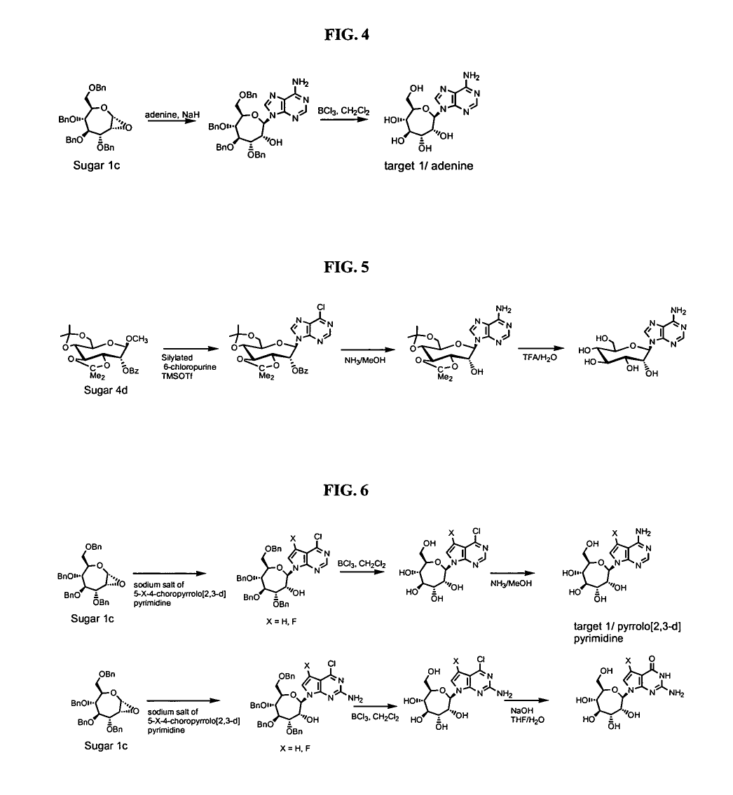

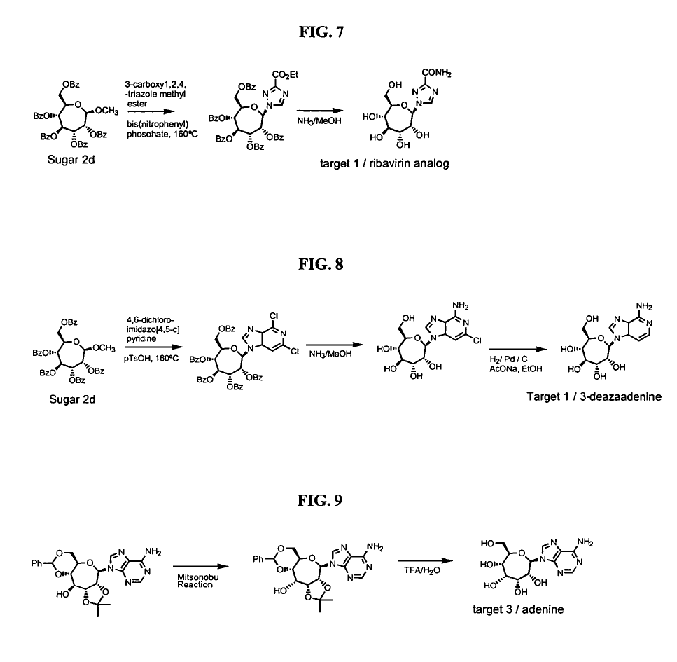

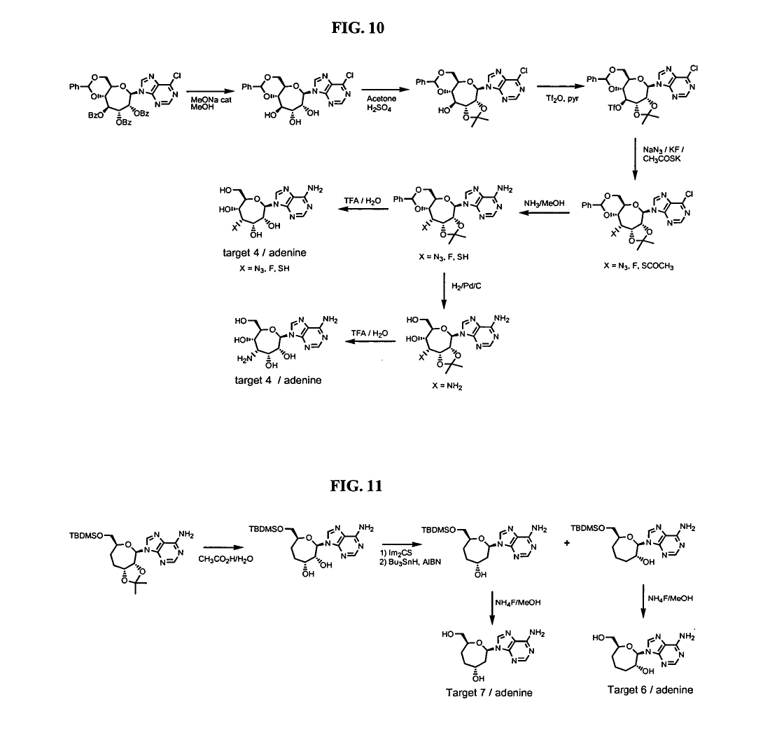

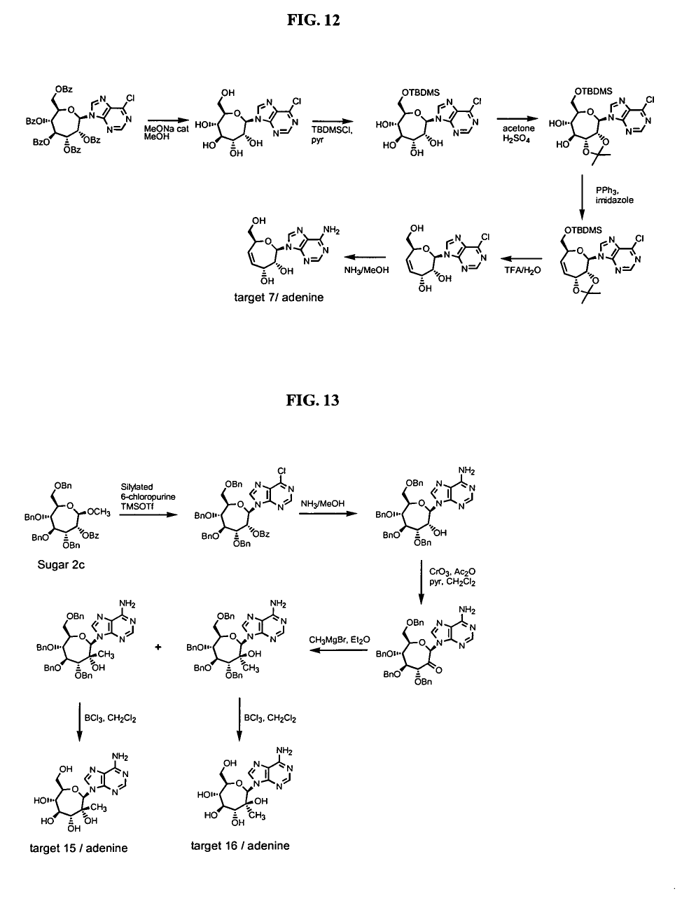

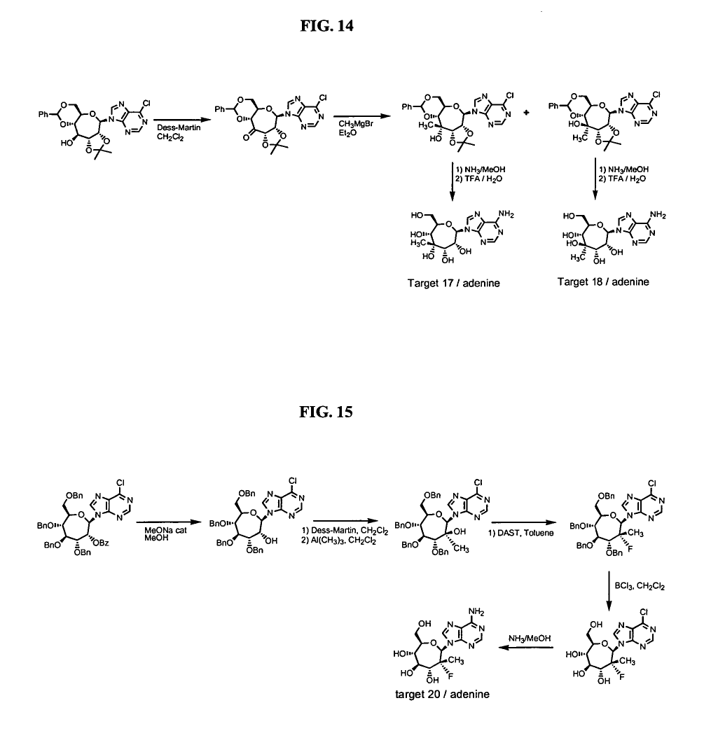

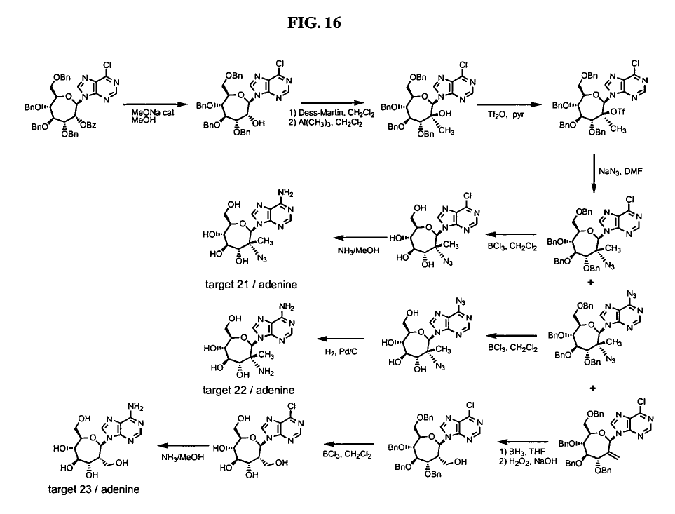

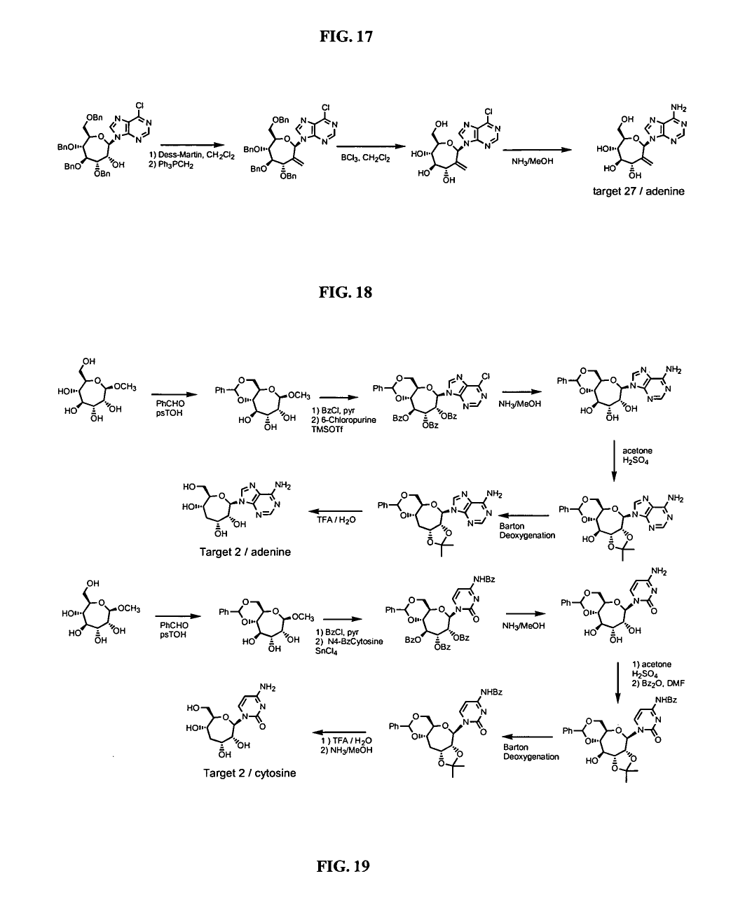

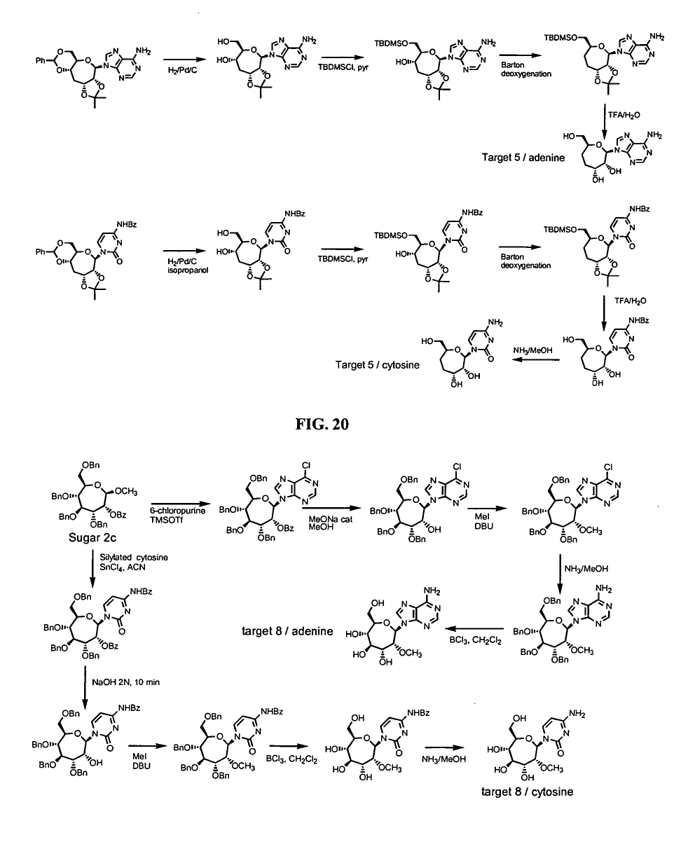

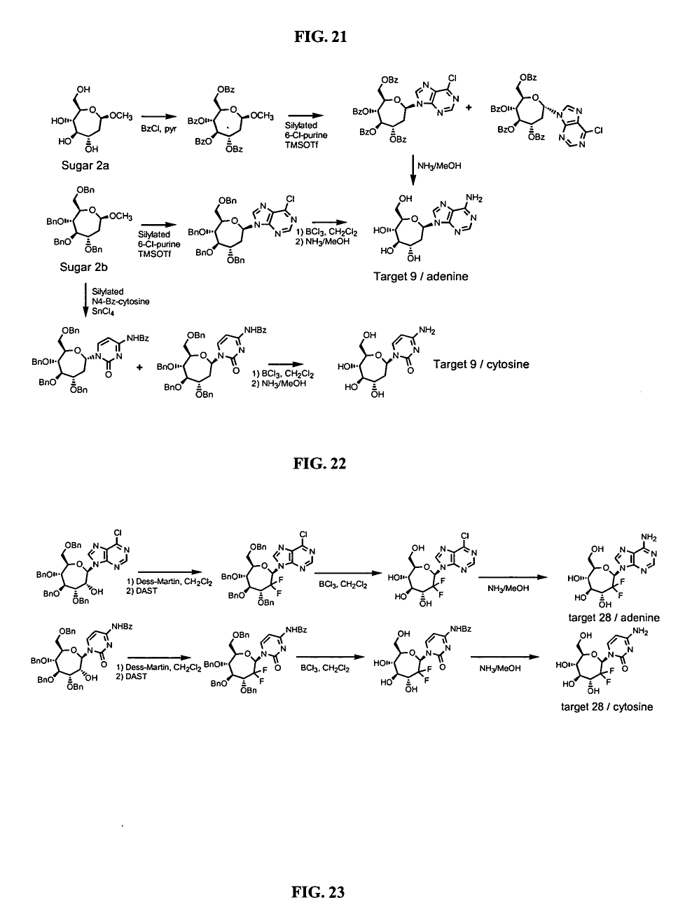

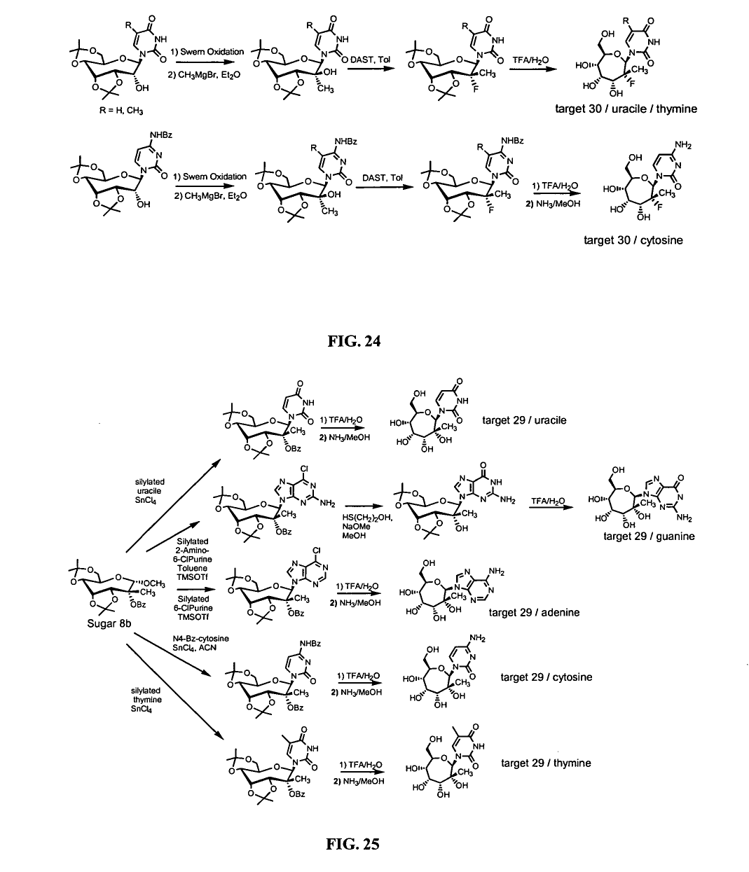

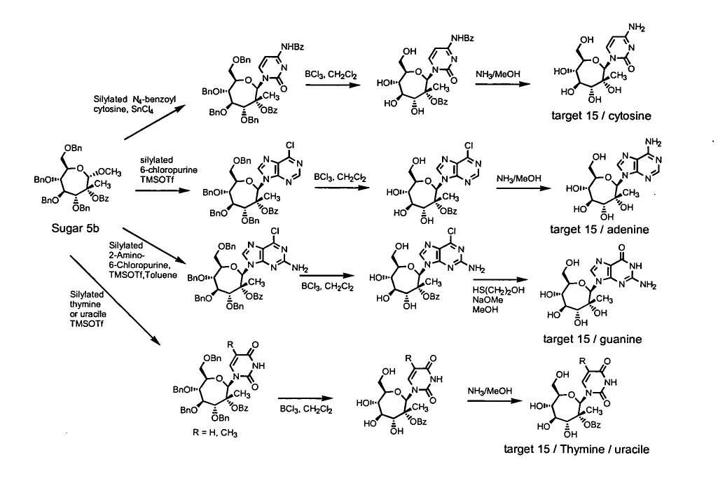

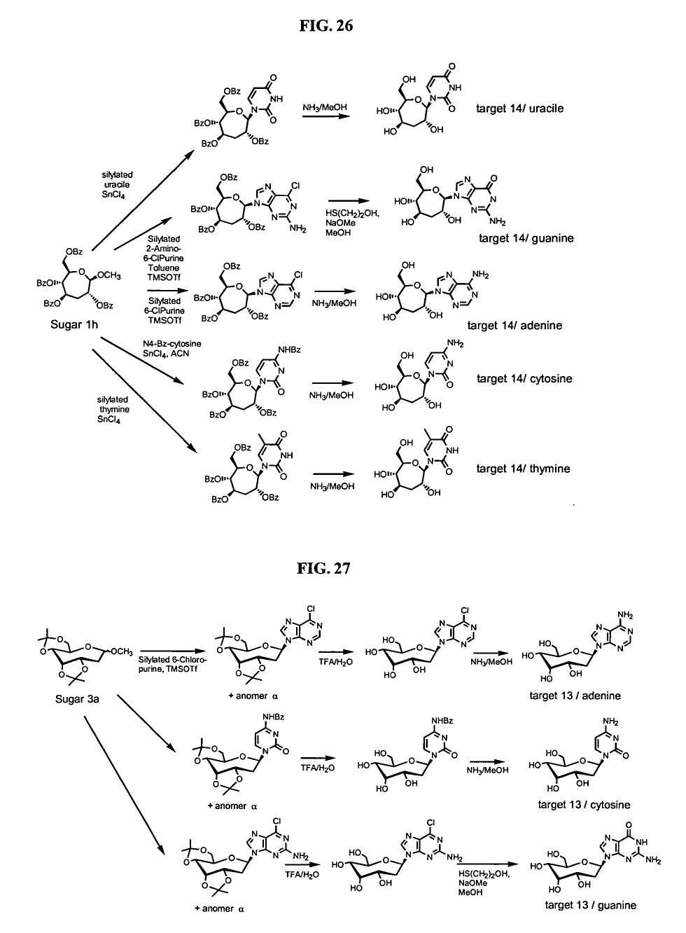

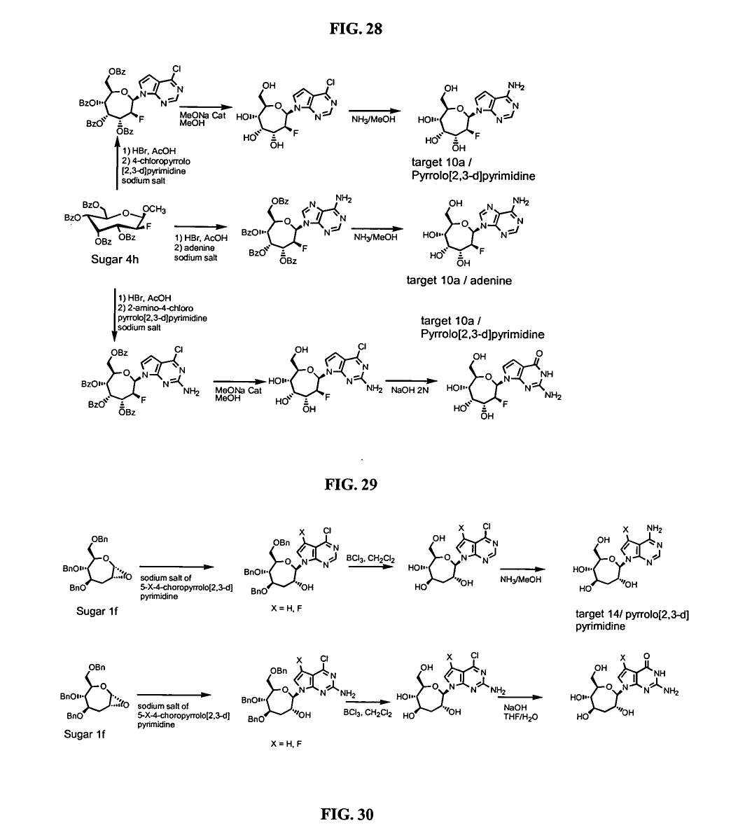

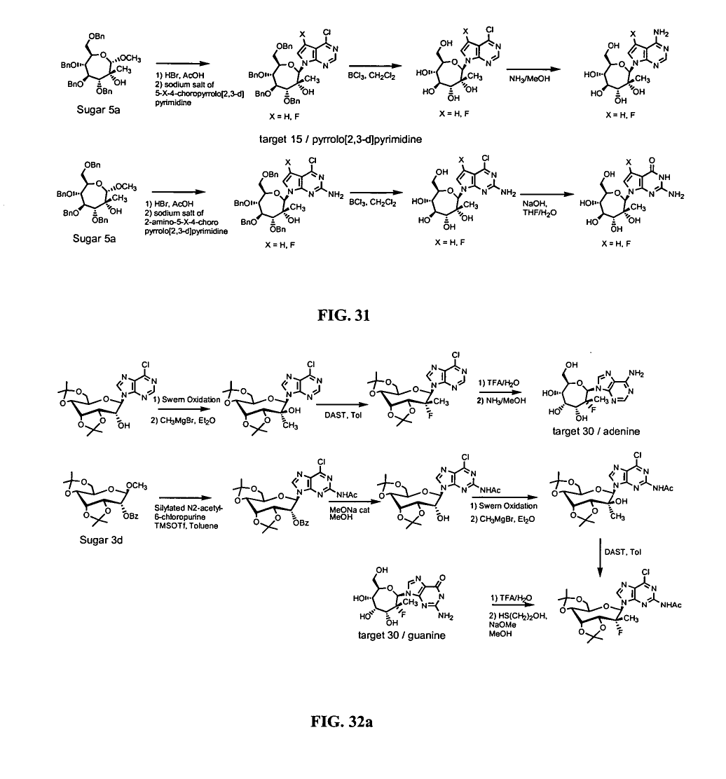

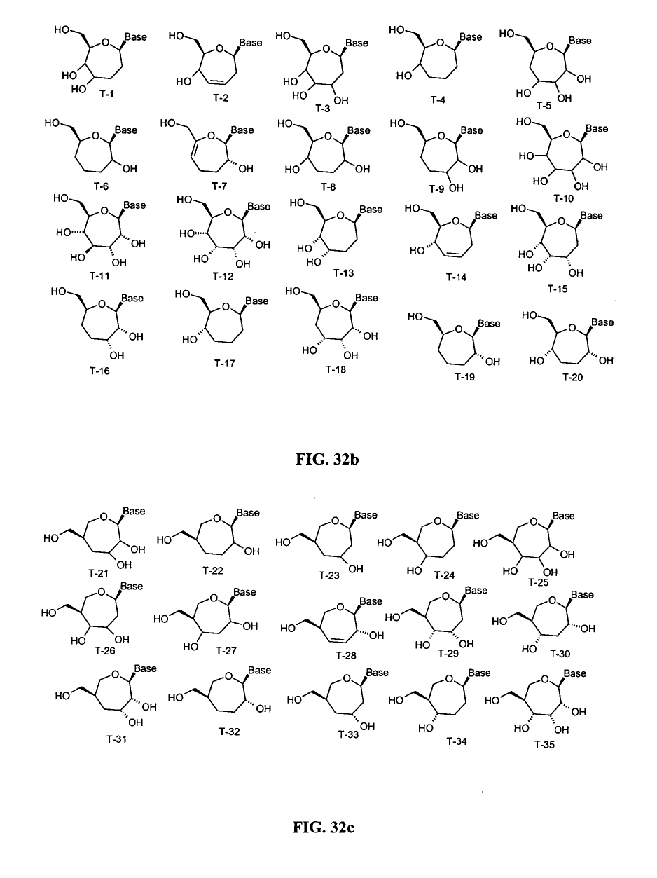

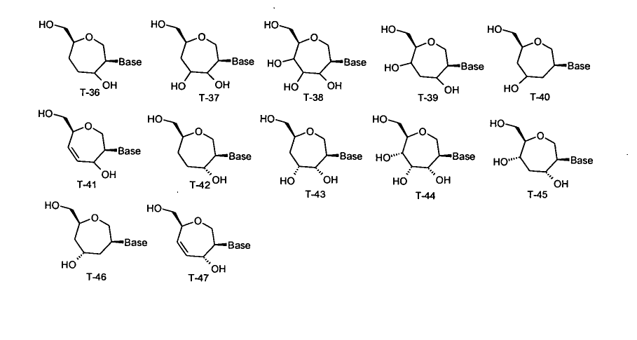

### DETAILED DESCRIPTION OF THE INVENTION

The present invention provides compounds, compositions, methods and uses of a nucleoside analogue for inhibiting replication of a Flaviviridae virus, including a pestivirus,flavivirus, or hepacivirus, and in particular HCV, and a method of use of the nucleoside analogue, as well as a derivative, pharmaceutically acceptable salt, ester or prodrug thereof, as a medicament in the treatment and/or prophylaxis of a host thus infected. The invention also provides processes for synthesis of the nucleoside analogue.

I. Embodiments of the Invention

The nucleoside analogues of the present invention comprise a compound of the structural Formula I: 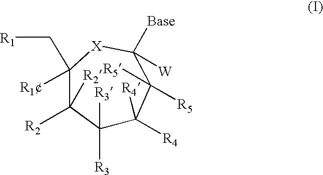
wherein:

X is O, S, SO₂, N—R, C(H)(R), or C(R)(R);

R is independently H; C₁₋₄ alkyl, C₂₋₄ alkenyl, or C₂₋₄ alkynyl, each of which may be optionally substituted; CN, N₃, halo, OH, CONH₂, NH₂, or amidino;

R₁ is OH, monophosphate, diphosphate, triphosphate, phosphonate, phosphoryl, a phosphate derivative, acyl, hydrogen, alkyl, O-acyl, O-alkyl, O-aryl, O-alkoxyalkyl, O-aryloxyalkyl, O-substituted alkyl, O-substituted alkenyl, O-substituted alkynyl, alkyl sulfonyl, aryl sulfonyl, alkenyl sulfonyl, aralkylsulfonyl, an amino acid residue, or any cleavable substitutent that in vivo provides OH;

R₂, R₃, R4 and R₅ each independently is H, OH, SH, NH₂, halo, C₁₋₁₀ alkylcarbonyl, monophosphate, diphosphate, triphosphate, phosphoryl such as phosphate, phosphonate, phosphinate, phosphonoamidate, carbamate, phosphorothioate, phosphorodithioate, carbonyl, thiocarbonyl, aminoacyl, amidino, NO₂, CN, N₃, sulfonyl, sulfoxido, sulfate, sulfonate, sulfamoyl, sulfonamide, C₁₋₆ alkyl, C₂₋₆ alkenyl, C₂₋₆ alkynyl, acyl, haloalkyl, haloalkenyl, haloalkynyl, cyclopropyl, O-alkyl, O-alkenyl; O-alkynyl; O-acyl; S-alkyl; S-alkenyl; S-alkynyl; S-acyl; NH-alkyl; N(alkyl)₂; NH(alkenyl); N(alkenyl)₂; NH(alkynyl); N(alkynyl)₂; NH(acyl); N(acyl)₂; CONH₂; COOH; CONH-alkyl; CON(alkyl)₂; COSH₂; COSH-alkyl; COS(alkyl)₂; C₁₋₆ alkyl-O—C₁₋₆ alkyl; C₁₋₆ alkyl-O-alkenyl; C₁₋₆ alkyl-O-alkynyl; C₁₋₆ alkyl-S-alkyl; C₁₋₆ alkyl-S-alkenyl; C₁₋₆ alkyl-S-alkynyl; CH₂CN; or CH₂N₃; and

Each of R_{1′}, R_{2′}, R_{3′}, R_{4′}, R_{5′} and W independently is H, OH, C₁₋₁₀ alkylcarbonyl, phosphoryl, phosphonate, phosphinate, phosphonoamidate, Cl, F, Br, I, CN, NO₂, N₃, NH₂, acylamino, amido, amidino, C₁₋₆ alkyl, C₂₋₆ alkenyl, C₂-₆ alkynyl, carbonyl, thiocarbonyl, acyl, haloalkyl, haloalkenyl, haloalkynyl, acyl, cyclopropyl, sulfonyl, sulfoxido, sulfate, sulfonate, sulfamoyl, sulfonamido, C₁₋₆ alkyl-O—C₁₋₆ alkyl, C₁₋₆ alkyl-O-alkenyl, C₁₋₆ alkyl-O-alkynyl, C₁₋₆ alkyl-S-alkyl, C₁₋₆ alkyl-S-alkenyl, C₁₋₆ alkyl-S-alkynyl, CONH₂, COOR, CH₂CN, or CH₂N₃; and

Each R₂, R₃, R₄ and R₅ and its corresponding R′ can form a spiro moiety;

Each R₂+R₃, R₃+R₄, or R₄+R₅ independently may join to form a 3, 4, 5 or 6 membered ring that optionally has 1, 2 or 3 heteroatoms;

Each R_{2′}+R_{3′}, R_{3′}+R_{4′}, or R_{4′}+R_{5′} independently may join to form a 3, 4, 5 or 6 membered ring that optionally has 1, 2 or 3 heteroatoms;

with the proviso that W is OH only when X is C(H)(R) or C(R)(R); and

with the further proviso that when any R₁, R₂, R₃, R4 or R₅ is OH or NH₂, then its corresponding R_{1′}, R_{2′}, R_{3′}, R_{4′} or R_{5′} may not also be OH or NH₂;

Base is selected from the group consisting of: 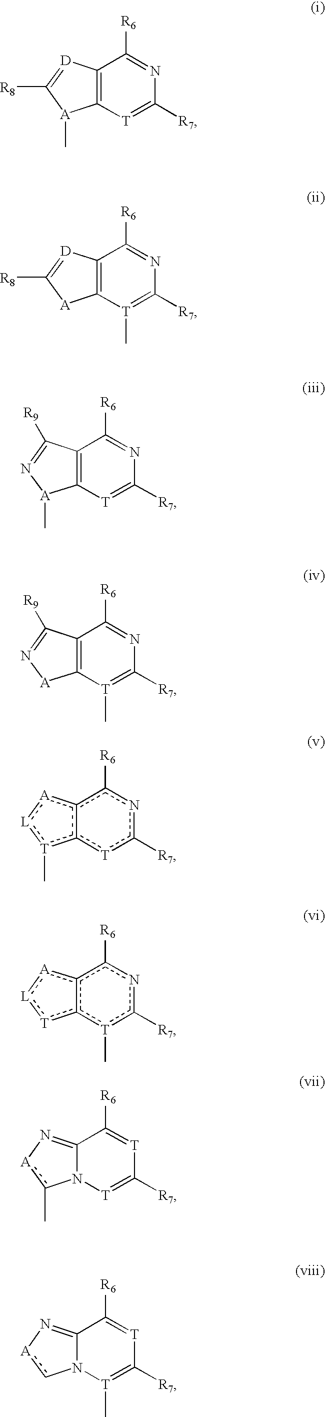 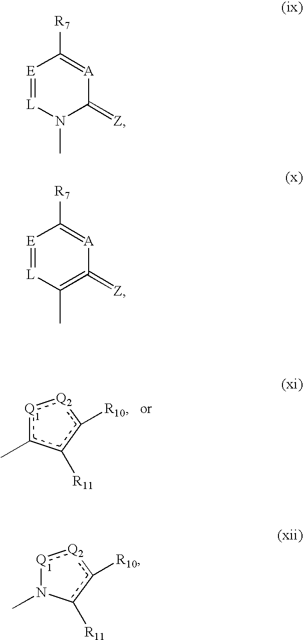
wherein:

Each occurrence of A, L, and T independently is C, CH, C(H)(R), N, N—R, C-alkyl, O or S depending upon correct valence; or C-halo, C—C₁₋₆ alkyl, C—C₂₋₆ alkenyl, C—C₂₋₆ alkynyl, C₁₋₆ alkylamino, C—CF₃, C—OH, C—NH₂, C—NO₂, C—CN, C—N₃, C—COOR, or C—CONH₂;

D is CH, C—CN, C—NO₂, N, C—C₁₋₆ alkyl, C—CONH₂, C—CONH—C₁₋₆alkyl, C—CON(C₁₋₆alkyl)(C₁₋₆alkyl), C—NH₂, C-alkoxy, C—OH, C-alkylamino, C—C(═NH)NH₂, C—COOH, C—COO-alkyl, C—CSNH₂, C—CSNH-alkyl, C—CSN(alkyl)₂, C-di(C₁₋₆ alkyl)amino, C-halo, C-heterocycle, wherein any alkyl optionally is substituted by from one to three substituents selected from the group consisting of alkoxy, hydroxyl, carboxy, halo and amino, and wherein heterocycle is a 5- or 6-membered ring having one to three heteroatoms;

E is N or C-halo, C—C₁₋₆ alkyl, C—C₂₋₆ alkenyl, C—C₂₋₆ alkynyl, C₁₋₆ alkylamino, C—CF₃, C—OH, C—NH₂, C—NO₂, C—CN, C—N₃, C—COOR, or C—CONH₂;

Z is O or S;

R₆, R₇, R₈ and R₉, each independently, is H, OH, SH, NH₂, NO₂, CN, N₃, C₁₋₆ alkyl, C₂₋₆ alkenyl, C₂₋₆ alkynyl, C₁₋₆ alkylamino, di(C₁₋₆ alkyl)amino, C₃₋₆ cycloalkylamino, C₃₋₆ cycloalkyl, halo, C₁₋₆ alkoxy, carboxy, C₁₋₆ alkoxycarbonyl, C₁₋₆ alkylthio, C₁₋₆ alkylsulfonyl, (C₁₋₆ alkyl)₀₋₂ aminomethyl, or CF₃;

R₁₀ and R₁₁ each independently is H, OH, SH, NH₂, halo, C₁₋₁₀ alkylcarbonyl, monophosphate, diphosphate, triphosphate, phosphoryl such as phosphate, phosphonate, phosphinate, phosphonoamidate, carbamate, phosphorothioate, phosphorodithioate, carbonyl, thiocarbonyl, aminoacyl, amidino, NO₂, CN, N₃, sulfonyl, sulfoxido, sulfate, sulfonate, sulfamoyl, sulfonamide, C₁₋₆ alkyl, C₂₋₆ alkenyl, C₂₋₆ alkynyl, acyl, haloalkyl, haloalkenyl, haloalkynyl, acyl, cyclopropyl, CONH₂, COOH, CONH-alkyl, CON(alkyl)₂, COSH₂, COSH-alkyl, COS(alkyl)₂, C₁₋₆ alkyl-O—C₁₋₆ alkyl, C₁₋₆ alkyl-O-alkenyl, C₁₋₆ alkyl-O-alkynyl, C₁₋₆ alkyl-S-alkyl, C₁₋₆ alkyl-S-alkenyl, C₁₋₆ alkyl-S-alkynyl, CH₂CN, or CH₂N₃;

Q₁ and Q₂ each independently is N, N—R, O, S, SO, SO₂, C(H)(R) or C(R)(R), depending upon the proper valence required;

[symbol] indicates the presence of a single or double bond; or

a pharmaceutically acceptable salt, ester or prodrug, or a or tautomeric form thereof. The structure as drawn includes all possible stereoisomers and geometric isomers.

The present invention also provides compounds of structural Formulae (IIa)-(IIf), 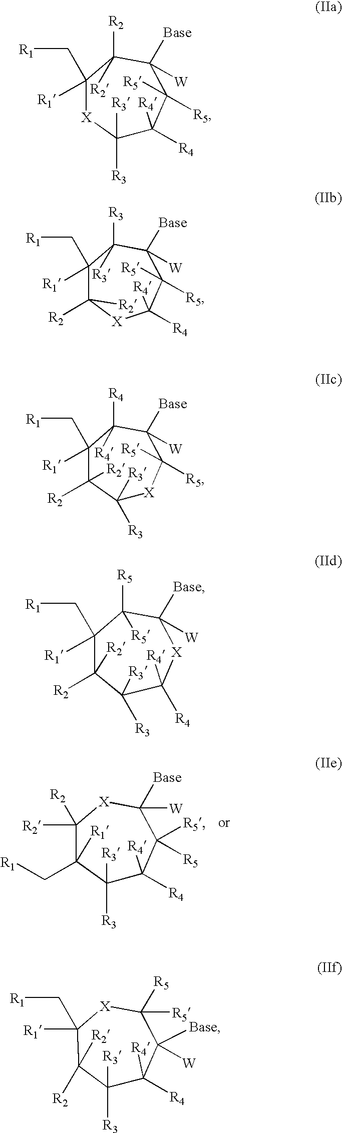
wherein:

R₁, R_{1′}, R₂, R_{2′}, R₃, R_{3′}, R₄, R_{4′}, R₅, R_{5′}, X, W and Base all are as defined for structural Formula (I) given above; with the proviso that in structural Formula (IId), W is OH only when X is C(R)(R); or

a pharmaceutically acceptable salt, ester or prodrug, or a or tautomeric form thereof. The structures as drawn include all possible stereoisomers and geometric isomers.

Compounds of the following structural Formula (IV) also are provided: 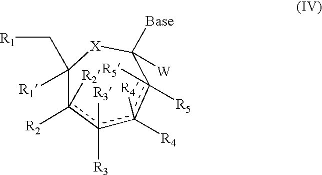
wherein:

each R₁, R_{1′}, R₂, R_{2′}, R₃, R_{3′}, R₄, R_{4′}, R₅, R_{5′}, X, W and Base is as defined above for the general Formula (I);

[symbol] denotes the presence of a single or double bond; and

where a double bond occurs, then the R and R′ substituents bound to the carbon atoms so joined are absent; or

a pharmaceutically acceptable salt, ester or prodrug, or a or tautomeric form thereof. The structures as drawn include all possible stereoisomers and geometric isomers.

In a first principal embodiment, a compound of Formula (I) or a pharmaceutically acceptable salt, ester or prodrug, or a tautomeric form thereof, is provided: 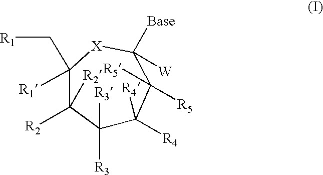
wherein:

X, R₁, R_{1′}, R₂, R_{2′}, R₃, R_{3′}, R₄, R_{4′}, R₅, R_{5′}, and W are defined above; and Base is: 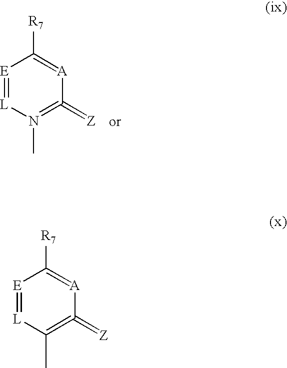
wherein A, L, E, Z and R₇ are defined above.

In a first subembodiment of the first principal embodiment of Formula (I), X is O; R₁, R₂, R₃, R₄ and R₅ all are OH; W, R_{1′}, R_{2′}, R_{3′}, R_{4′} and R_{5′} all are H; and Base is cytidine.

In a second subembodiment, X is O; R₁, R₂, R₃, R₄ and R₅ all are OH; W, R_{1′}, R_{2′}, R_{3′}, R_{4′} and R_{5′} all are H; and Base is thymidine.

In a third subembodiment, X is S; R₁, R₂, R₃, R4 and R₅ all are OH; W, R_{1′}, R_{2′}, R_{3′}, R_{4′} and R_{5′} all are H; and Base is thymidine.

In a fourth subembodiment, X is S; R₁, R₂, R₃, R₄ and R₅ all are OH; W, R_{1′}, R_{2′}, R_{3′}, R_{4′} and R_{5′} all are H; and Base is cytidine.

In a fifth subembodiment, X is O; R₁, R₄ and R₅ all are OH; R₂, R₃, R_{1′}, R_{2′}, R₃, R_{3′}, R_{4′} and R_{5′} all are H; W is methyl; and Base is thymidine.

In a sixth subembodiment, X is O; R₁, R₂, R₄ and R₅ all are OH; R₃ is halo; R_{1′}, R_{2′}, R_{3′}, R_{4′} and R_{5′} all are H; W is halo; and Base is cytidine.

In a seventh subembodiment, X is O; R₁ is OH; and any of R₂, R₃, R₄ or R₅ are O-acyl.

In an eighth subembodiment, at least two of R₂, R₃, R₄, R₅ and W must be OH.

In a second principal embodiment, a compound of Formula (I) or a pharmaceutically acceptable salt, ester or prodrug, or a tautomeric form thereof, is provided;

wherein X, R₁, R_{1′}, R₂, R_{2′}, R₃, R_{3′}, R₄, R_{4′}, R₅, R_{5′}, and W are defined above; and Base is: 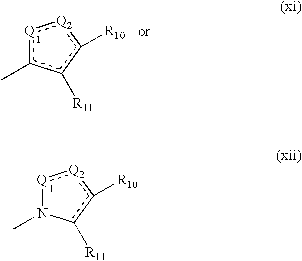
wherein R₁₀, R₁₁, Q₁ and Q₂ are defined above;

[symbol] indicates the presence of a single or double bond; or

a pharmaceutically acceptable salt, ester or prodrug, or a tautomeric form thereof.

In a subembodiment of the second principle embodiment of Formula (I), X is O; R₁, R₂, R₃, R₄ and R₅ all are OH; R_{1′}, R_{2′}, R_{3′}, R_{4′} and R_{5′} all are H; and Base is Formula (xi) wherein Q₁ is CH₂, Q₂ is CR and R is CONH₂, R₁₀ is H and R₁₁ is OH.

In a second subembodiment, X is O; R₂, R₃, R₄ and R₅ all are OH; R₁ is O-alkyl; R_{1′}, R_{2′}, R_{3′}, R_{4′} and R_{5′} all are H; and Base is Formula (xii) where Q₁ is CH₂, Q₂ is CR and R is CONH₂, R₁₀ is H and R₁₁ is CN.

In a third subembodiment, X is S; R₁, R₂, R₃, and R₅ all are OH; R₄ is halo; R_{1′}, R_{2′}, R_{3′}, R_{4′} and R_{5′} all are H; and Base is Formula (xi) wherein Q₁ is NH, Q₂ is CR and R is methyl, R₁₀ is H; and R₁₁ is CONH₂;

In a fourth subembodiment, X is S; R₁, R₂, R₃, R₄ and R₅ all are OH; R_{1′}, R_{2′}, R_{3′}, R_{4′} and R_{5′} all are H; and Base is Formula (xii) wherein Q₁ is NH, Q₂ is CR and R is CONH₂, R₁₀ is OH, and R₁₁ is H.

In a fifth subembodiment, X is NH; R₁, R₂, R₄ and R₅ all are OH; R₃ is N₃; R_{1′}, R_{2′}, R₃, R_{3′}, R_{4′} and R_{5′} all are H; and Base is Formula (xi) wherein Q₁ is CH₂, Q₂ is CR and R is amidino, R₁₀ and R₁₁ both are H.

In a sixth subembodiment, X is N; R₁, R₂, R₄ and R₅ all are OH; R₃ is halo; R_{1′}, R_{2′}, R_{3′}, R_{4′} and R_{5′} all are H; and Base is Formula (xii) wherein Q₁is CH₂, Q₂ is CR and R is CONH₂, R₁₀ is H, and R₁₁ is OH.

In a seventh subembodiment, X is O; R₁ is OH; and any of R₂, R₃, R₄ or R₅ are O-acyl.

In an eighth subembodiment, at least two of R₂, R₃, R4, R₅ and W must be OH.

In a third principal embodiment, a compound of Formula (I) or a pharmaceutically acceptable salt, ester or prodrug, or a tautomeric form thereof, is provided;

wherein X, R₁, R_{1′}, R₂, R_{2′}, R₃, R_{3′}, R₄, R_{4′}, R₅, R_{5′}, and W are defined above; and Base is selected from the group consisting of:

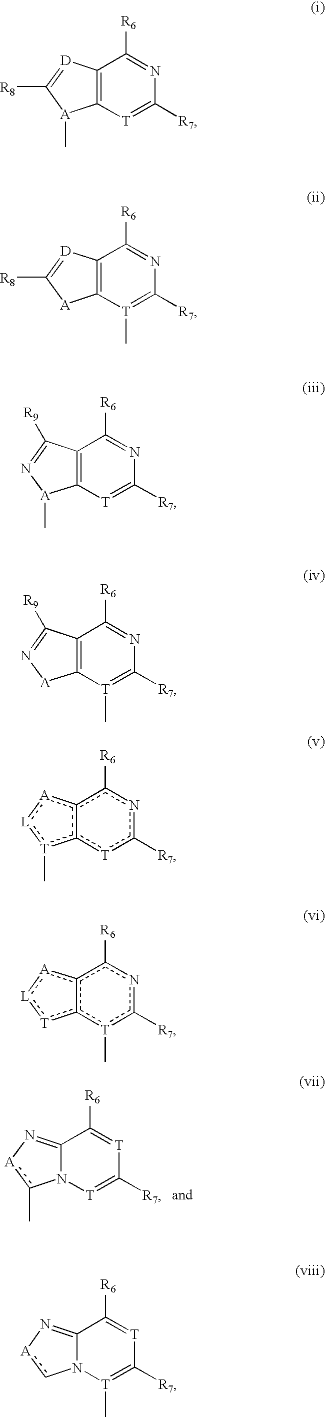

wherein A, L, T, D, R₆, R₇, R₈ and R₉, are defined above;

[symbol] indicates the presence of a single or double bond; or

a pharmaceutically acceptable salt, ester or prodrug, or a tautomeric form thereof.

In a first subembodiment of the third principle embodiment of Formula (I), X is O; R₁, R₂, R₃, R₄ and R₅ all are OH; R_{1′}, R_{2′}, R_{3′}, R_{4′} and R_{5′} all are H; W is halo;

and Base is adenine.

In a second subembodiment, X is O; R₁, R₂, R₃, R₄ and R₅ all are OH; R_{1′}, R_{2′}, R_{3′}, R_{4′} and R_{5′} all are H; W is methyl; and Base is guanine.

In a third subembodiment, X is S; R₁, R₂, R₃, R₄ and R₅ all are OH; W, R_{1′}, R_{2′}, R_{3′}, R_{4′} and R_{5′} all are H; and Base is pyrrolopyrimidine.

In a fourth subembodiment, X is S; R₁, R₂, R₃, R₄ and R₅ all are OH; W, R_{1′}, R_{2′}, R_{3′}, R_{4′}, and R_{5′} all are H; and Base is benzimidazole.

In a fifth subembodiment, X is O; R₁, R₄ and R₅ all are OH; W, R₂, R₃, R_{1′}, R_{2′}, R_{3′}, R_{3′}, R_{4′} and R_{5′} all are H; and Base is phenylthiazole.

In a sixth subembodiment, X is O; R₁, R₂, R₄ and R₅ all are OH; R₃ is halo; R_{1′}, R_{2′}, R_{3′}, R_{4′} and R_{5′} all are H; W is N₃, and Base is adenine.

In a seventh subembodiment, X is O; R₁ is OH; and any of R₂, R₃, R₄ or R₅ are O-acyl.

In an eighth subembodiment, at least two of R₂, R₃, R₄, R₅ and W must be OH.

In a fourth principal embodiment, a compound of Formula (IIa)- (IIf) or a pharmaceutically acceptable salt, ester or prodrug, or a tautomeric or polymorphic form thereof, is provided;

wherein X, R, R₁, R_{1′}, R₂, R₂′, R₃, R_{3′}, R₄, R_{4′}, R₅, R_{5′}, and W are defined above for Formula (I); with the proviso that in general structural Formula (IId) W is OH only when X is C(R)(R) for reasons of chemical stability; and

Each R₂, R₃, R₄ and R₅ and its corresponding R′ can form a spiro moiety;

Each R₂+R₃, R₃+R₄, or R₄+R₅ independently may join to form a 3-6 membered ring that optionally has 1, 2 or 3 heteroatoms;

Each R_{2′}+R_{3′}, R_{3′}+R_{4′}, or R_{4′}+R_{5′} independently may join to form a 3-6 membered ring that optionally has 1, 2 or 3 heteroatoms;

with the proviso that when any R₁, R₂, R₃, R₄ or R₅ is OH or NH₂, then its corresponding R_{1′}, R_{2′}, R_{3′}, R_{4′} or R_{5′} may not also be OH or NH₂; and

Base is selected from the group consisting of: 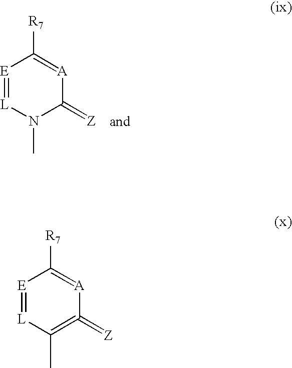
wherein A, L, E, Z and R₇ are defined above.

In a first subembodiment of the fourth principal embodiment of Formula (IIa)-(IIf), X is O; R₁, R₂, R₃, R₄ and R₅ all are OH; W, R_{1′}, R_{2′}, R_{3′}, R_{4′} and R_{5′} all are H; and Base is cytidine.

In a second subembodiment, X is O; R₁, R₂, R₃, R₄ and R₅ all are OH; W, R_{1′}, R_{2′}, R_{3′}, R_{4′} and R_{5′} all are H; and Base is thymidine.

In a third subembodiment, X is S; W, R₁, R₂, R₃, R₄ and R₅ all are OH; R_{1′}, R_{2′}, R_{3′}, R_{′} and R_{5′} all are H; and Base is thymidine.

In a fourth subembodiment, X is S; R₁, R₂, R₃, R₄ and R₅ all are OH; R_{1′}, R_{2′}, R_{3′}, R_{4′} and R_{5′} all are H; W is halo; and Base is cytidine.

In a fifth subembodiment, X is CH₂; R₁, R₄ and R₅ all are OH; R₂, R₃, R_{1′}, R_{2′}, R₃, R_{3′}, R_{4′} and R_{5′} all are H; W is methyl; Z is O, R₇ is H; and Base is triazine.

In a sixth subembodiment, X is O; R₁, R₂, R₄ and R₅ all are OH; R₃ is halo; R_{1′}, R_{2′}, R_{3′}, R_{4′} and R_{5′} all are H; Z is O; R₇ is OH; and Base is pyridine.

In a seventh subembodiment, X is O; R₁ is OH; and any of R₂, R₃, R₄ or R₅ are O-acyl.

In an eighth subembodiment, at least two of R₂, R₃, R₄, R₅ and W must be OH.

In a fifth principal embodiment, a compound of Formula (IIa)-(IIf) or a pharmaceutically acceptable salt, ester or prodrug, or tautomeric or polymorphic form thereof, is provided;

wherein X, R, R₁, R_{1′}, R₂, R₂′, R₃, R_{3′}, R₄, R_{4′}, R₅, R_{5′}, and W are defined above for Formula (I); with the proviso that in general structural Formula (IId) W is OH only when X is C(R)(R) for reasons of chemical stability; and

Each R₂, R₃, R₄ and R₅ and its corresponding R′ can form a spiro moiety;

Each R₂+R₃, R₃+R₄, or R₄+R₅ independently may join to form a 3-6 membered ring that optionally has 1, 2 or 3 heteroatoms;

Each R_{2′}+R_{3′}, R_{3′}+R_{4′}, or R_{4′}+R_{5′} independently may join to form a 3-6 membered ring that optionally has 1, 2 or 3 heteroatoms;

with the proviso that at least two of R₂, R₃, R₄, R₅ and W must be OH; and

with the further proviso that when any R₁, R₂, R₃, R₄ or R₅ is OH or NH₂, then its corresponding R_{1′}, R_{2′}, R_{3′}, R_{4′} or R_{5′} may not also be OH or NH₂; and

Base is selected from the group consisting of: 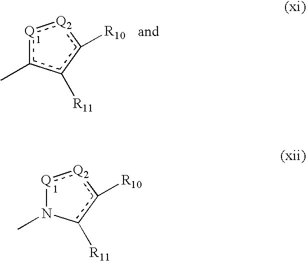
wherein R₁₀, R₁₁, Q₁ and Q₂ are defined above;

[symbol] indicates the presence of a single or double bond; or

a pharmaceutically acceptable salt, ester or prodrug, or a tautomeric form thereof. The structures as drawn include all possible stereoisomers and geometric isomers.

In a first subembodiment of the fifth principle embodiment of Formula (IIa)-(IIf), X is O; W, R₁, R₂, R₃, R₄ and R₅ all are OH; R_{1′}, R_{2′}, R_{3′}, R_{4′} and R_{5′} all are H; and Base is Formula (xi) wherein Q₁ is N, Q₂ is CR and R is CONH₂, R₁₀ is H and R₁₁ is OH.

In a second subembodiment, X is O; R₂, R₃, R₄ and R₅ all are OH; R₁ is monophosphate; R_{1′}, R_{2′}, R_{3′}, R_{4′} and R_{5′} all are H; and Base is Formula (xii) where Q₁ is CH₂, Q₂ is CR and R is CONH₂, R₁₀ is H and R₁₁ is CN.

In a third subembodiment, X is S; R₁ and R₅ are OH; R₃ is halo; R₄, R_{1′}, R_{2′}, R_{3′}, R_{4′} and R_{5′} all are H; and Base is Formula (xi) wherein Q₁ is CH₂, Q₂ is CR and R is amidino, R₁₀ is H; and R₁₁ is OH;

In a fourth subembodiment, X is S; R₁, R₂, R₃, R₄ and R₅ all are OH; R_{1′}, R_{2′}, R_{3′}, R_{4′} and R_{5′} all are H; and Base is Formula (xii) wherein Q, is CH₂, Q₂ is NH, R₁₀ is H, and R₁₁ is sulfonamide;

In a fifth subembodiment, X is NH; R₁, R₂, R₄ and R₅ all are OH; R₃ and R_{3′} join to form a spiro moiety; R_{1′}, R_{2′}, R_{4′} and R_{5′} all are H; and Base is Formula (xi) wherein Q, is CR and R is alkyl, Q₂ is CR and R is CONH₂, R₁₀ and R₁₁ both are OH.

In a sixth subembodiment, X is CH₂; R₁ is alkylsulfonyl; R₃ and R₄ are OH; R_{1′}, R₂, R_{2′}, R_{4′}, R₅, R_{5′} and W all are H; R₃ is NO₂; and Base is Formula (xii) wherein Q₁ is CH₂, Q₂ is N, R₁₀ is aminoalkyl, and R₁₁ is H.

In a seventh subembodiment, X is O; R₁ is OH; and any of R₂, R₃, R₄ or R₅ are O-acyl.

In an eighth subembodiment, at least two of R₂, R₃, R₄, R₅ and W must be OH.

In a sixth principal embodiment, a compound of Formula (IIa)-(IIf) or a pharmaceutically acceptable salt, ester or prodrug, or a tautomeric or polymorphic form thereof, is provided;

wherein X, R, R₁, R_{1′}, R₂, R₂′, R₃, R_{3′}, R₄, R_{4′}, R₅, R_{5′}, and W are defined above for Formula (I); with the proviso that in general structural Formula (IId) W is OH only when X is C(R)(R) for reasons of chemical stability; and

Each R₂, R₃, R₄ and R₅ and its corresponding R′ can form a spiro moiety;

Each R₂+R₃, R₃+R₄, or R₄+R₅ independently may join to form a 3-6 membered ring that optionally has 1, 2 or 3 heteroatoms;

Each R_{2′}+R_{3′}, R_{3′}+R_{4′}, or R_{4′}+R_{5′} independently may join to form a 3-6 membered ring that optionally has 1, 2 or 3 heteroatoms;

with the proviso that at least two of R₂, R₃, R₄, R₅ and W must be OH; and

with the further proviso that when any R₁, R₂, R₃, R₄ or R₅ is OH or NH₂, then its corresponding R_{1′}, R_{2′}, R_{3′}, R_{4′} or R_{5′} may not also be OH or NH₂; and

Base is selected from the group consisting of: 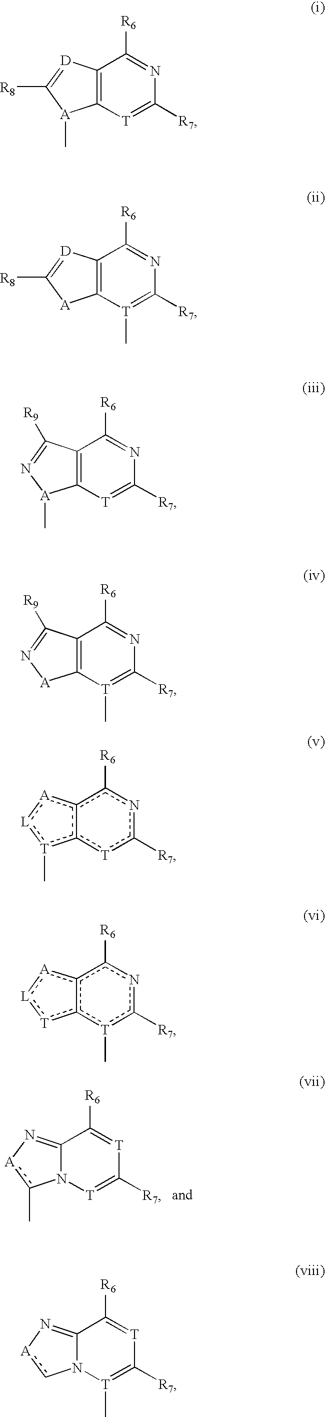
wherein:
A, L, T, D, R₆, R₇, R₈ and R₉, are defined above;

[symbol] indicates the presence of a single or double bond; or

a pharmaceutically acceptable salt, ester or prodrug, or a tautomeric form thereof.

In a first subembodiment of the sixth principle embodiment of Formula (IIa)-(IIf), X is O; R₁, R₂, R₃, R₄ and R₅ all are OH; W, R_{1′}, R_{2′}, R_{3′}, R_{4′} and R_{5′} all are H; and Base is adenine.

In a second subembodiment, X is O; W, R₁, R₂, R₃, R₄ and R₅ all are OH; R_{1′}, R_{2′}, R_{3′}, R_{4′} and R_{5′} all are H; and Base is guanine.

In a third subembodiment, X is S; R₁, R₂, R₃, R₄ and R₅ all are OH; R_{1′}, R_{2′}, R_{3′}, R_{4′} and R_{5′} all are H; W is halo; and Base is pyrrolopyrimidine.

In a fourth subembodiment, X is S; R₁, R₂, R₃, R₄ and R₅ all are OH; R_{1′}, R_{2′}, R_{3′}, R_{4′} and R_{5′} all are H; W is alkyl; and Base is phenylthiazole.

In a fifth subembodiment, X is O; R₁, R₄ and R₅ all are OH; W, R₂, R₃, R_{1′}, R_{2′}, R₃, R_{3′}, R_{4′} and R_{5′} all are H; and Base is benzimidazole.

In a sixth subembodiment, X is O; R₁, R₂, R₄ and R₅ all are OH; R₃ is halo; W, R_{1′}, R_{2′}, R_{3′}, R_{4′} and R_{5′} all are H; and Base is adenine.

In a seventh subembodiment, X is O; R₁ is OH; and any of R₂, R₃, R₄ or R₅ are O-acyl.

In an eighth subembodiment, at least two of R₂, R₃, R₄, R₅ and W must be OH.

In a seventh principal embodiment, a compound of Formula (IV) or a pharmaceutically acceptable salt, ester or prodrug, or a tautomeric form thereof, is provided;

wherein X, R₁, R_{1′}, R₂, R₂′, R₃, R_{3′}, R₄, R_{4′}, R₅, R_{5′}, and W are defined above for Formula (I); and

Base is: 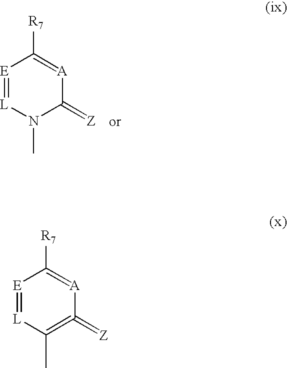
wherein A, L, E, Z and R₇ are defined above for Formula (I).

In a first subembodiment of the seventh principal embodiment of Formula (IV), X is O; R₁, R₄ and R₅ all are OH; R_{1′}, R_{4′} and R_{5′} all are H; a double bond exists between positions CR₂R_{2′} and CR₃R_{3′}; W is alkyl; and Base is cytidine.

In a second subembodiment, X is O; R₁, R₂, and R₃ all are OH; R_{1′}, R_{2′}, and R_{3′} all are H; a double bond exists between CR₄R_{4′} and CR₅R_{5′}; W is haloalkyl; and Base is thymidine.

In a third subembodiment, X is S; R₁, R₄ and R₅ all are OH; R_{1′}, R_{4′} and R_{5′} all are H; a double bond exists between positions CR₂R_{2′} and CR₃R_{3′}; W is acyl; and Base is thymidine.

In a fourth subembodiment, X is S; R₁, R₂, and R₃ all are OH; R_{1′}, R_{2′}, and R_{3′} all are H; a double bond exists between CR₄R_{4′} and CR₅R_{5′}; W is NO₂; and Base is cytidine.

In a fifth subembodiment, X is CH₂; R₁ is OH; R_{1′} is H; a double bond exists between CR₂R_{2′} and CR₃R_{3′} and between CR₄R_{4′} and CR₅R_{5′}; W is N₃; and Base is thymidine.

In a sixth subembodiment, X is O; R₁ is OH; R_{1′} is H; a double bond exists CR₂R_{2′} and CR₃R_{3′} and between CR₄R_{4′} and CR₅R_{5′}; W is halo; and Base is cytidine.

In a seventh subembodiment, X is O; R₁ is OH; and any of R₂, R₃, R₄ or R₅ are O-acyl.

In an eighth subembodiment, at least two of R₂, R₃, R₄, R₅ and W must be OH.

In an eighth principal embodiment, a compound of Formula (IV) or a pharmaceutically acceptable salt, ester or prodrug, or a tautomeric form thereof, is provided;

wherein X, R₁, R_{1′}, R₂, R₂′, R₃, R_{3′}, R₄, R_{4′}, R₅, R_{5′}, and W are defined above for Formula (I); and

Base is: 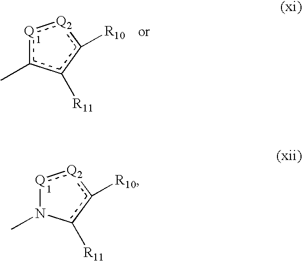
wherein R₁₀, R₁₁, Q₁ and Q₂ are defined above;

[symbol] indicates the presence of a single or double bond; or

a pharmaceutically acceptable salt, ester or prodrug, or a tautomeric form thereof.

In a first subembodiment of the eith principal embodiment of Formula (IV), X is O; a double bond exists between CR₃R_{3′} and R₄R_{4′}; R₁ is monophosphate; R_{1′} is alkyl; R₂ and R₅ are OH; W, R₃, R₄, R_{2′} and R_{5′} are H; Base is Formula (xi) wherein Q₁ is NH; Q₂ is CR and R is CONH₂; R₁₀ is H; and R₁₁ is NH.

In a second subembodiment, X is S; conjugated double bonds exist between CR₂R_{2′} and CR₃R_{3′} and between CR₄R_{4′} and CR₅R_{5′}; R₁, R₂, and R₄ all are OH; R_{1′} is alkyl; R₃ is halo; R₅ and W are H; and Base is Formula (xii) wherein Q₁ is N; Q₂ is N; R₁₀ is N₃; and R₁₁ is H.

In a third subembodiment, X is SO₂; a double bond exists between CR₄R_{4′} and CR₅R_{5′}; R₁, R₄ and R₅ all are OH; R_{1′}, R₂, R_{2′}, R₃ and R_{3′} all are H; W is acyl; and Base is Formula (xi) wherein Q₁ is CH₂; Q₂ is CR and R is amidino; R₁₀ is H; and R₁₁ is NH₂.

In a fourth subembodiment, X is N; R₁ is O-alkoxyalkyl; R₂, R₃ and R₄ are OH; R₅ is halo; R_{1′}, R_{2′}, and R_{3′} all are H; a double bond exists between CR₄R_{4′} and CR₅R_{5′}; W is NO₂; and Base is Formula (xii) wherein Q₁ is C—R and R is alkyl; Q₂ is N—R and R is CONH₂; R₁₀ is H; and R₁₁ is H.

In a fifth subembodiment, X is CH₂; R₁ is OH; R_{1′} is H; conjugated double bonds exist between CR₂R_{2′} and CR₃R_{3′} and between CR₄R_{4′} and CR₅R_{5′}; R₃ and R₅ are OH; R₂ and R₄ are H; W is N₃; and Base is Formula (xi) wherein Q₁ is C—R and R is acylamino; Q₂ is NH; R₁₀ is H; and R₁₁ is C₁₋₆ alkyl-O-C₁₋₆ alkyl.

In a sixth subembodiment, X is O; R₁ is OH; R_{1′} is H; a double bond exists between CR₂R_{2′} and CR₃R_{3′}; R₂ is H; R₃ is OH; R₄ and R_{4′} join to form a spiro group; W, R₅ and R_{5′} are H; and Base is Formula (xii) wherein Q₁ is C—R and R is COOH; Q₂ is CH₂; R₁₀ is halo; and R₁₁ is H.

In a seventh subembodiment, X is O; R₁ is OH; and any of R₂, R₃, R₄ or R₅ are O-acyl.

In an eighth subembodiment, at least two of R₂, R₃, R₄, R₅ and W must be OH.

In a ninth principal embodiment, a compound of Formula (IV) or a pharmaceutically acceptable salt, ester or prodrug, or a tautomeric or polymorphic form thereof, is provided;

wherein X, R₁, R_{1′}, R₂, R₂′, R₃, R_{3′}, R₄, R_{4′}, R₅, R_{5′}, and W are defined above for Formula (I); and

Base is selected from the group consisting of: 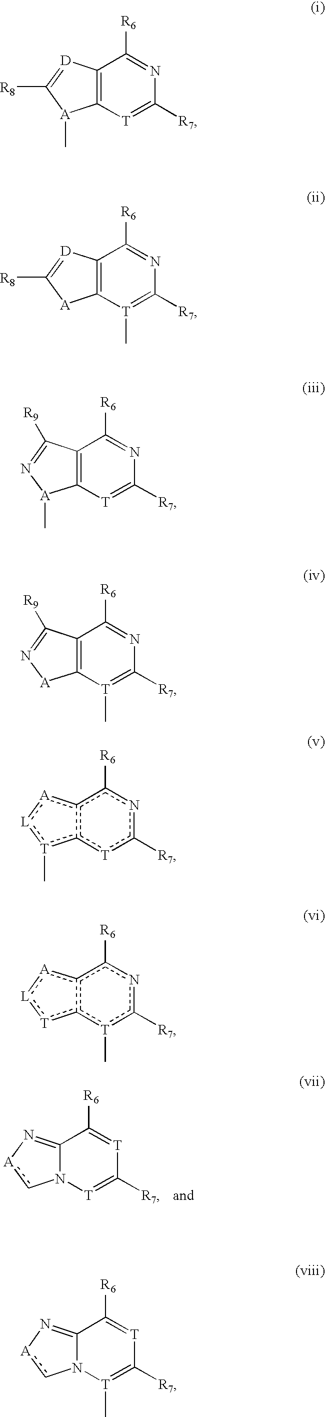
wherein A, L, T, D, R₆, R₇, R₈ and R₉, are defined above;

[symbol] indicates the presence of a single or double bond; or

a pharmaceutically acceptable salt, ester or prodrug, or a tautomeric form thereof.

In a first subembodiment of the ninth principal embodiment of Formula (IV), X is O; R₁, R₄ and R₅ all are OH; R_{1′}, R_{4′} and R_{5′} all are H; a double bond exists between positions CR₂R_{2′} and CR₃R_{3′}; R₂ and R₃ are H; W is halo; and Base is adenosine.

In a second subembodiment, X is N; R₁, R₂, and R₃ all are OH; R_{1′}, R_{2′}, and R_{3′} all are H; a double bond exists between CR₄R_{4′} and CR₅R_{5′}; R₄ and R₅ are H; W is alkyl; and Base is guanine.

In a third subembodiment, X is S; R₁, R₄ and R₅ all are OH; R_{1′}, R_{4′} and R_{5′} all are H; a double bond exists between positions CR₂R_{2′} and CR₃R_{3′}; R₂ and R₃ are H; W is acyl; and Base is pyrrolopyrimidine.

In a fourth subembodiment, X is S; R₁ is monophosphate; R₂ and R₃ are OH; R_{1′}, R_{2′}, R_{3′} and R_{5′} all are H; a double bond exists between CR₄R_{4′} and CR₅R_{5′}; R₄ is alkyl; W is alkyl; and Base is benzimidazole.

In a fifth subembodiment, X is CH₂; R₁ is OH; R_{1′} is H; conjugated double bonds exist between CR₂R_{2′} and CR₃R_{3′} and between CR₄R_{4′} and CR₅R_{5′}; R₂ and R₄ are H; R₃ is carbonyl; R₅ is OH; W is H; and Base is phenylthiazole.

In a sixth subembodiment, X is O; R₁ is diphosphate; R_{1′}, R_{4′}and W are H; conjugated double bonds exist CR₂R_{2′} and CR₃R_{3′} and between CR₄R_{4′} and CR₅R_{5′}; R₂ and R₅ are OH; R₃ is CONH-alkyl; and Base is adenosine.

In a seventh subembodiment, X is O; R₁ is OH; and any of R₂, R₃, R₄ or R₅ are O-acyl.

In an eighth subembodiment, at least two of R₂, R₃, R₄, R₅ and W must be OH.

In an alternate embodiment, a compound of Formula (III) or a pharmaceutically acceptable salt, ester or prodrug, or a tautomeric or polymorphic form thereof, is provided. The structure as drawn includes all possible stereoisomers and geometric isomers. Also provided is a method for the treatment of a host infected with a Flaviviridae comprising administering an effective treatment amount of compound of Formula (III): 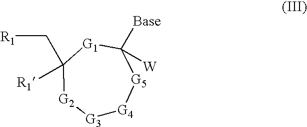
wherein:

each G₁, G₂, G₃, G₄ and G₅ independently is a heteroatom selected from the group consisting of O, S, N and P, or is CR₂R_{2′}, CR₃R_{3′}, CR₄R_{4′}, or CR₅R_{5′} where no more than three of G₁, G₂, G₃, G₄, or G₅ are heteroatoms and where no more than two heteroatoms are adjacent to each other; and

wherein R₁, R_{1′}, R₂, R₂′, R₃, R_{3′}, R₄, R_{4′}, R₅, R_{5′}, W, and Base are defined above for Formula (I).

In a first subembodiment of the alternate embodiment, G₁ is N, G₃ is O, and G₂, G₄ and G₅ are CR₂R_{2′}, CR₄R_{4′}, and CR₅R_{5′}; R₁, R₂, R₄, and R₅ all are OH; W, R_{1′}, R_{2′}, R_{4′} and R_{5′} all are H; and Base is adenosine.

In a second subembodiment, G₃ is O and G₅ is N; G₁, G₂, and G₄ are CR₁R_{1′}, CR₂R_{2′}, and CR₄R_{4′}; R₁, R₂, and R₄ all are OH; R_{1′}, R_{2′}, and R_{4′} all are H; W is alkyl; and Base is guanine.

In a third subembodiment, G₂ is S and G₄ is N; G₁, G₃ and G₅ are CR₁R_{1′}, CR₃R_{3′}, and CR₅R_{5′}; R₁, R₃, and R₅ all are OH; R_{1′}, R_{3′}, and R_{5′} all are H; W is halo; and Base is phenylthiazole.

In a fourth subembodiment, G₂ is S and G₃ is O; G₁, G₄ and G₅ are CR₁R_{1′}, CR₄R_{4′}, and CR₅R_{5′}; R₁, R₄ and R₅ all are OH; R_{1′}, R_{4′} and R_{5′} all are H; W is haloalkyl; and Base is benzimidazole.

In a fifth subembodiment, G₁ is P and G₄ is O; G₂, G₃ and G₅ are CR₂R_{2′}, CR₃R_{3′}, and CR₅R_{5′}; R₁, R₂, R₃ and R₅ all are OH; R_{1′}, R_{2′}, R_{3′}, and R_{5′} all are H; W is N₃; and Base is pyrrolopyrimidine.

In a sixth subembodiment, G₄ is N and G₁, G₂, G₃ and G₅ are CR₁R_{1′}, CR₂R_{2′}, CR₃R_{3′}, and CR₅R_{5′}; R₁, R₂, and R₅ all are OH; R_{1′}, R_{2′}, R_{3′}, and R_{5′} all are H; R₃ is halo; W is NO₂; and Base is adenosine.

In a seventh subembodiment, R₁ is OH; and any of R₂, R₃, R₄ or R₅ are O-acyl.

In a subembodiment, a compound of any of Formulas (I), (IIa)-(IIf), (III), or (IV), or a pharmaceutically acceptable salt or prodrug, or a tautomeric or polymorphic form thereof, X is selected from the group consisting of O, S, or NR.

In a another subembodiment, a compound of any of Formulas (I), (IIa)-(IIf), (III), or (IV), W is H.

In another subembodiment, a compound of any of Formulas (I), (IIa)-(IIf), (III), or (IV), R₁ is OH or mono, di or triphosphate.

In another subembodiment, a compound of any of Formulas (I), (IIa)-(IIf), (III), or (IV), at least two of R₁, R₂, R₃, R₄ and R₅ are OH, O-alkyl or O-acyl.

In another subembodiment, a compound of any of Formulas (I), (IIa)-(IIf), (III), or (IV), at least one of R₁ and R_{1′}, R₂ and R_{2′}, R₃ and R_{3′}, R₄ and R_{4′} or R₅ and R_{5′} are H.

In another subembodiment, a compound of any of Formulas (I), (IIa)-(IIf), (III), or (IV), one of R₅ or R_{5′} is methyl, alkynyl or fluoro.

In any of the embodiments described herein, any of R₁, R_{1′}, R₂, R_{2′}, R₃, R_{3′}, R₄, R_{4′}, R₅, R_{5′}, or W may be a pharmaceutically acceptable leaving group that provides the parent compound in vivo.

II. Definitions

The term “apionucleoside” as used herein, unless otherwise specified, refers to a nucleoside ring which retains the nucleobase adjacent to “X”, the carbon or heteroatom in the ring while the R₁ substituent, is displaced to various positions throughout the ring.

The term “isonucleoside” as used herein, unless otherwise specified, refers to a nucleoside ring in which the R₁ substituent, retains its position adjacent to the carbon or heteroatom “X” while the nucleobase is displaced to various positions throughout the ring.

The term “alkyl”, as used herein, unless otherwise specified, includes a saturated straight, branched, or cyclic, primary, secondary, or tertiary hydrocarbon of typically C₁ to C₁₀. The term includes both substituted and unsubstituted alkyl groups, and particularly includes halogenated alkyl groups, and even more particularly fluorinated alkyl groups. Non-limiting examples of moieties with which the alkyl group can be substituted are selected from the group consisting of halogen (fluoro, chloro, bromo or iodo), hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991, hereby incorporated by reference. The term specifically includes methyl, CF₃, CCl₃, CFCl₂, CF₂Cl, ethyl, CH₂CF₃, CF₂CF₃, propyl, isopropyl, cyclopropyl, butyl, isobutyl, secbutyl, t-butyl, pentyl, cyclopentyl, isopentyl, neopentyl, hexyl, isohexyl, cyclohexyl, cyclohexylmethyl, 3-methylpentyl, 2,2-dimethylbutyl, and 2,3-dimethylbutyl.

The term “lower alkyl”, as used herein, and unless otherwise specified, includes a C₁ to C₆ saturated straight, branched, or if appropriate, a cyclic (for example, cyclopropyl) alkyl group, including both substituted and unsubstituted moieties.

Whenever a range of carbon atoms is referred to, it includes independently and separately every member of the range. As a non-limiting example, the term “C₁-C₁₀ alkyl” is considered to include, independently, each member of the group, such that, for example, C₁-C₁₀ alkyl includes straight, branched and where appropriate cyclic C₁, C₂, C₃, C₄, C₅, C₆, C₇, C₈, C₉ and C₁₀ alkyl functionalities.

The term “alkylthio” includes a straight or branched chain alkylsulfide of the number of carbons specified, such as for example, C₁₋₄ alkylthio, ethylthio, —S-alkyl; —S-alkenyl, —S-alkynyl, etc.

The term “alkylamino” or “arylamino” includes an amino group that has one or two alkyl or aryl substituents, respectively. Unless otherwise specifically stated in this application, when alkyl is a suitable moiety, lower alkyl is. Similarly, when alkyl or lower alkyl is a suitable moiety, unsubstituted alkyl or lower alkyl is.

The term “alkylsulfonyl” means a straight or branched alkylsulfone of the number of carbon atoms specified, as for example, C₁₋₆ alkylsulfonyl or methylsulfonyl.

The term “alkoxycarbonyl” includes a straight or branched chain ester of a carboxylic acid derivative of the number of carbon atoms specified, such as for example, a methoxycarbonyl, MeOCO—.

As used herein, the term “nitro” means —NO₂; the term “sulfhydryl” means —SH; and the term “sulfonyl” means —SO₂.

The term “protected” as used herein and unless otherwise defined includes a group that is added to reactive group, including an oxygen, nitrogen, or phosphorus atom to prevent its further reaction or for other purposes. A wide variety of oxygen and nitrogen protecting groups are known to those skilled in the art of organic synthesis.

The term “aryl”, as used herein, and unless otherwise specified, includes phenyl, biphenyl, or naphthyl, which is optionally substituted. The aryl group can be substituted with any described moiety, including, but not limited to,one or more moieties selected from the group consisting of halogen (fluoro, chloro, bromo or 15 iodo), hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991.

The term “alkaryl” or “alkylaryl” includes an alkyl group with an aryl substituent. The term aralkyl or arylalkyl includes an aryl group with an alkyl substituent.

The term “aralkyl” as used herein includes an alkyl group substituted with an aryl group.

The term “cycloalkyl” means a cyclic ring of C₃₋₈, including but not limited to cyclopropyl, cyclobutyl, cyclopentyl, cyclohexyl, cycloheptyl and cyclooctyl.

The term “alkoxy” means a straight or branched chain alkyl group having an attached oxygen radical, the alkyl group having the number of carbons specified or any number within this range. For example, a C₁₋₄ alkoxy, methoxy, etc.

The term “halo”, as used herein, includes chloro, bromo, iodo, and fluoro.

The term “purine” or “pyrimidine” base includes, but is not limited to, adenine, N⁶-alkylpurines, N⁶-acylpurines (wherein acyl is C(O)(alkyl, aryl, alkylaryl, or arylalkyl), N⁶-benzylpurine, N⁶-halopurine, N⁶-vinylpurine, N⁶-acetylenic purine, N⁶-acyl purine, N⁶-hydroxyalkyl purine, N⁶-alkylaminopurine, N⁶-thioalkyl purine, N²-alkylpurines, N²-alkyl-6-thiopurines, thymine, cytosine, 5-fluorocytosine, 5-methylcytosine, 6-azapyrimidine, including 6-azacytosine, 2- and/or 4-mercaptopyrmidine, uracil, 5-halouracil, including 5-fluorouracil, C⁵-alkylpyrimidines, C⁵-benzylpyrimidines, C⁵-halopyrimidines, C⁵-vinylpyrimidine, C⁵-acetylenic pyrimidine, C⁵-acyl pyrimidine, C⁵-hydroxyalkyl purine, C⁵-amidopyrimidine, C⁵-cyanopyrimidine, C⁵-iodopyrimidine, C⁶-iodo-pyrimidine, C⁵-Br-vinyl pyrimidine, C⁶-Br-vinyl pyrimidine, C⁵-nitropyrimidine, C⁵-amino-pyrimidine, N²-alkylpurines, N²-alkyl-6-thiopurines, 5-azacytidinyl, 5-azauracilyl, triazolopyridinyl, imidazolopyridinyl, pyrrolopyrimidinyl, and pyrazolopyrimidinyl. Purine bases include, but are not limited to, guanine, adenine, hypoxanthine, 2,6-diaminopurine, and 6-chloropurine. Functional oxygen and nitrogen groups on the base can be protected as necessary or desired. Suitable protecting groups are well known to those skilled in the art, and include trimethylsilyl, dimethylhexylsilyl, t-butyldimethylsilyl, and t-butyldiphenylsilyl, trityl, alkyl groups, and acyl groups such as acetyl and propionyl, methanesulfonyl, and p-toluenesulfonyl.

The term “acyl” or “O-linked ester” includes a group of the formula C(O)R′, wherein R′ is an straight, branched, or cyclic alkyl (including lower alkyl), amino acid, aryl including phenyl, alkaryl, aralkyl including benzyl, alkoxyalkyl including methoxymethyl, aryloxyalkyl such as phenoxymethyl; or substituted alkyl (including lower alkyl), aryl including phenyl optionally substituted with chloro, bromo, fluoro, iodo, C₁ to C₄ alkyl or C₁ to C₄ alkoxy, sulfonate esters such as alkyl or aralkyl sulphonyl including methanesulfonyl, the mono, di or triphosphate ester, trityl or monomethoxy-trityl, substituted benzyl, alkaryl, aralkyl including benzyl, alkoxyalkyl including methoxymethyl, aryloxyalkyl such as phenoxymethyl. Aryl groups in the esters optimally comprise a phenyl group. In particular, acyl groups include acetyl, trifluoroacetyl, methylacetyl, cyclopropylacetyl, cyclopropyl carboxy, propionyl, butyryl, hexanoyl, heptanoyl, octanoyl, neo-heptanoyl, phenylacetyl, 2-acetoxy-2-phenylacetyl, diphenylacetyl, α-methoxy-α-trifluoromethyl-phenylacetyl, bromoacetyl, 2-nitro-benzeneacetyl, 4-chloro-benzeneacetyl, 2-chloro-2,2-diphenylacetyl, 2-chloro-2-phenylacetyl, trimethylacetyl, chlorodifluoroacetyl, perfluoroacetyl, fluoroacetyl, bromodifluoroacetyl, methoxyacetyl, 2-thiopheneacetyl, chlorosulfonylacetyl, 3-methoxyphenylacetyl, phenoxyacetyl, tert-butylacetyl, trichloroacetyl, monochloro-acetyl, dichloroacetyl, 7H-dodecafluoro-heptanoyl, perfluoro-heptanoyl, 7H-dodeca-fluoroheptanoyl, 7-chlorododecafluoro-heptanoyl, 7-chloro-dodecafluoro-heptanoyl, 7H-dodecafluoroheptanoyl, 7H-dodeca-fluoroheptanoyl, nona-fluoro-3,6-dioxa-heptanoyl, nonafluoro-3,6-dioxaheptanoyl, perfluoroheptanoyl, methoxybenzoyl, methyl 3-amino-5-phenylthiophene-2-carboxyl, 3,6-dichloro-2-methoxy-benzoyl, 4-(1,1,2,2-tetrafluoro-ethoxy)-benzoyl, 2-bromo-propionyl, omega-aminocapryl, decanoyl, n-pentadecanoyl, stearyl, 3-cyclopentyl-propionyl, 1-benzene-carboxyl, O-acetylmandelyl, pivaloyl acetyl, 1-adamantane-carboxyl, cyclohexane-carboxyl, 2,6-pyridinedicarboxyl, cyclopropane-carboxyl, cyclobutane-carboxyl, perfluorocyclohexyl carboxyl, 4-methylbenzoyl, chloromethyl isoxazolyl carbonyl, perfluorocyclohexyl carboxyl, crotonyl, 1-methyl-1H-indazole-3-carbonyl, 2-propenyl, isovaleryl, 1-pyrrolidinecarbonyl, 4-phenylbenzoyl. When the term acyl is used, it is meant to be a specific and independent disclosure of acetyl, trifluoroacetyl, methylacetyl, cyclopropylacetyl, propionyl, butyryl, hexanoyl, heptanoyl, octanoyl, neo-heptanoyl, phenylacetyl, diphenylacetyl, α-trifluoromethyl-phenylacetyl, bromoacetyl, 4-chloro-benzeneacetyl, 2-chloro-2,2-diphenylacetyl, 2-chloro-2-phenylacetyl, trimethylacetyl, chlorodifluoroacetyl, perfluoroacetyl, fluoroacetyl, bromodifluoroacetyl, 2-thiopheneacetyl, tert-butylacetyl, trichloroacetyl, monochloro-acetyl, dichloroacetyl, methoxybenzoyl, 2-bromo-propionyl, decanoyl, n-pentadecanoyl, stearyl, 3-cyclopentyl-propionyl, 1-benzene-carboxyl, pivaloyl acetyl, 1-adamantane-carboxyl, cyclohexane-carboxyl, 2,6-pyridinedicarboxyl, cyclopropane-carboxyl, cyclobutane-carboxyl, 4-methylbenzoyl, crotonyl, 1-methyl-1H-indazole-3-carbonyl, 2-propenyl, isovaleryl, 4-phenylbenzoyl.

The term “acylamino” means a group having a structure of —N(R)—C(═O)—R, wherein R is an straight, branched, or cyclic alkyl (including lower alkyl), amino acid, aryl including phenyl, alkaryl, aralkyl including benzyl, alkoxyalkyl including methoxymethyl, aryloxyalkyl such as phenoxymethyl; or substituted alkyl (including lower alkyl), aryl including phenyl optionally substituted with chloro, bromo, fluoro, iodo, C₁ to C₄ alkyl or C₁ to C₄ alkoxy, sulfonate esters such as alkyl or aralkyl sulphonyl including methanesulfonyl, the mono, di or triphosphate ester, trityl or monomethoxy-trityl, substituted benzyl, alkaryl, aralkyl including benzyl, alkoxyalkyl including methoxymethyl, aryloxyalkyl such as phenoxymethyl.

The term “carbonyl” means a group of the structure —C(═O)—X—R or X—C(═O)—R, where X is O, S or a bond, and R is as defined above.

The term “heteroatom” means an atom other than carbon or hydrogen, and particularly nitrogen, oxygen, sulfur, phosphorus or boron.

The term “amino acid” includes naturally occurring and synthetic α, β γ or δ amino acids, and includes but is not limited to, amino acids found in proteins, i.e. glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine. In one embodiment, the amino acid is in the L-configuration. Alternatively, the amino acid can be a derivative of alanyl, valinyl, leucinyl, isoleuccinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl, histidinyl, β-alanyl, β-valinyl, β-leucinyl, β-isoleuccinyl, β-prolinyl, β-phenylalaninyl, β-tryptophanyl, β-methioninyl, β-glycinyl, β-serinyl, β-threoninyl, β-cysteinyl, β-tyrosinyl, β-asparaginyl, β-glutaminyl, β-aspartoyl, β-glutaroyl, β-lysinyl, β-argininyl or β-histidinyl. When the term amino acid is used, it is considered to be a specific and independent disclosure of each of the esters of α, β γ or δ glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine in the D and L-configurations.

The term “amido” as used herein means an amino-substituted carbonyl, while the term “amidino” means a group having the structure —C(═NH)—NH₂.

Certain sulfur- and phosphorus-containing terms have the following structural significances: “sulfonate” means a group of the structure —S(═O)(═O)—OR wherein R is an straight, branched, or cyclic alkyl (including lower alkyl), amino acid, aryl including phenyl, alkaryl, aralkyl including benzyl, alkoxyalkyl including methoxymethyl, aryloxyalkyl such as phenoxymethyl; or substituted alkyl (including lower alkyl), aryl including phenyl optionally substituted with chloro, bromo, fluoro, iodo, C₁ to C₄ alkyl or C₁ to C₄ alkoxy, sulfonate esters such as alkyl or aralkyl sulphonyl including methanesulfonyl, the mono, di or triphosphate ester, trityl or monomethoxy-trityl, substituted benzyl, alkaryl, aralkyl including benzyl, alkoxyalkyl including methoxymethyl, aryloxyalkyl such as phenoxymethyl; “sulfate” means a group of the structure O—S(═O)(═O)—OR where R is as defined above; “sulfonamide” means a group of the structure —N(R)—S(═O)(═O)—R where R is as defined abover; “sulfamoyl” means a group of the structure —S(═O)(═O)—N(R)(R) where R is as defined above; “sulfoxido” means a group of the structure —S(═O)—R where R is as defined above; “phosphoryl” means a group of the structure —P(═O)—OR where R is as defined above; and “phosphoroamidate” means a group of the structure -Q-P(NR₁R₂)(═O)—OR where R is as defined above and .

The term “prodrug”, as used herein, includes a nucleoside analogue that has a biologically cleavable moiety at one or more positions, including, but not limited to an ester or acyl moiety.

As used herein, the term “substantially free of” or “substantially in the absence of” includes a nucleoside composition that includes at least 85 or 90% by weight, including 95%, 98%, 99% or 100% by weight, of the designated enantiomer of that nucleoside. In a embodiment, in the methods and compounds of this invention, the compounds are substantially free of enantiomers.

Similarly, the term “isolated” includes a nucleoside composition that includes at least 85%, 90%, 95%, 98%, 99%, or 100% by weight, of the nucleoside, the remainder comprising other chemical species or enantiomers.

The term “host”, as used herein, includes an unicellular or multicellular organism in which the virus can replicate, including cell lines and animals, including a human. Alternatively, the host can be carrying a part of the Flaviviridae viral genome, whose replication or function can be altered by the compounds of the present invention. The term host specifically includes infected cells, cells transfected with all or part of the Flaviviridae genome and animals, in particular, primates (including chimpanzees), mammals and humans. In most animal applications of the present invention, the host is a human patient. Veterinary applications, in certain indications, however, are clearly anticipated by the present invention (such as chimpanzees).

III. Prodrugs and Derivatives

The active compound can be administered as any salt, ester or prodrug that upon administration to the recipient is capable of providing directly or indirectly the parent compound, or that exhibits activity itself. Nonlimiting examples are the pharmaceutically acceptable salts (alternatively referred to as “physiologically acceptable salts”), esters, and a compound, which has been alkylated, acylated, or otherwise modified at the 5′-position, or on the purine or pyrimidine base (a type of “pharmaceutically acceptable prodrug”). Further, the modifications can affect the biological activity of the compound, in some cases increasing the activity over the parent compound. This can easily be assessed by preparing the salt, ester or prodrug and testing its antiviral activity according to the methods described herein, or other methods known to those skilled in the art.

Pharmaceutically Acceptable Salts

The term “pharmaceutically acceptable salt, ester or prodrug” is used throughout the specification to describe any pharmaceutically acceptable form (such as an ester, phosphate ester, salt of an ester or a related group) of a nucleoside compound which, upon administration to a patient, provides the nucleoside compound. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. Pharmaceutically acceptable prodrugs refer to a compound that is metabolized, for example hydrolyzed or oxidized, in the host to form the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound. The compounds of this invention possess antiviral activity against a Flaviviridae, or are metabolized to a compound that exhibits such activity. In cases where compounds are sufficiently basic or acidic to form stable nontoxic acid or base salts, administration of the compound as a pharmaceutically acceptable salt may be appropriate. Examples of pharmaceutically acceptable salts are organic acid addition salts formed by addition of acids, which form a physiological acceptable anion, for example, tosylate, methanesulfonate, acetate, citrate, malonate, tartarate, succinate, benzoate, ascorate, α-ketoglutarate, α-glycerophosphate, formate, fumarate, propionate, glycolate, lactate, pyruvate, oxalate, maleate, sulfonate and salicylate. Suitable inorganic salts may also be formed, including, sulfate, nitrate, bicarbonate, carbonate salts, hydrobromate, hydrobromide, hydroiodide and phosphoric acid. In a embodiment, the salt is a mono- or di-hydrochloride salt.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made. In one embodiment, the salt is a hydrochloride salt of the compound. In another embodiment, the pharmaceutically acceptable salt is a dihydrochloride salt.

Nucleotide Prodrug Formulations

The nucleosides described herein can be administered as a nucleotide prodrug to increase the activity, bioayailability, stability or otherwise alter the properties of the nucleoside. A number of nucleotide prodrug ligands are known. In general, alkylation, acylation or other lipophilic modification of the mono-, di- or triphosphate of the nucleoside reduces polarity and allows passage into cells. Examples of substituent groups that can replace one or more hydrogens on the phosphate moiety are alkyl, aryl, steroids, carbohydrates, including sugars, 1,2-diacylglycerol and alcohols. Many are described in R. Jones and N. Bischoferger, Antiviral Research, 1995, 27:1-17. Any of these can be used in combination with the disclosed nucleosides to achieve a desired effect.

In an alternative embodiment, the nucleoside is delivered as a phosphonate or a SATE derivative.

The active nucleoside can also be provided as a 2′, 3′ and/or 5′-phosphoether lipid or a 2′, 3′ and/or 5′-ether lipid. Non-limiting examples are described include the following references, which are incorporated by reference herein: Kucera, L. S., N. Iyer, E. Leake, A. Raben, Modest E. K., D. L. W., and C. Piantadosi. 1990, AIDS Res. Hum. Retro Viruses. 6:491-501; Piantadosi, C., J. Marasco C. J., S. L. Morris-Natschke, K. L. Meyer, F. Gumus, J. R. Surles, K. S. Ishaq, L. S. Kucera, N. Iyer, C. A. Wallen, S. Piantadosi, and E. J. Modest. 1991, J. Med. Chem. 34:1408.1414; Hosteller, K. Y., D. D. Richman, D. A. Carson, L. M. Stuhmiller, G. M. T. van Wijk, and H. van den Bosch. 1992, Antimicrob. Agents Chemother. 36:2025.2029; Hosetler, K. Y., L. M. Stuhmiller, H. B. Lenting, H. van den Bosch, and D. D. Richman, 1990, J. Biol. Chem. 265:61127.

Nonlimiting examples of U.S. patents that disclose suitable lipophilic substituents that can be covalently incorporated into the nucleoside, including at the 2′, 3′ and/or 5′-OH position of the nucleoside or lipophilic preparations, include U.S. Pat. Nos. 5,149,794, 5,256,641, 5,543,389, 5,543,390, and 5,543,391, all to Yatvin et al; U.S. Pat. Nos. 5,194,654, 5,223,263, 5,411,947, and 5,463,092, all to Hostetler et al.; and U.S. Pat. No. 5,554,728 to Basava et al., all of which are incorporated herein by reference. Foreign patent applications that disclose lipophilic substituents that can be attached to the nucleosides of the present invention, or lipophilic preparations, include WO 89/02733, WO 90/00555, WO 91/16920, WO 91/18914, WO 93/00910, WO 94/26273, WO 96/15132, EP 0 350 287, EP 93917054.4, and WO 91/19721.

Aryl esters, especially phenyl esters, are also provided. Nonlimiting examples are disclosed in DeLambert et al., J. Med. Chem. 37: 498 (1994). Phenyl esters containing a carboxylic ester ortho to the phosphate are also provided (Khamnei and Torrence, J. Med. Chem.; 39:4109-4115 (1996)). In particular, benzyl esters, which generate the parent compound, in some cases using substituents at the ortho- or para-position to accelerate hydrolysis, are provided. Examples of this class of prodrugs are described by Mitchell et al., J. Chem. Soc. Perkin Trans. 12345 (1992); Brook, et al. WO 91/19721; and Glazier et al. WO 91/19721.

Cyclic and noncyclic phosphonate esters are also provided. Nonlimiting examples are disclosed in Hunston et al., J. Med. Chem. 27: 440-444 (1984) and Starrett et al. J. Med. Chem. 37: 1857-1864 (1994). Additionally, cyclic 3′,5′-phosphate esters are disclosed in Meier et al. J. Med. Chem. 22: 811-815 (1979) as non-limiting examples. Cyclic 1′,3′-propanyl phosphonate and phosphate esters, such as ones containing a fused aryl ring like the cyclosaligenyl ester, are provided by Meier et al., Bioorg. Med. Chem. Lett. 7: 99-104 (1997). Unsubstituted cyclic 1′,3′-propanyl esters of the monophosphates are provided by Farquhar et al., J. Med. Chem. 26: 1153 (1983) and, J. Med. Chem. 28: 1358 (1985). In addition, cyclic 1′,3′-propanyl esters substituted with a pivaloyloxy methyloxy group at C-1′ are provided by Freed et al., Biochem. Pharmac. 38: 3193 (1989) and Biller et al., U.S. Pat. No. 5,157,027.

Cyclic phosphoramidates are known to cleave in vivo by an oxidative mechanism. Therefore, in one embodiment of the present invention, a variety of substituted 1′,3′ propanyl cyclic phosphoramidates are provided. Non-limiting examples are disclosed by Zon, Progress in Med. Chem. 19, 1205 (1982). Additionally, a number of 2′- and 3′-substituted proesters are provided Wherein 2′-substituents include methyl, dimethyl, bromo, trifluoromethyl, chloro, hydroxy, and methoxy, and 3′-substituents include phenyl, methyl, trifluoromethyl, ethyl, propyl, i-propyl, and cyclohexyl. 1′-substituted analogs are also provided.

Cyclic esters of phosphorus-containing compounds are given in the following non-limiting examples:
[1] di- and tri-esters of phosphoric acids reported in Nifantyev et al., Phosphorus, Sulfur Silicon and Related Eelements, 113: 1 (1996) and in Wijnberg et al., EP-180276 A1;
[2] phosphorus (III) acid esters reported by Kryuchkov et al., Izv. Akad. Nauk SSSR, Ser. Khim. 6: 1244 (1987) and Sylvain et al., DE3512781 A1;
[3] phosphoramidates reported by Shih et al., Bull. Inst. Chem. Acad. Sin, 41: 9 (1994) and Edmundson et al., J. Chem. Res. Synop. 5: 122 (1989); and
[4] phosphonates, reported by Neidlein et al., Heterocycles 35: 1185 (1993).

Further examples of prodrugs falling within the invention include prodrugs disclosed in U.S. Pat. Nos. 6,284,748 and 6,312,662, 6,967,193, 6,946,115, 6,752,981, 6,965,033, 6,919,322, as well as patent publications nos. 2002/0040014, WO 99/45016, WO 00/52015, WO 03/095665, WO 04/037161, WO 04/041834, WO 04/041837, WO 01/18013, WO 98/39344, and EP 1 634 886. The prodrugs and technology described in any of these applications and patents, incorporated by reference, can be used with the 7-membered ring nucleosides disclosed herein. As a non-limiting example, the prodrugs of the present invention include compounds of the structure 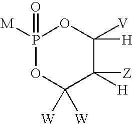
wherein:
V, W and W′ are independently selected from the group consisting of —H, alkyl, aralkyl, alicyclic, aryl, substituted aryl, heteroaryl, substituted heteroaryl, 1-alkenyl, and 1-alkynyl; or
together V and Z are connected via an additional 3-5 atoms to form a cyclic group containing 5-7 atoms, optionally 1 heteroatom, substituted with hydroxy, acyloxy, alkoxycarbonyloxy, or aryloxycarbonyloxy attached to a carbon atom that is three atoms from both O groups attached to the phosphorus; or
together V and Z are connected via an additional 3-5 atoms to form a cyclic group, optionally containing 1 heteroatom, that is fused to an aryl group at the beta and gamma position to the O attached to the phosphorus; or
together V and W are connected via an additional 3 carbon atoms to form an optionally substituted cyclic group containing 6 carbon atoms and substituted with one substituent selected from the group consisting of hydroxy, acyloxy, alkoxycarbonyloxy, alkylthiocarbonyloxy, and aryloxycarbonyloxy, attached to one of said carbon atoms that is three atoms from an O attached to the phosphorus;
together Z and W are connected via an additional 3-5 atoms to form a cyclic group, optionally containing one heteroatom, and V must be aryl, substituted aryl, heteroaryl, or substituted heteroaryl;
together W and W′ are connected via an additional 2-5 atoms to form a cyclic group, optionally containing 0-2 heteroatoms, and V must be aryl, substituted aryl, heteroaryl, or heteroaryl;
Z is selected from the group consisting of —CHR²OH, —CHR²OC(O)R³, —CHR²OC(S)R³, —CHR²OC(S)OR³, —CHR²OC(O)SR³, —CHR²OCO₂R³, —OR², —SR, —CHR²N₃, —CH₂aryl, —CH(aryl)OH, —CH(CH═CR²₂)OH, —CH(C≡CR²)OH, —R², —NR²₂, —OCOR³, —OCO₂R³, —SCOR³, —SCO₂R³, —NHCOR², —NHCO₂R³, —CH₂NHaryl, —(CH₂)ₚ—OR 2, and —(CH₂)ₚ—SR¹²;
p is an integer 2 or 3;
R² is selected from the group consisting of R³ and —H;
R³ is selected from the group consisting of alkyl, aryl, alicyclic, and aralkyl;
R¹² is selected from the group consisting of —H, and lower acyl;
M is selected from the group that attached to PO₃²⁻—, P₂O₆³⁻—or P_{3O}₉⁴⁻ is a the 2′-branched nucleoside, and is attached to the phosphorus via a carbon, oxygen, sulfur or nitrogen atom.
   IV. Combination or Alternation Therapy

The active compounds of the present invention can be administered in combination or alternation with another anti-Flaviviridae virus agent, including anti-flavivirus or pestivirus agent, or in particular an anti-HCV agent to treat any of the conditions described herein. In combination therapy, effective dosages of two or more agents are administered together, whereas in alternation or sequential-step therapy, an effective dosage of each agent is administered serially or sequentially. The dosages given will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions. In certain embodiments, an anti-HCV (or anti-pestivirus or anti-flavivirus) compound that exhibits an EC₅₀ of less than 15, 10, 5 or 1 μM is desirable.

Drug-resistant variants of Flaviviridae viruses, including flaviviruses, pestiviruses or HCV can emerge after prolonged treatment with an antiviral agent. Drug resistance most typically occurs by mutation of a gene that encodes for an enzyme used in viral replication. The efficacy of a drug against the viral infection can be prolonged, augmented, or restored by administering the compound in combination or alternation with a second, and perhaps third, antiviral compound that induces a different mutation from that caused by the principle drug. Alternatively, the pharmacokinetics, biodistribution or other parameter of the drug can be altered by such combination or alternation therapy. In general, combination therapy is typical because it induces multiple simultaneous stresses on the virus.

Examples of classes of drugs that are being developed to treat Flaviviridae infections include:
(1) Protease inhibitors

Substrate-based NS3 protease inhibitors including but not limited to those disclosed by Attwood et al.in WO 98/22496, 1998, in Antiviral Chemistry and Chemotherapy 1999, 10, 259-273, and in, DE 19914474; and WO 98/17679 to Tung et al., that discloses alphaketoamides and hydrazinoureas;

Substrate inhibitors that terminate in an electrophile such as a boronic acid or phosphonate including but not limited to those shown by Llinas-Brunet et al, in WO 99/07734;.

Non-substrate-based NS3 protease inhibitors including but not limited to those such as 2,4,6-trihydroxy-3-nitro-benzamide derivatives and RD3-4082 and RD3-4078, the former substituted on the amide with a 14 carbon chain and the latter having a para-phenoxyphenyl group, shown by Sudo K. et al., Biochemical and Biophysical Research Communications, 1997, 238, 643-647, and in Antiviral Chemistry and Chemotherapy, 1998, 9, 186;

Sch 68631, a phenanthrenequinone, including but not limited to those disclosed by Chu M. et al., Tetrahedron Letters 37:7229-7232, 1996; Sch 351633, isolated from the fungus Penicillium griseofulvum, disclosed by Chu M. et al., Bioorganic and Medicinal Chemistry Letters 9:1949-1952;

Eglin c, a macromolecule isolated from leech, that exhibits nanomolar potency inhibition against several serine proteases such as S. griseus proteases A and B, α-chymotrypsin, chymase and subtilisin, as disclosed by Qasim M.A. et al., Biochemistry 36:1598-1607, 1997;

A class of cysteine protease inhibitors for inhibiting HCV endopeptidase 2 including but not limited to those disclosed in U.S. Pat. No. 6,004,933 to Spruce et al.;

Synthetic inhibitors of hepatitis C virus NS3 protease or the NS4A cofactor that are subsequences of substrates utilized by the protease and/or cofactor including but not limited to those shown in U.S. Pat. No. 5,990,276 to Zhang et al.;

Restriction enzymes to treat HCV including but not limited to those disclosed in U.S. Pat. No. 5,538,865 to Reyes et al.;

Peptides like NS3 serine protease inhibitors of HCV including but not limited to those disclosed in WO 02/008251 to Corvas International, Inc, and WO 02/08187 and WO 02/008256 to Schering Corporation;

HCV inhibitor tripeptides including but not limited to those shown in U.S. Pat. Nos. 6,534,523, 6,410,531, and 6,420,380 to Boehringer Ingelheim and WO 02/060926 to Bristol Myers Squibb;

Diaryl peptides like NS3 serine protease inhibitors of HCV including but not limited to those disclosed in WO 02/48172 to Schering Corporation;

Imidazoleidinones such as NS3 serine protease inhibitors of HCV including but not limited to those disclosed in WO 02/08198 to Schering Corporation and WO 02/48157 to Bristol Myers Squibb;

HCV protease inhibitors including but not limited to those shown in WO 98/17679 to Vertex Pharmaceuticals and WO 02/48116 to Bristol Myers Squibb;
(2) Thiazolidine derivatives that show relevant inhibition in a reverse-phase HPLC assay with an NS3/4A fusion protein and NS5A/5B substrate, including but not limited to those demonstrated by Sudo K. et al., Antiviral Research, 1996, 32, 9-18, especially compound RD-1-6250 that possesses a fused cinnamoyl moiety substituted with a long alkyl chain, RD4 6205 and RD4 6193;
(3) Thiazolidines and benzanilides including but not limited to those identified by Kakiuchi N. et al., J. EBS Letters 421, 217-220, and Takeshita N. et al. Analytical Biochemistry, 1997, 247, 242-246;
(4) Helicase inhibitors including but not limited to those disclosed by Diana G. D. et al., in U.S. Pat. No. 5,633,358 and in WO 97/36554);
(5) Nucleotide polymerase inhibitors or gliotoxin including but not limited to those shown by Ferrari R. et al. Journal of Virology, 1999, 73, 1649-1654;
(6) Cerulenin, a natural product shown by Lohmann V. et al., Virology, 1998, 249, 108-118;
(7) Antisense phosphorothioate oligodeoxynucleotides (S-ODN) complementary to sequence stretches in the 5′ non-coding region (NCR) of the Flaviviridae virus including but not limited to those demonstrated by Alt M. et al., Hepatology, 1995, 22, 707-717;
(8) Nucleotides 326-348 comprising the 3′ end of the NCR and nucleotides 371-388 located in the core coding region of the HCV RNA, including but not limited to those shown by Alt M. et al., Archives of Virology, 1997, 142, 589-599, and by Galderisi U. et al., Journal of Cellular Physiology, 1999, 181, 251-257;
(9) Inhibitors of IRES-dependent translation including but not limited to those disclosed by Ikeda N et al., Japanese Patent Pub. JP-08268890, and Kai Y. et al. Japanese Patent Pub. JP-10101591);
(10) Ribozymes, such as nuclease-resistant ribozymes including but not limited to those shown by Maccjak, D. J. et al., Hepatology 1999, 30, abstract 995; Barber et al. in U.S. Pat. No. 6,043,077; and Draper et al. in U.S. Pat. Nos. 5,869,253 and 5,610,054;
(11) Nucleoside analogs including the use of branched nucleosides in the treatment of flaviviruses, pestiviruses, and hepacivirus, including but not limited to those shown by Idenix Pharmaceuticals in WO 01/92282, U.S. 6,812,219, WO 01/90121 (U.S. Pat. No. 6,914,054), and U.S. 2004/0077587 where a method is disclosed for the treatment of hepatitis C, pestivirus and/or flavivirus infection in humans and other host animals that includes administering an effective amount of a biologically active 1′, 2′, 3′ or 4′-branched β-D or β-L nucleosides or a pharmaceutically acceptable salt, ester or derivative thereof, administered either alone or in combination with another antiviral agent, optionally in a pharmaceutically acceptable carrier; including but not limited to those disclosed in WO 01/32153 and WO 01/60315 filed by BioChem Pharma, Inc. (now Shire Biochem, Inc.); U.S. Pat. No. 6,777,395, U.S.2004/0110717, WO 02/057425, WO 04/000858, WO 04/003138, 04/007512 and WO 02/057287 filed by Merck & Co., Inc.; WO 02/18404, U.S. 2004/0110718, U.S. Pat. No. 6,660,721, U.S. Pat. No. 784,166, and U.S. Pat. No. 6,846,810 filed by Roche; and WO 01/79246, WO 02/32920, U.S. 2004/0002476, U.S. 2005/0009737 and WO 02/48165 filed by Pharmasset, Ltd.; WO 03/051899, WO 03,061385, 03/061576, WO 03/062255 and U.S. 2004/0023921 filed by Ribapharm; U.S. Pat. No. 6,495,677, and U.S. 2002/0095033 filed by ICN Pharmaceuticals; U.S. 2004/0063658 and U.S. 2004/0147464 filed by Genelabs Technologies; and U.S. Pat. No. 6,348,587, WO 99/43691 filed by Emory University all disclose the use of certain 2′-fluoronucleosides to treat HCV; and 2′-modified nucleosides for inhibition of HCV including but not limited to those described by Eldrup et al.; nucleoside analogues as possible inhibitors of HCV RNA replication were disclosed by Bhat et al. where at .p A75, the authors report that 2′-modified nucleosides demonstrate potent inhibitory activity in cell-based replicon assays; and effects of 2′-modified nucleosides on HCV RNA replication were reported by Olsen et al., all at the Oral Session V, Hepatitis C Virus, Flaviviridae; 16^{th} International Conference on Antiviral Research, Apr. 27, 2003, Savannah, Ga.;
(12) Miscellaneous compounds including but not limited to 1-amino-alkylcyclohexanes shown in U.S. Pat. No. 6,034,134 to Gold et al.; alkyl lipids, vitamin E and other antioxidants including but not limited to those described in U.S. Pat. No. 5,922,757 to Chojkier et al.; squalene, amantadine, and bile acids including but not limited to those found in U.S. Pat. No. 5,846,964 to Ozeki et al.; N-(phosphonoacetyl)-L-aspartic acid and piperidines including but not limited to those shown in U.S. Pat. No. 5,830,905 to Diana et al.; benzenedicarboxamides including but not limited to those disclosed in U.S. Pat. No. 5,633,388 to Diana et al; polyadenylic acid derivatives including but not limited to those described in U.S. Pat. No. 5,496,546 to Wang et al.; 2′,3′-dideoxyinosine including but not limited to those disclosed in U.S. Pat. No. 5,026,687 to Yarchoan et al.; benzimidazoles including but not limited to those demonstrated in U.S. Pat. No. 5,891,874 to Colacino et al.; and plant extracts including but not limited to those shown in U.S. Pat. No. 5,837,257 to Tsai et al., U.S. Pat. No. 5,725,859 to Omer et al., and U.S. Pat. No. 6,056,961.;
(13) Compounds currently in preclinical or clinical development for treatment of hepatitis C virus including but not limited to: Interleukin-IO by Schering-Plough, IP-501 by Intemeuron, Merimebodib (VX497) by Vertex, AMANTADINE® (Symmetrel) by Endo Labs Solvay, HEPTAZYME® by RPI, IDN-6556 by Idun Pharma., XTL-002 by XTL., HCV/MF59 by Chiron, CIVACIR® (Hepatitis C Immune Globulin) by NABI, LEVOVIRIN® by ICN/Ribapharm, VIRAMIDINE® by ICN/Ribapharm, now Valeant, ZADAXIN® (thymosin alpha-1) by Sci Clone, thymosin plus pegylated interferon by Sci Clone, CEPLENE® (histamine dihydrochloride) by Maxim, VX 950/LY 570310 by Vertex/Eli Lilly, ISIS 14803 by Isis Pharmaceutical/Elan, IDN-6556 by Idun Pharmaceuticals, Inc., JTK 003 by AKROS Pharma, BILN-2061 by Boehringer Ingelheim, CellCept (mycophenolate mofetil) by Roche, T67, a β-tubulin inhibitor, by Tularik, a therapeutic vaccine directed to E2 by Innogenetics, FK788 by Fujisawa Healthcare, Inc., IdB 1016 (Siliphos, oral silybin-phosphatdylcholine phytosome), the RNA replication inhibitor VP50406 by ViroPharma/Wyeth, therapeutic vaccines by Intercell and Epimmune/Genencor, IRES inhibitor by Anadys, ANA 245 and ANA 246 by Anadys, immunotherapy “Therapore” by Avant, protease inhibitors by Bristol Myers Squibb/Axys and Corvas/Schering, helicase inhibitor by Vertex, fusion inhibitor by Trimeris, T cell therapy by CellExSys, polymerase inhibitor by Biocryst, targeted RNA chemistry by PTC Therapeutics, Dication by Immtech, Int., protease inhibitors by Agouron and by Chiron/Medivir, antisense therapy by AVI BioPharma and Hybridon, hemopurifier by Aethlon Medical, a therapeutic vaccine by Merix, “Chron-VacC”, a therapeutic vaccine, by Tripep, UT 231 B by United Therapeutics, protease, helicase and polymerase inhibitors by Genelabs Technologies, IRES inhibitors by Immusol, R803 by Rigel Pharmaceuticals, INFERGEN® (interferon alphacon-1) by InterMune, OMNIFERON® (natural interferon) by Viragen, ALBUFERON® by Human Genome Sciences, REBIF® (interferon beta-1a) by Ares-Serono, Omega Interferon by BioMedicine, Oral Interferon Alpha by Amarillo Biosciences, and interferon gamma, interferon tau, and interferon gamma-1b by InterMune; and
(14) Nucleoside prodrugs described for the treatment of other forms of hepatitis, including 2′-deoxy-β-L-nucleosides and their 3′-prodrugs for the treatment of HBV including but not limited to those disclosed in WO 00/09531 and WO 01/96353 to Idenix Pharmaceuticals; and therapeutic esters of acyclovir including but not limited to those shown in U.S. Pat. No. 4,957,924 to Beauchamp.

Any of the viral treatments described herein can be used in combination or alternation with the compounds described in this specification. Nonlimiting examples include:
1) Protease inhibitor;
2) Thiazolidine derivative;
3) Helicase inhibitor;
4) Benzanilide;
5) Phenanthrenequinone;
6) Polymerase inhibitors or gliotoxin;
7) Antisense phosphorothioate oligodeoxynucleotide (S-ODN);
8) Inhibitors of IRES-dependent translation;
9) Ribozyme;
10) Nucleoside analogue;
11) Disubstituted nucleoside analogues, as described by Idenix Pharmaceuticals in WO 01/90121 and WO 01/92282;
12) 2′-fluoronucleoside analogues;
13) 1-amino-alkylcyclohexanes;
14) Alkyl lipids;
15) Vitamin E and other antioxidants;
16) Squalene, amantadine and bile acids;
17) N-(phosphonoacetyl)-L-aspartic acid;
18) Benzenedicarboxamides;
19) Polyadenylic acid derivatives;
20) Benzimidazoles;
21)2′, 3′-dideoxyinosine;
22) Plant extracts;
23) Piperidines; and
   V. Pharmaceutical Compositions

Hosts, including humans, infected with Flaviviridae virus, including pestivirus, flavivirus, HCV infection, or any other condition described herein, or another organism replicating through a RNA-dependent RNA viral polymerase, or for treating any other disorder described herein, can be treated by administering to the patient an effective amount of the active compound or a pharmaceutically acceptable salt, ester or prodrug, thereof in the presence of a pharmaceutically acceptable carrier or dilutent. The active materials can be administered by any appropriate route, for example, orally, parenterally, intravenously, intradermally, subcutaneously, or topically, in liquid or solid form.

A typical dose of the compound for Flaviviridae virus, including pestivirus, flavivirus or HCV will be in the range from about 1 to 50 mg/kg, or 1 to 20 mg/kg, of body weight per day, more generally 0.1 to about 100 mg per kilogram body weight of the recipient per day. Lower doses are contemplated, for example doses of0.5-100 mg, 0.5-50 mg, 0.5-10 mg, or 0.5-5 mg per kilogram body weight per day. Even lower doses may be useful, and thus ranges can include from 0.1-0.5 mg per kilogram body weight per day. The effective dosage range of the pharmaceutically acceptable salts, esters and prodrugs can be calculated based on the weight of the parent nucleoside to be delivered. If the salt, ester or prodrug exhibits activity in itself, the effective dosage can be estimated as above using the weight of the salt, ester or prodrug, or by other means known to those skilled in the art.

The compound is conveniently administered in unit any suitable dosage form, including but not limited to one containing 7 to 3000 mg, such as 70 to 1400 mg of active ingredient per unit dosage form. An oral dosage of 50-1000 mg is usually convenient, including in one or multiple dosage forms of 50, 100, 200, 250, 300, 400, 500, 600, 700, 800, 900 or 1000 mgs. Lower doses, for example from 10-100 or 1-50 mg, are contemplated. Also contemplated are doses of 0.1-50 mg, or 0.1-20 mg or 0.1-10.0 mg. Furthermore, lower doses may be utilized in the case of administration by a non-oral route, as, for example, by injection or inhalation.

Ideally the active ingredient should be administered to achieve peak plasma concentrations of the active compound of from about 0.2 to 70 μM, including about 1.0 to 10 μM. This may be achieved, for example, by the intravenous injection of a 0.1 to 5% solution of the active ingredient, optionally in saline, or administered as a bolus of the active ingredient.

The concentration of active compound in the drug composition will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

A mode of administration of the active compound is oral. Oral compositions will generally include an inert diluent or an edible carrier. They may be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can e included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

The compound can be administered as a component of an elixir, suspension, syrup, wafer, chewing gum or the like. A syrup may contain, in addition to the active compounds, sucrose as a sweetening agent and certain preservatives, dyes and colorings and flavors.

The compound or a pharmaceutically acceptable salt, ester or prodrug thereof can also be mixed with other active materials that do not impair the desired action, or with materials that supplement the desired action, such as antibiotics, antifungals, anti-inflammatories, or other antivirals, including other nucleoside compounds. Solutions or suspensions used for parenteral, intradermal, sucutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, carriers can be physiological saline or phosphate buffered saline (PBS).

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters and polylactic acid. Methods for preparation of such formulations will be apparent to those skilled in the art. The materials can also be obtained commercially from Alza Corporation.

Liposomal suspensions (including liposomes targeted to infected cells with monoclonal antibodies to viral antigens) are also typical as pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound or its monophosphate, diphosphate, and/or triphosphate derivatives is then introduced into the container. The container is then swirled by hand to free lipid material from the sides of the container and to disperse lipid aggregates, thereby forming the liposomal suspension.

VI Processes for the Preparation of Active Compounds

The nucleoside analogues of the present invention can be synthesized by any means known in the art. The following non-limiting embodiments illustrate methodology to obtain the nucleoside analogues of the present invention.

The following reference provides exemplary information pertaining to possible nonlimiting routes of synethesis of the described compounds: Dounay, A. B., et al., Expedient Access to the Okadaic Acid Architecture: A Novel Synthesis of the C1-C27 Domain. J. Org. Chem., 2001. 66(3): p. 925-938.

A. Synthetic Schemes

I. Synthetic Methods for thePreparation of Compounds of the General Formulae I, III and IV:

Approach A

General Route A: 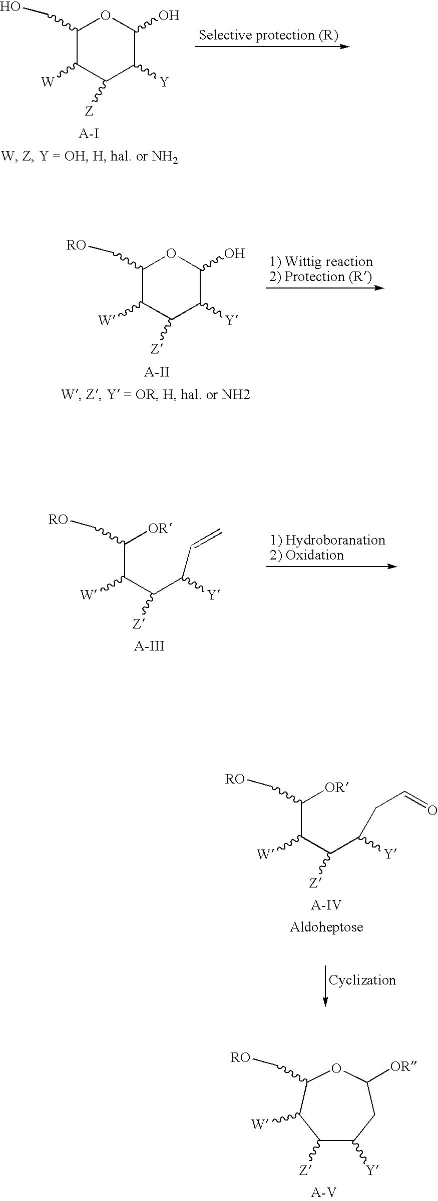
A six-membered ring sugar I is protected by known protecting groups to afford compound II. Extension of the sugar II is carried out by a Wittig reaction followed by suitable protection. Hydroboranation of compound III followed by an oxidation step gives an aldoheptose IV. Cyclization of the latter gave a seven membered ring sugar V. 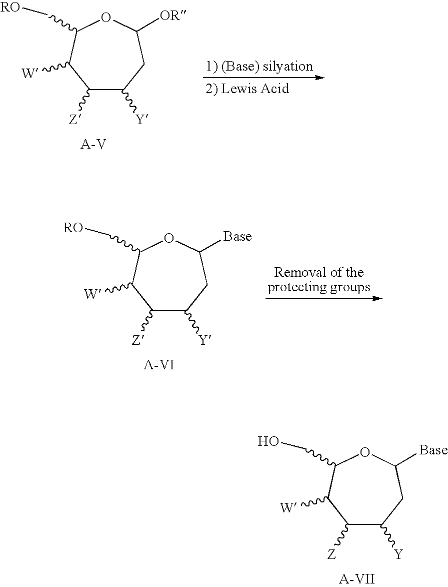
Coupling step between seven membered ring sugar V and silylated heterobase in presence of a Lewis acid gives a protected seven membered ring nucleoside VI.

Removal of protecting groups afforded the desired seven membered ring nucleoside VII. 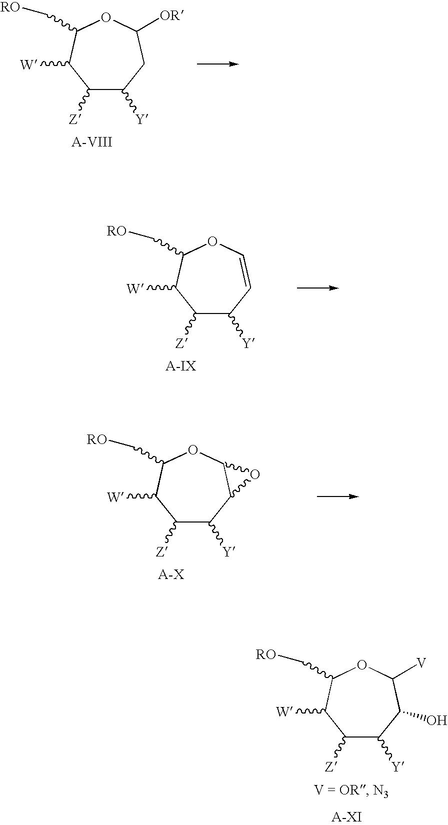
Action of a Lewis acid on A-VIII affords a seven membered glycal sugar A-IX. Epoxidation followed by treatment with alcohols (R″OH, R″=alkyl, Ar) or azide ion gave compound A-XI. 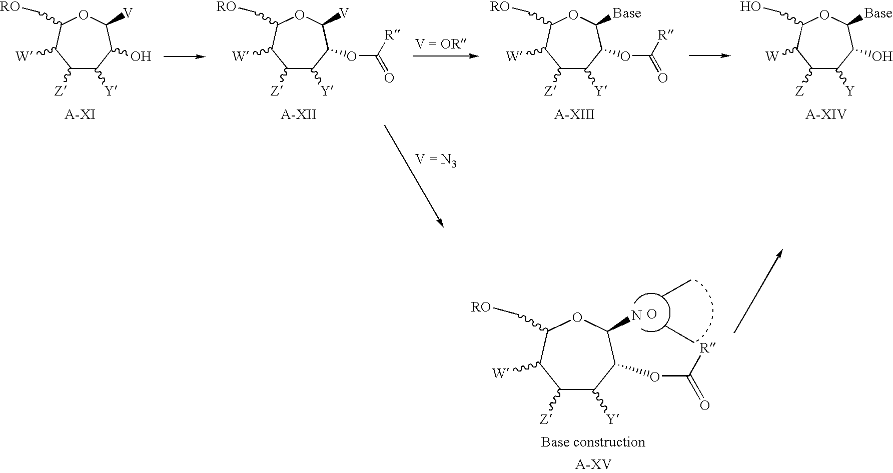
Protection of compound A-XI with an acyl group afforded compound A-XII with a participating at 2′ position. Coupling step with heterobases using known conditions gives the nucleoside A-XIII and subsequent removal of the protecting groups gives the desired seven membered ring nucleoside. Base construction from the azido derivative A-XII (V=N₃) affords compound A-XV. Removal of protecting groups leads to the seven membered ring nucleoside A-XIV.
Approach B

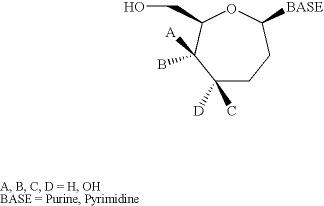

General Route B: 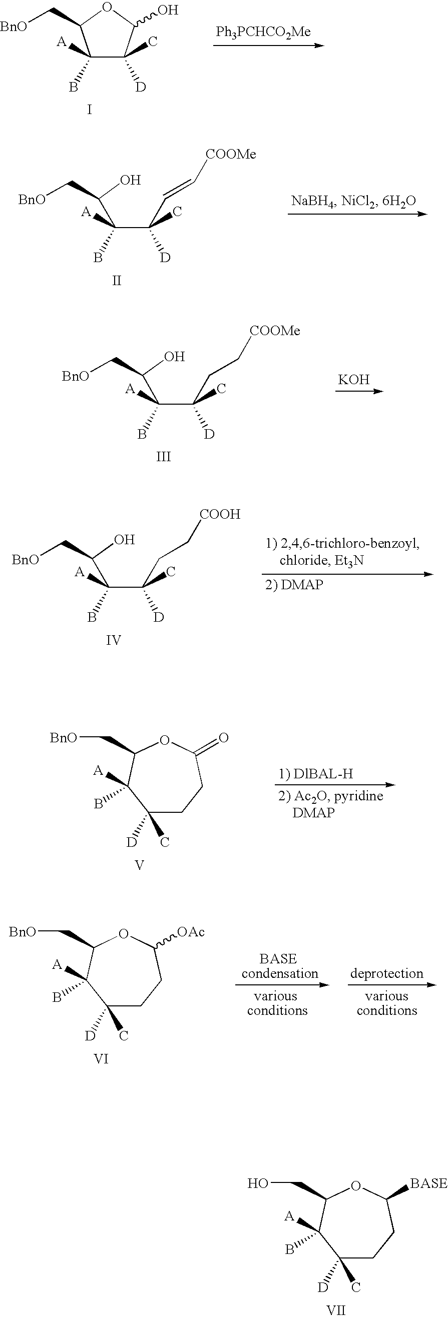
This route commences with a Wittig reaction on tribenzylated sugar I to get II. After reduction of the double bond, ester III is obtained and leads to lactone V via acid IV. After reduction and acetylation, acetate VI is obtained. The latter potentially leads to various nucleosides of general structure VII after condensation with a purine/pyrimidine base and deprotection.
Approach C:

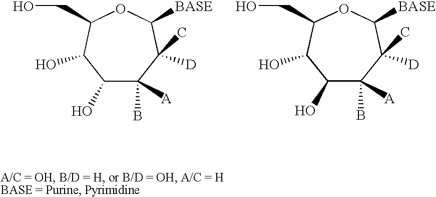

General Route C: 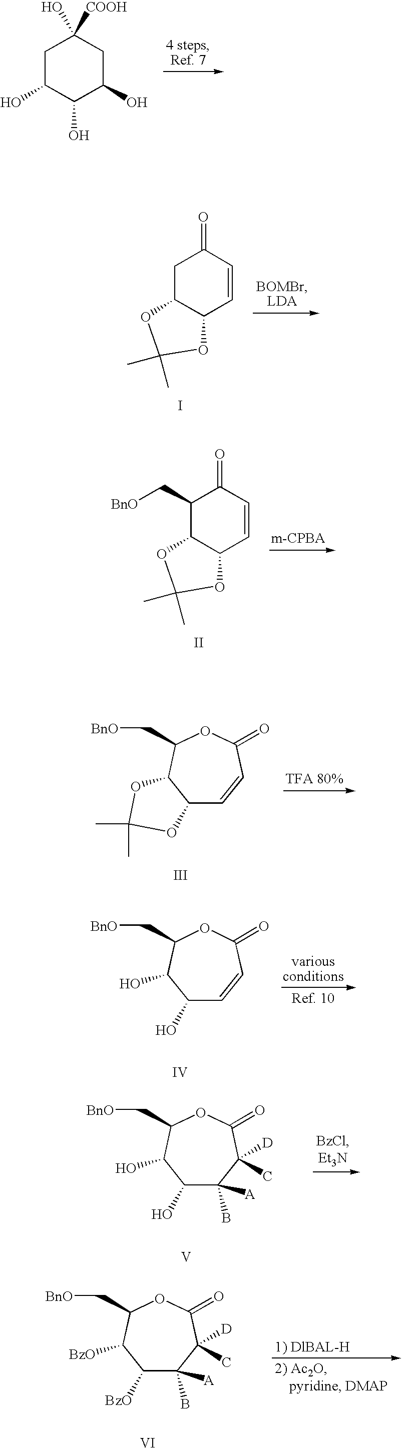 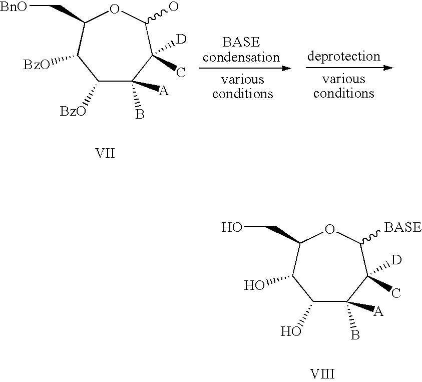
This route commences with commercially available (−)-quinic acid that is converted in α,β-unsaturated ketone I in 4 steps according to Barros, M. T.; Maycock, C. D.; Ventura, M. R. J. Org. Chem., 1997, 62, 3984-3988. The α-alkylation step on the less hindered face of I with benzyloxymethyl bromide to give II is adapted from Gosselin, G.; Griffe, L.; Meillon, J.-C.; Storer, R. Tetrahedron, 2006, 62, 906-914. A Baeyer-Villiger oxidation on II leads to ketone III with the good regiochemistry according to the observations made in Floresca, R.; Kurihara, M.; Watt, D. S.; Demir, A. J. Org. Chem., 1997, 62, 2196-2200. Removal of the acetonide group gives IV, which is submitted to osmium-promoted dihydroxylation to give V. Depending on the conditions, the dihydroxylation takes place in syn or anti fashion with respect to the hydroxyl neighbors (Donohoe, T. J.; Blades, K.; Moore, P. R.; Waring, M. J.; Winter, J. J. G.; Helliwell, M.; Newcombe, N. J.; Stemp, G. J. Org. Chem., 2002, 67, 7946-7956). Perbenzoylation of V gives lactone VI that is reduced and acetylated to obtain VII following the conditions given in Rychnovsky, S. D.; Dahanukar, V. H. J. Org.

Chem., 1996, 61, 7648-7649. The latter potentially leads to various nucleosides of general structure VIH after condensation with a purine/pyrimidine base and deprotection.
Approach D: 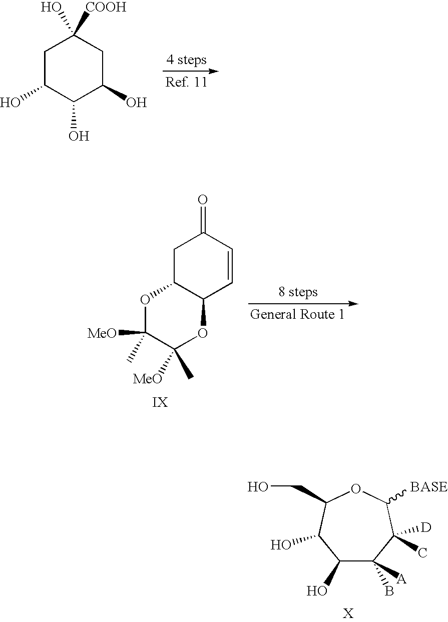
General Route D:

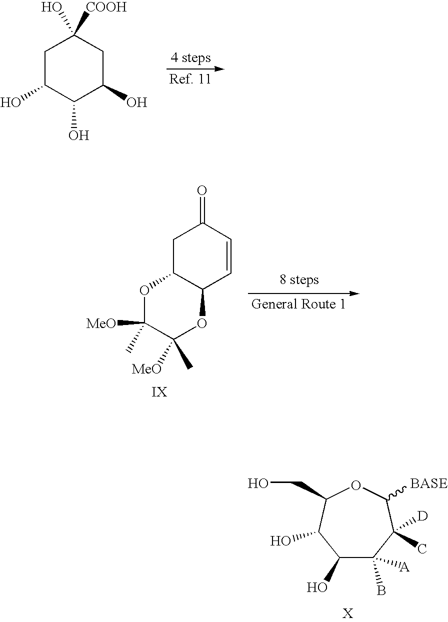

α,β-Unsaturated ketone IX is prepared in 4 steps from (−)-quinic acid according to Murray, L. M.; O'Brien, P.; Taylor, R. J. K. Org. Lett., 2003, 5, 1943-1946. The rest of the synthesis is identical to General Route 1* and can lead to nucleosides of general structure X.
Note: “General Route” and “General Route 1” are identical to the “General Route” given under Approach C above.
   Approach E:
   General Synthesis of 7-membered Ring Sugar Nucleosides
   7-membered ring sugar nucleosides of the following structures: 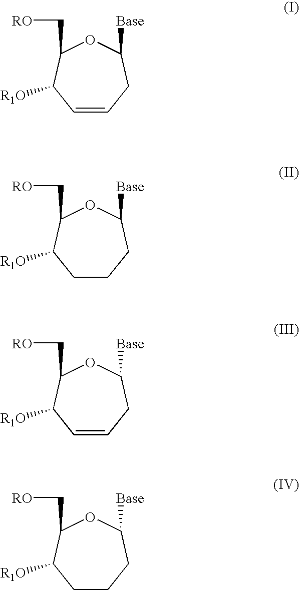
   wherein R, R₁ and Base are all as described above, can be prepared according to the following general methods.

7-membered ring sugar nucleosides may be prepared according to a ring expansion of cyclopropanated sugar 1. This compound is prepared from D-Glucal according to the procedures provided in Journal of Organic Chemistry, 1997, 62, 19, 6615-6618 and in Carbohydrate Research, 1997, 300, 365-367. The 4,6-O-(di-tert-butylsilanediyl)-D-glucal 1 was treated with a silylated Base using TMSOTf as catalyst. This reaction involves loss of acetate and ring expansion to give the seven-membered ring sugar nucleosides 2 according to the procedure provided in Tetrahedron Letters, 2003, 44, 9043-9045. Deprotection of the compound 2 is achieved using TBAF in THF to produce the nucleoside 3. Hydrogenation of 3 leads to the nucleoside analogues 4. Treatment of 3 and 4 by protecting groups furnishes the protected 7-membered ring sugar nucleosides I, H, III and IV. 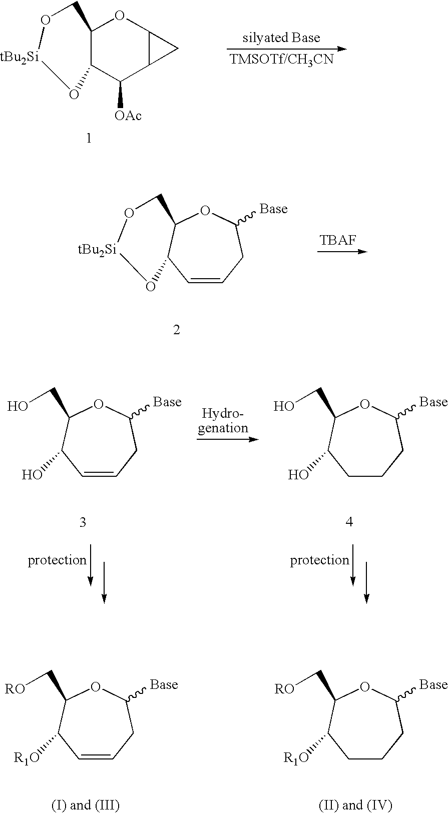
Approach F:
General Synthesis of 7-membered Ring Sugar Nucleosides from D-gulo Heptonolactone
Illustration 1:

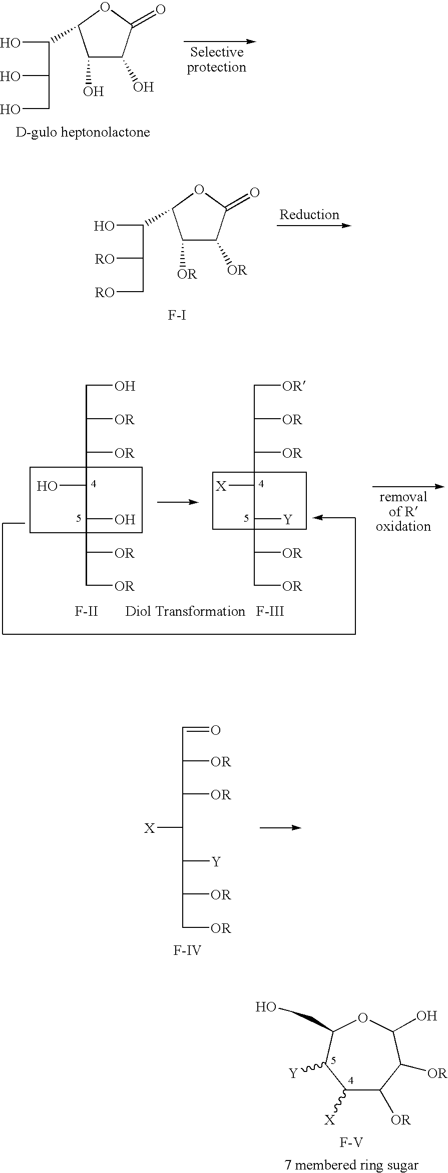

D-guloheptonolactone is selectively protected (F-I) and then totally reduced into its linear form F-I1. The remaing primary alcohol of compound F-II is protected (R′ protecting group) and the diol bearing by the carbons 4 and 5 is converted into compound F-III. Removal of the protecting group R′ followed by oxidation step gives compound F-IV. Acidic treatment of compound F-IV gives the seven membered ring sugar F-V.
Illustration2: some (C4-X;C5-Y)

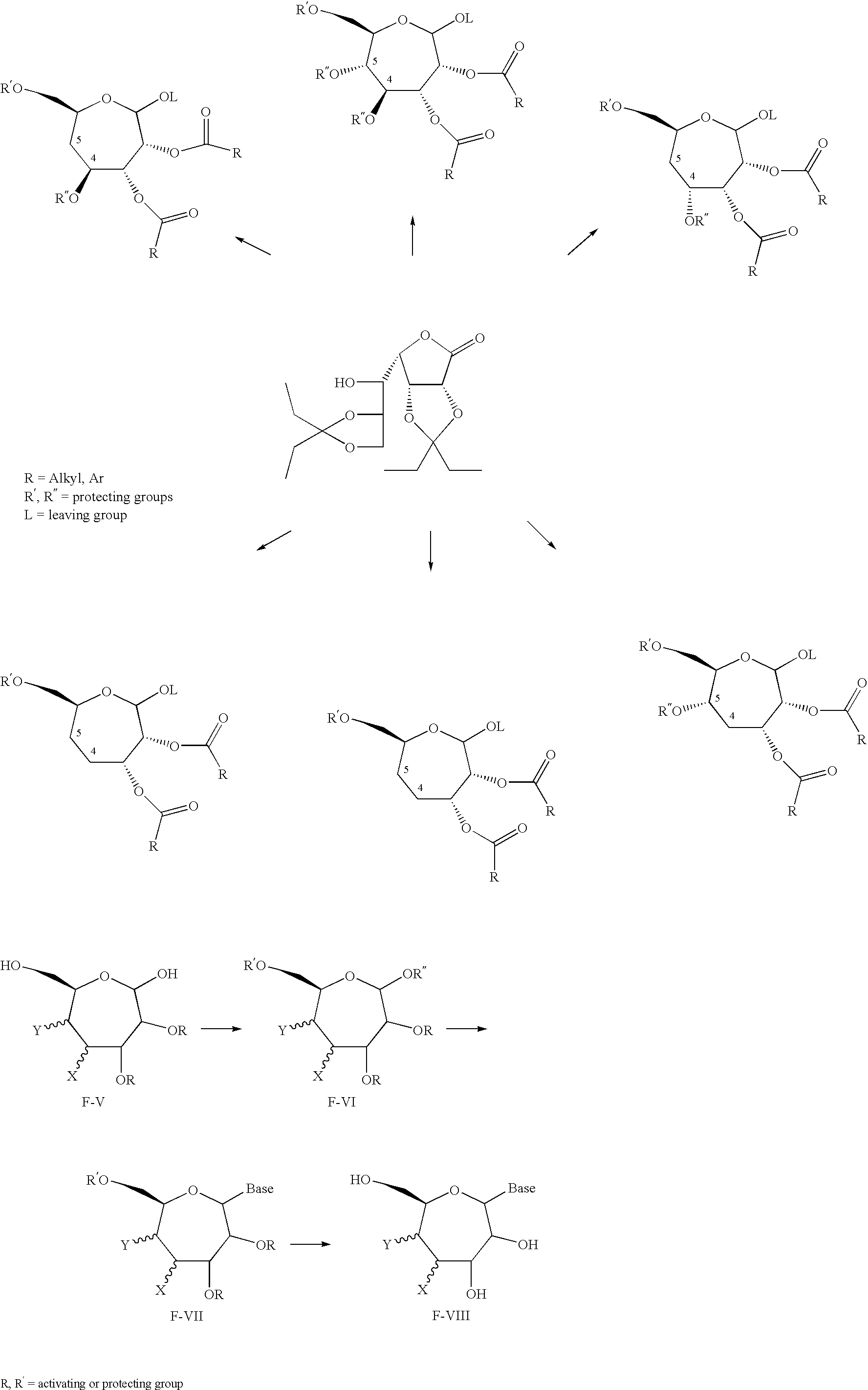

The primary hydroxyl function is selectively protected (R′) and the anomeric position is activated with a leaving group (R″) to afford compound F-VI. Coupling of the latter with silylated heterobases gives compound F-VII. Removal of the protecting groups leads to the seven membered ring nucleoside F-VIII.
Approach F′: 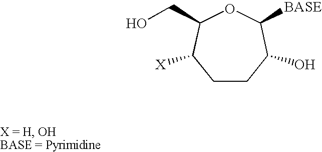
General Route F-I: 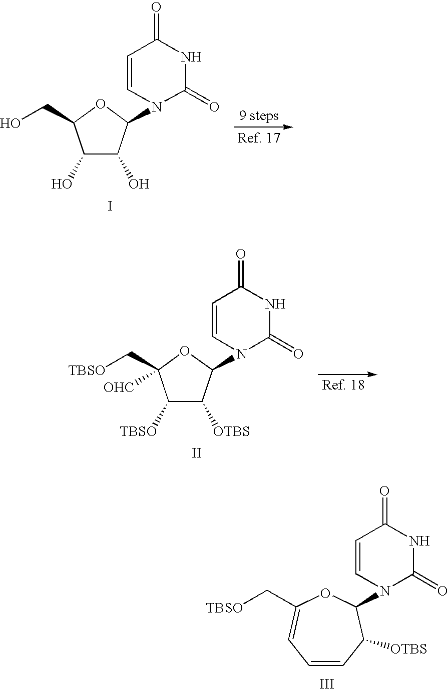
Uridine I is converted in 9 steps into nucleoside II according to Nomura, M.; Shuto, S.; Tanaka, M.; Sasaki, T.; Mori, S.; Shigeta, S.; Matsuda, A. J. Med. Chem., 1999, 42, 2901-2908. The preparation of III in 1 step from II is described in Nomura, M.; Endo, K.; Shuto, S.; Matsuda, A. Tetrahedron, 1999, 55, 14847-14854.
General Route F-II: 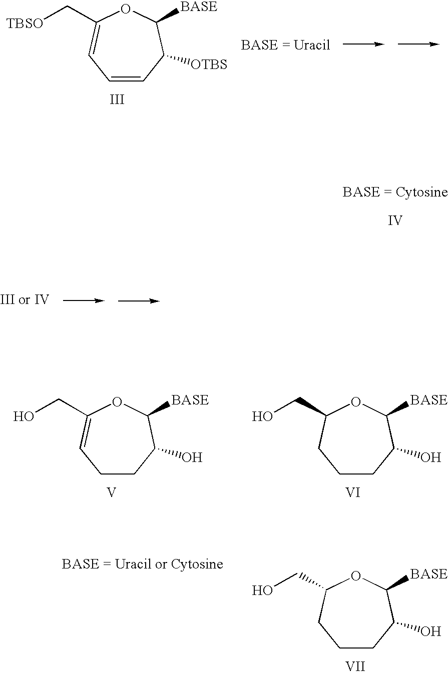
The nucleobase of III can be converted to cyctosine according to Miah, A.; Reese, C.

B.; Song, Q.; Sturdy, Z.; Neidle, S.; Simpson, I. J.; Read, M.; Rayner, E. J. Chem. 10 Soc., Perkin Trans. I, 1988, 3277-3284. Either III or IV can be conver ted to nucleosides V-VII by deprotection followed by a partial or total hydrogenation.
General Route F-III: 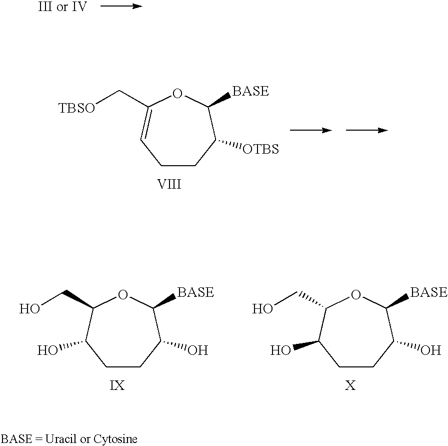
Selective reduction of either IHI or IV leads to VIII. The latter is submitted to hydroboration/oxidation followed by deprotection conditions to get IX and X.
II. Synthetic Methods for the Preparation of Compounds of the General Formulae II.
Approach G
General Route G-I:

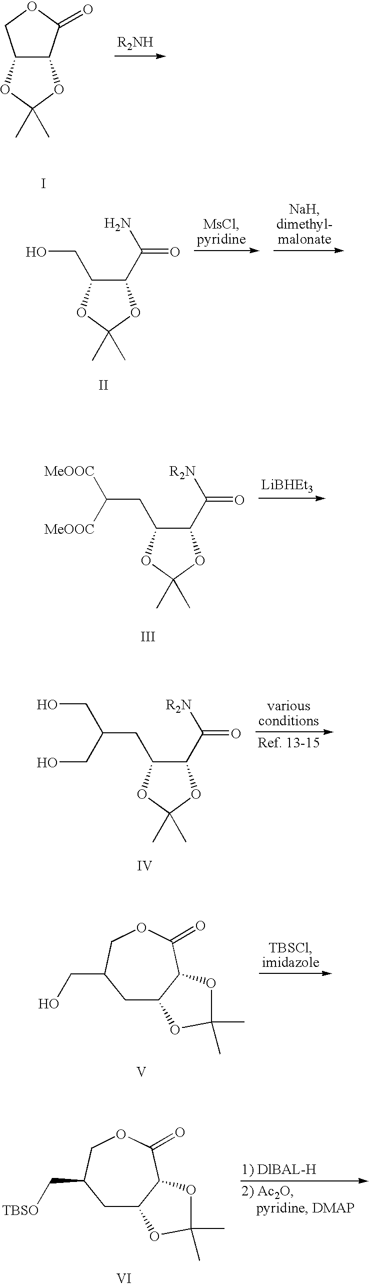

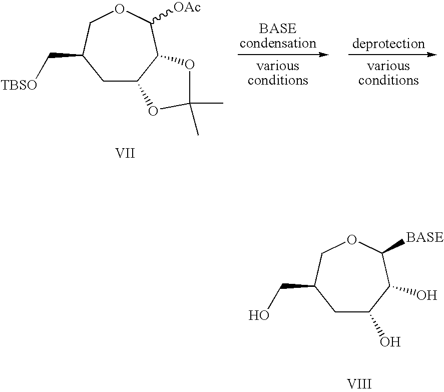

The synthesis commences with commercially available 3,4-O-isopropylidene-(D)-erythronolactone I that is opened with an amine to get amide II following the conditions given in reference 12. The primary alcohol is then activated and substituted with a dimethylmalonate salt to obtain III. The latter is reduced to diol IV, which is submitted to the conditions described in references 13-15 to produce lactone V as a mixture of epimers. The primary alcohol of V is protected and the epimers separated to give VI, which is then reduced and acetylated to give VII following the conditions given in Rychnovsky, S. D.; Dahanukar, V. H. J. Org. Chem., 1996, 61, 7648-7649. The latter potentially leads to various nucleosides of general structure VIII after condensation with a purine/pyrimidine base and deprotection.
General Route G-II:

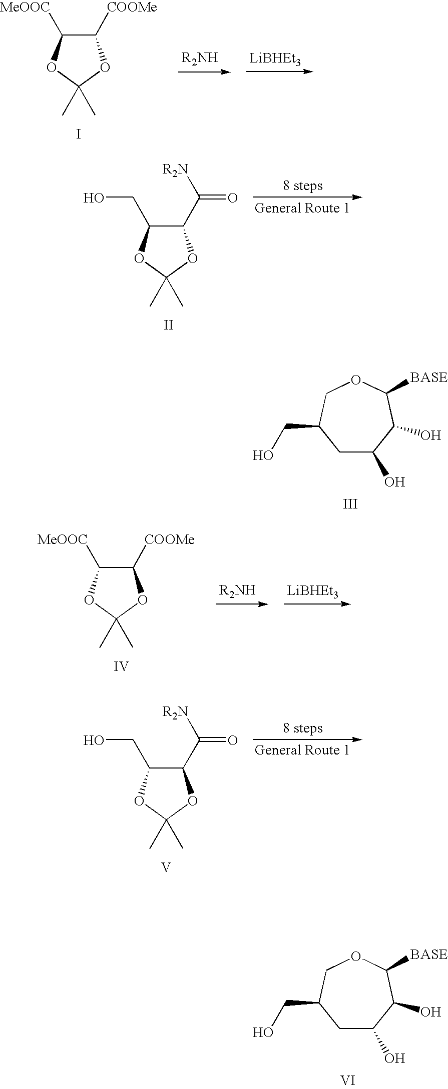

The routes commences with a monoamidation of 2,3-O-isopropylidene-(L)-dimethyl tartrate I, followed by a reduction of the remaining ester to get amide II. The rest of the synthesis is identical to General Route 1 and potentially leads to nucleosides of general structure III. When 2,3-O-isopropylidene-(D)-dimethyl tartrate IV is used as the starting material, nucleosides of general structure VI are obtained.
Approach H 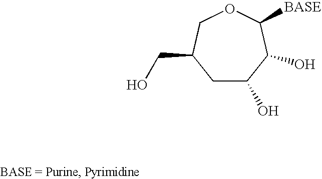
General Route H:

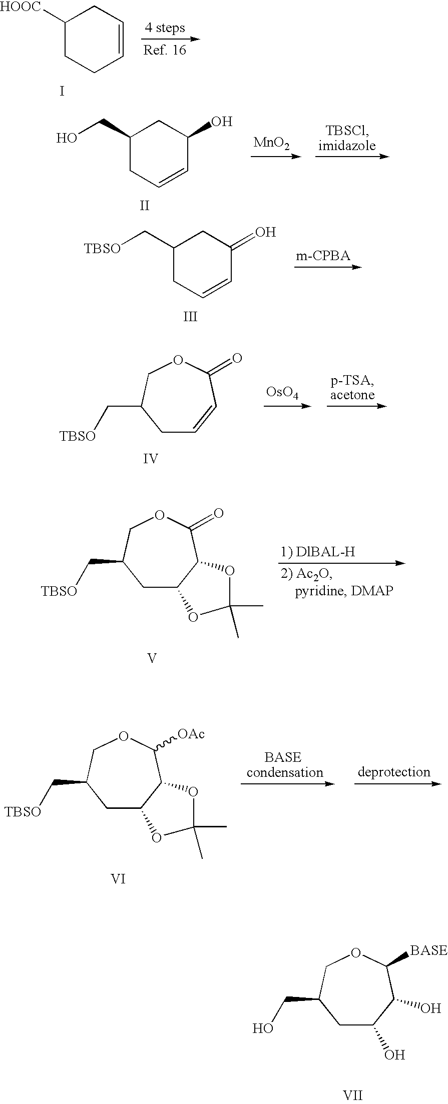

Commercially available acid I is converted in 4 steps to diol H according to reference 16. Oxidation of the allylic alcohol and protection of the primary alcohol leads to ketone III. A Baeyer-Villiger oxidation gives lactone IV, which is dihydroxylated on its less hindered face in presence of osmium(IV) and protected as an acetonide to obtain V. The lactone is then reduced and acetylated to get VI following the conditions given in Rychnovsky, S. D.; Dahanukar, V. H. J. Org. Chem., 1996, 61, 7648-7649. The latter potentially leads to various nucleosides of general structure VII after condensation with a purine/pyrimidine base and deprotection.

Approach I

Apio-7-membered Ring Sugar Nucleosides of the Following Structures: 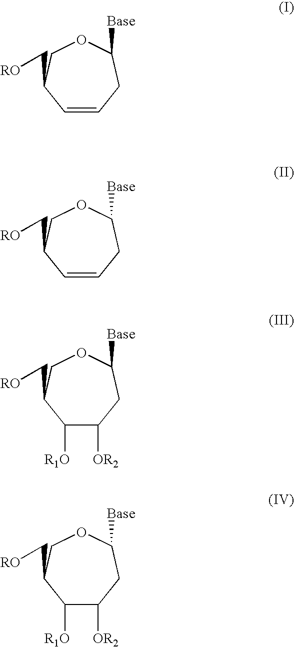
wherein R, R₁ and R₂ and Base are all as described above, can be prepared according to the following general methods.

The apio-7-membered ring sugar nucleosides I and II may be prepared according to the following synthesis (scheme 1) using compound 1 as starting material. This compound is prepared according to the procedure provided in 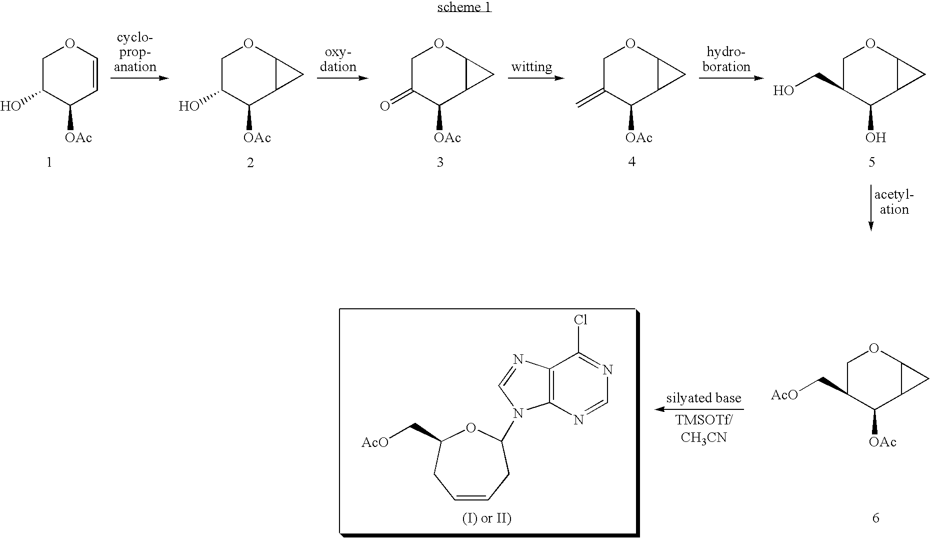
The apio-7-membered ring sugar nucleosides III and IV may be prepared according to the following synthesis (schema 1): 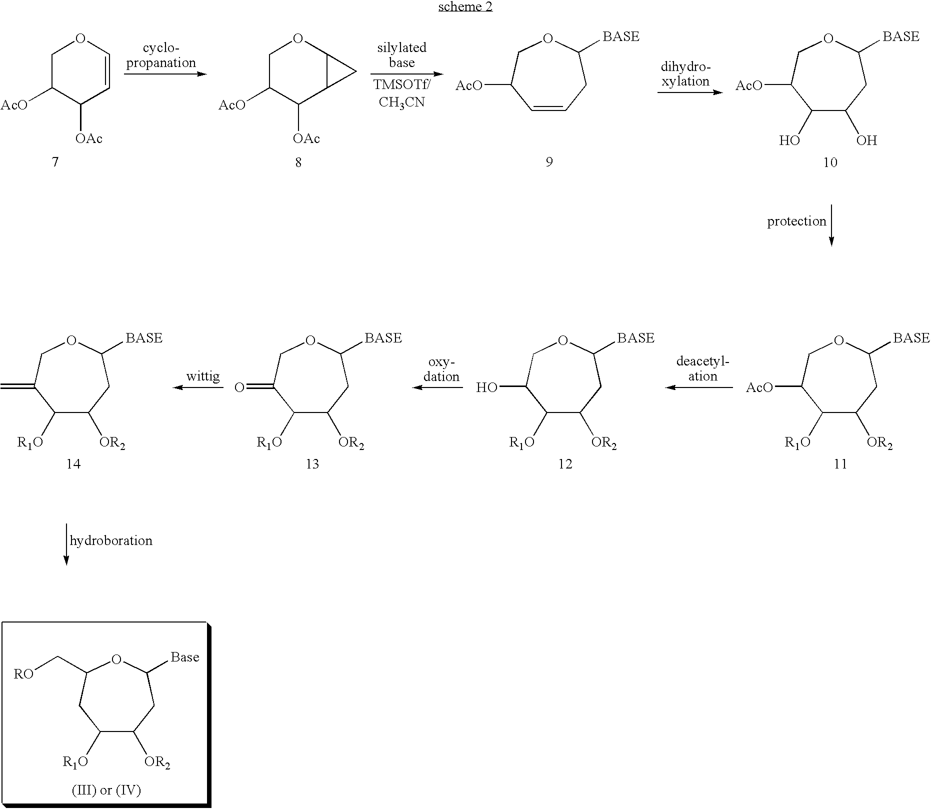
I.a. Prior Art SugarSyntheses
Prior Art Starting Material Sugars:
Sugars 1/1a/1b/1c/1e/2a/2b: 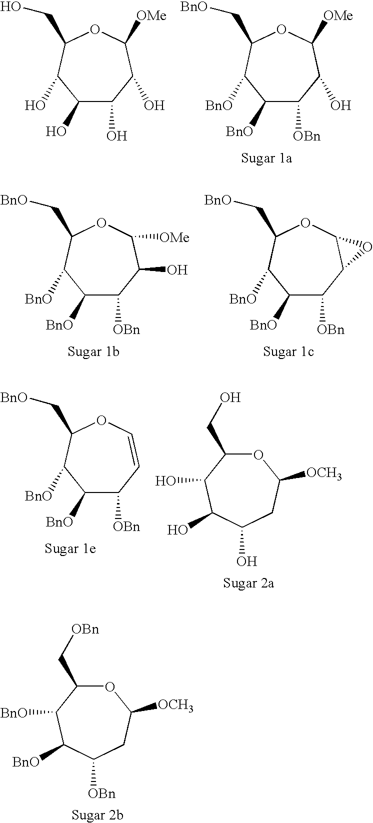 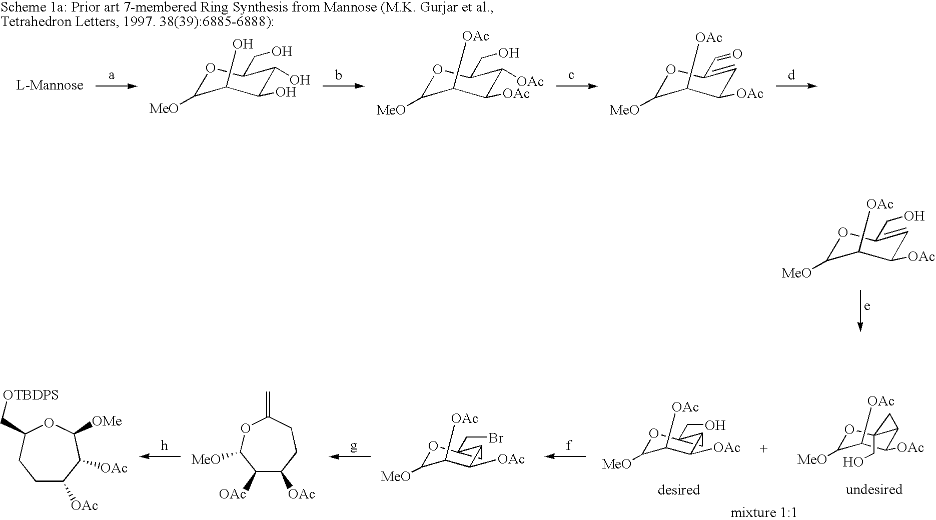
Conditions and reagents: (a) HCl, MeOH; (b) i. TrCl, Pyridine, 100° C., 3 h then
   R.T. overnight; ii. Ac₂O, 24h, R.T; iii. Silica gel [Gros, E. G. and E. M. GruNeiro, Methylation of carbohydrates bearing base-labile substituents, with diazomethane-boron trifluoride etherate: Part IV. An improved procedure for the synthesis of 6-O-methyl-mannose. Carbohydrate Research, 1970. 14(3): p. 409-411]; (c) PCC, Toluene, Δ, 1 h (70%); (d) NaBH₄, IR 120(H+) resin, MeOH, 0° C., 5 min. (85%); (e) CH₂I₂, Et₂Zn, CH₂Cl₂, −20° C., 10 h (74%); (f) Ph₃P, CBr₄, Imid., CH₂ClR.T., 1 h (92%); (g) Bu₃SnH, AIBN, Toluene, Δ, 30 min. (37%); (h) i. 9-BBN, THF, 4 h, 1M NaOAc, H₂O₂, 0° C., 3 h; ii. TBDPSCI, Imid., DMAP, CH₂Cl₂, R.T., 4 h. 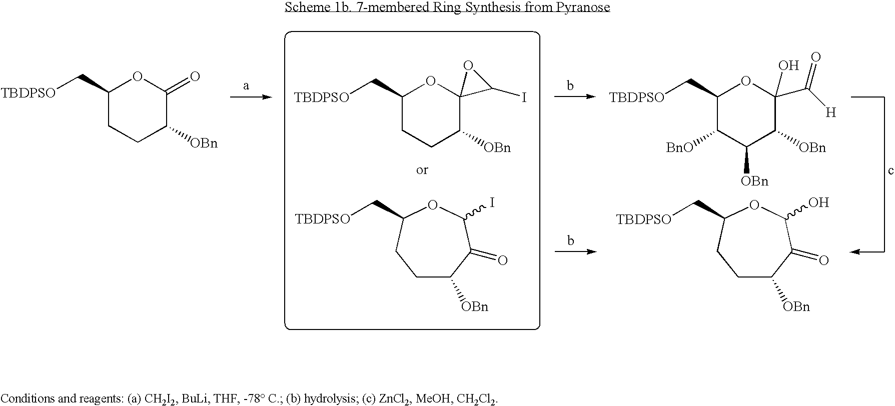 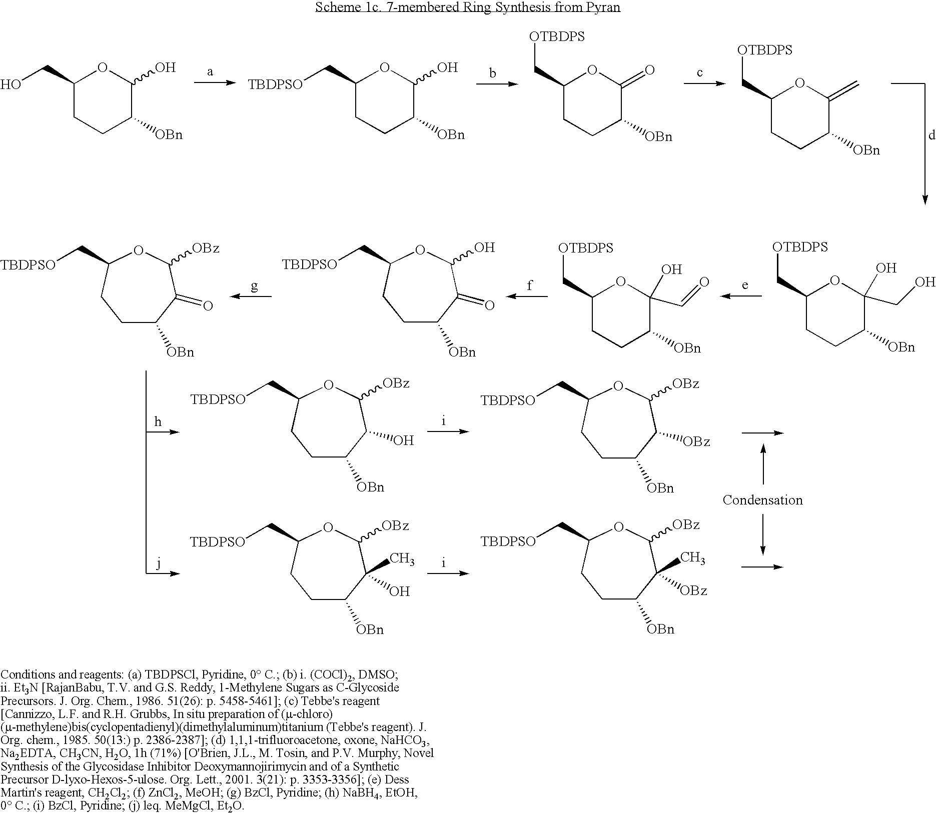
   [Isobe, Minoru, et al., Synthetic studies toward marine toxic polyethers (1) a stereocontrolled synthesis of the C-segnent of okadaic acid. Tetrahedron Letters, 1984. 25(33): p. 3607-3610]; (c) i. LiA1H₄; ii. H₂/Pd-C; iii. TBDPSCI, Pyridine; iv. BnBr [Isobe, Minoru, Y. Ichikawa, and T. Goto, Synthetic studies toward marine toxic polyethers (3) stereocontrol for segment-A1 of okadaic acid by means of oxymercuration and epoxydation. Tetrahedron Letters, 1985. 26(42): p. 5199-5202]; (d) i. I₂/aq. NaHCO₃; ii. PCC, CH₂Cl₂ (61%) [Sabes, S. F., R. A. Urbanek, and C. J. Forsyth, Efficient Synthesis of Okadaic Acid. 2. Synthesis of the C1-C14 Domain and Completion of the Total Synthesis. J. Am. Chem. Soc., 1998. 120(11): p. 2534-2542].
   II. Sugar Syntheses 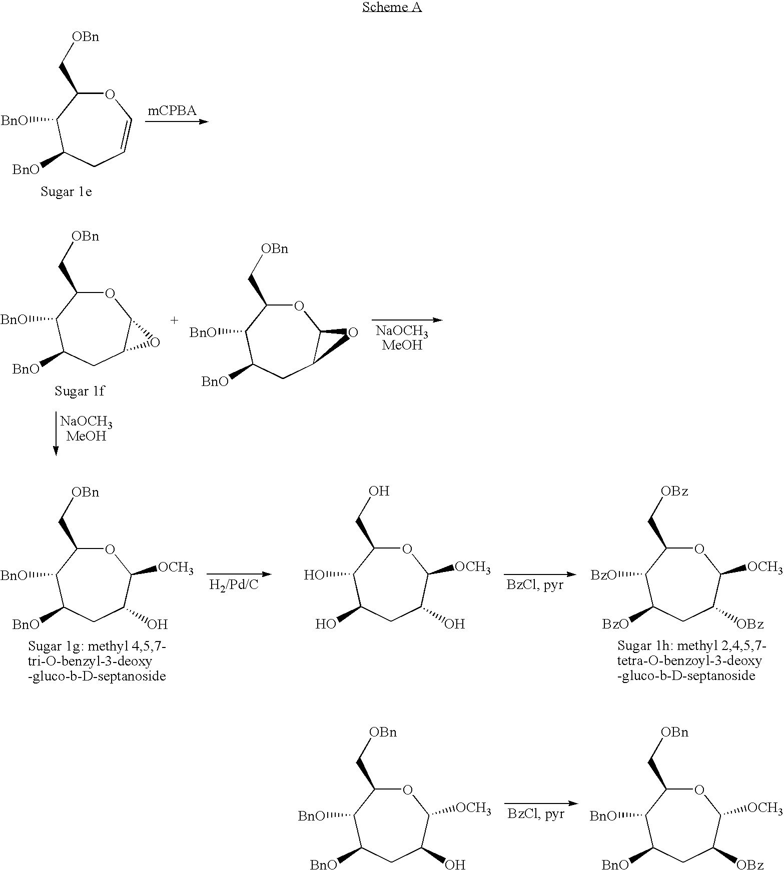 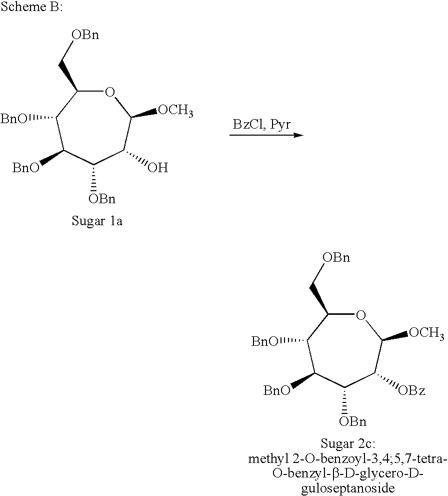 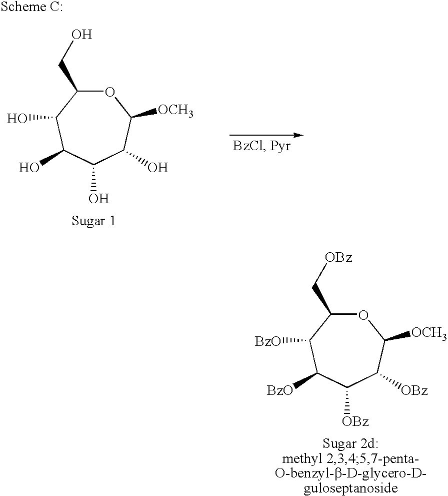 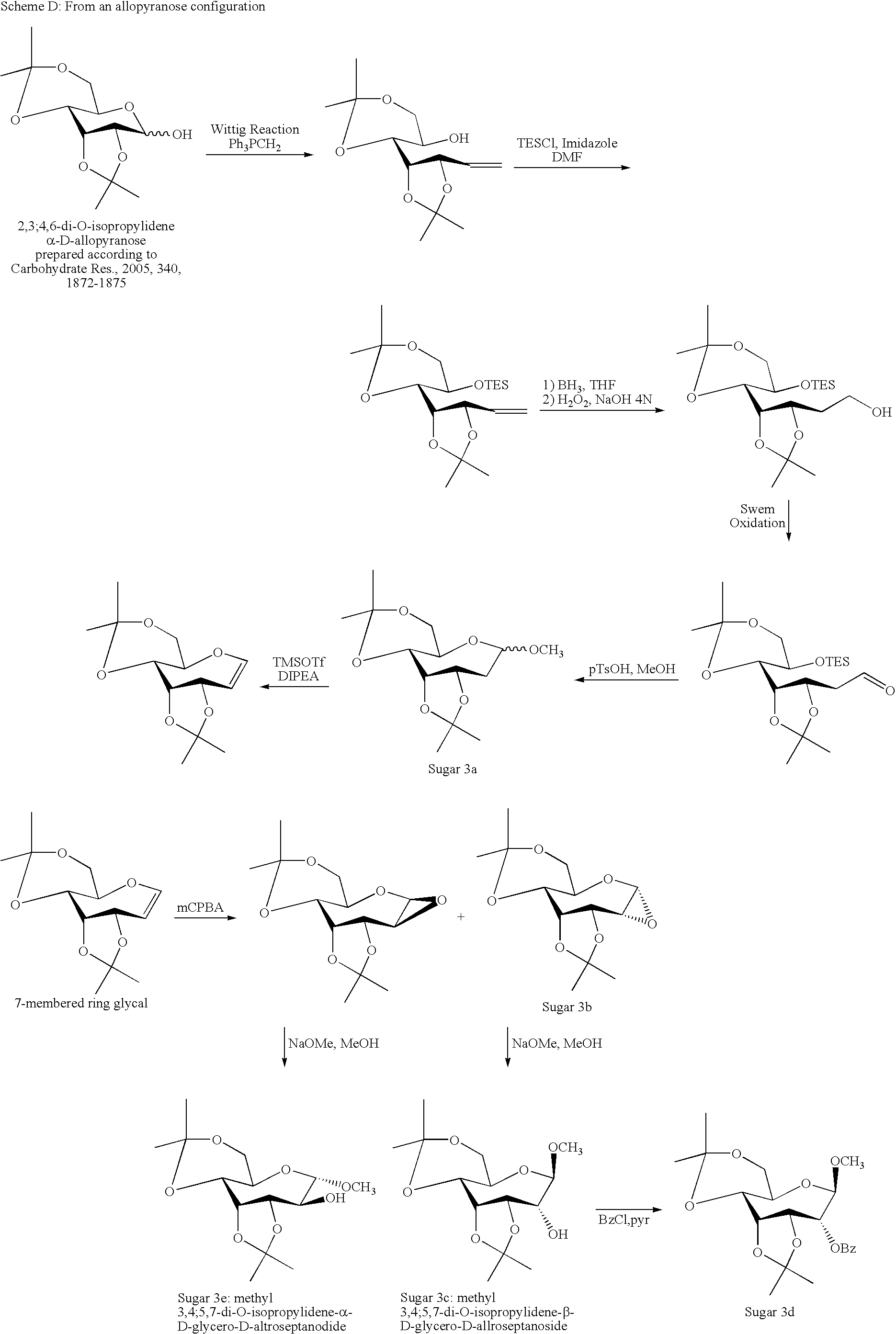 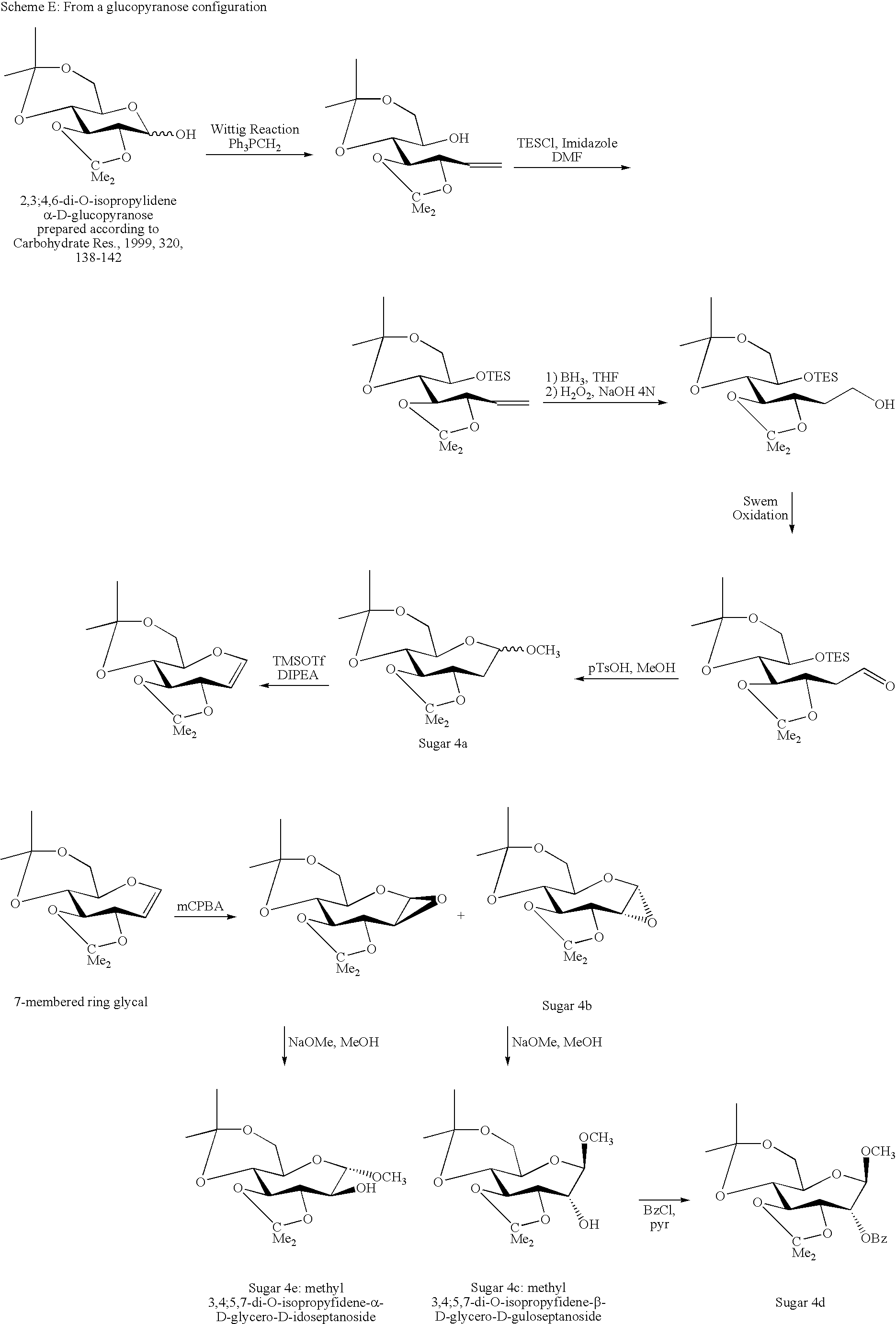 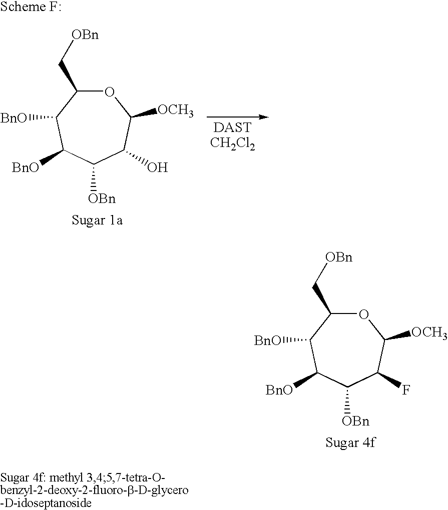 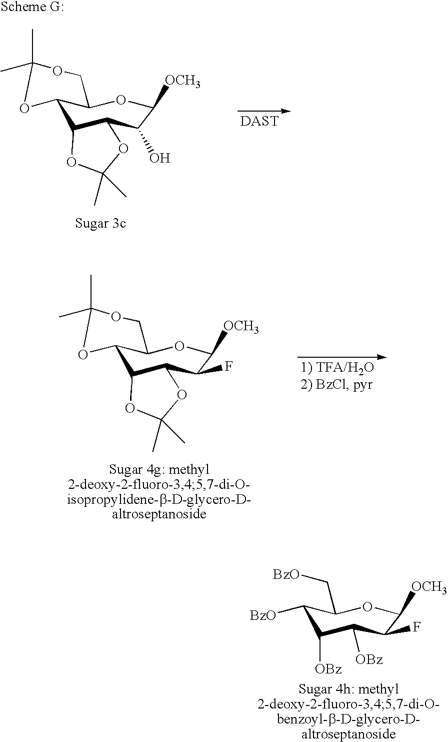 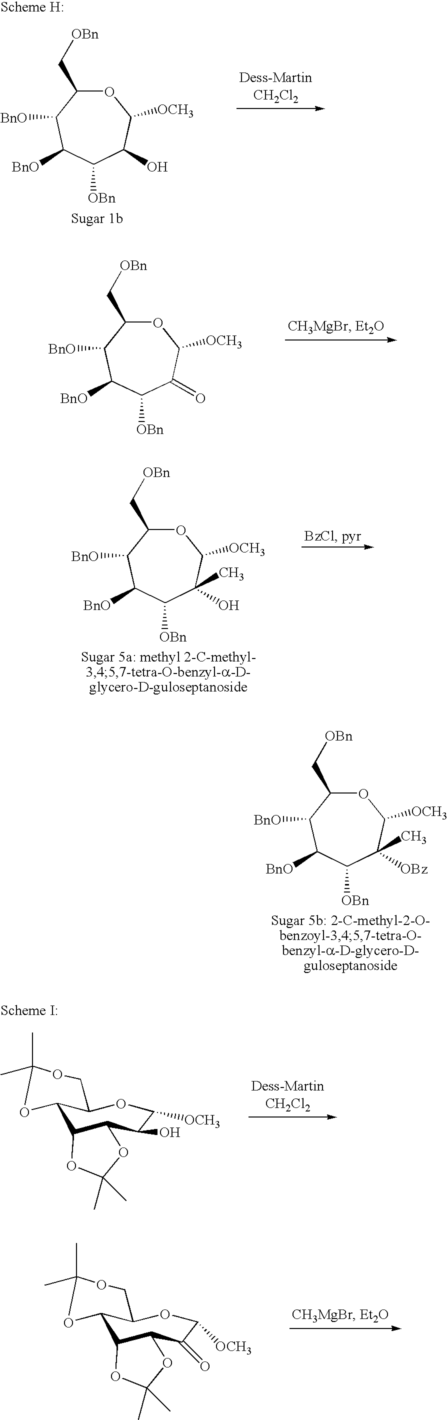 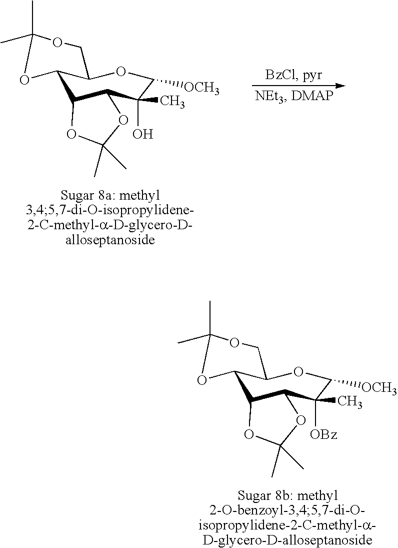
   Schematics of Nucleoside Analogue Syrntheses: 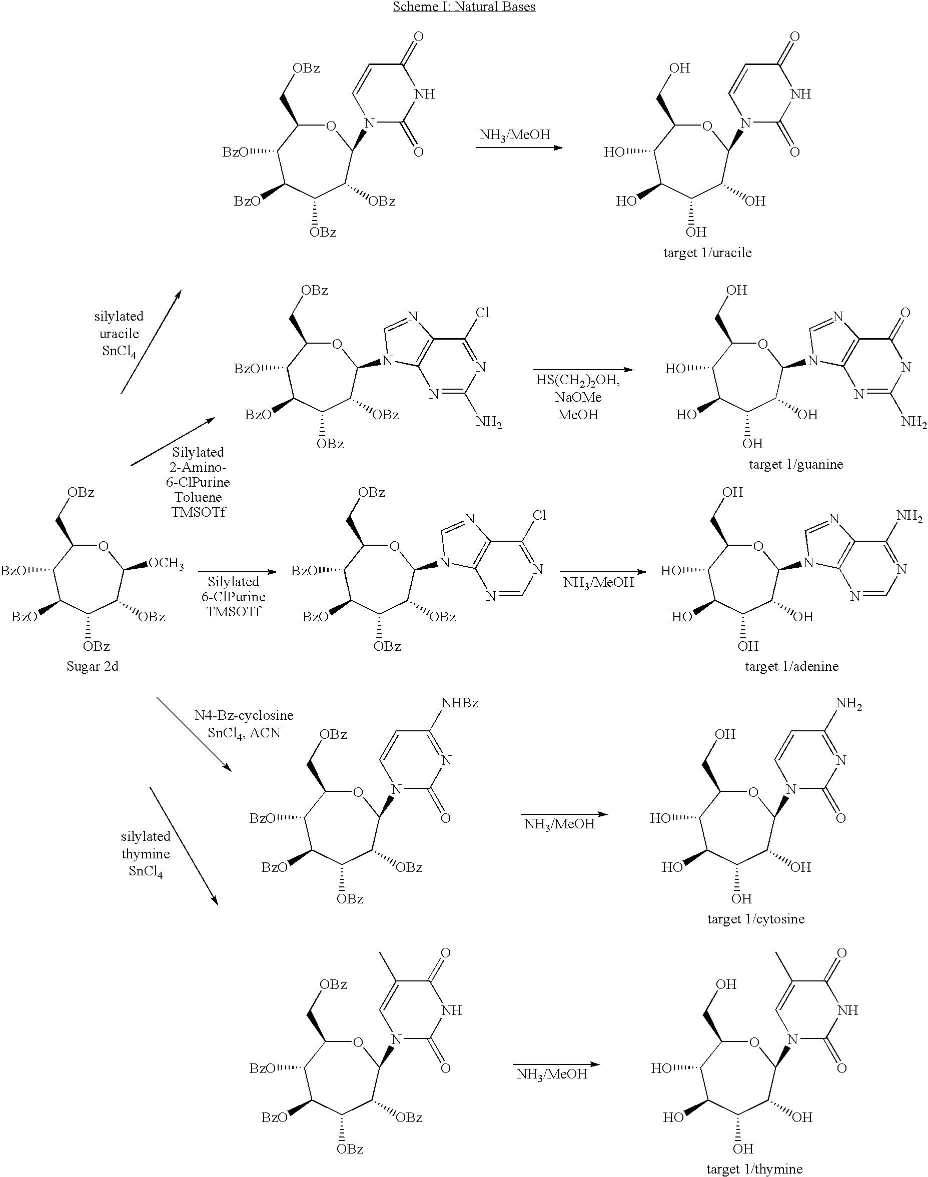 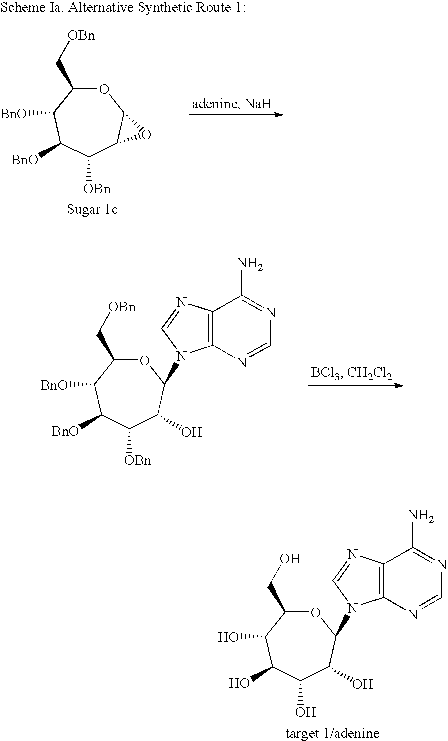 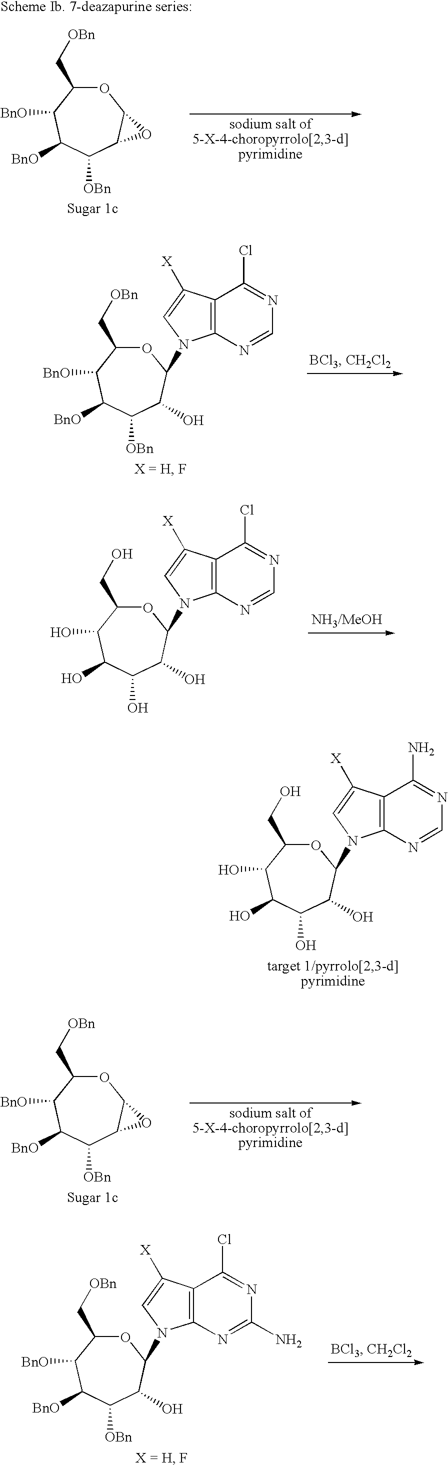 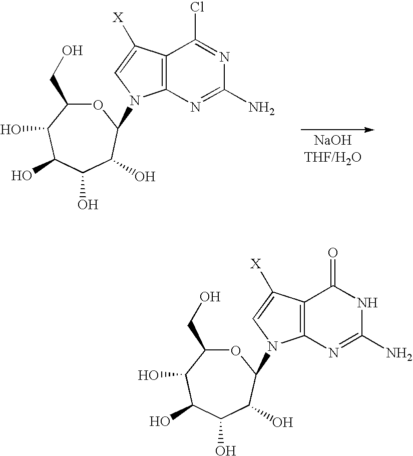 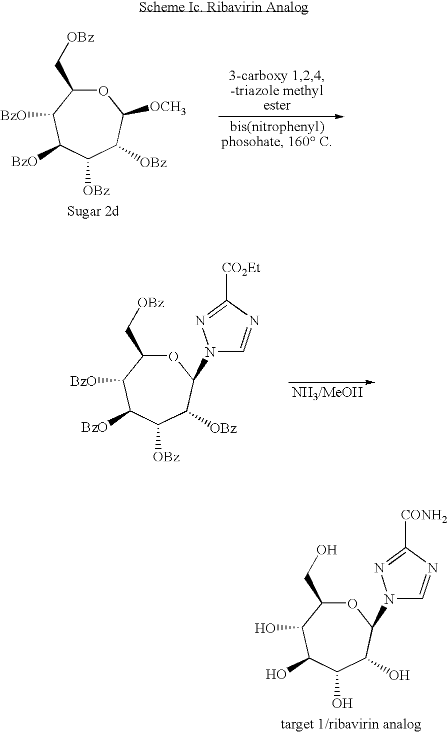 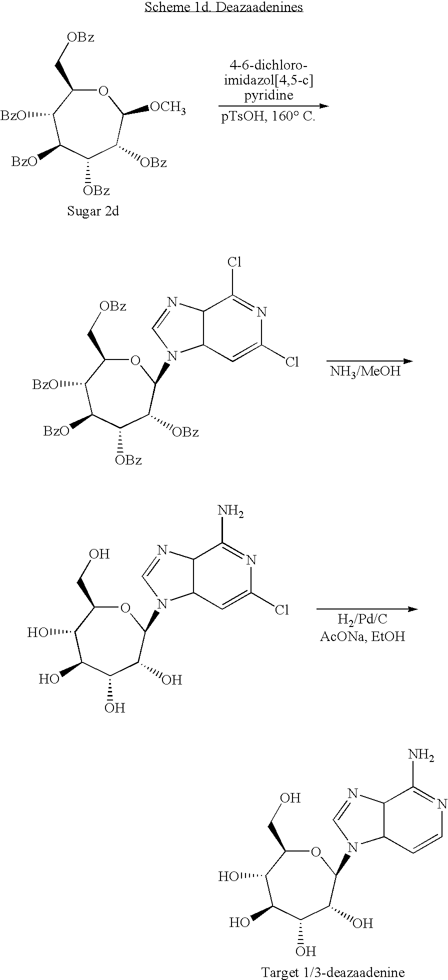
   Scheme II: Natural Bases

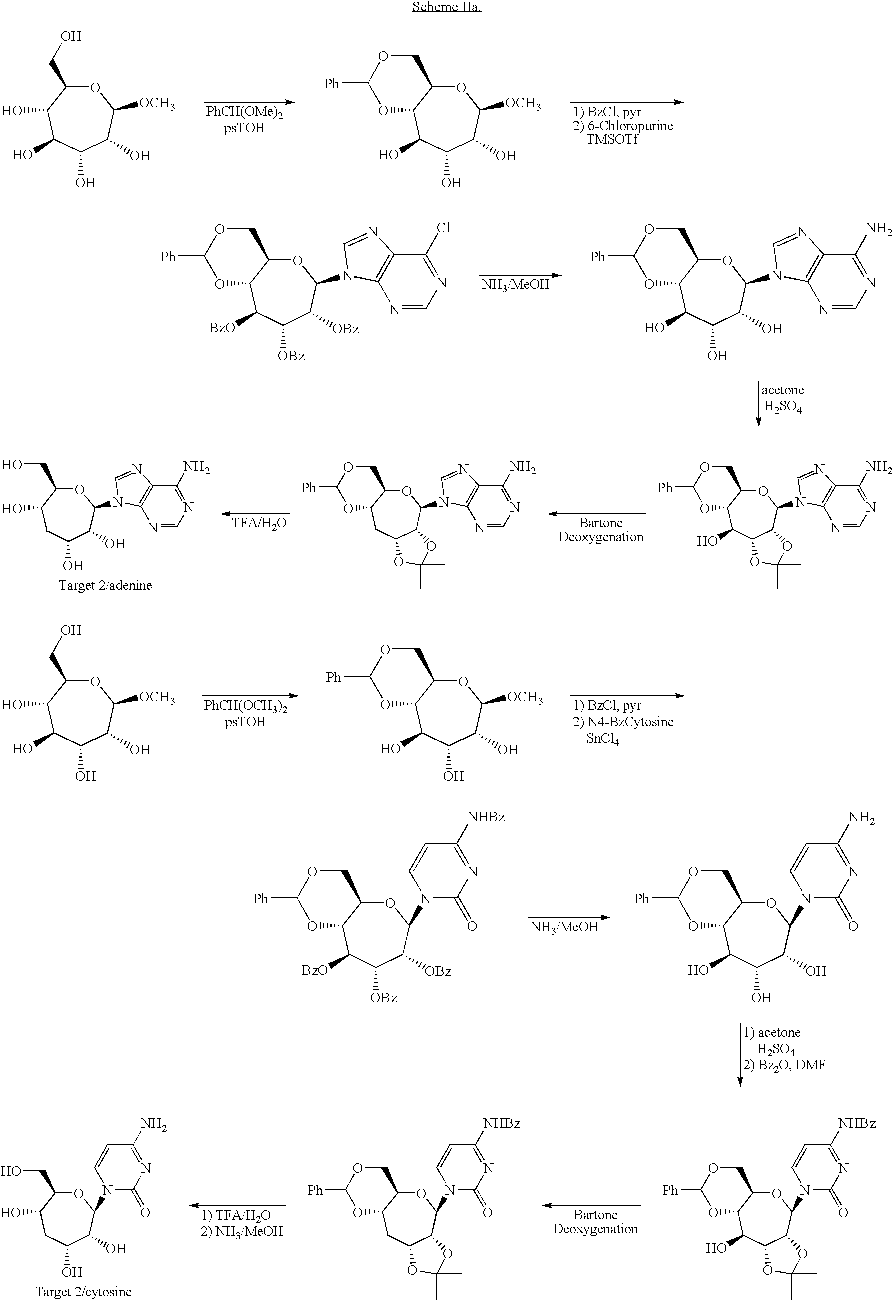

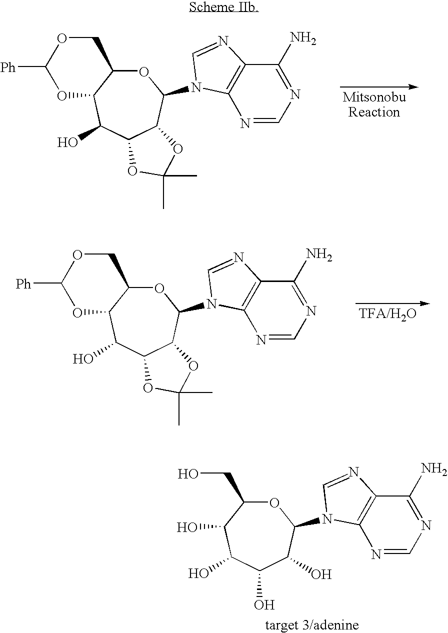

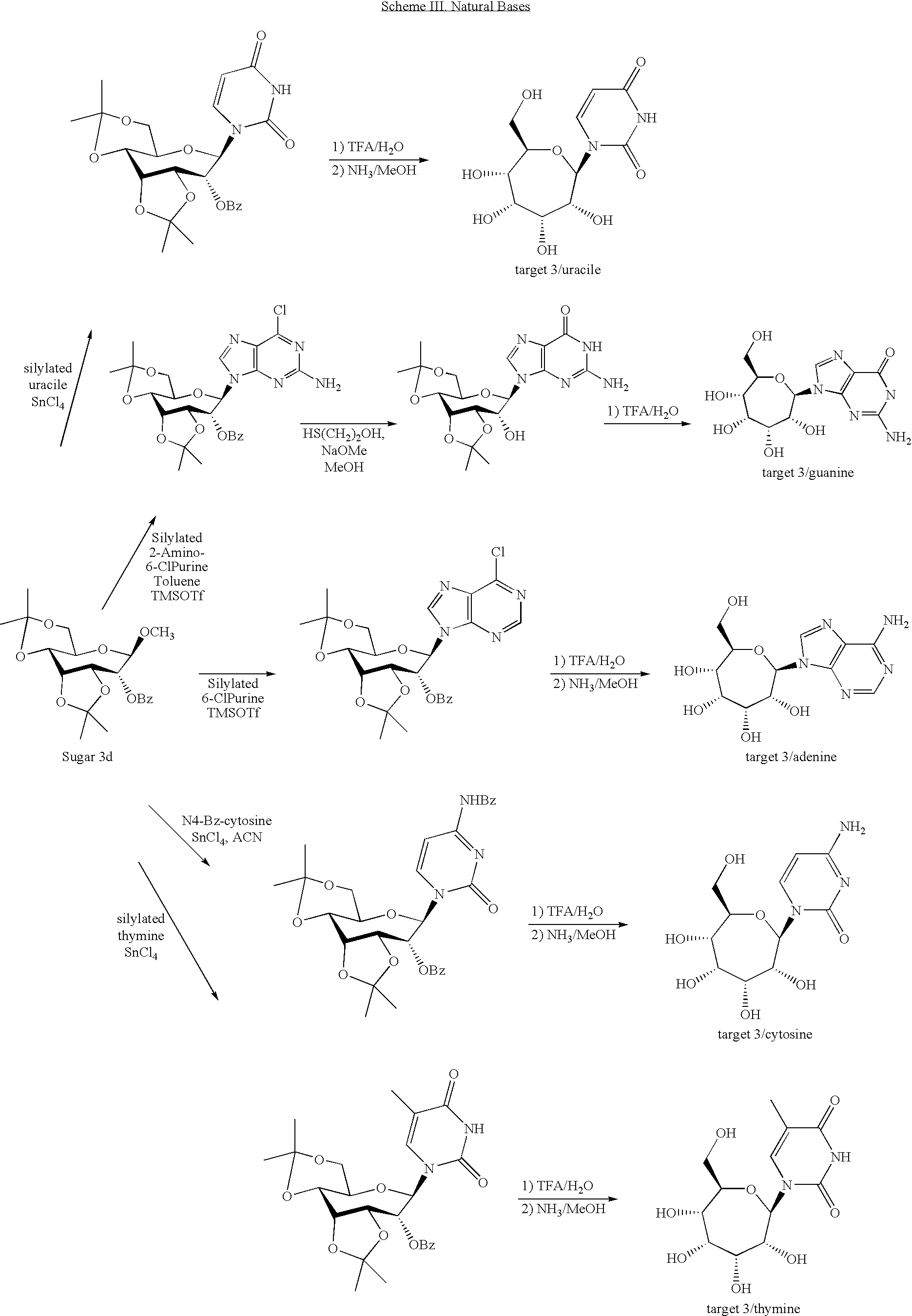

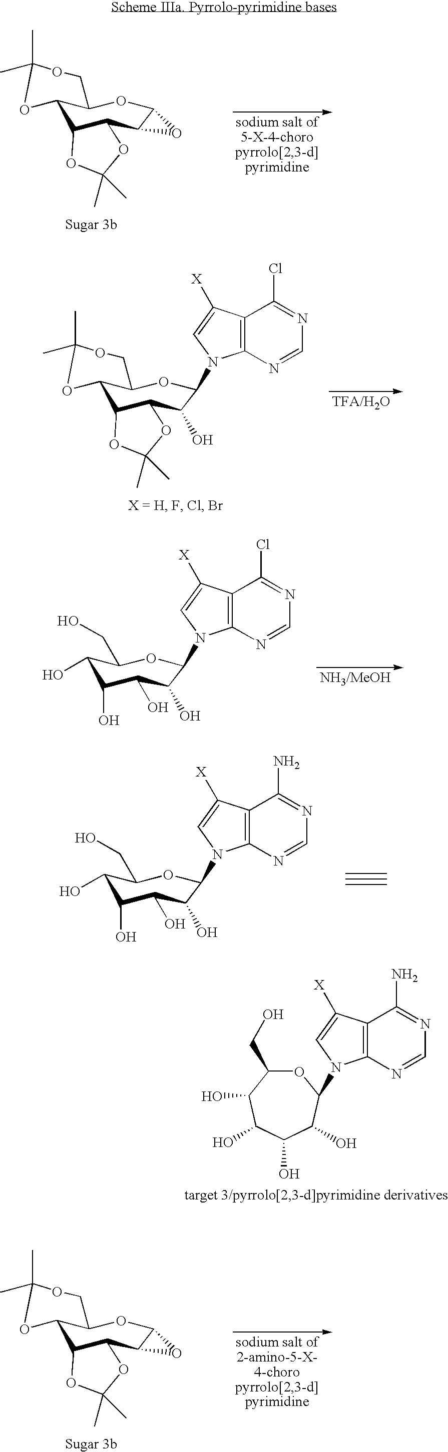

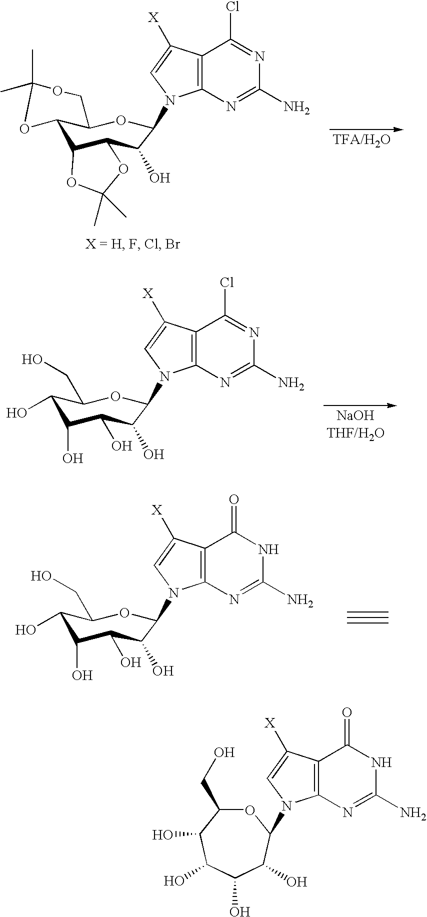

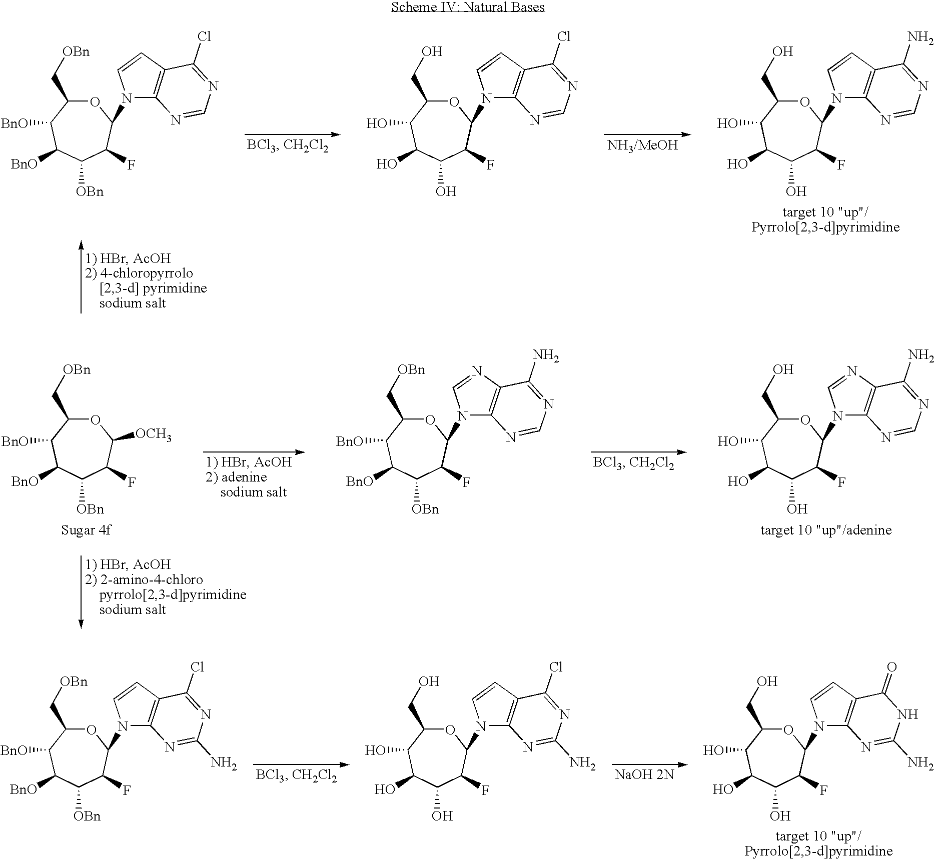

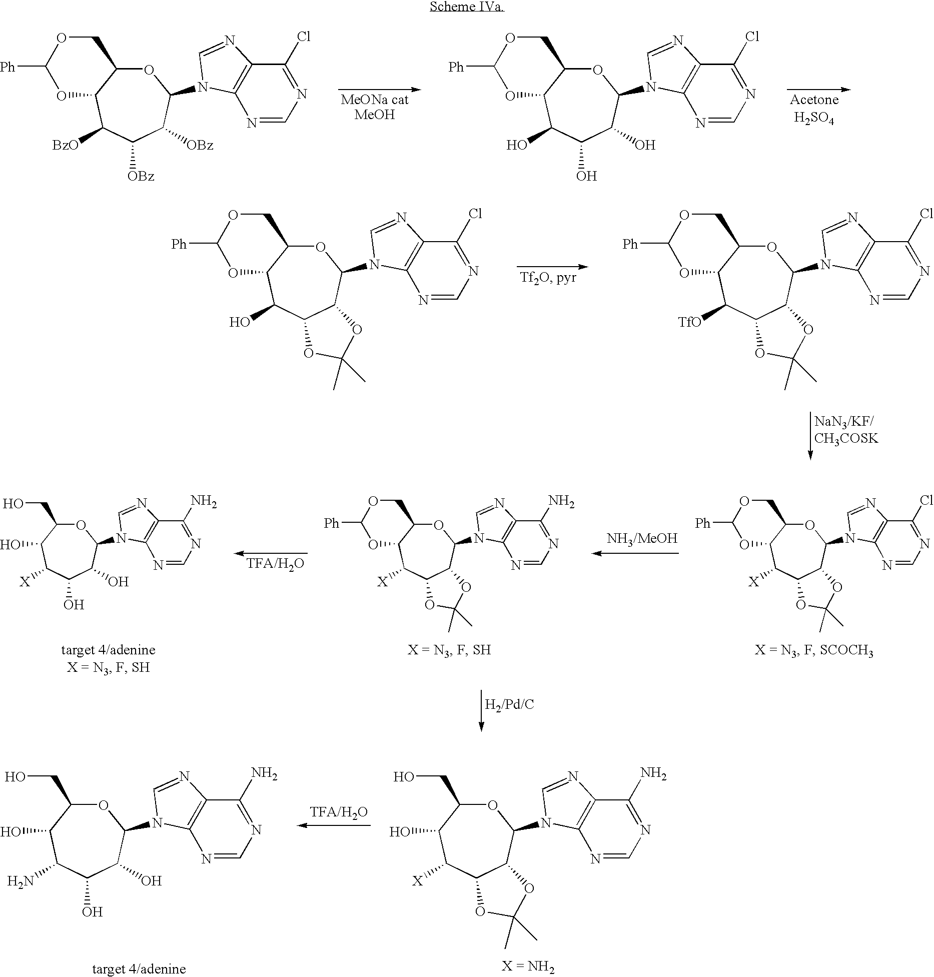

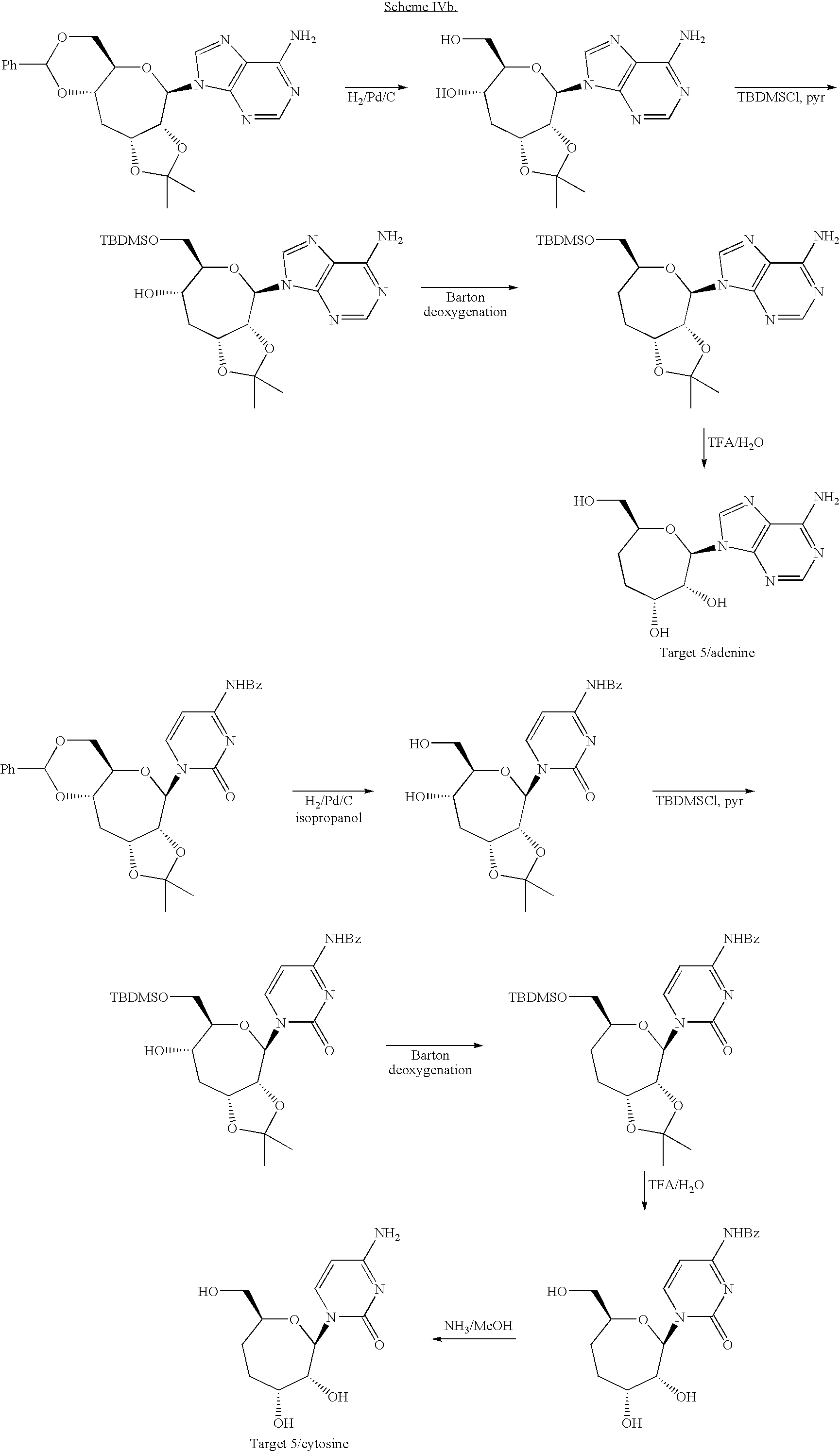

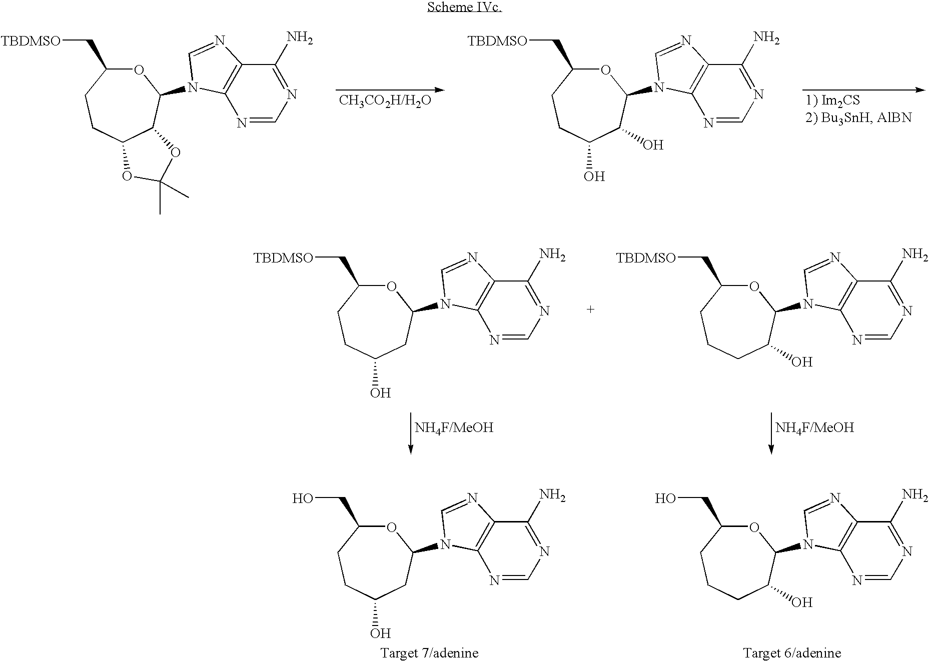

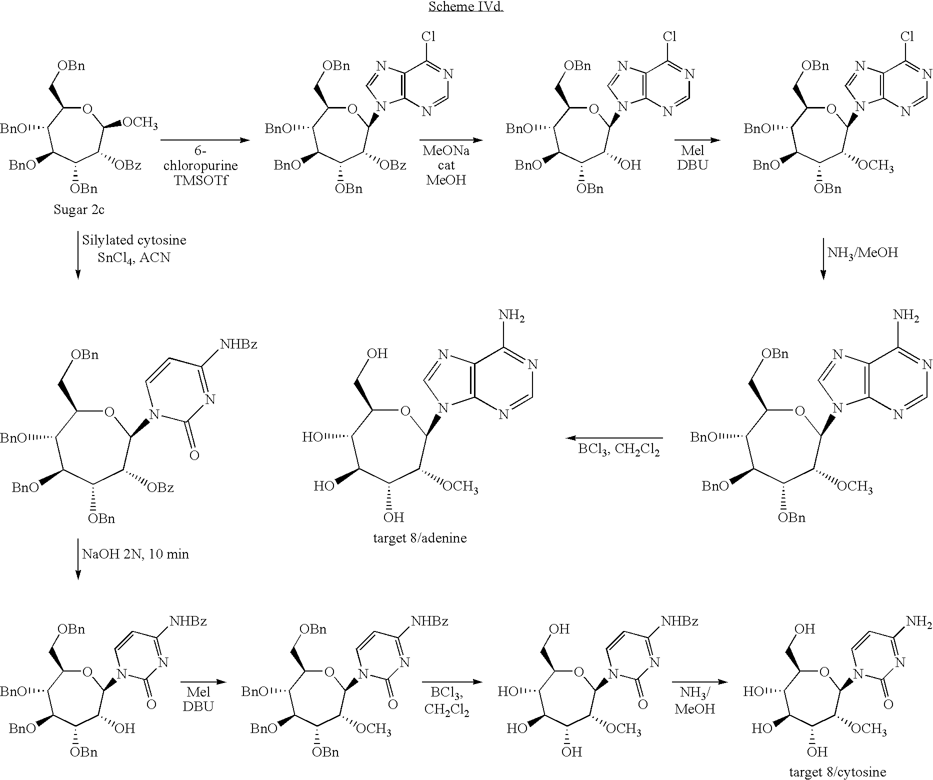

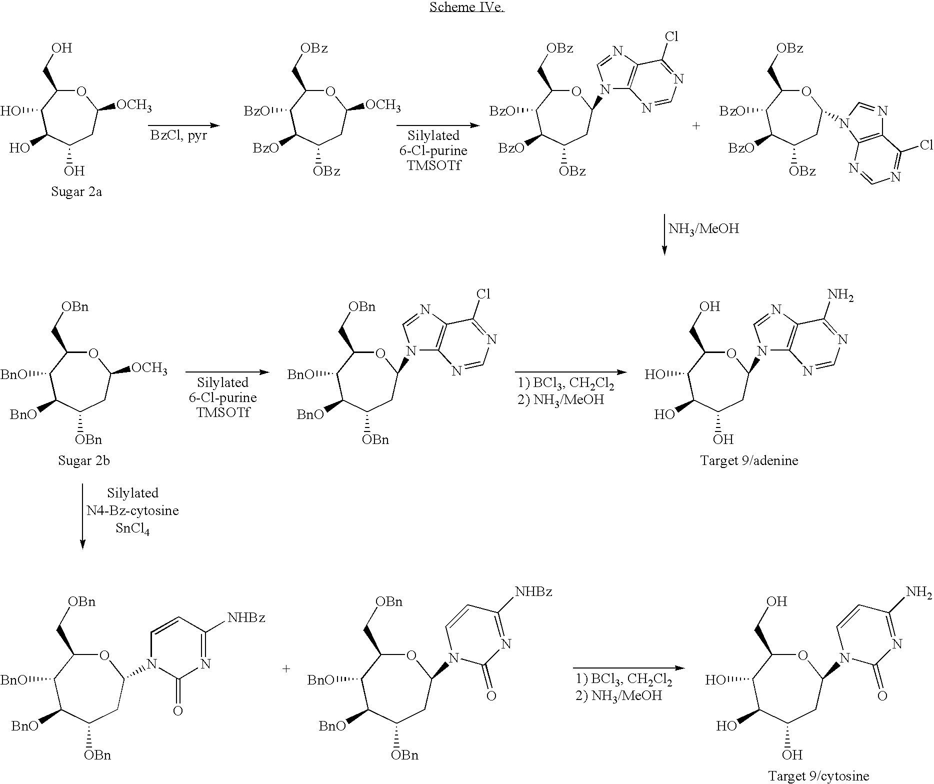

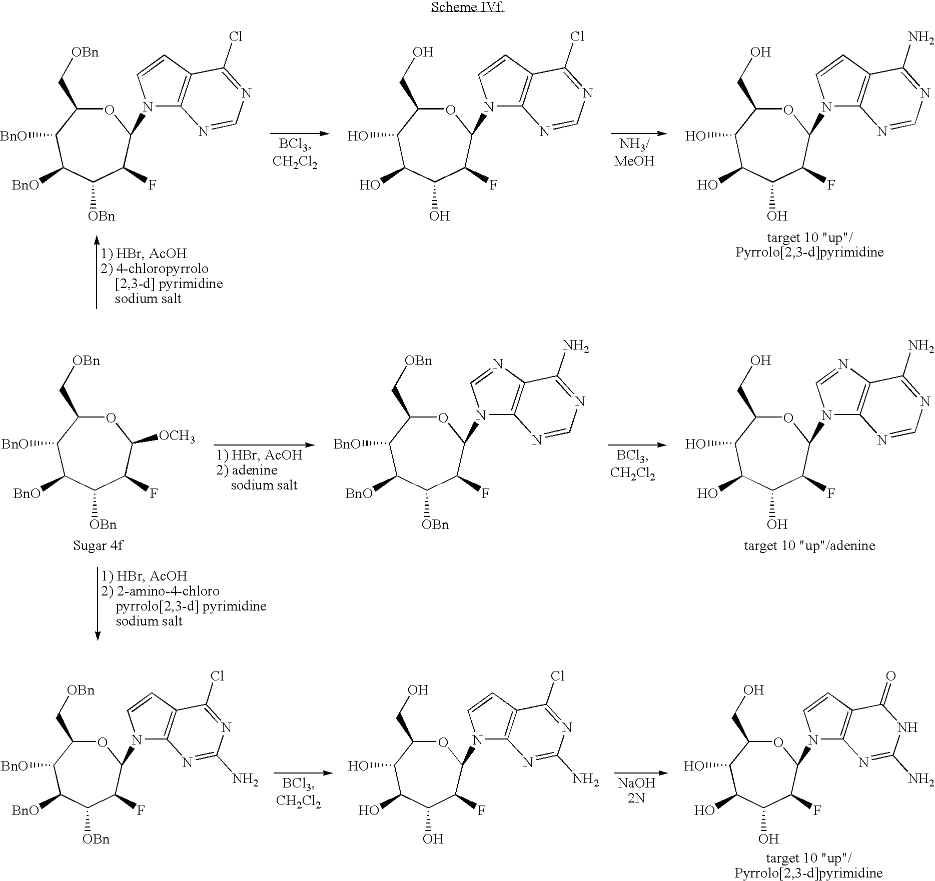

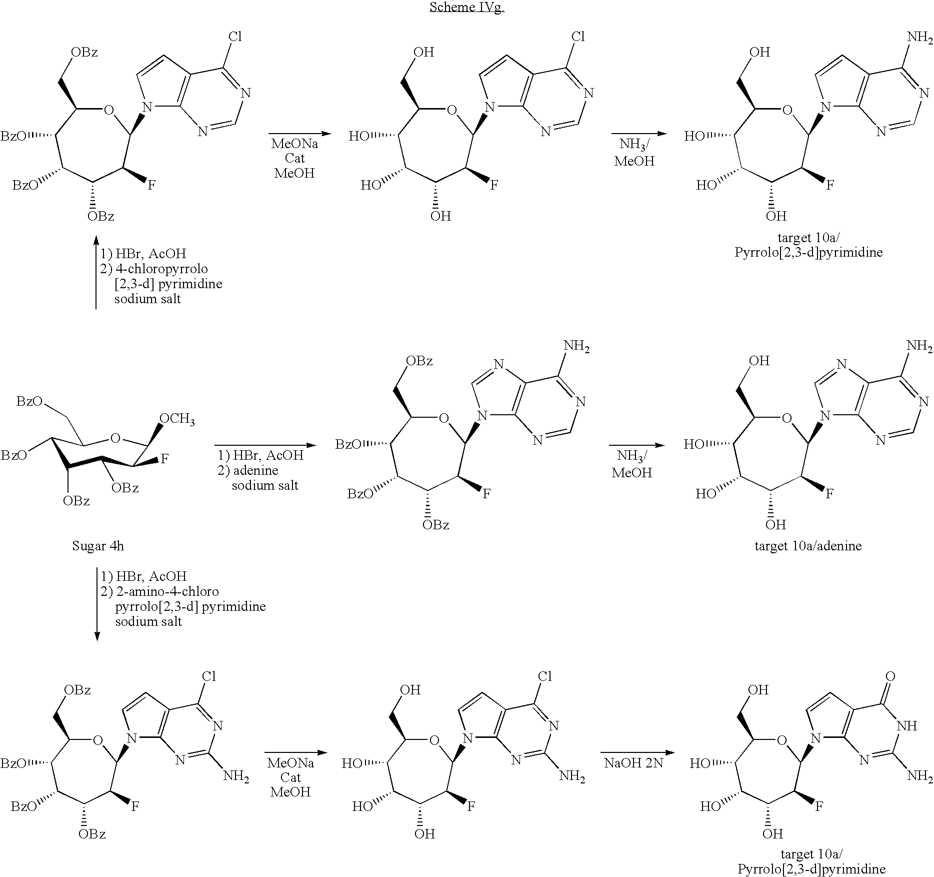

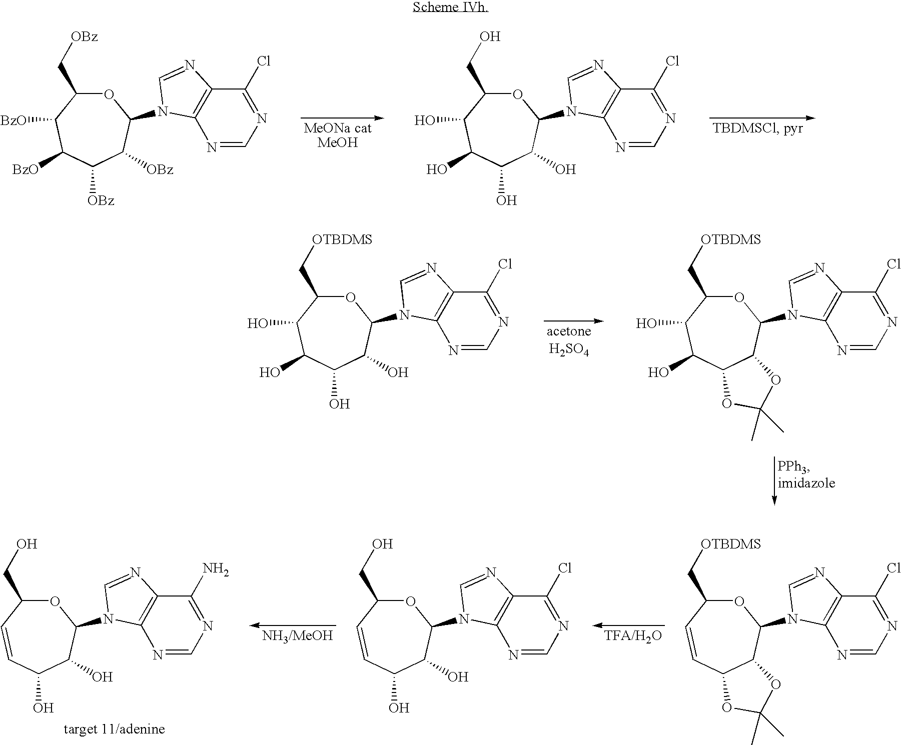

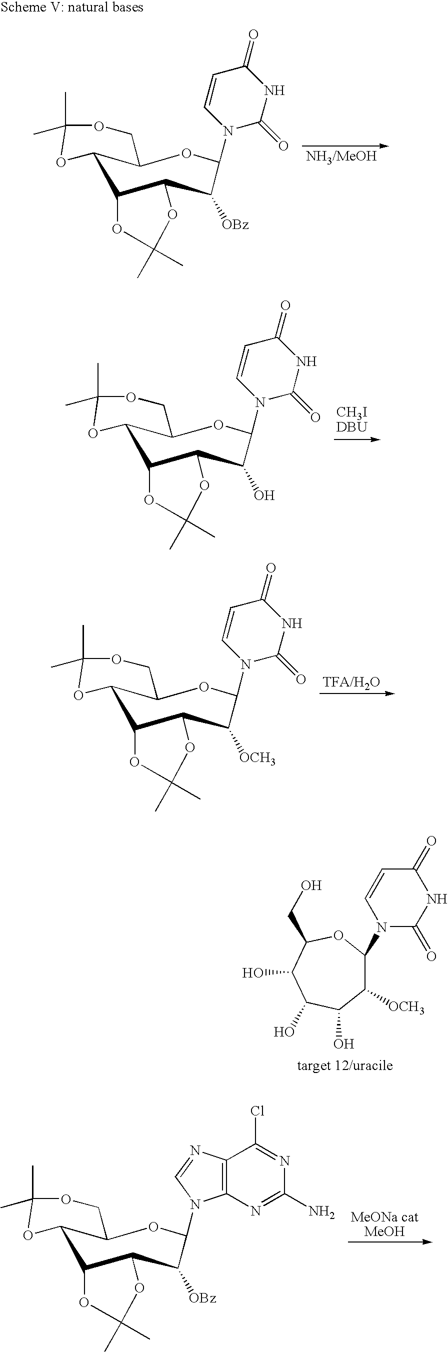

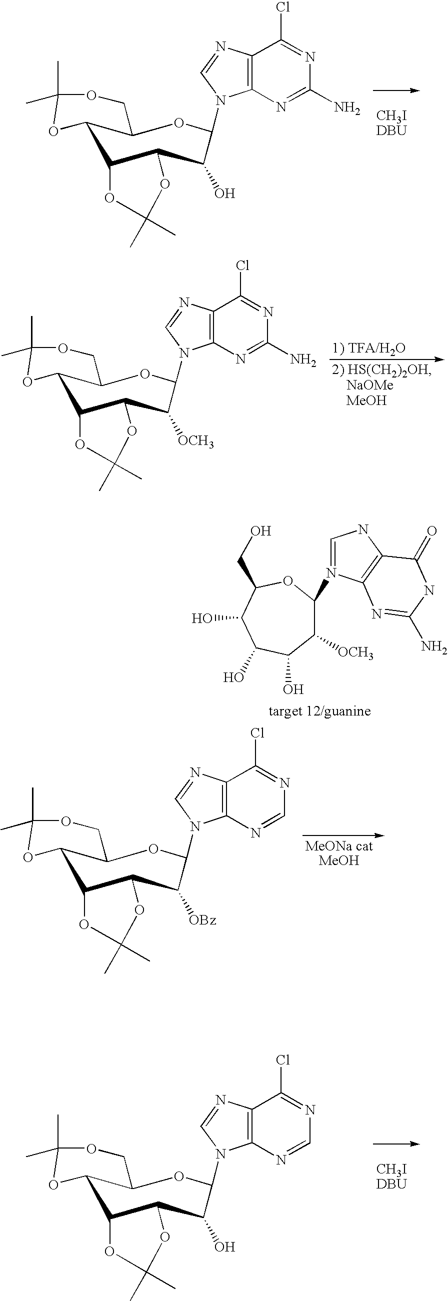

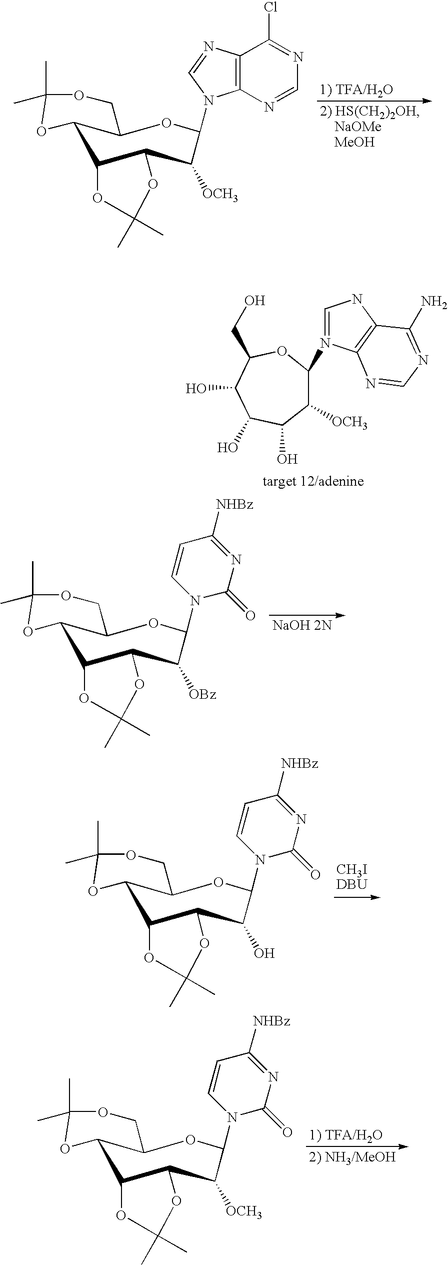

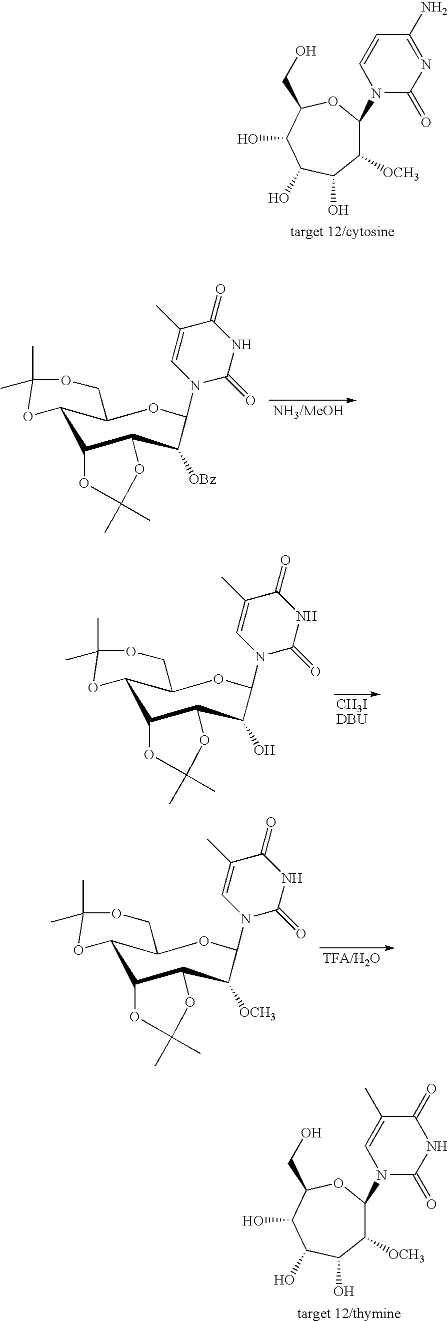

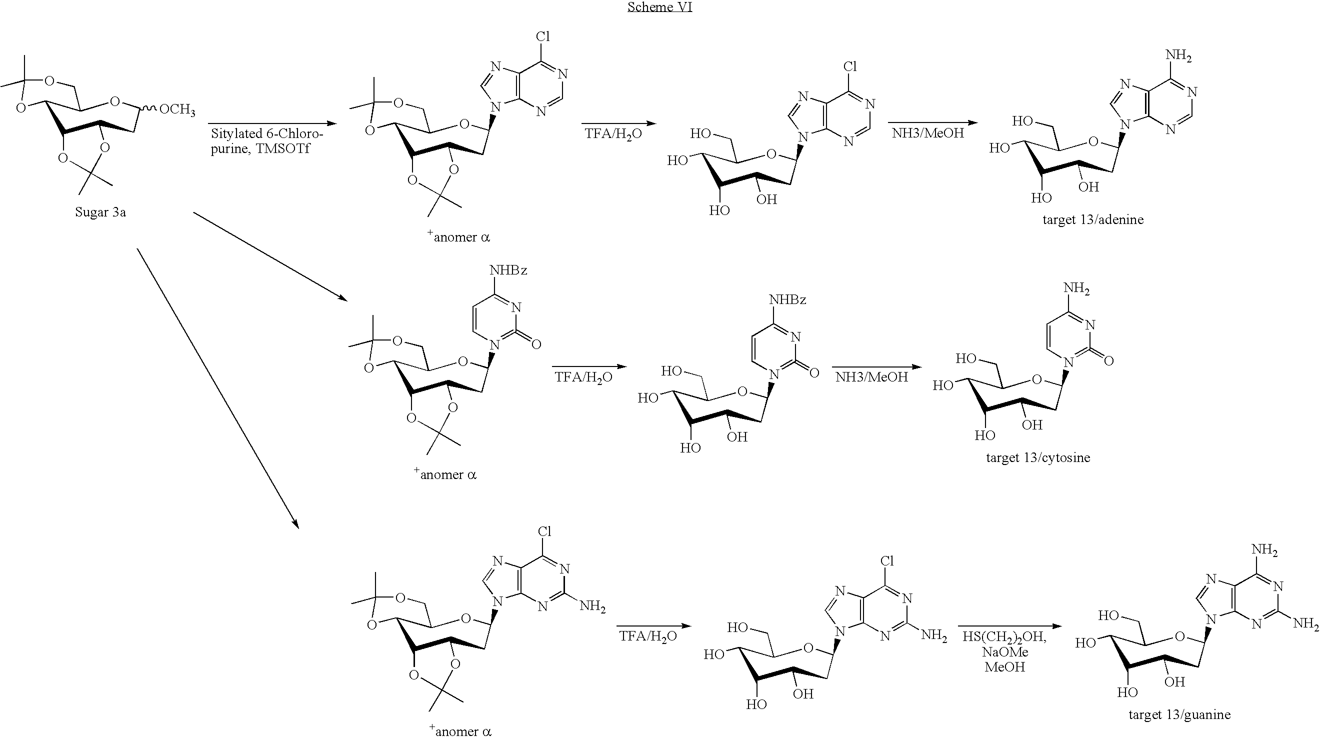

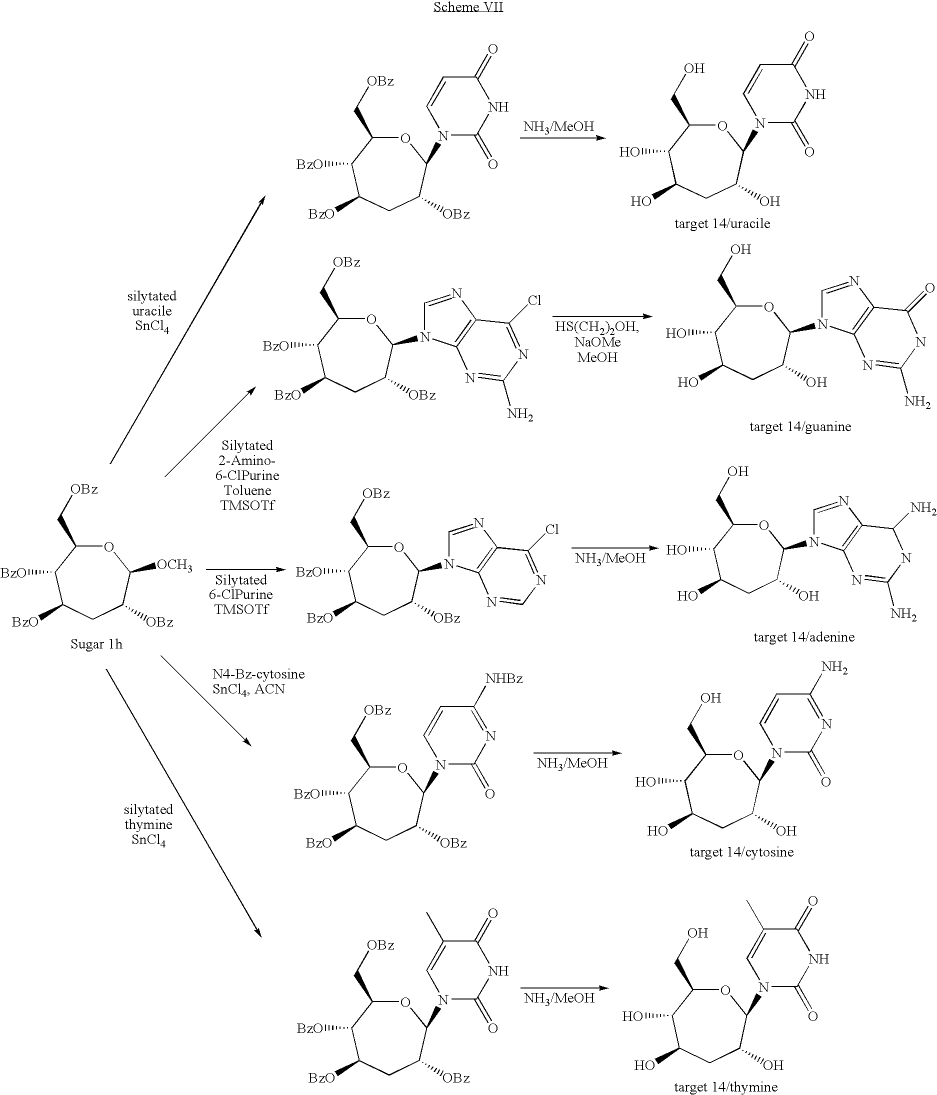

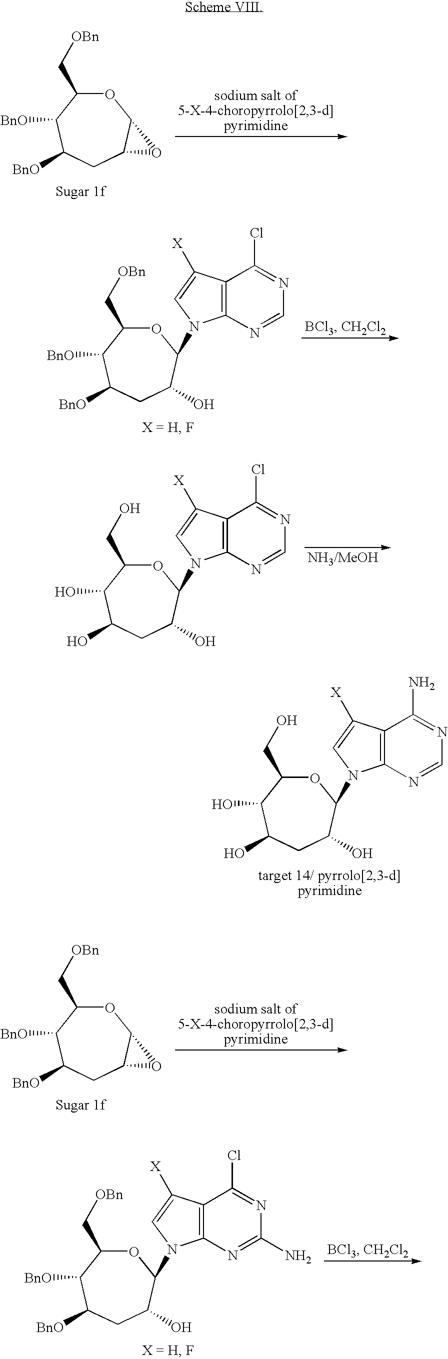

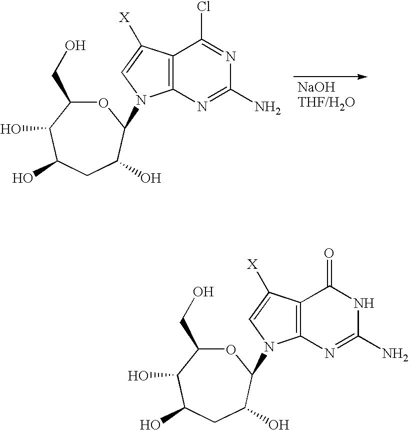

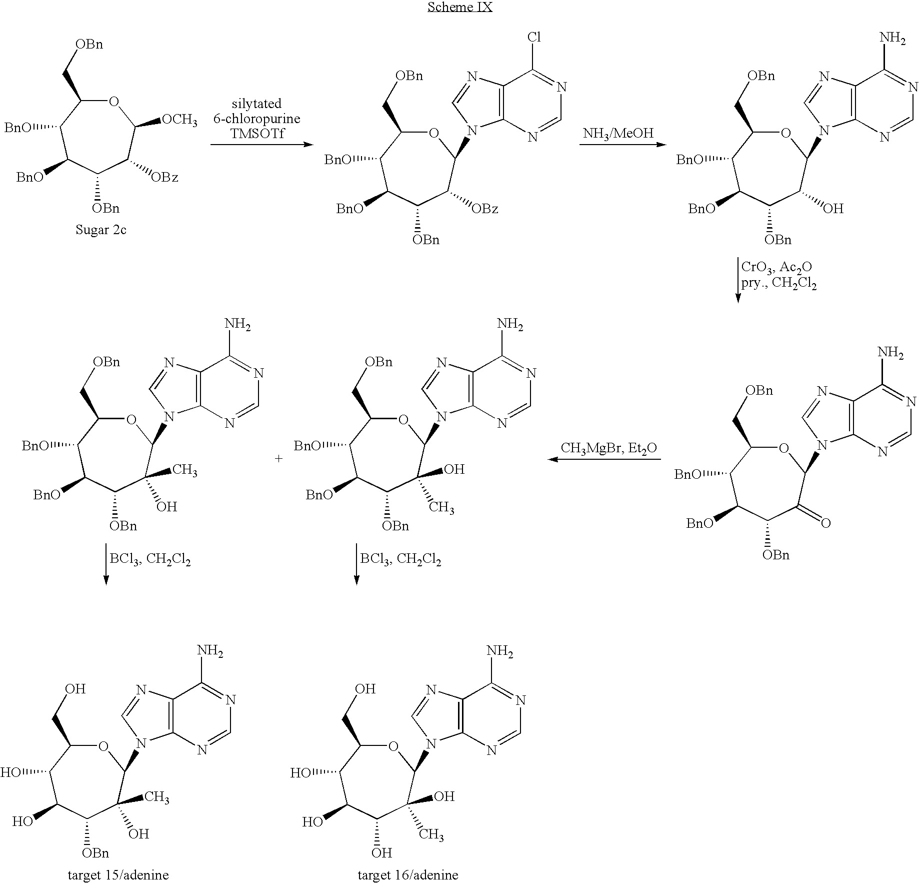

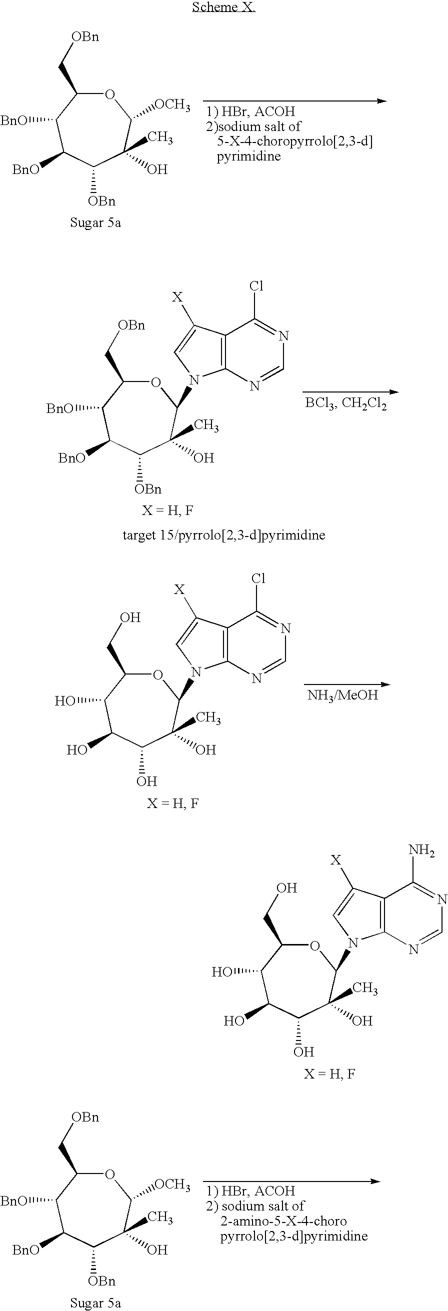

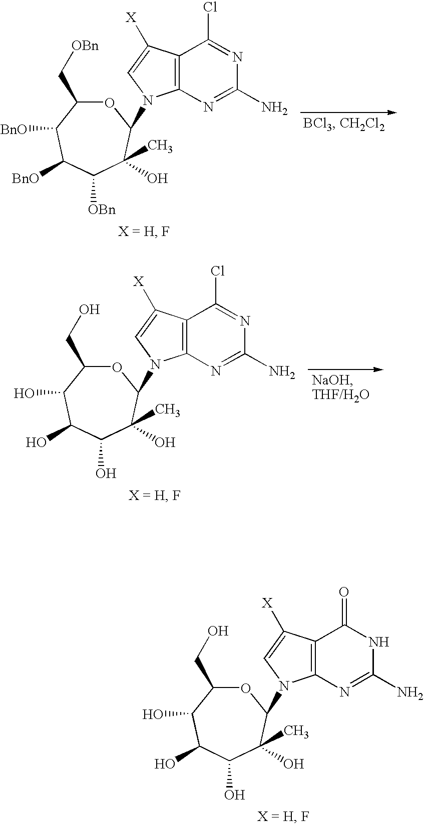

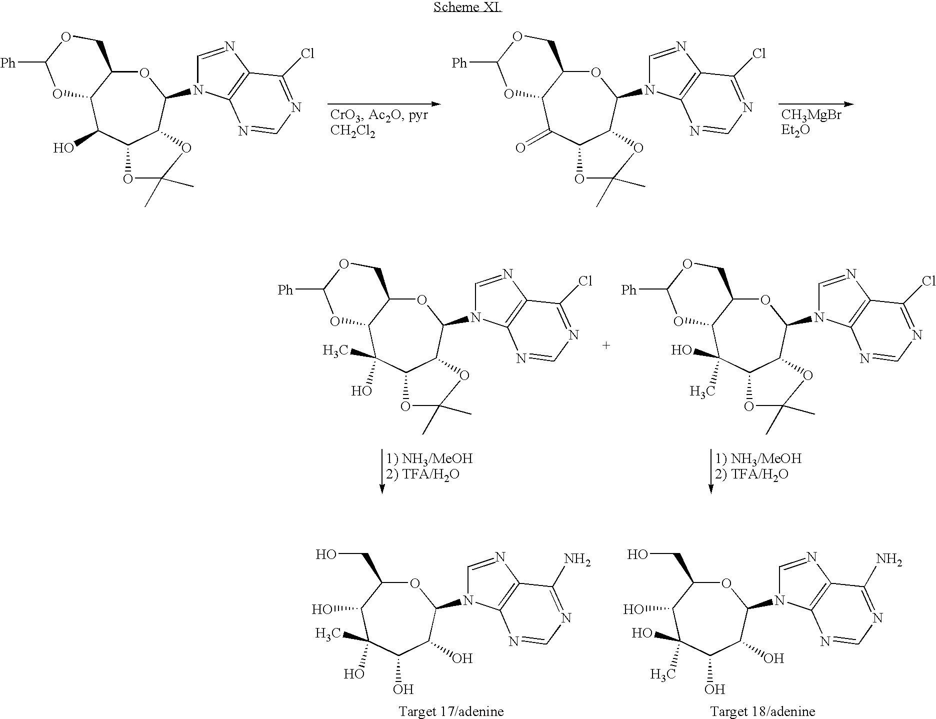

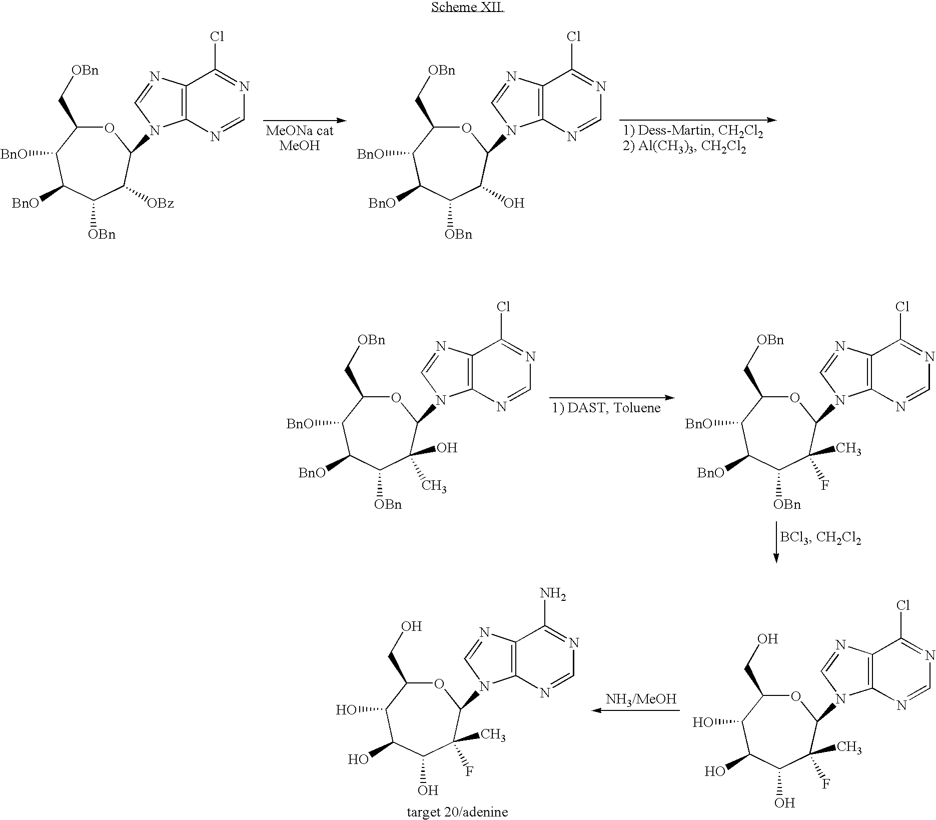

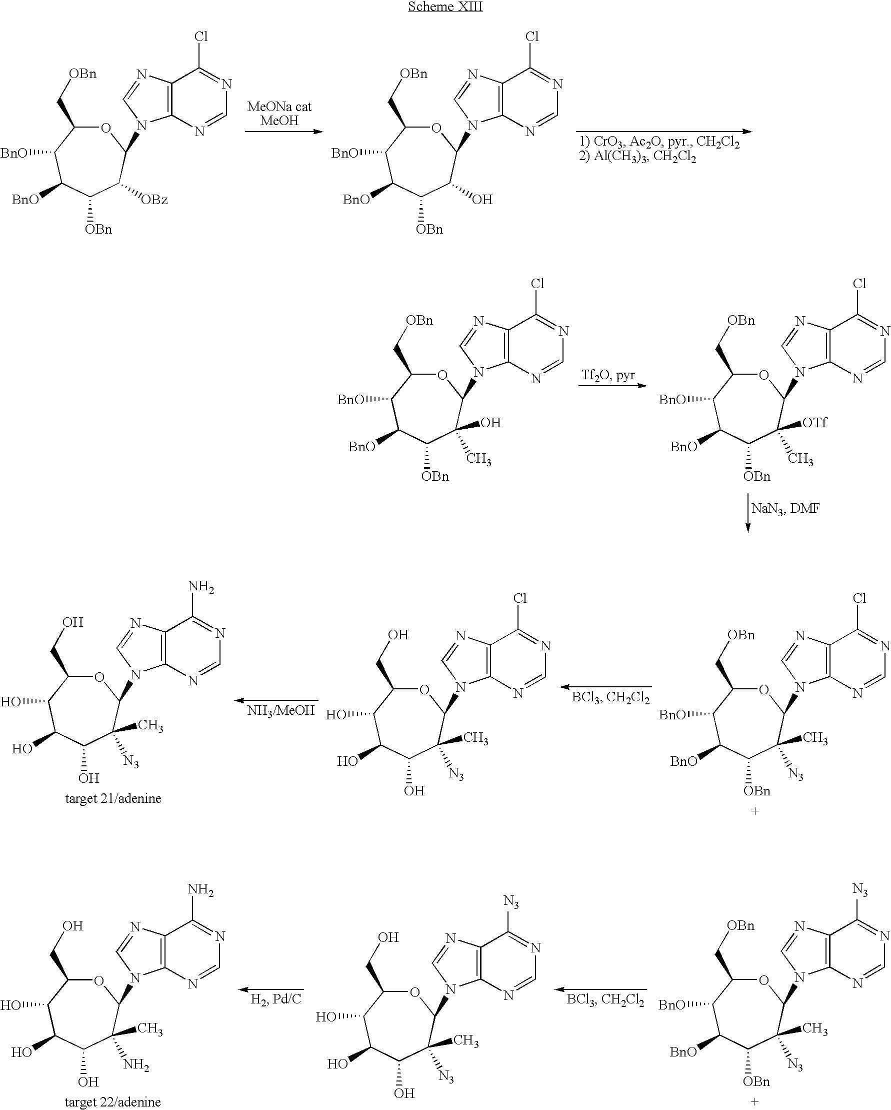

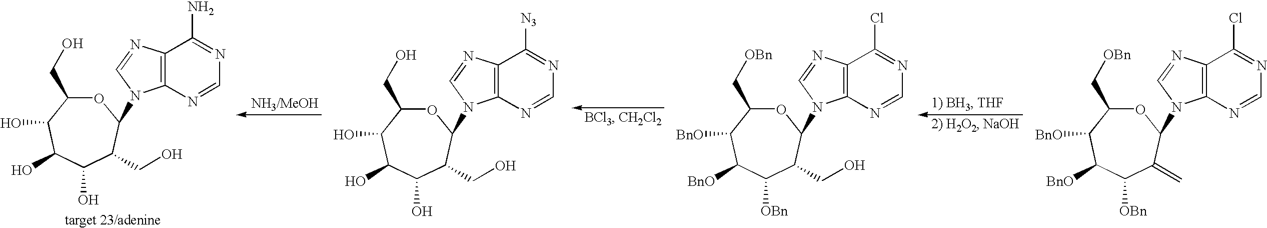

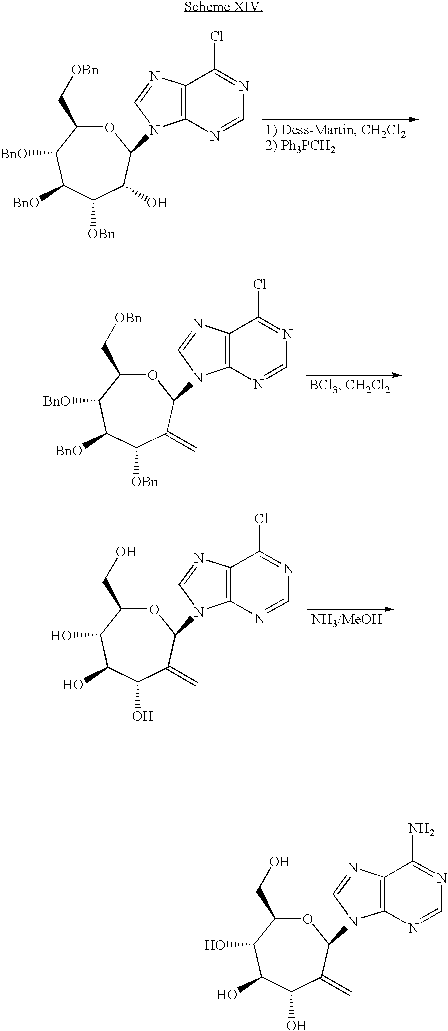

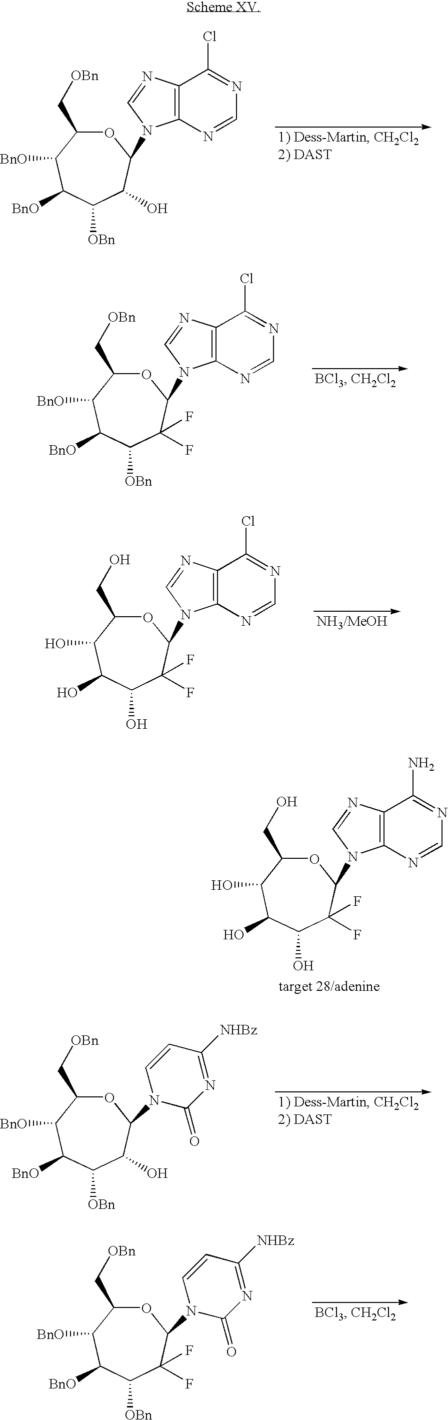

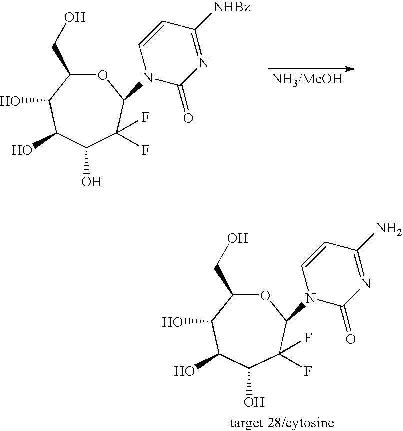

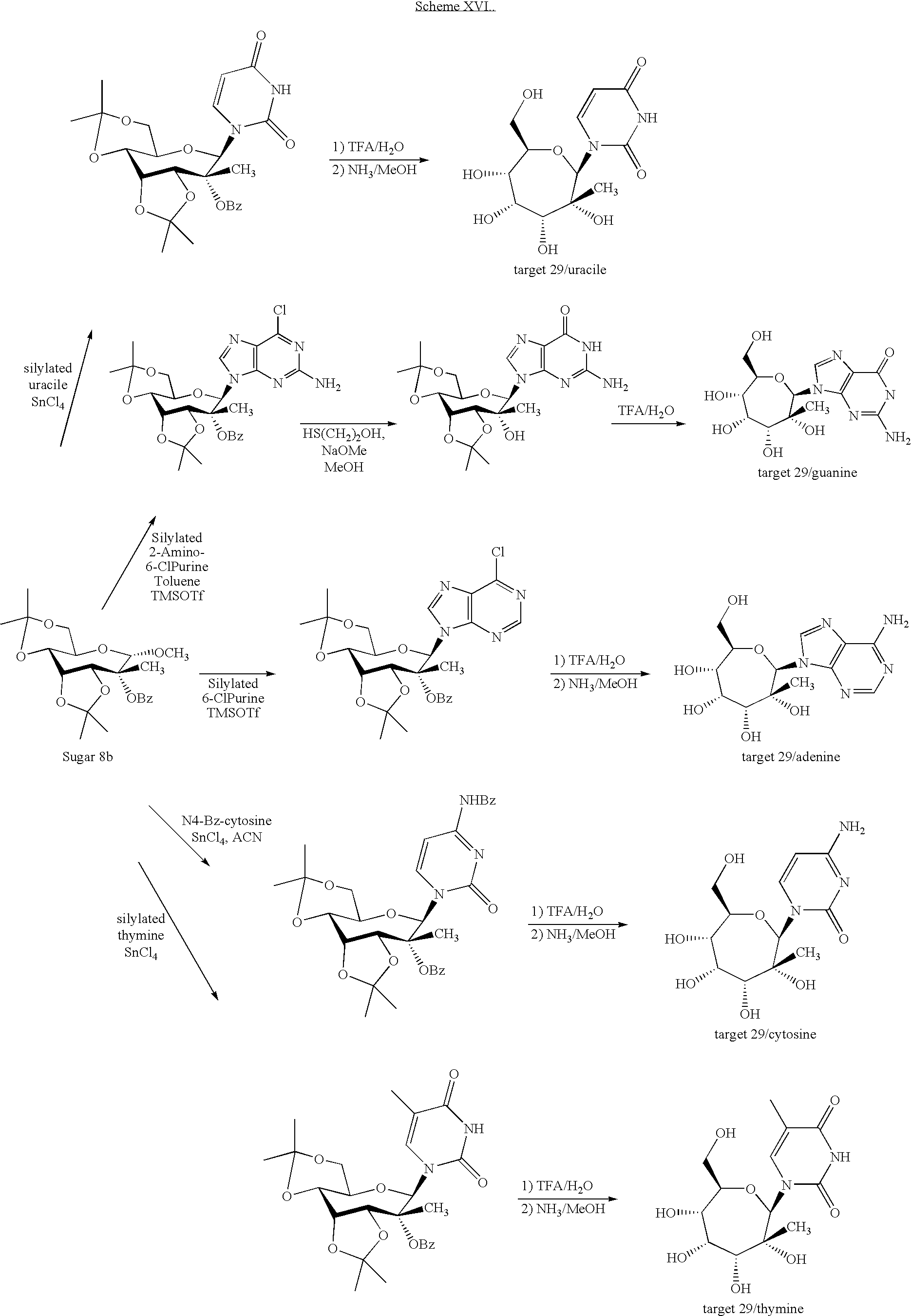

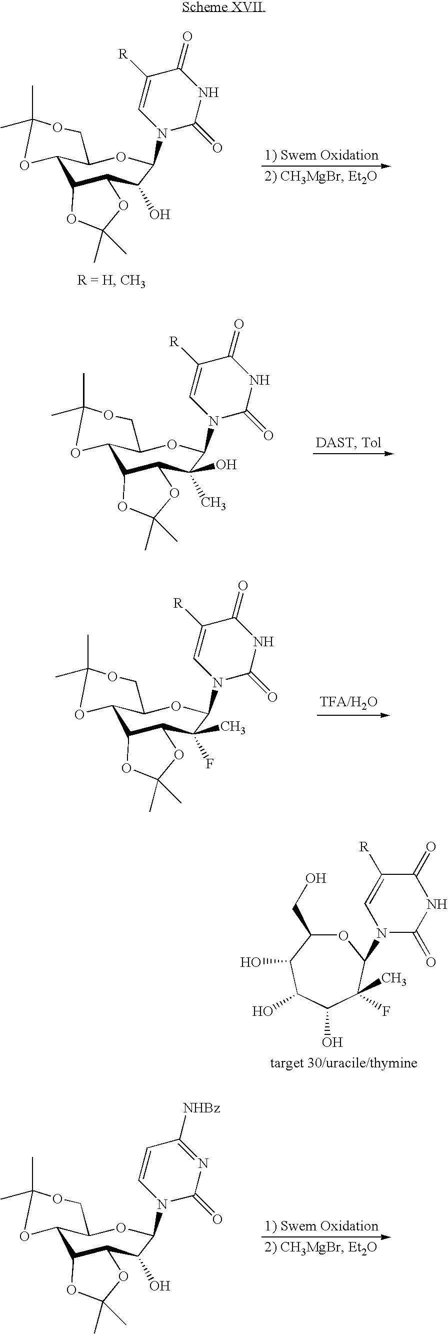

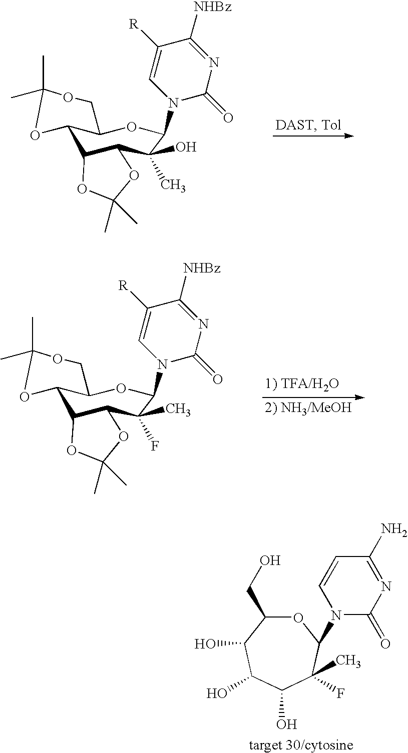

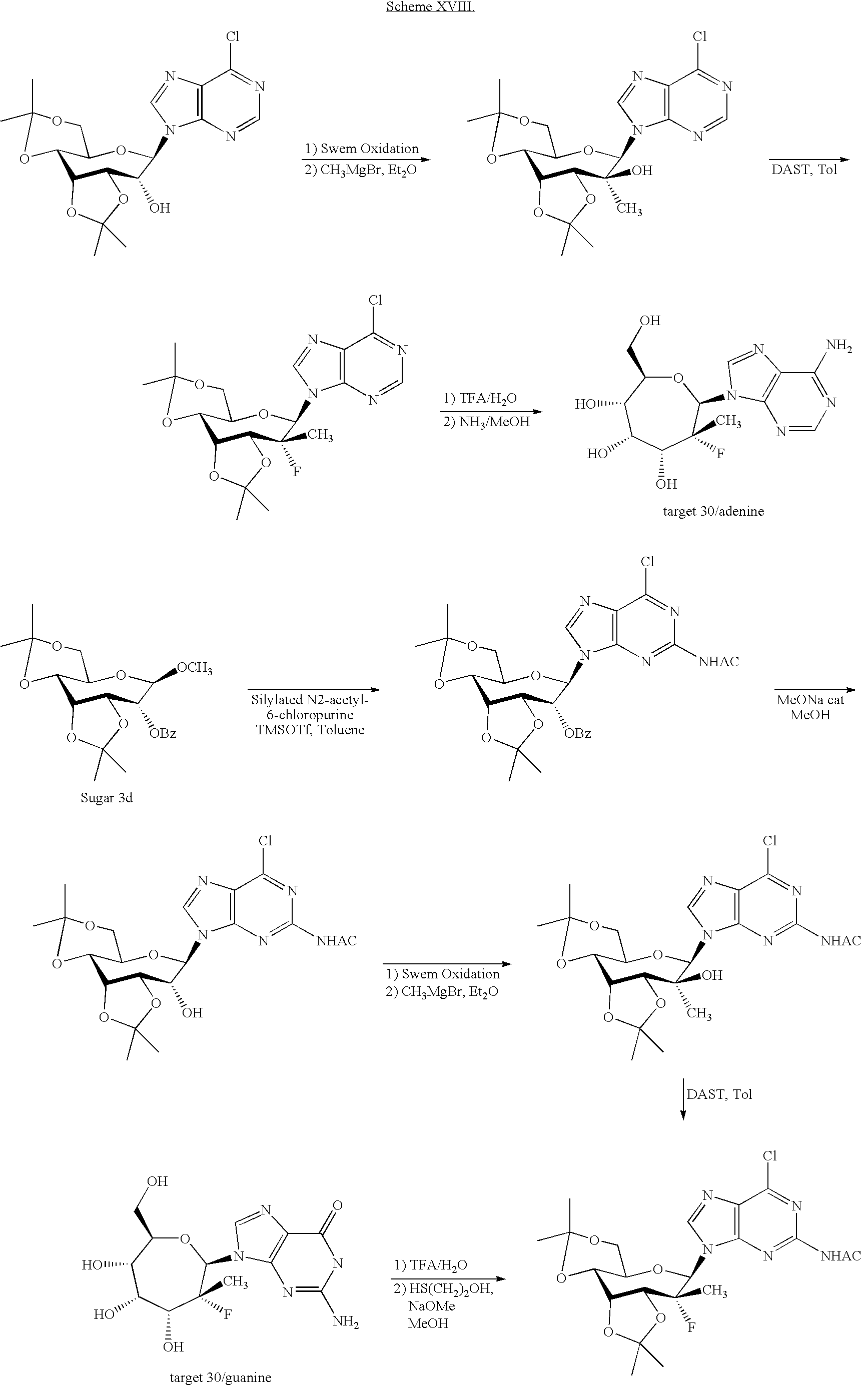

It is understood that the above syntheses are only representative, non-limiting examples of syntheses of 7-membered ring nucleosides and starting material sugars, and that any syntheses obvious to those of skill in the art for preparing such sugars and nucleosides are included within the scope of the present invention.

### EXAMPLES

### Example 1

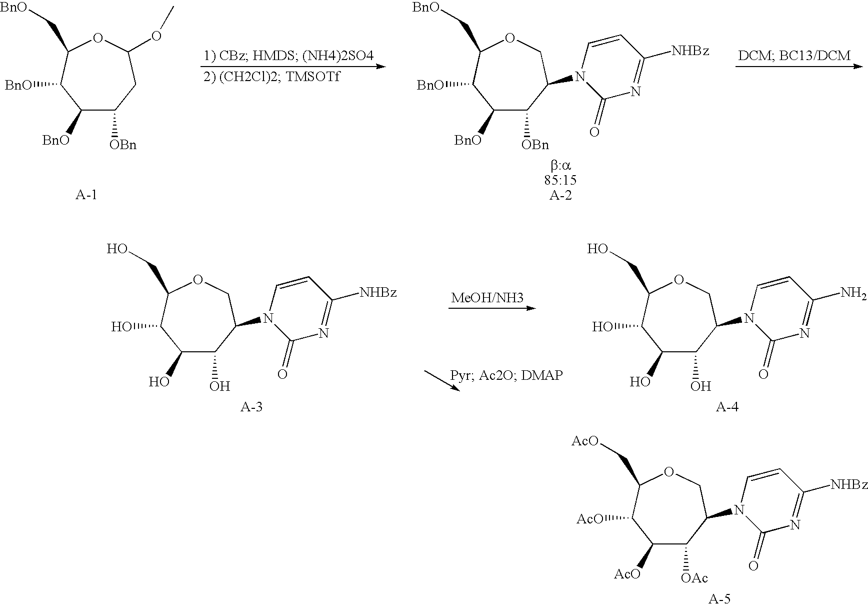

Compound A-1: was prepared according to: Castro S., Peczuh M. W., Journal of Organic Chemistry, 2005, 70, 3312-15. Molecular Formula: C₃₆H₄₀O₆.¹H NMR (400 MHz, CDCl₃) δ 7.40-7.20 (m, 20 H, 4Bn), 5.05 (d, 1H, J=10.8 Hz, H1′), 4.90-4.60 (m, 8H, 4CH₂Bn), 3.90 (m, 1H), 3.80 (m, 1H), 3.70-3.60 (m, 4H), 3.40 (s, 3H, OCH₃), 2.20 (dd, 1H, J₁=5.6 Hz, J₂=14.8 Hz, H2a′), 1.95 (m, 1H, H2b′)

Compound A-2. Silylation of N4-benzoylcytosine: a suspension of N4-benzoylcytosine (450 mg, 2.10 mmol) in 1,1,1,3,3,3-hexamethyldisilazane (HMDS, 20 ml) and a catalytic quantity of ammonium sulfate, was heated with stirring at reflux for 20 hours under argon. The resulting solution was then allowed to cool to room temperature and concentrated under vacuo under argon. Condensation step: to a solution of silylated N4-benzoylcytosine in anhydrous 1,2-dichloroethane (20 ml) was sequentially added compound A-1 (1.0 g, 1.76 mmol) and trimethylsilyltrifluoromethanesulfonate (TMSOTf, 0.51 ml, 2.64 mmol). The reaction mixture was stirred at reflux during 2 hours under argon. The resulting solution was diluted with ethyl acetate and neutralized with a 5% solution of sodium bicarbonate. The organic layer was washed once with brine, then dried over sodium sulfate and evaporated under reduced pressure. The crude material was purified on silica gel column chromatography (eluent : 1% of methanol in dichloromethane) to give compound A-2 (600 mg, 45%). Molecular Formula: C₄₆H₄₅N₃O₇. ¹H NMR (300 MHz, CDCl₃), δ 11.50 (s, 1H, NH), 8.10-7.90 (m, 3H, H5+2H of Bz), 7.50-7.10 (m, 24H, H6+3H of Bz, 4Bn), 6.00 (dd, 1H, J₁=3.18 Hz, J₂=10.9 Hz, H1′), 5.00-4.55 (m, 6H, 3 CH₂Bn), 4.45 (m, 2H, CH₂Bn), 4.20 (m, 1H, H5′), 3.70 (m, 3H, H3′+H7a′+H7b′), 3.50 (m, 2H, H4′+H6′), 2.60 (m, 1H, H2b′), 2.10 (m, 1H, H2a′). LC/MS: t=22.6 min; MS ES+=752 (M+H)⁺, UV λ max=259 nm.

Compound A-3. To a solution of A-2 (300 mg, 0.4 mmol) in anhydrous dichloromethane (10 ml) at −78° C. was added boron trichloride (1M in dichloromethane, 4 ml, 4 mmol) dropwise. The mixture was stirred at −78° C. for 3 hours then allowed to rise about −30° C. and stirred for 3 h. The reaction was quenched by addition of methanol/dichloromethane (1/1) and stirred for 30 min at −15° C., then neutralized at 0° C. with aqueous ammonia and stirred at room temperature for 15 min. The solid was filtered through a pad of celite and washed with dichloromethane/methanol (1/1). The filtrate was evaporated to dryness and the residue was purified on a silica gel column chromatography (eluent: dichloromethane/methanol (9/1)) to give compound A-3 (105 mg, 67 %). Molecular Formula: C₁₈H₂₁N₃O₇. ¹H NMR (300 MHz, DMSO d6): δ 11.10 (bs, 1H, NH), 8.20 (d, 1H, J=7.5 Hz, H5), 8.00 (m, 2H, Bz), 7.70-7.45 (m, 3H, Bz), 7.35 (d, 1H, J=7.4 Hz, H6), 5.85 (t, 1H, J=4.9 Hz, H1′), 5.00 (m, 2H, 20H′), 4.80 (d, 1H, J=3.6 Hz, OH′), 4.50 (t, 1H, J=5.8 Hz, OH7′), 3.70 (m, 3H), 3.40 (under water, 3H), 2.10 (m, 2H, H2b′+H2a′). LC/MS: t=8.2 min; MS ES+=392 (M+H)⁺, UV λ max=260nm.

Compound A-4. To a solution of A-3 (180 mg, 0.46 mmol) in methanol (4 ml) was added sodium methoxide (126 mg, 2.6 mmol). The reaction mixture was stirred at 45° C. overnight. The mixture was neutralized with an aqueous solution of HCl (1M) and then evaporated to dryness. The residue was purified by reverse phase (C 18) silica gel column chromatography (eluent: water), then on a silica gel column chromatography (eluent: dichloromethane/methanol (8/2)) to fumish compound A4 (35 mg, 26 %), □/□ anomers: 85/15. Molecular Formula: C₁₁H₁₇N₃O₆. Major compound (□): ¹H NMR (300 MHz, DMSO dc6): δ 7.65 (d, 1H, J=7.4 Hz, H6), 7.20-7.00 (bd, 2H, NH2), 5.75 (m, 2H, H1′+H5), 5.00 (m, 2H, 2OH′), 4.80 (m, 1H, OH′), 4.45 (m, 1H, OH7′), 3.70 (m, 3H), 3.40 (under water, 3H), 2.00 (m, 2′H, H2a′+H2b′). LC/MS: t=1.2 min; MS ES+=288 (M+H)⁺, UV λ max=271 nm.

Compound A-5. To a solution of A-3 (105 mg, 0.268 mmol) in anhydrous pyridine (1 ml) was added under argon at 0° C. acetic anhydride (0.25 ml, 2.68 mmol) and a catalytic amount of dimethylaminopyridine (DMAP). The reaction mixture was stirred at room temperature for 2 days, diluted in ethyl acetate and sequentially washed with 1M HCl solution, a 5% aqueous solution of sodium bicarbonate and brine. The organic layer was dried (Na₂SO₄) and concentrated under vacuo. The crude was purified on a silica gel column chromatography (eluent: from 1 to 2% of ethanol in dichloromethane) to afford compound A-5 (73 mg, 49 %). Molecular Formula: C₂₆H₂₉N₃O₁₁. ¹H NMR (300 MHz, DMSO d₆): δ 11.30 (bs, 1H, NH), 8.40 (d, 1H, H5), 8.00 (m, 2H, Bz), 7.70-7.50 (m, 3H, Bz), 7.40 (d, 1H, H6), 6.00 (m, 1H, H1′), 5.30 (m, 1H, H4′), 5.10 (m, 2H, H3′+H5′), 4.40 (m, 1H, H6′), 4.20-4.00 (m, 2H, H7a′+H7b′), 2.60 (m, 1H, H2b′), 2.20 (m, 1H, H2b′), 2.00 (m, 12H, 4OAc). HPLC: t=10.0 min, UV λ max=261 nm.

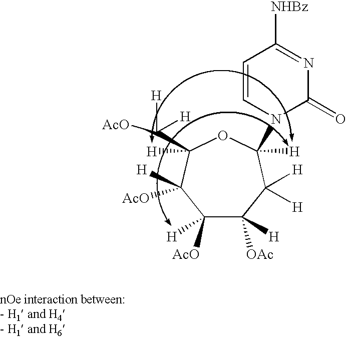

### Example 2

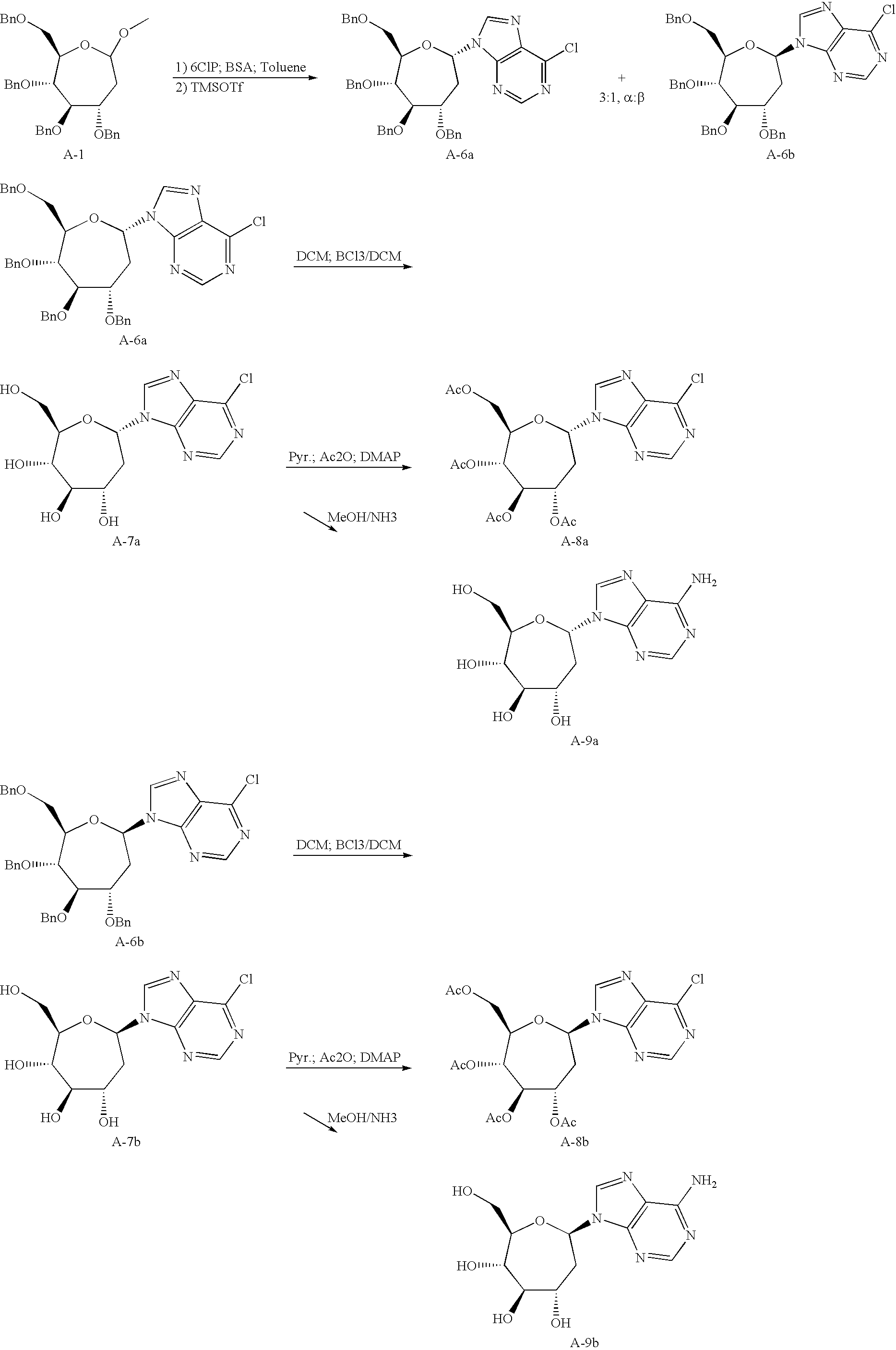

Compound A-6a and A6-b. To a suspension of 6-chloropurine (1.31 g, 8.45 mmol) in anhydrous toluene (25 ml) was added N,O-bis(trimethylsilyl)acetamide (3.49 ml, 14.08 mmol). The reaction mixture was stirred at reflux during 20 min. At room temperature and under argon, to this mixture was added compound A-1 (4.0 g, 7.04 mmol) and trimethylsilyltrifluoromethanesulfonate (TMSOTf, 2.72 ml, 14.08 mmol), the reaction mixture was stirred at reflux for 2 hours, then, at room temperature was diluted with ethyl acetate, washed successively with a saturated aqueous solution of sodium bicarbonate, brine, dried over sodium sulfate and concentrated under vacuo. Purification on a silica gel column chromatography (eluent: petroleum ether/diethyl ether (1/1)) afforded compound A-6a (2.85 g, 58 %) and compound A-6b (1.7 g, 35%). Molecular Formula: C₄₀H₃₉ClN₄O₅. A-6a: ¹H NMR (300 MHz, CDCl₃): δ 8.75 (s, 1H, H8), 8.40 (s, 1H, H2), 7.40-7.00 (m, 20H, 4Bn), 6.20 (dd, 1H, J₁=5.2 Hz, J₂=6.5 Hz, H1′), 5.10-4.50 (m, 6H, 3 CH₂Bn), 4.45 (m, 1H, H6′), 4.30 (m, 2H, CH₂Bn), 3.90 (m, 2H, H3′+H4′), 3.70 (m, 1H, H5′), 3.50 (m, 2H, H7a′+H7b′), 3.20 (m, 1H, H2b′), 2.55 (dd, 1H, H2a′). HPLC: t=17.8 min, UV λ max =265 nm. A-6b: ¹H NMR (300 MHz, CDCl₃): δ 8.60 (s, 1H, H8), 8.25 (s, 1H, H2), 7.40-7.00 (m, 20H, 4Bn), 6.20 (dd, 1H, J₁=2.5 Hz, J₂=10 Hz, H1′), 4.70-4.20 (m, 9H, 4CH₂Bn+H6′), 3.95 (m, 2H, H4′+H5′), 3.80 (dd, 1H, J₁=1.4 Hz, J₂=9.6 Hz, H3′), 3.75-3.45 (m, 2H, H7a′+H7b′), 2.70 (m, 1H, H2b′), 2.40 (m, 1H, H2a′). HPLC: t=17.6 min, UV λ max=264 nm.

Compound A-7a. To a solution of A-6a (1.0 g, 1.45 mmol) in anhydrous dichloromethane (30 ml) at −78° C. was added boron trichloride (1M in dichloromethane, 14.5 ml, 14.5 mmol) dropwise. The mixture was stirred at −78° C. for 3 hours then allowed to rise about −30° C. and stirred for 3 hours. The reaction was quenched by addition of methanol/dichloromethane (1/1) and stirred for 30 min at −15° C., then neutralized at 0° C. with aqueous ammonia and stirred at room temperature for 15 min. The solid was filtered through a pad of celite and washed with dichloromethane/methanol (1/1). The filtrate was evaporated to dryness and the residue was purified on silica gel column chromatography (eluent: from 8 to 14% of methanol in dichloromethane) to give compound A-7a (434 mg, 91 %). Molecular Formula: C₁₂H₁₅ClN₄O₅. ¹H NMR (300 MHz, DMSO d₆): δ 9.10 (s, 1H, H8), 8.80 (s, 1H, H2), 6.30 (dd, 1H, J₁=5.3 Hz, J₂=11.8 Hz, H1′), 5.20 (d, 1H, J=4, 1 Hz, OH′), 5.05 (d, 1H, J=4.7 Hz, OH′), 4.75 (d, 1H, J=3.1 Hz, OH3′), 4.15 (m, 1H, OH7′), 4.05 (m, 1H, H6′), 3.60 (m, 2H, H7a′+H3′), 3.30-3.00 (m, 4H, H7b′+H5′+H4′+H2b′), 2.30 (dd, 1H, J₁=5.4 Hz, J₂=13.7 Hz, H2a′). LC/MS: t=1.5 min; MS ES+=331 (M+H)⁺, UV λ max=265 nm.

Compound A-8a. To a solution of A-7a (40 mg, 0.12 mmol) in anhydrous pyridine (0.50 ml), was added under argon at 0° C. acetic anhydride (0.114 ml, 1.2 mmol) and a catalytic amount of dimethylaminopyridine (DMAP). The reaction mixture was stirred at room temperature overnight, diluted in ethyl acetate and sequentially washed with a 1M HCI solution, a saturated aqueous solution of sodium bicarbonate and brine. The organic layer was dried (Na₂SO₄) and concentrated under vacuo. The crude was purified on a silica gel column chromatography (eluent: 1% of ethanol in dichloromethane) to afford compound A-8a (15 mg, 25 %). Molecular Formula: C₂₀H₂₃ClN₄0₉. ¹H NMR (300 MHz, CDCQ): δ 8.75 (s, 1H, H8), 8.20 (s, 1H, H2), 6.20 (dd, 1H, J₁=5.5 Hz, J₂=11.7 Hz, H1′), 5.45 (m, 1H, H4′), 5.15-4.85 (m, 3H, H3′+H5′+H6′), 4.00-3.70 (m, 3H, H7a′+H2b′+H7b′), 2.40 (m, 1H, H2a′), 2.10-2.00 (m, 12H, 4OAc). MS: FAB+(matrice GT) m/z 499 (M+H)⁺. UV: λ max=265 nm.

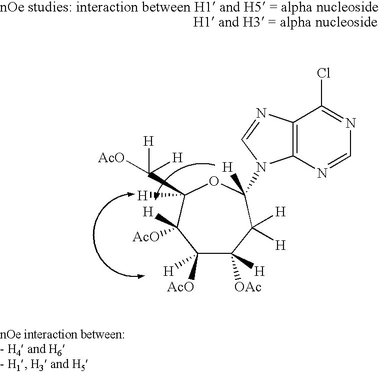

Compound A-9a. A solution of A-7a (100 mg, 0.30 mmol) in a saturated solution of ammonia in methanol (10 ml) was placed in a steel bomb and heated at 90° C. during 4 hours, then the resulting solution was evaporated to dryness and purified on a silica gel column chromatography (eluent: dichloromethane/methanol (8/2)) to furnish compound A-9a (60 mg, 64 %). Molecular Formula: C₁₂H₁₇N₅O₅. ¹H NMR (300 MHz, DMSO d6): δ 8.50 (s, 1H, H8), 8.15 (s, 1H, H2), 7.25 (s, 2H, NH₂), 6.10 (dd, 1H, J₁=4.9 Hz, J₂=11.6 Hz, H1′), 5.05 (m, 1H, OH4′), 4.95 (d, 1H, J=4.4 Hz, OH5′), 4.70 (m, 1H, OH3′), 4.20 (m, 1H, OH7′), 4.00 (t, 1H, J=7, H6′), 3.60-3.50 (m, 2H, H3′+H7a′), 3.30-3.10 (m, 3H, H7b′+H4′+H5′), 3.00 (m, 1H, H2a′), 2.15 (dd, 1H, J₁=4.9 Hz, J₂=14.7 Hz, H2b′), MS: FAB+(matrice GT) m/z 312 (M+H)⁺. UV: λ max=265 nm.

Compound A-7b: To a solution of A-6b (1.04g, 1.51 mmol) in anhydrous dichloromethane (30 ml) at −78° C. was added boron trichloride (1M in dichloromethane, 14.5 ml, 14.5 mmol) dropwise. The mixture was stirred at −78° C. for 3 h then allowed to rise about −30° C. and stirred for 3 h. The reaction was quenched by addition of methanol/dichloromethane (1/1) and stirred for 30 min at −15° C., then neutralized at 0° C. with aqueous ammonia and stirred at room temperature for 15 min. The solid was filtered through a pad of celite and washed with dichloromethane/methanol (1/1). The filtrate was evaporated to dryness and the residue was purified on a silica gel column chromatography (eluent: dichloromethane/methanol (9/1)) to give compound A-7b (282 mg, 57 %). Molecular Formula: C₁₂H₁₅ClN₄O₅. ¹H NMR (300 MHz, DMSO d₆): δ 9.00 (s, 1H, H8), 8.90 (s, 1H, H2), 6.25 (dd, 1H, J₁=4.1 Hz, J₂=7.3 Hz, H1′), 5.00 (bs, 3H, 3OH′), 4.50 (bs, 1H, 1OH′), 4.00 (m, 1H, H3′), 3.90 (m, 1H, H6′), 3.70 (m, 1H, H7a′), 3.60 ( m, 1H, H4′), 3.45 (m, 2H and water, H5′+H7b′), 2.75 (m, 1H, H2b′), 2.40 (m, 1H, H2a′). LC/MS: t=5.6 min; MS ES+=331 (M+H)⁺, UV λ max=265 nm.

Compound A-8b. To a solution of A-7b (70 mg, 0.21 mmol) in anhydrous pyridine (1 ml), was added under argon at 0° C. acetic anhydride (0.2 ml, 2.1 mmol) and a catalytic amount of dimethylaminopyridine (DMAP). The reaction mixture was stirred at room temperature for Ih, diluted in ethyl acetate and sequentially washed with 1M HCl solution, a saturated aqueous solution of sodium bicarbonate and brine. The organic layer was dried (Na₂SO₄) and concentrated under vacuo. The crude was purified on a silica gel column chromatography (eluent: 1% of ethanol in dichloromethane) to afford compound A-8b (64 mg, 61%). Molecular Formula: C₂₀H₂₃ClN₄O₉. ¹H NMR (300 MHz, CDCl₃): δ 8.70 (s, 1H, H8), 8.40 (s, 1H, H2), 6.20 (dd, 1H, J₁=3.8 Hz, J₂=8.3 Hz, H1′), 5.45-5.25 (m, 2H, H4′+H3′), 5.10 (dd, 1H, J₁=4.3, J₂=9.7, H5′), 4.30 (m, 1H, H6′), 4.05 (m, 2H, H7a′+H7b′), 2.80-2.70 (m, 1H, H2b′), 2.60-2.50 (m, 1H, H2a′), 2.20-1.90 (m, 12H, 4OAc ). nOe studies: interaction between H1′ and H6′=beta nucleoside. UV: λ max=265 nm.

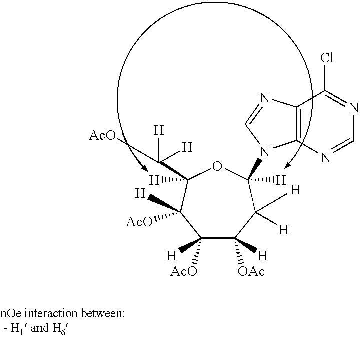

Compound A-9b. A solution of A-7b (137 mg, 0.41 mmol) in a saturated solution of ammonia in methanol (10 ml) was placed in a steel bomb and heated at 90° C. during 7 hours, then the resulting solution was evaporated to dryness and purified by reverse phase (C 18) silica gel column chromatography (eluent: water) to obtain compound A-9b (60 mg, 47 %). Molecular Formula: C₁₂H₁₇N₅O₅. ¹H NMR (300 MHz, DMSO d₆: δ 8.30 (s, 1H, H8), 8.10 (s, 1H, H2), 7.30 (s, 2H, NH₂), 6.00 (m, 1H, H1′), 5.00 (m, 3 H, OH4′+OH3′+OH5′), 4.60 (m, 1H, OH7′), 3.90 (m, 1H, H3′), 3.80 (m, 1H, H6′), 3.70 (m, 1H, H7a′), 3.55 (m, 1H, H4′), 3.40 (m, 2H, H7b′+H5′), 2.60 (m, 1H, H2a′), 2.30 (m, 1H, H2b′). LC/MS: t=1.3 min; MS ES+=312 (M+H)⁺, UV λ max=260 nm.

### Example 3

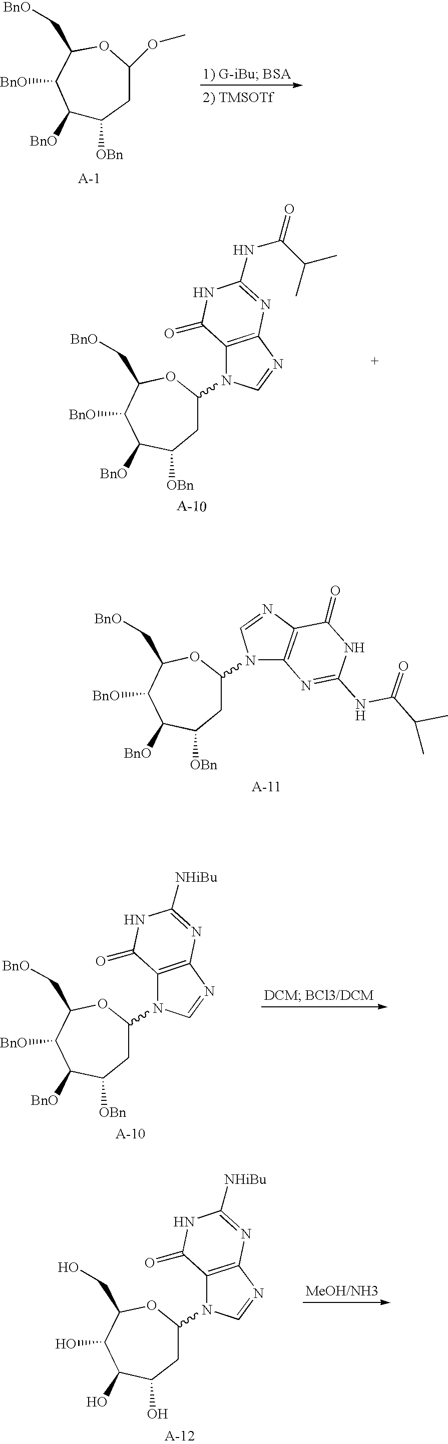

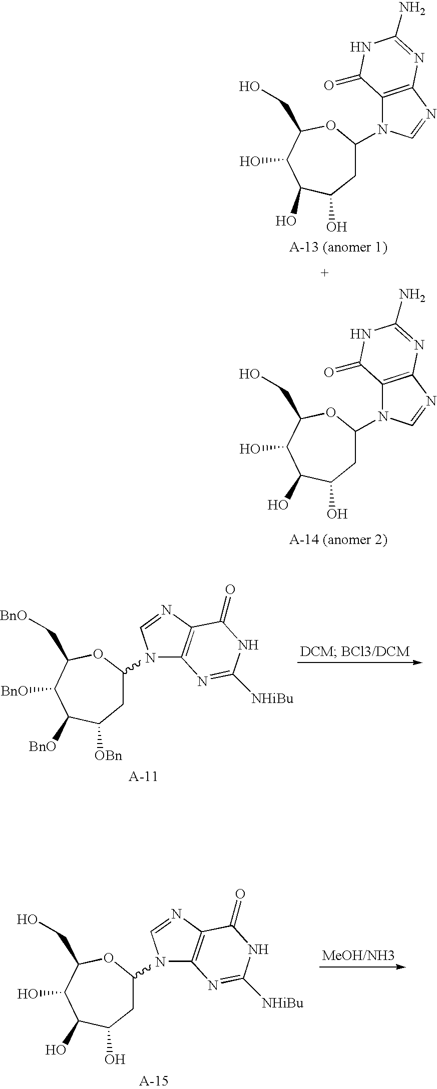

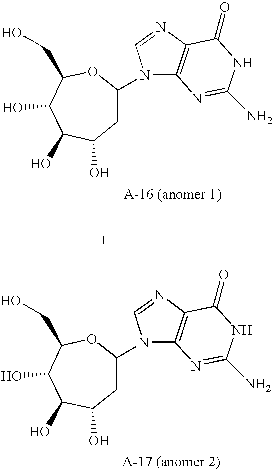

Compounds A-10 and A-11. To a suspension of N2-isobutyrylguanine (932 mg, 4.22 mmol) in anhydrous toluene (7 ml) was added N,O-bis(trimethylsilyl)acetamide (3.49 ml, 14.07 mmol). The reaction mixture was stirred at reflux during 3 hours. To the previous reaction mixture was added compound A-1 (2.0 g, 3.52 mmol) in solution in anhydrous toluene (13 ml), trimethylsilyltrifluoromethanesulfonate (TMSOTf, 2.72 ml, 14.07 mmol) and the reaction mixture was stirred at reflux for 40 min. At room temperature the reaction mixture was diluted with ethyl acetate, neutralized with a saturated aqueous solution of sodium bicarbonate and the mixture was filtrated through a pad of celite. The organic layer was washed with brine, dried over sodium sulfate (Na₂SO₄) and evaporated to dryness. The crude material was purified on a silica gel column chromatography (eluent: from 2 to 2.5% of ethanol in diethyl ether) to give compound A-10 (630 mg, 24%) and compound A-11 (390 mg, 15%). Molecular Formula: C₄₁H₄₇N₅O₇. A-10 (major anomer): ¹H NMR (400 MHz, DMSO d6): δ 12.15 (bs, 1H, NH), 9.25 (bs, 1H, NHiBu), 8.40 (s, 1H, H8), 7.40-7.10 (m, 20H, 4Bn), 6.45 (dd, 1H, H1′), 5.10-4.20 (m, 9H, 4CH₂Bn+H), 4.00-3.50 (m, 5H), 3.90-3.50 (m, 3H, H2a′+H2b′+CH iBu), 1.25 (m, 6H, 2CH₃ iBu). LC/MS: t=21.45 min; MS ES+=758 (M+H)⁺, UV λ max=265 nm. A-11: (major anomer) ¹H NMR (400 MHz DMSO d₆): δ 12.10 (bs, 1H, NH), 8.20 (bs, 1H. NHiBu), 7.90 (s, 1H, H8), 7.40-7.10 (m, 20H, 4Bn), 6.00 (m, 1H, H1′), 4.80-4.30 (m, 8H, 4CH₂Bn), 4.10 (m, 1H), 3.90-3.60 (m, 5H) 2.90-2.30 (m, 3H, H2a′+H2b′+CH iBu), 1.30 (m, 6H, 2CH₃ iBu). LC/MS: t=21 min; MS ES+=758 (M+H)⁺., UV λ max=259 nm.

Compound A-12. To a solution of compound A-10 (246 mg, 0.325 mmol) in anhydrous dichloromethane (3.15 ml) at −78° C. was added boron trichloride (1M in dichloromethane, 1.62 ml, 1.62 mmol) dropwise. The mixture was stirred at −78° C. for 2 hours then allowed to rise about −40° C. and stirred for 2 hours. The reaction was quenched by addition of methanol/dichloromethane (1.05 ml) (1/1) and stirred for 30 min at −20° C., then neutralized at 0° C. with aqueous ammonia and stirred at room temperature for 15 min. The solid was filtered through a pad of celite and washed with dichloromethane/methanol (1/1). The filtrate was evaporated to dryness and the residue was purified on a silica gel column chromatography (eluent: dichloromethane/methanol (8/2)) to give compound A-12 (114 mg, 88%). Molecular Formula: C₁₆H₂₃N₅O₇. (major anomer) ¹H NMR (400 MHz, DMSO d₆): δ 12.00 (bs, 1H, NH), 9.50 (bs, 1H. NHiBu), 8.70 (s, 1H, H8), 6.40 (dd, 1H, J₁=5.2 Hz, J₂=11.9 Hz, H1′), 5.15 (bs, 1H, OH′), 5.05 (bs, 1H, OH′), 4.75 (bs, 1H, OH′), 4.20 (d, 2H, 1OH+H′), 3.95 (m, 1H), 3.40 (m under water, 4H), 2.75 (m, 2H, H2a′+CH iBu), 2.20 (dd, 1H, J₁=5.2 Hz, J₂=13.6 Hz, H2b′), 1.15 (d, 6H, J=6.8 Hz, 2CH₃ iBu). LC/MS: t=6.8 min; MS ES+=398 (M+H)⁺., UV λ max=265 nm.

Compounds A-13 and A-14: A-12 (89 mg, 0.22 mmol) was suspended in solution with a saturated solution of ammonia in methanol (4.4 ml), the mixture was stirred at room temperature overnight and evaporated to dryness. The crude contained final product but also starting material so it was suspended in solution with a saturated solution of ammonia in methanol (4.4 ml), the mixture was placed into a steel bomb and heated at 90° C. for 2 hours, then concentrated under vacuo, diluted with water, washed twice with ethyl acetate and the aqueous phase was evaporated to dryness. The residue was purified by reverse phase (C 18) silica gel column chromatography (eluent: water) to afford compound A-13 (anomer 1) (23 mg, 32%) and compound A-14 (anomer 2) (2.6 mg, 4%). Molecular Formula: C₁₂H₁₇N₅O₆. A-13: ¹H NMR (200 MHz, DMSO d₆): δ 10.80 (bs, 1H, NH), 8.50 (s, 1H, H8), 6.30 (dd, 1H, J₁=5 Hz, J₂=11.9 Hz, H1′), 6.20 (bs, 2H, NH2), 5.10 (bs, 1H, OH′), 4.90 (d, 1H, J=4.4 Hz, 1OH′), 4.70 (d, 1H, J=3.1 Hz, 1OH′), 4.20 (t, 1H, J=5.6 Hz, OH7′) 3.90 (m, 1H), 3.40 (m under water, 5H), 2.80 (m, 1H, H2a′), 2.20 (dd, 1H, H2b′). LC/MS: t=1.08 min; MS ES+=328 (M+H)⁺., UV λ max=285 nm. A-14: UV X max =286 mu.

Compound A-15. To a solution of compound A-11 (476 mg, 0.628 mmol) in anhydrous dichloromethane (6.1 ml) at −78° C. was added boron trichloride (1M in dichloromethane, 3.14 ml, 3.14 mmol) dropwise. The mixture was stirred at −78° C. for 2 hours then allowed to rise about −40° C. and stirred for 2 hours. The reaction was quenched by addition of methanol/dichloromethane (2.03 ml) (1/1) and stirred for 30 min at −20° C., then neutralized at 0° C. with aqueous ammonia and stirred at room temperature for 15 min. The solid was filtered through a pad of celite and washed with dichloromethane/methanol (1/1). The filtrate was evaporated to dryness and the residue was purified on silica gel column chromatography (eluent: from 10 to 30% of methanol in dichloromethane) to give compound A-15 (315 mg, >100% because of presence of silica gel). Molecular Formula: C₁₆H₂₃N₅O₇. LC/MS: t=6.8 min; MS ES+=398 (M+H)⁺., UV λ max=260 nm.

Compounds A-16 and A-17. A-15 (315 mg, 0.79 mmol) in solution with a saturated solution of ammonia in methanol (16 ml) was placed in a steel bomb and heated at 100° C. for 1 hour then evaporated to dryness. The residue was dissolved in water and the aqueous layer was washed twice with ethyl acetate. The aqueous layer was concentrated under vacuo. The residue was purified by reverse phase (C 18) silica gel column chromatography (eluent: water) to afford compound A-16 (anomer 1) (165 mg, 64 %) and compound A-17 (anomer 2) (8 mg, 3%). Molecular Formula: C₁₂H₁₇N₅O₆. A-16: ¹H NMR (200 MHz, DMSO d₆): δ 10.70 (bs, 1H, NH), 8.10 (s, 1H, H8), 6.50 (bs, 2H, NH2), 5.90 (dd, 1H, J₁=4.9 Hz, J₂=11.7 Hz, H1′), 5.10-4.80 (m, 2H, 20H′), 4.70 (bs, 1H, 1OH′), 4.20 (t, 1H, J=5.6 Hz, OH7′) 3.90 (m, 1H), 3.40 (m under water, 5H), 2.75 (m, 1 H, H2a′), 2.10 (dd, 1H, J₁=5.4 Hz, J₂=14.2Hz, H2b′). LC/MS: t=1.28 min; MS ES+=328 (M+H)⁺, UV λ max=253 nm. A-17: ¹H NMR (200 MHz, DMSO d₆): δ10.60 (bs, 1H, NH), 7.85 (s, 1H, H8), 6.50 (bs, 2H, NH2), 5.85 (m, 1H, H1′), 5.00 (bs, 3H, 3OH′), 4.50 (m, 1H, OH7′), 3.90-3.60 (m, 3H), 3.40 (under water, 3H), 2.40 (m, 1H, H2a′), 2.20 (m, 1H, H2b′). HPLC: t=1.11 min, UV λ max=253 nm.

### Example 4

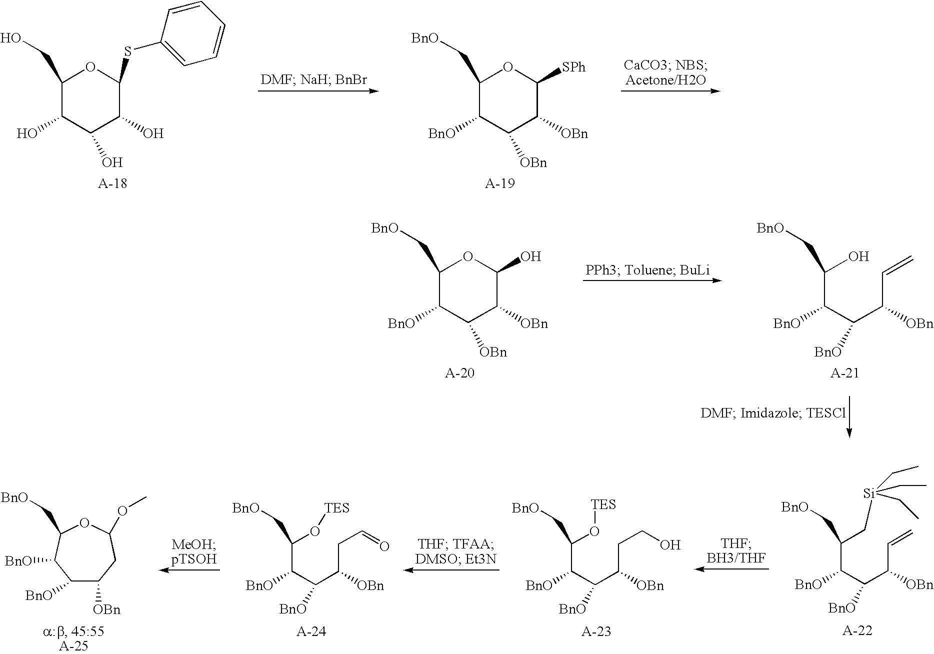

Compound A-18. was prepared according to: Gomez A. M.; Compagny M. D.; Agos A.; Uriel C.; Valverde S.; Lopez J. C., Carbohydrate Res., 2005, 340, 1872-75. Molecular Formula: C₁₂H₁₆O₅S.

Compound A-19. To a solution of A-18 (34.0 g, 126 mmol) in dimethylformamide (DMF, 690 ml) at 0° C. was added sodium hydride 60% (30.2 g, 756 mmol), the mixture was stirred for 2h at the same temperature, then benzylbromide (89.5 ml, 756 mmol) was added at 0° C., and the reaction mixture was stirred at the same 20 temperature during 3 hours. The mixture was diluted with ethyl acetate and poured into water. The organic phase was sequentially washed with a saturated aqueous solution of sodium bicarbonate and water and then dried over sodium sulfate and concentrated under vacuo. The crude material was purified on a silica gel column chromatography (eluent: petroleum ether/diethyl ether (8/2)) to afford compound A-19 (60.8 g, 76%). Molecular Formula: C₄₀H₄₀O₅S. ¹H NMR (250 MHz, CDCl₃): δ7.40-7.10 (m, 25H, 4Bn+SPh), 5.25 (d, 2H, J=9.36 Hz), 4.80 (d, 2H, J=5.4 Hz), 4.60 (m, 2H), 4.60 (s, 1H), 4.40 (s, 2H), 4.10 (m, 2H), 3.75 (m, 2H), 3.50 (dd, 1H, J₁=2.5 Hz, J₂=9.8 Hz), 3.35 (dd, 1H, J₁=2.2 Hz, J₂=9.6 Hz).

Compound A-20. Calcium carbonate (46.2 g, 461 mmol) and N-bromosuccinimide (32.9 g, 185 mmol) were added to a solution of A-19 in acetone:water (9:1) (237 ml), the reaction mixture was stirred at room temperature for 3 h, then filtrated. The filtrate was diluted with ethyl acetate and washed with a saturated aqueous solution of sodium bicarbonate, the organic layer was filtrated once again, dried over sodium sulfate and concentrated under vacuo. The crude material was purified on a silica gel column chromatography (eluent: petroleum ether/diethyl ether (75/25), (1/1)) to afford compound A-20 (37.7 g, 73%). Molecular Formula: C₃₄H₃₆O₆. ¹H NMR (250 MHz, DMSO d₆): δ 7.40-7.20 (m, 20H, 4Bn), 6.60 (d, 1H, J=4.5 Hz, OH), 5.25 (m, 1H, H1′), 4.90-4.80 (m, 8H, 4CH₂Bn), 3.85 (m, 2H), 3.60 (m, 2H), 3.40 (m, 2H). MS: ESI m/z 523 (S)⁺, m/z 558 (M+NH₄⁺).

Compound A-21. To a solution of triphenylphosphonium bromide (49.6 g, 138.7 mmol) in anhydrous toluene (500 ml) was added butyllithium (1.6M in hexanes, 84 ml, 134.1 mmol) at 0° C., under argon. The mixture was stirred at room temperature for 1 h, to this reaction mixture was added under argon A-20 (50 g, 92.48 mmol) in solution in a minimum volume of toluene. The all was stirred at room temperature during two days. The mixture was diluted with water and diethyl ether, the aqueous layer was extracted twice with diethyl ether and the combined organic layer was dried over sodium sulfate and concentrated under vacuo. The crude product A-21 was directly used in the next step. Molecular Formula: C₃₅H₃₈O₅. ¹H NMR (400 MHz, DMSO dm₆): δ 7.40-7.20 (m, 20H, 4 Bn), 5.90 (m, 1H), 5.30 (dd, 1H, J₁=2.2 Hz, J₂=10.5 Hz), 5.15 (dd, 1H, J₁=1.9 Hz, J₂=17.3 Hz), 5.10 (d, 1H, J=5.1 Hz, OH), 4.70 (m, 2H), 4.55 (m, 3H), 4.50 (s, 2H), 4.40 (d, 1H, J=12.1 Hz), 4.20 (dd, 1H, J₁=3.2 Hz, J₂=7.8 Hz), 4.10 (m, 1H), 3.90 (dd, 1H, J₁=3.2 Hz, J₂=7.8 Hz), 3.60-3.45 (m, 3H). MS: ESI m/z 539 (M+H)⁺, m/z 1077 (2M+H)⁺.

Compound A-22. A-21 (49.83 g, 52.75 mmol) and dimethylformamide (DMF, 122 ml) were combined under argon and to this mixture was added imidazole (18.9 g, 277.44 mmol) followed by the addition of chlorotriethylsilane (TESCI) (16.55 ml, 9.11 mmol) dropwise. This solution was stirred at 40° C. for 2 hours. The reaction mixture was diluted with ethyl acetate and sequentially washed twice with a saturated aqueous solution of sodium bicarbonate, and twice with brine. The organic layer was dried over sodium sulfate and concentrated under vacuo. Impurities were crystallized into hexanes, filtered and washed with diethyl ether. The filtrate was concentrated under reduced pressure and the crude material was purified on a silica gel column chromatography (eluent: hexanes/ethyl acetate (99/1)) to afford compound A-22 (30.04 g, 50%). Molecular Formula: C₄₀H49O₅Si.

Compound A-23. To a solution of A-22 (30 g, 46 mmol) in tetrahydrofurane (THF) (213 ml), at 0° C. was added dropwise a solution of borane (1M in THF, 165 ml). The mixture was allowed to warm to room temperature and stirred under argon. After 3 hours, the reaction was quenched with dropwise addition of water (87.2 ml) at 0° C. To the mixture was added a 4M aqueous solution of sodium hydroxide (220.5 ml) dropwise and then 35 % hydrogen peroxide (62.6 ml) at the same temperature. The mixture was stirred at room temperature overnight. A saturated aqueous solution of sodium bicarbonate was added and tetrahydrofurane was removed under reduced pressure. The residue was extracted twice with dichloromethane, the combined organic layer was washed twice with water and dried over sodium sulfate and concentrated under vacuo. Purification on a silica gel column chromatography (eluent: from 5 to 20% of ethyl acetate in petroleum ether) gave compound A-23 (19.2 g, 70%). Molecular Formula: C₄₁H₅₄O₆Si. ¹H NMR (400 MHz, CDCl₃): δ 7.40-7.20 (m, 20H, 4 Bn), 4.80 (t, J=12 Hz, 2H), 4.70-4.45 (m, 6H), 4.35 (m, 1H), 4.00 (m, 2H), 3.70-3.55 (m, 5H), 1.95 (m, 1H), 1.60 (m, 2H), 0.95 (m, 9H, 3×CH₃ TES), 0.65 (m, 6H, 3×CH₂ TES). MS: ESI m/z 671 (M+H)⁺.

Compound A-24. A solution of tetrahydrofurane (40 ml) and trifluoroacetic anhydride (4.52 ml, 32.51 mmol) was placed at −60° C. Dimethylsulfoxide (7.65 ml, 107.67 mmol) was then added to the mixture and it was stirred for 2 min then compound A-23 (11.36, 16.93 mmol) in solution with tetrahydrofurane (80 ml) was added and the mixture was allowed to stir for 15 min. Triethylamine (13.43 ml, 96.5 mmol) was added and the reaction mixture was stirred for 5 min and then warmed to room temperature over 1 hour. Water was added to the mixture and the aqueous phase was extracted three times with dichloromethane. The combined organic layer was washed with brine, dried over sodium sulfate and concentrated under vacuo. Purification on a silica gel column chromatography (eluent: from 5 to 6% of ethyl acetate in cyclohexane) gave compound A-24 (10.37 g, 92%). Molecular Formula: C₄₁H₅₂O₆Si. ¹H NMR (400 MHz, DMSO d₆): δ 9.50 (m, 1H, CHO), 7.30-7.10 (m, 20H, 4Bn), 4.75-4.35 (m, 8H, CH₂Bn), 4.30-4.15 (m, 2H), 3.95 (m, 1H), 3.60 (m, 1H), 3.50 (m, 2H), 2.60 (m, 1H, H2a′), 2.25 (m, 1H, H2b′), 0.85 (m, 9H, 3×CH₃ TES), 0.50 (m, 6H, 3×CH₂ TES). MS: ESI m/z 669.2 (M+H)⁺.

Compound A-25. A-24 (10.37 g, 15.51 mmol) was dissolved in methanol (250 ml). To this solution was added p-toluenesulfonic acid monohydrate (7.08 g, 37.24 mmol) and the mixture was stirred at room temperature for 2 hours. A saturated aqueous solution of sodium bicarbonate was added and the mixture was extracted three times with dichloromethane. The combined organic layer was washed with brine, dried over sodium sulfate and concentrated under vacuo. Purification on a silica gel column chromatography (eluent: from 5 to 20% of ethyl acetate in cyclohexane) gave compound A-25 (3.32 g, 38%) (α/β=45/55). Molecular Formula: C36H₄₀O₆. ¹H NMR (400 MHz, CDCl₃): δ 7.40-7.20 (m, 20Hα, 4Bn+20Hβ, 4Bn), 4.80-4.30 (m, 8Hα, 4CH₂Bn+8Hβ, 4CH₂Bn), 4.20 (m, 1Hα, H1+1Hβ, H1), 4.10 (m, 2Hβ), 4.05 (m, 1Hα), 3.80 (m, 1Hα), 3.70 (m, 2Hβ+1Hα), 3.60-3.50 (m, 2Hβ+1Hα), 3.45-3.35 (m, 2Hα), 3.30 (s, 3H, OCH₃β), 3.25 (s, 3H, OCH₃α), 2.60 (m, 1H, H2aα), 2.15 (m, 1H, H2bα+1H, H2aβ), 1.95 (m, 1H, H2bβ). MS: ESI m/z 537.4 (S)⁺, m/z 586.39 (M+NH₄⁺).

### Example 5

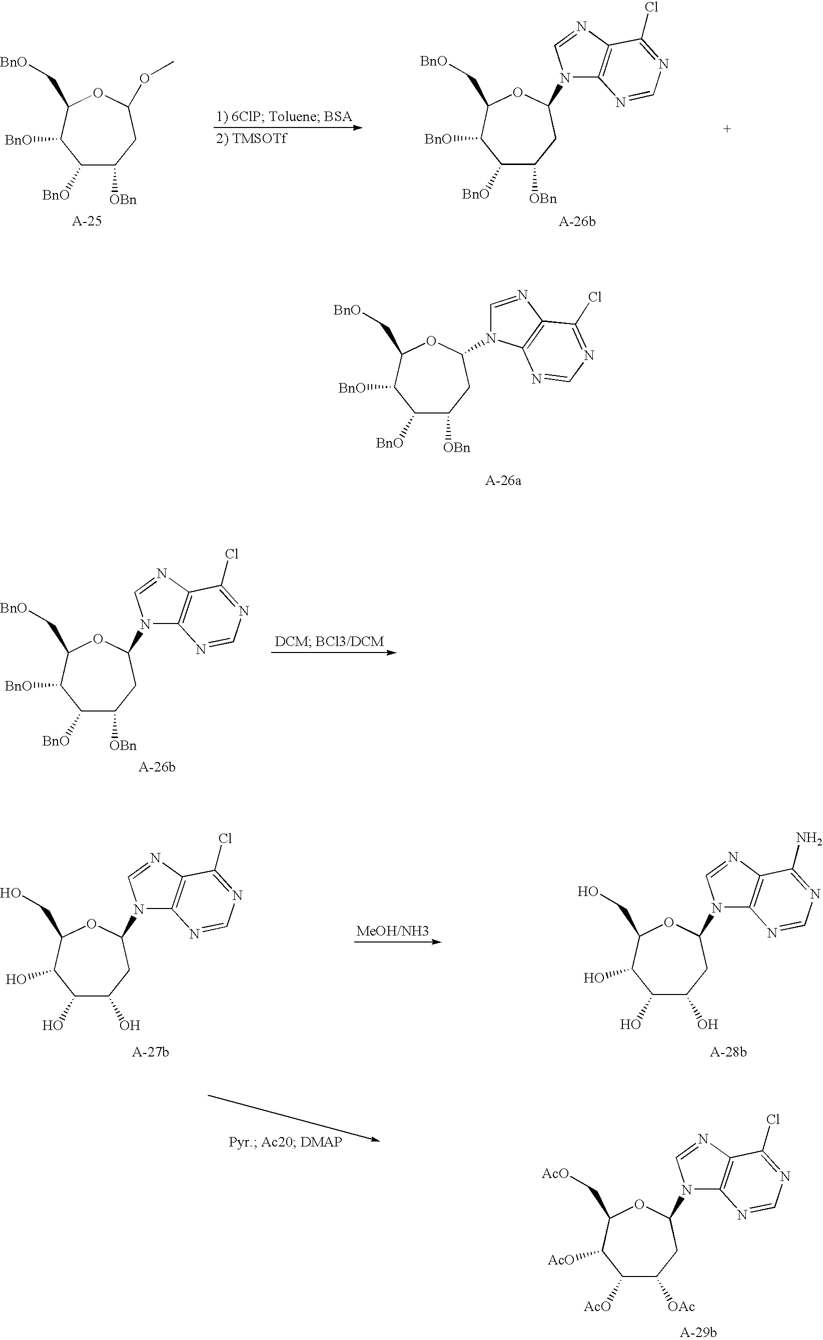

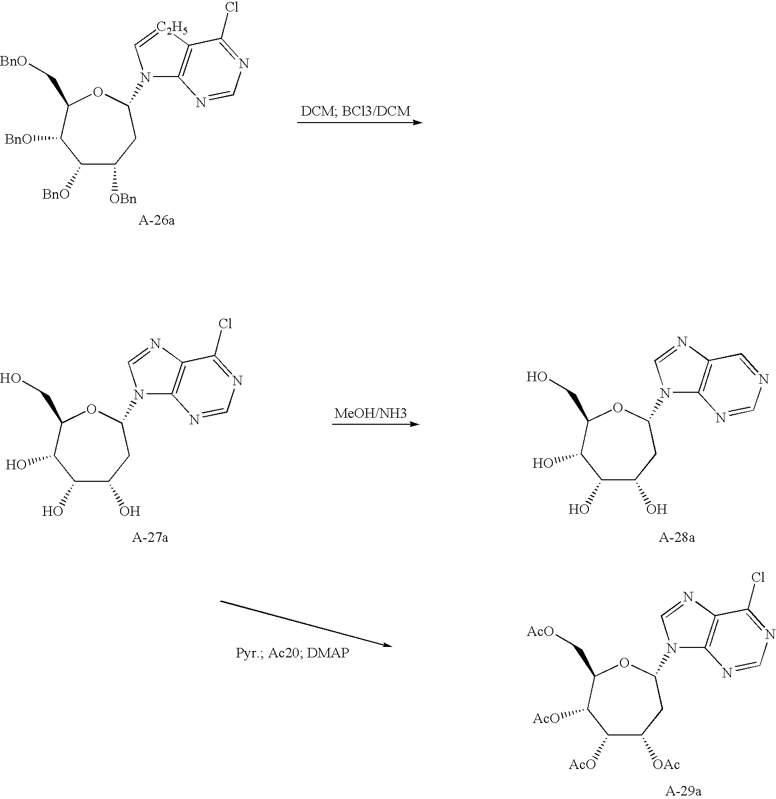

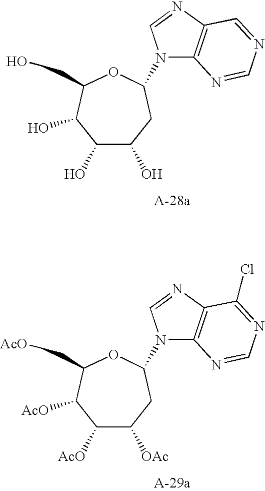

Compounds A-26b and A-26a: To a suspension of 6-chloropurine (1.63 g, 10.54 mmol) in anhydrous toluene (45 ml) was added N,O-bis(trimethylsilyl)acetamide (4.37 ml, 17.6 mmol). The reaction mixture was stirred at reflux during 15 min. At room temperature and under argon, to this mixture was added compound A-25 (5.0 g, 8.8 mmol) and trimethylsilyltrifluoromethanesulfonate (TMSOT, 3.4 ml, 17.6 mmol), the reaction mixture was stirred at reflux for 2 hours, then, at room temperature was diluted with ethyl acetate, washed successively with a 5% aqueous solution of sodium bicarbonate, brine, dried over sodium sulfate and concentrated under vacuo. Purification on a silica gel column chromatography (eluent: petroleum ether/diethyl ether (1/1)) afforded compound A-26b (1.51 g, 25%) and compound A-26a (1.05 g, 17%). Molecular Formula: C₄₀H₃₉ClN₄O₅. A-26b: ¹H NMR (300 MHz, CDCl₃): δ 8.60 (s, 1H, H8), 8.20 (s, 1H, H2), 7.40-7.00 (m, 20H, 4Bn), 6.40 (dd, 1H, J₁=5.6 Hz, J₂=10 Hz, H1′), 4.80 (m, 2H, CH₂Bn), 4.50-4.00 (m, 8H), 3.80 (m, 1H), 3.50 (m, 3H), 2.65 (m, 1H, H2b′), 2.50 (m, 1H, H2a′). LC/MS: t=23.3 min; MS ES+=691 (M+H)⁺, UV □max=265 nm. A-26a: ¹H NMR (300 MHz, CDCl₃): δ 8.60 (s, 1H, H8), 8.35 (s, 1H, H2), 7.40-7.00 (m, 20H, 4Bn), 6.20 (dd, 1H, J₁=4.6 Hz, J₂=10.8 Hz, H1′), 4.85 (m, 2H, 1CH₂Bn), 4.50-4.10 (m, 8H), 3.70 (m, 1H), 3.50 (m, 3H), 3.30 (m, 1H, H2b′), 2.35 (m, 1H, H2a′). LC/MS: t=22.9 min; MS ES+=691 (M+H)⁺, UV λmax=265 nm.

Compound A-27b. To a solution of A-26b (1.5 g, 2.17 mmol) in anhydrous dichloromethane (20 ml) at −78° C. was added boron trichloride (1M in dichloromethane, 10.8 ml, 10.8 mmol) dropwise. The mixture was stirred at −78° C. for 3 hours then allowed to rise about −40° C. and stirred for 2 hours. The reaction was quenched by addition of methanol/dichloromethane (1/1) and stirred for 30 min at −30° C., then neutralized at 0° C. with aqueous ammonia and stirred at room temperature for 15 min. The solid was filtered through a pad of celite and washed with dichloromethane/methanol (1/1). The filtrate was evaporated to dryness and the residue was purified on a silica gel column chromatography (eluent: dichloromethane/methanol (8/2)) to afford compound A-27b (178 mg, 25%). Molecular Formula: C₁₂H₁₅ClN₄O₅. ¹H NMR (300 MHz, DMSO d₆): δ 9.00 (s, 1H, H2), 8.80 (s, 1H, H8), 6.30 (m, 1H, H1′), 5.30 (d, 1H, J=4 Hz, OH′), 5.00 (m, 2H, 2OH′), 4.50 (m, 1H, OH7′), 4.15 (m, 1H, H4′), 4.00 (m, 2H, H3′+H5′), 3.75 (m, 1H, H6′), 3.60 (m, 1H, H7a′), 3.40 (m under water, 1H, H7b′), 2.80 (m, 1H, H2a′), 2.25 (m, 1H, H2b′). HPLC: t=3.8 min, UV λmax=265 nm.

Compound A-28b. A solution of A-27b (160 mg, 0.49 mmol) in a saturated solution of ammonia in methanol (25 ml) was placed in a steel bomb and heated at 110° C. during 5 hours, then the resulting solution was evaporated to dryness and purified by reverse phase (C 18) silica gel column chromatography (eluent: water) to furnish compound A-28b (61 mg, 40%). Molecular Formula: C₁₂H₁₇N₅O₅. ¹H NMR (300 MHz, DMSO d₆): δ 8.35 (s, 1H, H8), 8.10 (s, 1H, H2), 7.25 (s, 2H, NH₂), 6.15 (dd, 1H, J₁=5.1 Hz, J₂=9.8 Hz, H1′), 5.20 (bs, 1H, OH′), 4.95 (bs, 2H, 2OH′), 4.60 (m, 1H, OH′), 4.05 (t, 1H, J=7.2 Hz, H3′), 3.95 (s, 1H, H4′), 3.75 (m, 1H, H6′), 3.55 (m, 1H, H7a′), 3.50-3.40 (m, 2H, H5′+H7b′), 2.70 (m, 1H, H2a′), 2.20 (m, 1H, H2b′). LC/MS: t=1.5 min; MS ES+=312 (M+H)⁺, UV λmax=260 nm.

Compound A-29b. A-27b (230 mg, 0.70 mmol) was dissolved with anhydrous pyridine (20 ml) then acetic anhydride (0.65 ml, 7 mmol) and a catalytic amount of dimethylaminopyridine were added. The reaction mixture was stirred at room temperature overnight. The mixture was diluted with ethyl acetate and washed sequentially with an aqueous solution of HCl (1M) and a 5% aqueous solution of sodium bicarbonate, then the organic layer was dried over sodium sulfate and concentrated under vacuo. Purification on a silica gel column chromatography (eluent: from 1 to 5% of ethanol in dichloromethane) gave compound A-29b (44 mg, 13%). Molecular Formula: C₂₀H₂₃ClN₄O₉. ¹H NMR (300 MHz. CDCl₃): δ 8.70 (s, 1H, H2), 8.40 (s, 1H, H8), 6.30 (dd, 1H, J₁=5.7 Hz, J₂=9 Hz, H1′), 5.65 (m, 1H, H4′), 5.45 (m, 1H, H3′), 5.05 (dd, 1H, J₁=3 Hz, J₂=10 Hz, H5′), 4.25 (m, 1H, H6′), 4.05 (m, 2H, H7a′+H7b′), 2.80 (m, 1H, H2a′), 2.55 (m, 1H, H2b′), 2.20 (m, 3H, OAc), 2.00 (m, 9H, 3OAc). nOe studies: interaction between H1′ and H6′=beta nucleoside, HPLC: t=8.9 min, UV λmax=265 nm.

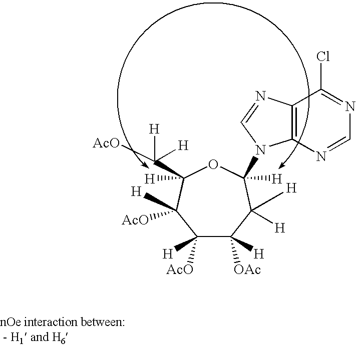

Compound A-27a. To a solution of A-26a (1.04 g, 1.5 mmol) in anhydrous dichloromethane (15 ml) at −78° C. was added boron trichloride (1M in dichloromethane, 7.5 ml, 7.5 mmol) dropwise. The mixture was stirred at −78° C. for 3 hours then allowed to rise about −40° C. and stirred for 2 hours. The reaction was quenched by addition of methanol/dichloromethane (1/1) and stirred for 30 min at −30° C., then neutralized at 0° C. with aqueous ammonia and stirred at room temperature for 15 min. The solid was filtered through a pad of celite and washed with dichloromethane/methanol (1/1). The filtrate was concentrated to dryness and the crude material was purified on a silica gel column chromatography (eluent: dichloromethane/methanol (8/2)) to afford compound A-27a (212 mg, 64%). Molecular Formula: C₁₂H₁₅ClN₄O₅. ¹H NMR (300 MHz, DMSO d₆): δ 9.00 (s, 1H, H2), 8.80 (s, 1H, H8), 6.25 (dd, 1H. J₁=4.9 Hz, J₂=11.6 Hz, H1′), 4.95 (d, 1H, J=3.3 Hz, OH4′), 4.85 (d, 1H, J=3.8 Hz, OH3′), 4.70 (d, 1H, J=7.2 Hz, OH5′), 4.20 (m, 2H, OH7′+H6′), 3.90 (m, 2H, H4′+H3′), 3.55 (m, 1H, H7b′), 3.40 (m under water, 1H, H2b′), 3.30 (m under water, 1H, H7a′), 2.00 (m, 1H, H2a′). HPLC: t=3.3 min, UV λmax=265 nm.

Compound A-28a. A solution of A-27a (190 mg, 0.58 mmol) in a saturated solution of ammonia in methanol (15 ml) was placed in a steel bomb and heated at 90° C. during 7 h, then the resulting solution was evaporated to dryness and purified by reverse phase (C 18) silica gel column chromatography (eluent: water) to furnish compound A-28a (82 mg, 45%). Molecular Formula: C₁₂H₁₇N₅O₅. ¹H NMR (300 MHz, DMSO d₆): δ 8.35 (s, 1H, H8), 8.10 (s, 1H, H2), 7.25 (s, 2H, NH₂), 6.05 (dd, 1H, J₁=4.1 Hz, J₂=11.4 Hz, H1′), 4.80 (m, 2H, OH4′+OH3′), 4.65 (m, 2H, OH5′+OH7′), 4.10 (m, 1H, H6′), 3.90 (m, 2H, H4′+H3′), 3.60-3.40 (m, 3H, H5′+H7a′+H7b′), 3.15 (m, 1H, H2a′), 2.00 (m, 1H, H2b′). LC/MS: t=1.3 min, MS ES+=312 (M+H)⁺, UV λmax=260 nm.

Compound A-29a. A-27a (130 mg, 0.39 mmol) was dissolved with anhydrous pyridine (10 ml) then acetic anhydride (0.37 ml, 3.9 mmol) and a catalytic amount of dimethylaminopyridine were added. The reaction mixture was stirred at room temperature overnight. The mixture was diluted with ethyl acetate and washed sequentially with an aqueous solution of HCl (1M) and a 5% aqueous solution of sodium bicarbonate, then the organic layer was dried over sodium sulfate and concentrated under vacuo. Purification on a silica gel column chromatography (eluent: 1% of methanol in dichloromethane) gave compound A-29a (42 mg, 22%). Molecular Formula: C₂₀H₂₃ClN₄O₉. ¹H NMR (300 MHz, CDCl₃): δ 8.75 (s, 1H, H2), 8.20 (s, 1H, H8), 6.20 (dd, 1H, J₁=5.7 Hz, J₂=11.6 Hz, H1′), 5.65 (m, 1H, H4′), 5.25 (m, 1H, H3′), 4.90 (m, 2H, H5′+H6′), 4.10 (m, 1H, H2b′), 3.90 (m, 2H, H7a′+H7b′), 2.20 (m, 4H, H2a′+OAc), 1.95 (m, 6H, 2OAc), 1.50 (m, 3H, OAc). nOe studies: interaction between H1′ and H3′=alpha nucleoside. HPLC: t=9 min, UV λmax=265 nm.

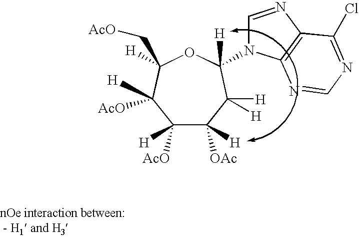

### Example 6

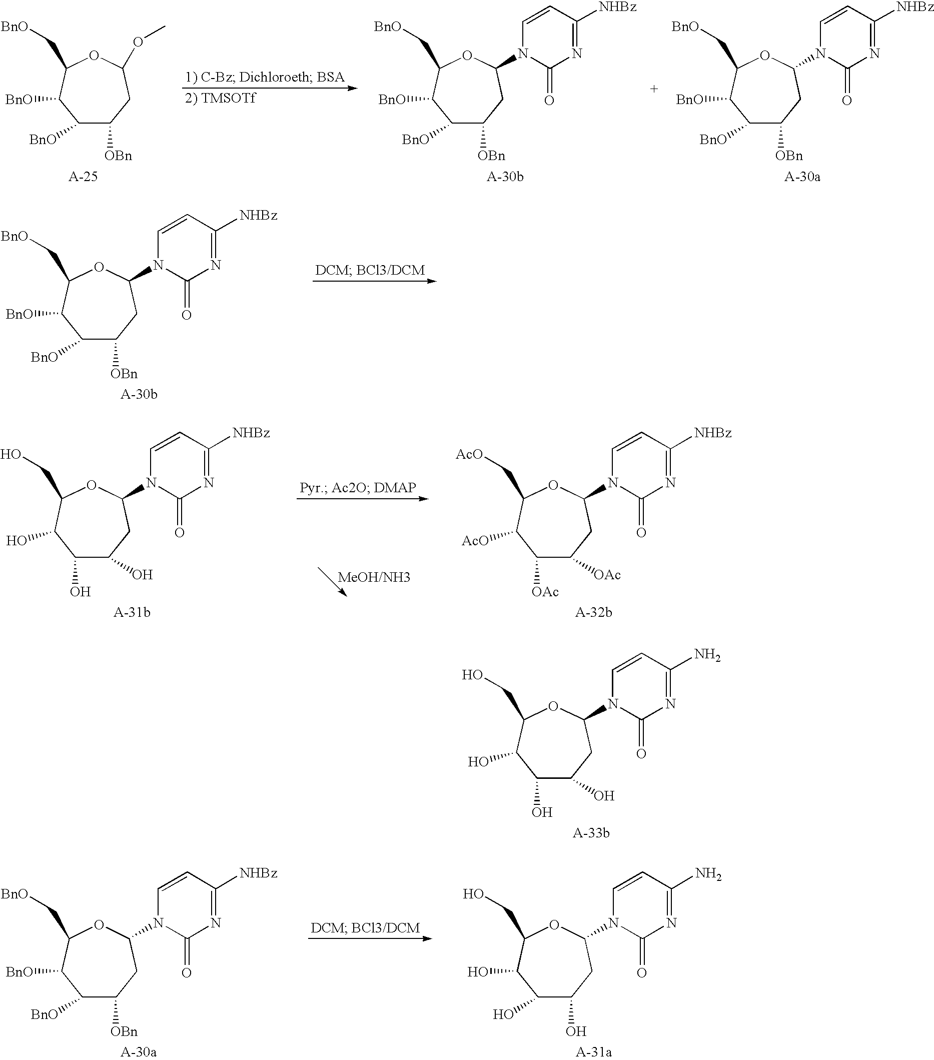

Compounds A-30b and A-30a. To a suspension of N4-benzoylcytosine (454 mg, 2.1 mmol) in anhydrous 1,2-dichloroethane (4 ml) was added N,O-bis(trimethylsilyl)acetamide (1.57 ml, 6.33 mmol). The reaction mixture was stirred at reflux during 30 min. To the previous reaction mixture was added 9 (1.0 g, 1.76 mmol) in solution in anhydrous 1, 2-dichloroethane (6 ml), trimethylsilyltrifluoromethanesulfonate (TMSOTf, 2.72 ml, 14.07 mmol) and the reaction mixture was stirred at reflux for 3 h and overnight at room temperature. At room temperature the reaction mixture was diluted with ethyl acetate, neutralized with a saturated aqueous solution of sodium bicarbonate and the mixture was filtrated through a pad of celite. The organic layer was washed with brine, dried over sodium sulfate (Na₂SO₄) and evaporated to dryness. The crude material was purified on a silica gel column chromatography (eluent: from 20 to 50% of ethyl acetate in cyclohexane) to afford compound A-30b (422 mg, 30%) and compound A-30a (85 mg, 6%). Molecular Formula: C₄₆H₄₋₅N₃O₇. A-30b: ¹H NMR (400 MHz CDCl₃): δ8.80 (s, 1H, N−H), 7.90-7.25 (m, 7H, 1Bz+H5+H6), 7.20-7.00 (m, 20H, 4Bn), 6.25 (dd, 1H, J₁=5.6 Hz, J₂=9.2 Hz, H1′), 4.90-4.70 (dd, 2H, J₁=12.2 Hz, J₂=57.4 Hz, 1CH₂Bn), 4.40-3.90 (m, 8H, 3CH₂Bn+2H), 3.65 (m, 1H), 3.50 (m, 2H), 3.40 (dd, 1H, J₁=2.1 Hz, J₂=10 Hz), 2.60 (m, 1H, H2b′), 2.00 (m, 1H, H2a′). UV λmax=260 nm, 301 nm. A-30a: ¹H NMR (400 MHz CDCl₃): δ8.90 (bs, 1H, N−H), 8.20 (d, 1H, J=7.3 Hz, H6), 7.90 (d, 2H, J=7.2), 7.60-7.40 (m, 4H), 7.40-7.05 (m, 20H, 4Bn), 6.00 (d, 1H, J=8.9 Hz, H1′), 4.90-4.70 (dd, 1H, J₁=12.1 Hz, J₂=21.3 Hz, CH₂Bn), 4.50-4.30 (m, 6H, 3CH₂Bn), 4.25-4.10 (m, 2H), 3.70-3.50 (m, 4H), 2.45-2.25 (m, 1H, H2b′), 1.70-1.50 (m, 1H, H2a′). LC/MS: t=22.7 min; MS ES+=752 (M+H)⁺. UV λmax=260 nm, 301 nm.

Compound A-31b. To a solution of A-30b (412 mg, 0.548 mmol) in anhydrous dichloromethane (5.3 ml) at −78° C. was added boron trichloride (1M in dichloromethane, 2.74 ml, 2.74 mmol) dropwise. The mixture was stirred at −78° C. for 2 hours then allowed to rise about −35° C. and stirred for 2 hours. The reaction was quenched by addition of methanol/dichloromethane (1.8 ml) (1/1) and stirred for 30 min at −20° C., then neutralized at 0° C. with aqueous ammonia and stirred at room temperature for 15 min. The solid was filtered through a pad of celite and washed with dichloromethane/methanol (1/1). The filtrate was concentrated to dryness and the crude material was purified on a silica gel column chromatography (eluent: dichloromethane/methanol (85/15)) to afford compound A-31b (95 mg, 44%). Molecular Formula: C₁₋₈H₂₁N₃O₇. ¹H NMR (400 MHz, DMSO d₆): δ11.20 (s, 1H, N−H), 8.25 (d, 1H, J=7.5 Hz, H6), 8.05 (m; 2H, Bz), 7.65-7.50 (m, 3H, Bz), 7.35 (d, 1H, J=7.4Hz, H5), 6.10 (dd, 1H, J₁=5.3Hz, J₂=9.1 Hz, H1′), 5.10 (d, 1H, J=4.3 Hz, OH′), 4.90 (d, 2H, J=5.6 Hz, 2OH′), 4.60 (t, 1H, J=5.8 Hz, OH7′), 3.90 (m, 2H), 3.60 (m, 2H), 3.50 (m under water, 2H), 2.20 (m, 1H, H2b′), 2.00 (m, 1H, H2a′). LC/MS: t=8.3 min; MS ES+=392 (M+H)⁺. UV λmax=260 nm, 301 nm.

Compound A-32b. A-31b (20 mg, 0.051 mmol) was dissolved with anhydrous pyridine (0.25 ml) then acetic anhydride (0.048 ml, 0.511 mmol) and a catalytic amount of dimethylaminopyridine were added. The reaction mixture was stirred at room temperature overnight. The mixture was diluted with ethyl acetate and washed sequentially with an aqueous solution of HCl (1M) and brine, then the organic layer was dried over sodium sulfate and concentrated under vacuo. Purification on a silica gel column chromatography (eluent: 5% of ethanol in dichloromethane) gave compound A-32b (26 mg, 91%). Molecular Formula: C₂₆H₂₉N₃O₁₁. ¹H NMR (400 MHz CDCl₃): δ 8.80 (bs, 1H, NH), 7.90 (m, 3H, H6+Bz), 7.60-7.40 (m, 4H, H5+Bz), 6.20 (dd, J₁=5.5 Hz, J₂=8.9 Hz, H1′), 5.50 (d, J=2.3 Hz, H4′), 5.30 (dd, J₁=7 Hz, J₂=7.9 Hz, H3′), 5.05 (dd, J₁=2.8 Hz, J₂=9.5 Hz, H5′), 4.10 (m, 3H, H6′+H7a′+H7b′), 2.55 (m, 1H, H2a′), 2.20 (m, 1H, H2b′), 2.15 (s, 3H, OAc), 2.00 (m, 9H, 3OAC). nOe studies: interaction between H1′ and H6′=beta nucleoside. LC/MS: t=13.12 min; MS ES+=560 (M+H)⁺, UV λmax=260 nm, 301 nm.

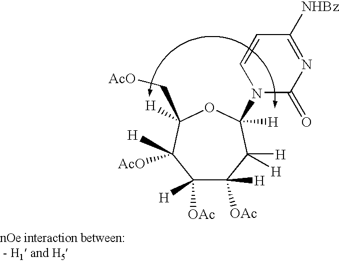

Compound A-33b. A-32b (66 mg, 0.169 mmol) was put in solution with a saturated solution of ammonia in methanol (10 ml), the mixture was stirred at room temperature overnight then concentrated under vacuo, diluted with water, washed twice with ethyl acetate and the aqueous phase was evaporated to dryness. The residue was purified by reverse phase (C 18) silica gel column chromatography (eluent: water) to afford compound A-33b (31 mg, 64%). Molecular Formula: C₁₁H₁₇N₃O₆. ¹H NMR (400 MHz, DMSO d₆): d 7.65 (d, 1H, J=7.4 Hz, H6), 7.10 (bd, 2H, NH₂), 6.00 (m, 1H, H1′), 5.75 (d, 1H, J=7.4 Hz, H5), 5.00 (d, 1H, J=4.4 Hz, OH′), 4.80 (m, 2H, 2OH′), 4.45 (t, 1H, J=5.6 Hz, OH7′), 4.90 (m, 2H), 3.50 (m under water, 4H), 2.00 (m, 2H, H2a′+H2b′). LC/MS: t=1.2 min; MS ES+=288 (M+H)⁺, UV λmax=271 nm.

Compound A-31a. To a solution of A-30a (85 mg, 0.113 mmol) in anhydrous dichloromethane (1.09 ml) at −78° C. was added boron trichloride (1M in dichloromethane, 0.565 ml, 0.565 mmol) dropwise. The mixture was stirred at −78° C. for 2 hours then allowed to rise about −35° C. and stirred for 2 hours. The reaction was quenched by addition of methanol/dichloromethane (1/1) and stirred for 30 min at −20° C., then neutralized at 0° C. with aqueous ammonia and stirred at room temperature for 15 min. The solid was filtered through a pad of celite and washed with dichloromethane/methanol (1/1). The filtrate was concentrated to dryness and the crude material was purified on silica gel column chromatography (eluent: dichloromethane/methanol (85/15)) to afford compound A-31a (20 mg, 62%). Molecular Formula: C₁₁H₁₇N₃O₆. ¹H NMR (200 MHz, DMSO d₆): δ 7.80 (d, 1H, J=7.4 Hz, H6), 7.20-7.00 (m, 2H, NH2), 5.80 (m, 1H, H1′), 5.70 (d, 1H, J=7.4 Hz, H5), 4.90 (m, 2H, 20H′), 4.70 (m, 1H, OH′), 4.30 (m, 1H, OH′), 3.90 (m, 2H), 3.60 (m underwater, 4H), 2.35 (m, 1H, H2b′), 1.65 (m, 1H, H2a′). LC/MS: t=1.12 min; MS ES+=288 (M+H)⁺, UV λmax=271 nm.

### Example 7

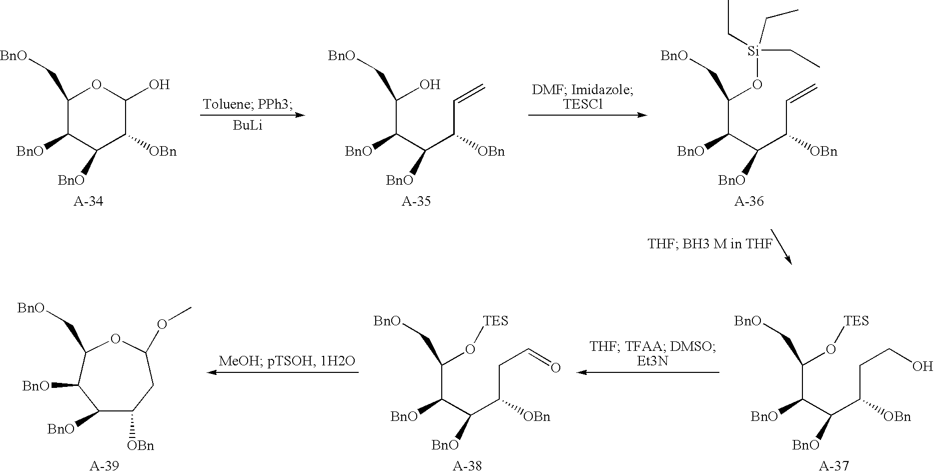

Compound A-35. To a solution of triphenylphosphonium bromide (28.27 g, 79.1 mmol) in anhydrous toluene (285 ml) was added butyllithium (1.6M in hexanes, 47.8 ml, 76.5 mmol) at 0° C., under argon. The mixture was stirred at room temperature for 1 h, to this reaction mixture was added under argon 2,3,4,6-tetra-O-benzyl-D-galactopyranose (A-34) (commercially available) (14.25 g, 26.4 mmol) in solution with a minimum volume of toluene. The all was stirred at room temperature during two days. The mixture was diluted with water and diethyl ether, the aqueous layer was extracted twice with diethyl ether and the combined organic layer was dried over sodium sulfate and concentrated under vacuo. The crude product A-35 was directly used in the next step. Molecular Formula: C₇H₁₄O₅.

Compound A-36. A-35 (28.42 g, 52.75 mmol) and dimethylformamide (DMF, 70 ml) were combined under argon and to this mixture was added imidazole (10.78 g, 158.24 mmol) followed by the addition of chlorotriethylsilane (TESCl) (9.44 ml, 55.39 mmol) dropwise. This solution was stirred at 40° C. for 2 h. The reaction mixture was diluted with ethyl acetate and sequentially washed twice with a saturated aqueous solution of sodium bicarbonate, and twice with brine. The organic layer was dried over sodium sulfate and concentrated under vacuo. Impurities were crystallized into hexanes, filtered and washed with diethyl ether. The filtrate was concentrated under reduced pressure and the crude material was purified on a silica gel column chromatography (eluent: hexanes/ethyl acetate (99/1)) to afford compound A-36 (27.53 g, 80%). Molecular Formula: C₄₀H₄₉O₅Si.

Compound A-37. To a solution of A-36 (27.53 g, 42.22 mmol) in tetrahydrofurane (THF) (196 ml), at 0° C. was added dropwise a solution of boran (1M in THF, 151.5 ml). The mixture was allowed to warm to room temperature and stirred under argon. After 3 h, the reaction was quenched with dropwise addition of water (80 ml) at 0° C. To the mixture was added a 4M solution of sodium hydroxide (202.3 ml) dropwise and then a 35% hydrogen peroxide solution (57 ml) at the same temperature. The mixture was stirred at room temperature overnight. Saturated aqueous solution of sodium bicarbonate was added and the tetrahydrofurane was removed under reduced pressure. The residue was extracted twice with dichloromethane, the combined organic layer was washed twice with water and dried over sodium sulfate and concentrated under vacuo. Purification on a silica gel column chromatography (eluent: from 5% to 20% of ethyl acetate in petroleum ether) gave compound A-37 (22.29 g, 79%). Molecular Formula: C₄₁H₅₄O₆Si. ¹H NMR (400 MHz, DMSO d₆): δ 7.40-7.20 (m, 20H, 4Bn), 5.20 (t, 1H, J=5.73 Hz, OH), 4.75-4.50 (m, 8H, CH₂Bn), 4.20 (m, 1H), 3.90 (m, 1H), 3.80 (m, 2H), 3.65 (m, 1H), 3.55 (m, 1H), 1.90 (m, 2H, H2a′+H2b′), 0.85 (m, 9H, 3CH₃ TES), 0.50 (m, 6H, 3CH₂ TES).

Compound A-38. A solution of tetrahydrofurane (45 ml) and trifluoroacetic anhydride (5.75 ml, 45.5 mmol) was placed at −60° C. Dimethylsulfoxide (9.77 ml, 137.63 mmol) was then added to the mixture and it was stirred for 2 min then A-37 (14.52 g, 21.64 mmol) in solution in tetrahydrofurane (100 ml) was added and the mixture was allowed to stir for 15 min. Triethylamine (17.15 ml, 123.35 mmol) was added and the reaction mixture was stirred for 5 min and then warmed to room temperature over 1 h. Water was added to the mixture and the aqueous phase was extracted three times with dichloromethane. The combined organic layer was washed with brine, dried over sodium sulfate and concentrated under vacuo. Purification on a silica gel column chromatography (eluent: from 5% to 6% of ethyl acetate in cyclohexane) gave compound A-38 (13.73 g, 95%). Molecular Formula: C₄₁H₅₂O₆Si. ¹H NMR (400 MHz, DMSO d₆): δ 9.70 (m, 1H, CHO), 7.40-7.20 (m, 20H, 4Bn), 4.75-4.50 (m, 8H, CH₂Bn), 4.35 (m, 1H), 4.15 (m, 1H), 3.80 (m, 2H), 3.60 (m, 2H), 2.80 (m, 2H, H2a′+H2b′), 0.85 (m, 9H, 3CH₃ TES), 0.50 (m, 6H, 3CH₂ of TES).

Compound A-39. A-38 (13.73 g, 20.54 mmol) was dissolved in methanol (330 ml). To this solution was added p-toluenesulfonic acid monohydrate (9.38 g, 49.3 mmol) and the mixture was stirred at room temperature for 1 h. A saturated aqueous solution of sodium bicarbonate was added and the mixture was extracted three times with dichloromethane. The combined organic layer was washed with brine dried over sodium sulfate and concentrated under vacuo. Purification on a silica gel column chromatography (eluent: from 5% to 20% of ethyl acetate in cyclohexane) gave compound A-39 (5.53 g, 47%). Molecular Formula: C₃₆H₄₀O₆. ¹H NMR (400 MHz, DMSO d₆): δ7.40-7.20 (m, 20H, 4Bn), 4.90-4.70 (m, 3H, CH₂Bn+H1′), 4.70-4.40 (m, 6H, 3CH₂Bn), 4.00 (q, 1H, J=7.1 Hz), 3.90 (d, 2H, J=3.2 Hz), 3.80-3.60 (m, 2H), 3.45 (d, 2H, J=6.7 Hz), 3.20 (s, 3H, OCH₃), 2.20 (m, 1H, H2a′), 1.80 (m, 1H, H2b′). MS: ESI m/z 569.48 (M+H)⁺.

### Example 8

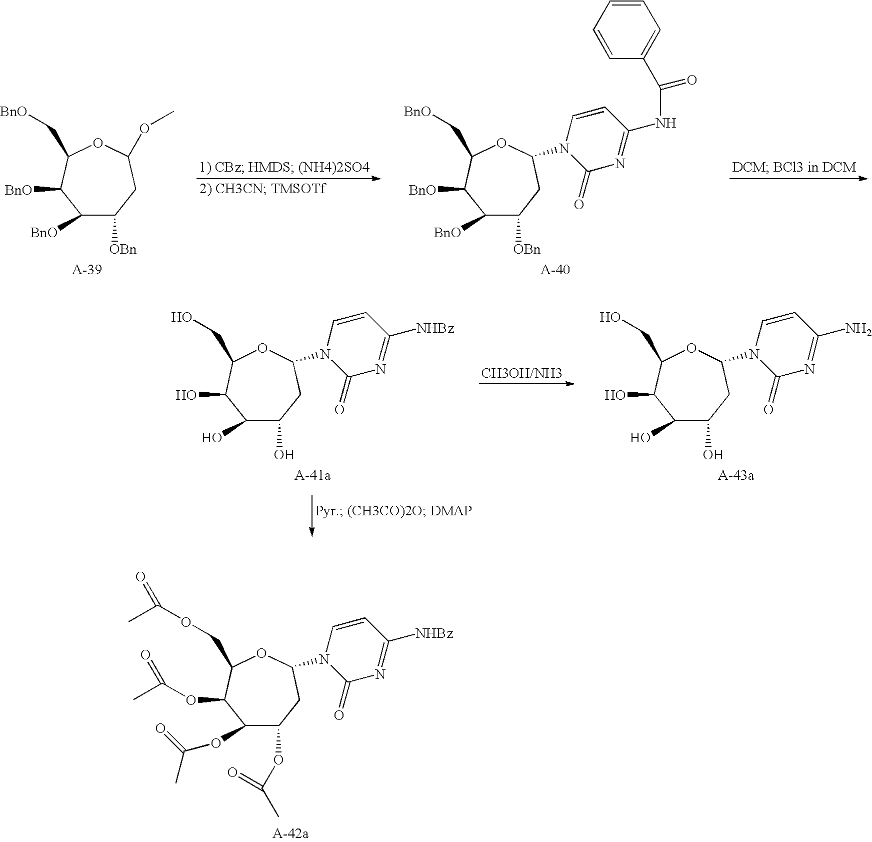

Compound A-40. Silylation of N4-benzoylcytosine: a suspension of N4-benzoylcytosine (454 mg, 2.11 mmol) in 1,1,1,3,3,3-hexamethyldisilazane (HMDS, 30 ml) and a catalytic quantity of ammonium sulfate was heated with stirring at reflux overnight under argon. The resulting solution was then allowed to cool to room temperature and concentrated under vacuo under argon. Condensation step: to a solution of silylated N4-benzoylcytosine in anhydrous acetonitrile (30 ml) was sequentially added compound A-39 (1.0 g, 1.76 mmol) and trimethylsilyltrifluoromethanesulfonate (TMSOTf, 0.51 ml, 2.64 mmol). The reaction mixture was stirred at room temperature during 2 hours under argon. The resulting solution was diluted with dichloromethane and neutralized with a saturated solution of sodium bicarbonate. The organic layer was washed once with water, then dried with sodium sulfate and evaporated under reduced pressure. The crude material was purified on a silica gel column chromatography (eluent: 1% of methanol in dichloromethane) to give compound A-40 (1.0 g, 70%). Molecular Formula: C₄₆H₄₅N₃O₇. ¹H NMR (200 MHz. CDCl₃): δ 8.60 (s, 1H, N−H), 8.10-7.90 (m, 3H, H6+2H, Bz), 7.70-7.50 (m, 3H, 3H, Bz), 7.40-7.10 (m, 21H, 3Bn+H5), 6.30 (d, 1H, J=9 Hz, H1′), 5.00-4.40 (m, 8H, 4×CH₂ of Bn), 4.25 (m, 1H, H6′), 4.05 (m, 2H, H7a′+H7b′), 4.80-4.40 (m, 3H, H3′+H4′+H5′), 2.65 (m, 1H, H2′a) 1.90 (m, 1H, H2′b). MS: FAB+(matrice GT) m/z 752 (M+H)⁺, FAB−(matrice GT) m/z 750 (M−H)⁻. UV: λmax=260 nm, 301 nm.

Compound A-41a. To a solution of A-40 (900 mg, 1.2 mmol) in dichloromethane (12 ml) at −78° C. was added boron trichloride (1M in dichloromethane, 6 ml, 6 mmol) dropwise. The mixture was stirred at −78° C. for 1 h then allowed to rise to −30° C. and stirred for 2 hours. The reaction was quenched by addition of methanol/dichloromethane (1/1) (6 ml) and stirred for 30 min at −20° C., then neutralized at 0° C. with aqueous ammonia and stirred at room temperature for 15 min. The solid was filtered through a pad of celite and washed with dichloromethane/methanol (1/1). The filtrate was evaporated to dryness and the residue was purified on a silica gel column chromatography (eluent: dichloromethane/methanol (85/15)) to give compound A-41a (220 mg, 40%). Molecular Formula: C₁₈H₂₁N₃O₇. ¹H NMR (200 MHz, DMSO d₆): δ11.15 (s, 1H, N−H), 8.30 (d, 1H, J=6.6 Hz, H6), 7.95 (d, 2H, J=7.8 Hz, 2H, Bz), 7.70-7.50 (m, 3H, 3HBz), 7.30 (d, 1H, J=6.6 Hz, H5), 6.10 (m, 1H, H1′), 5.00-4.60 (m, 4H, 4OH′), 4.10 (m, 1H, H3′), 3.60-3.90 (m, 2H, H4′+H5′), 3.50 (under water H6′), 3.30-3.10 (m, 2H, H7a′+H7b′), 2.00 (m, 2H, H2′a+H2′b). MS: FAB+(matrice GT) m/z 392 (M+H)⁺, FAB−(matrice GT) m/z 390 (M−H)⁻. UV: λmax=260 nm, 301 nm.

Compound A-42a. To a solution of A-41a (50 mg, 0.128 mmol) in anhydrous pyridine (1.28 ml), was added under argon at 0° C. acetic anhydride (60 □1, 0.64 mmol) and a catalytic amount of dimethylaminopyridine (DMAP). The reaction mixture was stirred at room temperature for 4 h, diluted in dichloromethane and sequentially washed with a 1M HCl solution, a saturated aqueous solution of sodium bicarbonate and brine. The organic layer was dried (Na2SO4) and concentrated under vacuo. The crude was purified on a silica gel column chromatography (eluent: from 1 to 2% of methanol in dichloromethane) to afford compound A-42a (12 mg). Molecular Formula: C₂₆H₂₉N₃O₁₁. ¹H NMR (400 MHz CDCl₃): δ8.70 (s, 1H, N−H), 7.85 (m, 2H, Bz), 7.75 (d, 1H, J=7.4 Hz, H5), 7.55 (d, 1H, J=7.4 Hz, H6), 7.50 (m, 3H, Bz), 6.10 (dd, 1H, J=2.1 Hz, J=10.9 Hz, H1′), 5.50 (t, 1H, J=2.5 Hz, H5′), 5.40 (m, 1H, H3′), 5.15 (dd, 1H, J=2.2 Hz, J=10.2 Hz, H4′), 4.60 (m, 1H, H6′), 4.20-4.00 (m, 2H, H7a′+H7b′) 2.50-2.40 (m, 1H, H2′a), 2.35-2.20 (m, 1H, H2′b), 2.20 (s, 3H, 1OAc), 2.00-1.90 (m, 9H, 3×OAc). nOe studies: interaction between H1′ and H3′=alpha nucleoside. UV: λmax=260 nm, 301 nm.

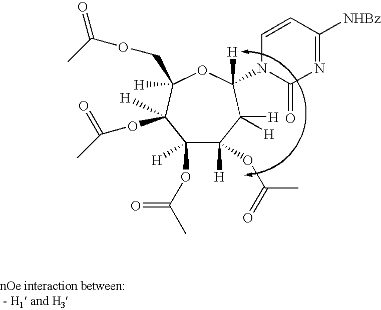

Compound A-43a. A-41a (170 mg, 0.435 mmol) in solution with a saturated solution of ammonia in methanol (6.5 ml) was stirred at room temperature overnight, then concentrated under vacuo. Compound A-43a(34 mg, 30%) was crystallized in water. Molecular Formula: C₁₁H₁₇N₃O₆. ¹H NMR (250 MHz DMSO d₆): δ7.75 (d, 1H, J=7.4 Hz, H5), 7.00 (m, 2H, NH₂), 6.05 (dd, 1H, J=2.5 Hz, J=10.6 Hz, H1′), 5.70 (d, 1H, J=7.4 Hz, H6), 4.80-4.55 (m, 4H, 40H′), 3.95 (m, 1H, H3′), 3.85-3.65 (m, 2H, H4′+H5′), 3.50 (m under water, 3H, H6′+H7a′+H7b′), 2.10-1.80 (m, 2H, H2′a+H2′b). UV: λmax=271 nm.

### Example 9

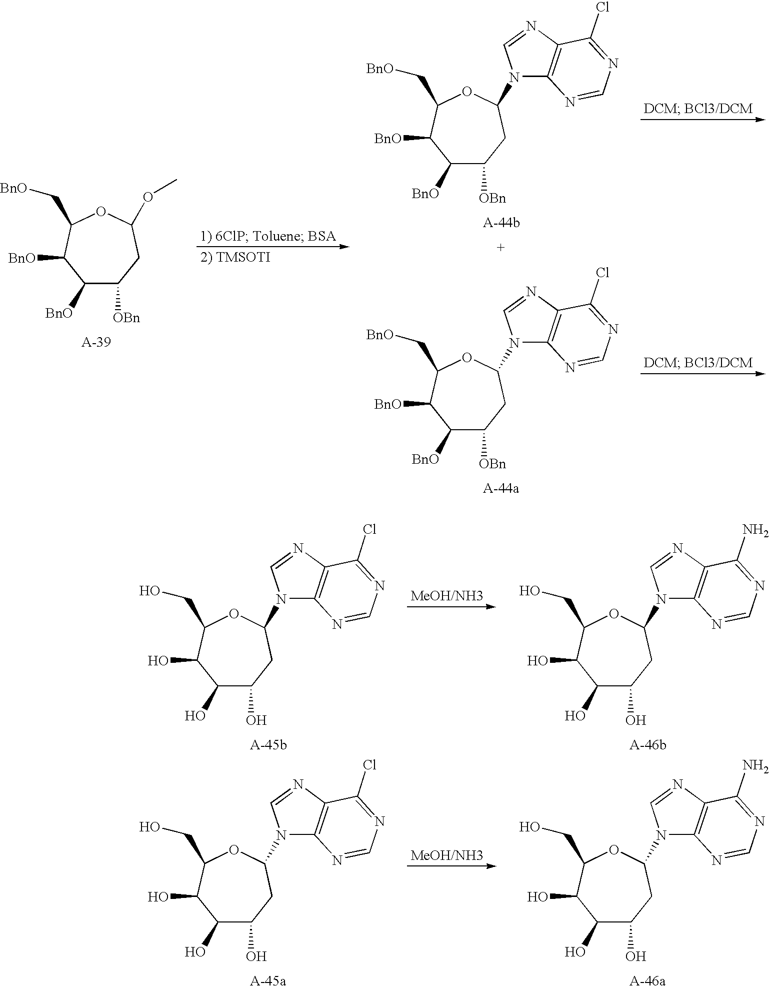

Compound A-44b and A-44a. To a suspension of 6-chloropurine (0.99 g, 6.44 mmol) in anhydrous toluene (85 ml) was added N,O-bis(trimethylsilyl)acetamide (2.85 ml, 11.5 mmol). The reaction mixture was stirred at reflux during 45 min. At room temperature and under argon, to this mixture was added A-39 (3.06 g, 5.37 mmol) and trimethylsilyltrifluoromethanesulfonate (TMSOTf, 1.55 ml, 8.04 mmol), the reaction mixture was stirred at 70° C. for 5 hours, then, at room temperature was diluted with dichloromethane, washed successively with a saturated aqueous solution of sodium bicarbonate, water, dried over sodium sulfate and concentrated under vacuo. Purification on a silica gel column chromatography (eluent: from 0 to 2% of methanol in dichloromethane) gave compound A-44b (0.322 g, 7%) and compound A-44a (1.66 g, 37%). Molecular Formula: C₄₀H₃₉ClN₄O₅. A-44b: ¹H NMR (200 MHz, CDCl₃): δ8.75 (s, 1H, H2), 8.40 (s, 1H, H8), 7.50-7.15 (m, 20H, 4Bn), 6.15 (m, 1H, H1′), 5.00-4.40 (m, 8H, 4×CH₂Bn), 4.20-3.90 (m, 3H, H3′+H4′+H5′+H6′), 3.55 (d, 2H, J=6.4 Hz, H7a′+H7b′), 3.90-3.75 (m, 1H, H2a′), 3.65-3.40 (m, 1H, H2b′). UV: λmax=265 nm. A-44a: ¹H NMR (200 MHz, CDCl₃): δ8.75 (s, 1H, H2), 8.30 (s, 1H, H8), 7.50-7.10 (m, 20H, 4Bn), 6.35 (m, 1H, H1′), 5.10-4.65 (m, 6H, 3CH₂Bn), 4.35 (s, 2H, CH₂Bn), 4.30-4.10 (m, 3H), 3.80 (m, 1H), 3.65-3.40 (m, 2H, H7′+H7″), 2.70-2.55 (m, 2H, H2a′+H2b′). UV: λmax=265 nm.

Compound A-45b. To a solution of A-44b (0.322 g, 0.466 mmol) in anhydrous dichloromethane (5 ml) at −78° C. was added boron trichloride (1M in dichloromethane, 2.33 ml, 2.33 mmol) dropwise. The mixture was stirred at −78° C. for 2 hours then allowed to rise about −30° C. and stirred for 2 hours. The reaction was quenched by addition of methanol/dichloromethane (1/1) and stirred for 30 min at −20° C., then neutralized at 0° C. with aqueous ammonia and stirred at room temperature for 15 min. The solid was filtered through a pad of celite and washed with dichloromethane/methanol (1/1). The filtrate was concentrated to dryness, the residue was dissolved with water and washed with dichloromethane, and the aqueous layer was evaporated to dryness. The crude material compound A-45b was directly used in the next step. Molecular Formula: C₁₂H₁₅ClN₄O₅.

Compound A-46b. A-45b (73 mg, 0.221 mmol) was dissolved with saturated solution of ammonia in methanol (5 ml), placed in a steel bomb and heated at 100° C. overnight, then evaporated to dryness. The residue was dissolved with water and washed with dichloromethane. The aqueous layer was concentrated under vacuo, then purified by reverse phase (C 18) on a silica gel column chromatography (eluent: water) to obtain compound A-46b (12 mg, 8%). Molecular Formula: C₁₂H₁₇N₅O₅. ¹H NMR (250 MHz DMSO d₆): δ8.30 (s, 1H, H2), 8.10 (s, 1H, H8), 7.30 (s, 2H, NH₂), 6.05 (m, 1H, H1′), 4.95 (bs, 1H, OH′), 4.80-4.60 (bs, 3H, 3OH′), 3.95-3.75 (m, 3H), 3.50 (under water, 3H), 2.35-2.15 (m, 2H, H2a′, H2b′). UV: λmax=265 nm.

Compound A-45a. To a solution of A-44a (1.636 g, 2.36 mmol) in anhydrous dichloromethane (23.6 ml) at −78° C. was added boron trichloride (1M in dichloromethane, 10 ml, 10 mmol) dropwise. The mixture was stirred at −78° C. for 2 hours then allowed to rise about −30° C. and stirred for 2 hours. The reaction was quenched by addition of methanol/dichloromethane (12 ml) (1/1) and stirred for 30 min at −20° C., then neutralized at 0° C. with aqueous ammonia and stirred at room temperature for 15 min. The solid was filtered through a pad of celite and washed with dichloromethane/methanol (1/1). The filtrate was concentrated to dryness, the residue was dissolved with water and washed with dichloromethane and the aqueous layer was evaporated to dryness. The crude material compound A-45a was directly used in the next step. Molecular Formula: C₁₂H₁₅ClN₄O₅.

Compound A-46a. A-45a (450 mg, 1.36 mmol) was dissolved with saturated solution of ammonia in methanol (27 ml), placed in a steel bomb and heated at 110° C. overnight, then evaporated to dryness. The residue was dissolved with water and washed with dichloromethane. The aqueous layer was concentrated under vacuo, then purified by reverse phase (C 18) on a silica gel column chromatography (eluent: water) to obtain compound A-46a (27 mg, 4%). Molecular Formula: C₁₂H₁₇N₅O₅. ¹H NMR (400 MHz, DMSO d₆): δ8.40 (s, 1H, H2), 8.10 (s, 1H, H8), 7.20 (s, 2H, NH₂), 6.15 (dd, 1H, J₁=3.9 Hz, J₂=11.5 Hz, H1′), 4.70 (bs, 2H, OH4′+OH5′), 4.60 (bs, 1H, OH3′), 4.15 (t, 1H, J=6.6 Hz, H5′), 3.85 (m, 2H, H3′+OH5′), 3.40 (m under water, 4H, H4′+H6′+H7a′+H7b′), 2.80 (m, 1H, H2a′), 2.21 (m, 1H, H2b′). nOe studies: interaction between H1′ and H3′=alpha nucleoside. UV: λmax=260 nm.

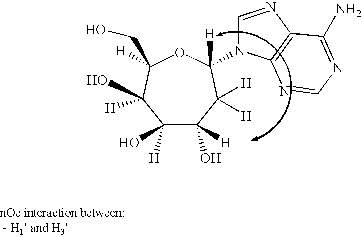

### Example 10

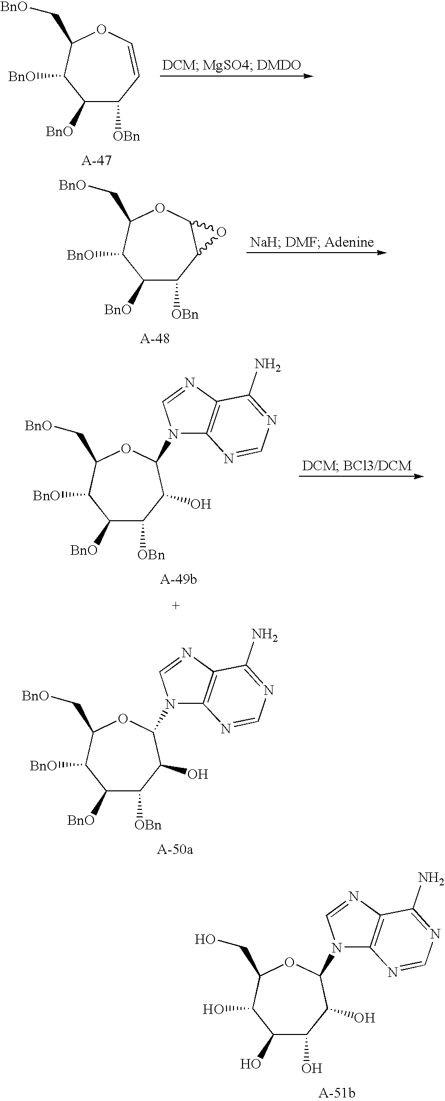

Compound A-47. Was prepared according to Peczuh M. W.; Snyder N. L., Tetrahedron Letters 2003, 44, 4057-61. Molecular Formula: C₃₅H₃₆O₅.

Compound A-48. To a stirring solution of A-47 (1.22 g, 2.78 mmol) in dichloromethane (40 ml) and some magnesium sulfate (MgSO₄), Dimethyldioxirane in acetone (Dimethyldioxirane in acetone was prepared according to: Adam W.; Bialas J.; Hadjiarapoglou L., Chem. Ber. 124 (1991) 2377) (7 ml) was added by portions at room temperature until a complete conversion. The reaction mixture was filtered, evaporated to dryness, the residue was dissolved with toluene, MgSO₄ was added, this mixture was filtered and the filtrate was concentrated under vacuo. The crude compound A-48 was quickly used in the next step. Molecular Formula: C₃₅H₃₆O₆.

Compounds A-49b and A-50a. To a suspension of sodium hydride (NaH 60% dispersion in mineral oil, 176 mg, 4.42 mmol) in dimethylformamide (DMF, 36 ml) was added adenine (895 mg, 6.63 mmol). The mixture was stirred at 75° C. for 30 min. At room temperature was added A-48 (1.22 g, 2.21 mmol) in solution with some dimethylformamide. The reaction mixture was stirred at 75° C. two days. The mixture was diluted with ethyl acetate, washed sequentially with an aqueous solution of HCl (1M) and brine. The organic phase was dried over sodium sulfate and concentrated under vacuo. The crude material was purified on a silica gel column chromatography (eluent: ethyl acetate) to afford compound A-49b (50 mg, 3.3%) and compound A-50a (5 mg, 0.3%). Molecular Formula: C₄₀H₄₁N₅O₆. A-49b (major anomer): ¹H NMR (300 MHz, CDCl₃): δ 8.15 (s, 1H, H8), 7.85 (s, 1H, H2), 7.40-6.90 (m, 20H, 4Bn), 6.15 (bs, 2H, NH₂), 5.70 (d, 1H, J=9 Hz, H1′), 5.00-4.40 (m, 6H, 3CH₂Bn), 4.35-4.10 (m, 2H, 1CH₂Bn), 3.90-3.65 (m, 4H), 3.60-3.35 (m, 4H). LC/MS: t=18.7 min; MS ES+=688 (M+H)⁺, UV λmax=260 nm.

Compound A-51b. To a solution of A-49b (50 mg, 0.073 mmol) in anhydrous dichloromethane (1.5 ml) at −78° C. was added boron trichloride (1M in dichloromethane, 0.7 ml, 0.7 mmol) dropwise. The mixture was stirred at −78° C. for 2 hours then allowed to rise about −30° C. and stirred for 2 hours. The reaction was quenched by addition of methanol/dichloromethane (1/1) and stirred for 30 min at −15° C., then neutralized at 0° C. with aqueous ammonia and stirred at room temperature for 15 min. The solid was filtered through a pad of celite and washed with dichloromethane/methanol (1/1). The filtrate was concentrated to dryness, the residue was purified by reverse phase (C 18) silica gel column chromatography (eluent: water) to give compound A-51b (11 mg, 46%). Molecular Formula: C₁₂H₁₇N₅O₆. ¹H NMR (300 MHz, DMSO d₆): δ 8.40 (s, 1H, H8), 8.20 (s, 1H, H2), 7.30 (bs, 2H, NH₂), 5.75 (d, 1H, J=9.2 Hz, H1′), 5.30 (d, 1H, J=4.9 Hz, OH₂′), 5.10 (m, 3H, 30H′), 4.50 (m, 2H, OH′30 H2′), 3.95 (m, 1H, H6′), 3.50 (m under water, 5H, H3′+H4′+H5′+H7a′+H7b′). LC/MS: t=1.2 min; MS ES+=328 (M+H)⁺, UV λmax=260 nm.

### Example 11

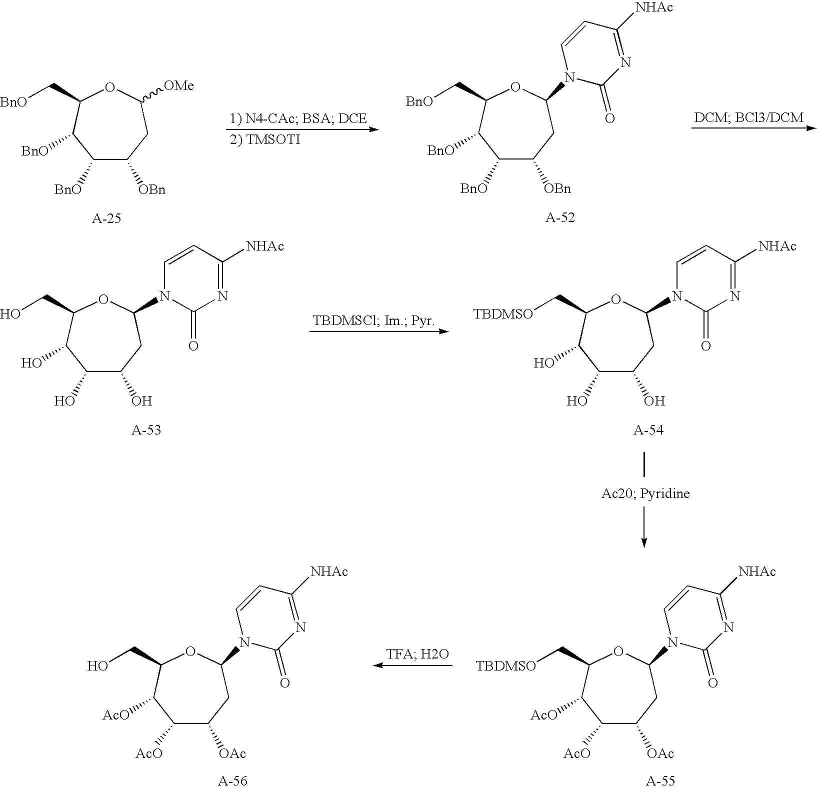

Compound A-52. To a suspension of N4-acetylcytosine (2.24 g, 14.78 mmol) in anhydrous 1,2-dichloroethane (28 ml) was added N,O-bis(trimethylsilyl)acetamide (BSA) (11 ml, 44.3 mmol). The reaction mixture was stirred at reflux during 2 hours. A-25 (7 g, 12.308 mmol) in solution with 1,2-dichloroethane (42 ml) and trimethylsilyltrifluoromethanesulfonate (TMSOTf) (4.76 ml, 21.62 mmol) were added. The reaction mixture was stirred at reflux for 1 h. TMSOTf was added every hour until complete conversion (7 ml in 4 hours). At room temperature the mixture was diluted with ethyl acetate, washed with a saturated aqueous solution of sodium bicarbonate, filtered through a pad of celite, the organic layer was washed with water, dried over sodium sulfate and concentrated under vacuo. Purification on a silica gel column chromatography (eluent: from 0 to 2% of methanol in dichloromethane) gave compound A-52 (2.89 g, 34%). Molecular Formula: C₄₁H₄₃N₃O₇. ¹H NMR (250 MHz, CDCl₁₃): δ 9.10 (bs, 1H, NH), 7.85 (d, 1H, J=7.6 Hz, H5), 7.40-7.10 (m, 21H, 4Bn+H6), 6.35 (dd, 1H, H1′), 5.004.70 (dd, 2H, J₁=12.2 Hz, J₂=38.3 Hz), 4.60-4.40 (m, 3H), 4.35 (m, 2H), 4.30 (m, 1H), 4.20 (m, 1H), 4.05 (m, 1H), 3.75 (m, 1H), 3.65 (m, 2H), 3.60 (m, 1H), 2.70 (m, 1H, H2a′), 2.20 (s, 3H, Ac), 2.10 (m, 1H, H2b′). LC/MS: t=20.7 min; MS ES+=690 (M+H)⁺, UV λmax=248 nm, 298 nm.

Compound A-53. To a solution of A-52 (2.89 g, 4.19 mmol) in anhydrous dichloromethane (37.2 ml) at −78° C. was added boron trichloride (1M in dichloromethane, 19.06 ml, 19.06 mmol) dropwise. The mixture was stirred at −78° C. for 2 hours then allowed to rise about −40° C. and stirred for 2 hours. The reaction was quenched by addition of methanoydichloromethane (13.5 ml) (1/1) and stirred for 30 min at −20° C., then neutralized at 0° C. with aqueous ammonia and stirred at room temperature for 15 min. The solid was filtered through a pad of celite and washed with dichloromethane/methanol (1/1). The filtrate was evaporated to dryness and the residue was purified on silica gel column chromatography (eluent: from 20 to 30% of methanol in dichloromethane) to give compound A-53 (1.49 g, >100% because of presence of silica gel). Molecular Formula: C₁₃H₁₉N₃O₇. ¹H NMR (200 MHz, DMSO d₆): δ 10.90 (s, 1H, NH), 8.20 (d, 1H, J=7.5 Hz, H5), 7.10 (d, 1H, J=7.4 Hz, H6), 6.10 (dd, 1H, J₁=5.4 Hz, J₂=9 Hz, H1′), 5.20 (m, 1H, OH), 4.90 (m, 2H, 2OH), 4.50 (m, 1H, OH), 3.90 (m, 2H), 3.60 (2H), 3.50 (m under water, 2H), 2.20 (m, 1H, H2a′), 2.10 (s, 3H, Ac), 1.95 (m, 1H, H2b′). LC/MS: t=5.2 min; MS ES+=330 (M+H)⁺, UV λmax=248 nm, 298 nm.

Compound A-54. To a solution of A-53 (1.27 g, 3.86 mmol) in pyridine (25.47 ml) were added tert-butyldimethylsilylchloride (610 mg, 4.05 mmol) and imidazole (788 mg, 11.57 mmol). The reaction was stirred at room temperature for 3 h, and quenched by dilution with ethyl acetate. The mixture was washed successively with HCl 1 M and brine. The organic layer was dried over sodium sulfate and concentrated under vacuo. The crude material was purified on a silica gel column chromatography (eluent: from 4 to 10% of ethanol in dichioromethane) to afford compound A-54 (545 mg, 32%). Molecular Formula: C₁₉H₃₃N₃O₇Si. ¹H NMR (200 MHz, DMSO d₆): δ 10.90 (s, 1H, NH), 8.10 (d, 1H, J=7.5 Hz, H5), 7.30 (d, 1H, J=7.4 Hz, H6), 6.10 (m, 1H, H1′), 5.20 (d, 1H, J=4.2 Hz, OH), 4.90 (m, 2H, 2OH), 3.90-3.50 (m, 6H), 2.10 (m, 3H, Ac), 2.05 (m, 2H, H2a′+H2b′), 0.80 (s, 9H, C(CH₃)₃), 0.00 (2s, 6H, Si(CH₃)₂). LC/MS: t=12.3 min; MS ES+=444 (M+H)⁺, UV λmax=248 nm, 298 nm.

Compound A-55. To a solution of A-54 (325 mg, 0.733 mmol) in anhydrous pyridine (3.25 ml), acetic anhydride (550 μl, 5.86 mmol) was added under argon at 0° C. The reaction mixture was stirred at room temperature overnight, diluted in ethyl acetate and sequentially washed with a 1M HCl solution, a saturated aqueous solution of sodium bicarbonate and brine. The organic layer was dried (Na₂SO₄) and concentrated under vacuo. The crude was purified on a silica gel column chromatography (eluent: 5% of ethanol in dichloromethane) to afford compound A-55 (363 mg, 87%). Molecular Formula: C₂₅H₃₉N₃O₁₀Si. ¹H NMR (200 MHz CDCl₃): δ 8.90 (s, 1H, NH), 8.00 (d, 1H, J=7.5 Hz, H5), 7.50 (d, 1H, J=7.4 Hz, H6), 6.30 (dd, 1H, J₁=5.4 Hz, J₂=8.7 Hz, H1′), 5.70 (d, 1H, J=2.7 Hz), 5.30 (m, 1H), 5.10 (dd, J₁=3 Hz, J₂=9.5 Hz, 1H), 4.00 (m, 1H), 3.70 (m, 2H), 2.60 (m, 1H, H2a′), 2.30 (m, 6H, 2OAc), 2.10 (m, 7H, 2OAc+H2b′), 0.90 (s, 9H, C(CH₃)₃), 0.00 (2s, 6H, Si(CH₃)₂). LC/MS: t=9.17 min; MS (M-TBDMS)+=456, UV λmax=248 nm, 298 nm.

Compound A-56. A-55 (310 mg, 0.54 mmol) was dissolved in a mixture of trifluoroacetic acid/water (9.5/0.5) (4.95 ml).The reaction mixture was stirred at room temperature for 30 min. The mixture was evaporated to dryness and purified on a silica gel column chromatography (eluent: from 5 to 15% of methanol in dichloromethane) to furmish compound A-56 (132 mg, 50%). Molecular Formula: C₁₉H₂₅N₃O₁₀. ¹H NMR (200 MHz DMSO d₆): δ 10.90 (s, 1H, NH), 8.30 (d, 1H, J=7.5 Hz, H5), 7.30 (d, 1H, J=7.5 Hz, H6), 6.10 (dd, 1H, J₁=5.2 Hz, J₂=8 Hz, H1′), 5.50 (d, 1H, J=2.7 Hz), 5.30 (m, 1H), 5.10 (dd, J₁=3.1 Hz, J₂=9.3 Hz, 1H), 4.90 (t, 1H, OH5′), 3.90 (m, 1H), 3.50 (under water, 2H), 2.40-2.20 (m, 2H, H2a′+H2b′), 2.10 (2s, 6H, 1Ac), 2.00 (2s, 6H, 2Ac). LC/MS: t=8.37 min; MS ES+=456 (M+H)⁺, UV λmax=248 nm, 298 nm.

### Example 12

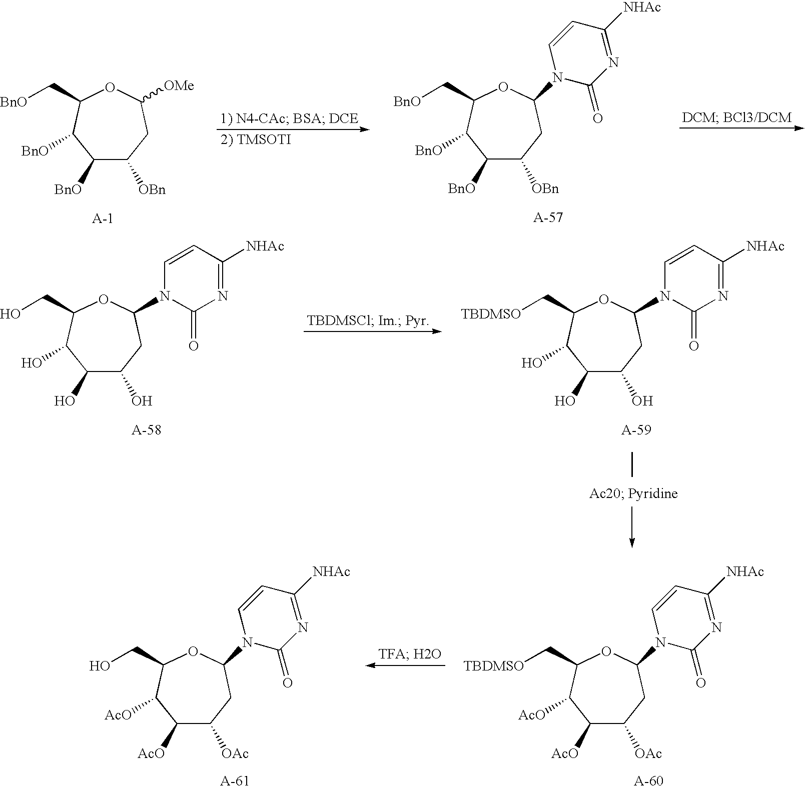

Compound A-57. To a suspension of N4-acetylcytosine (2.71 g, 16.25 mmol) in anhydrous 1,2-dichloroethane (34 ml) was added N, O-bis(trimethylsilyl)acetamide (BSA) (10.82 ml, 53.2 mmol). The reaction mixture was stirred at reflux during 2 h. A-1 (8.2 g, 14.77 mmol) in solution with 1,2-dichloroethane (42 ml) and trimethylsilyltrifluoromethanesulfonate (TMSOTf) (5.7 ml, 29.54 mmol) were added. The reaction mixture was stirred at reflux for 1 hour. TMSOTf was added every hour until complete conversion (5.7 ml in 2 hours). At room temperature the mixture was diluted with ethyl acetate, washed with a saturated aqueous solution of sodium bicarbonate, filtered through a pad of celite, the organic layer was washed with water, dried over sodium sulfate and concentrated under vacuo. Purification on a silica gel column chromatography (eluent: from 0 to 2% of methanol in dichloromethane) to afford compound A-57 (5.01 g, 49%). Molecular Formula: C₄₁H₄₃N₃O₇. ¹H NMR (200 MHz CDCl₃): δ9.00 (bs, 1H, NH), 8.00 (d, 1H, J=7.5 Hz, H5), 7.40-7.20 (m, 21H, 4Bn+H6), 6.10 (m, 1H, H1′), 4.80-4.40 (m, 9H), 4.00-3.80 (m, 2H), 3.70 (m, 3H), 2.50 (m, 1H, H2a′), 2.30 (s, 3H, Ac), 2.10 (m, 1H, H2b′). MS: FAB+(matrice GT) m/z 690 (M+H)⁺, FAB−(matrice GT) m/z 688 (M−H)⁻. UV: λmax=248 nm, 298 nm.

Compound A-58. To a solution of A-57 (5.01 g, 7.263 mmol) in anhydrous dichloromethane (71 ml) at −78° C. was added boron trichloride (1M in dichloromethane, 36.3 ml, 36.3 mmol) dropwise. The mixture was stirred at −78° C. for 2 hours then allowed to rise about 40° C. and stirred for 2 hours. The reaction was quenched by addition of methanol/dichloromethane (23.3 ml) (1/1) and stirred for 30 min at −20° C., then neutralized at 0° C. with aqueous ammonia and stirred at room temperature for 15 min. The solid was filtered through a pad of celite and washed with dichloromethane/methanol (1/1). The filtrate was evaporated to dryness and the residue was purified on silica gel column chromatography (eluent: from 20 to 30% of methanol in dichloromethane) to give compound A-58 (2.12 g, 88%). Molecular Formula: C₁₃H₁₉N₃O₇. ¹H NMR (200 MHz, DMSO d₆): δ 10.90 (s, 1H, NH), 8.20 (d, 1H, J=7.5 Hz, H5), 7.20 (d, 1H, J=7.6 Hz, H6), 5.90 (m, 1H, H1′), 5.10 (m, 2H, 2OH), 4.90 (m, 1H, OH), 4.50 (m, 1H, OH), 4.20 (m, 1H), 3.60-3.30 (m under water, 5H), 2.10 (m, 5H, H2a′+H2b′+Ac). LC/MS: t=1.2 min; MS ES+=330 (M+H)⁺, UV λmax=248 nm, 298 nm. Compound A-59. To a solution of A-58 (1.89 g, 5.74 mmol) in pyridine (38 ml) were added tert-butyldimethylsilylchloride (908 mg, 6.03 mmol) and imidazole (1.17 mg, 17.22 mmol). The reaction was stirred at room temperature for 3 hours, and quenched by dilution with ethyl acetate. The mixture was washed successively with HCl 1 M and brine. The organic layer was dried over sodium sulfate and concentrated under vacuo. The crude material was purified on a silica gel column chromatography (eluent: from 4 to 10% of ethanol in dichloromethane) to afford compound A-59 (191 mg, 8%). Molecular Formula: C₁₉H₃₃N₃O₇Si. ¹H NMR (200 MHz, DMSO d₆): δ 10.80 (s, 1H, NH), 8.10 (d, 1H, J=7.5 Hz, H5), 7.20 (d, 1H, J=7.4 Hz, H6), 5.90 (m, 1H, H1′), 5.20 (m, 3H, 3OH), 3.90-3.50 (m, 6H), 2.10 (m, 5H, Ac+H2a′+H2b′), 0.80 (s, 9H, C(CH₃)₃), 0.00 (2s, 6H, Si(CH₃)₂). LC/MS: t=12.05 min; MS ES+=444 (M+H)⁺, UV λmax=248 nm, 298 nm.

Compound A-60. To a solution of A-59 (191 mg, 0.43 mmol) in anhydrous pyridine (1.9 ml), acetic anhydride (324 □1, 3.445 mmol) was added under argon at 0° C. The reaction mixture was stirred at room temperature overnight, diluted in ethyl acetate and sequentially washed with a 1M HCl solution, a saturated aqueous solution of sodium bicarbonate and brine. The organic layer was dried (Na₂SO₄) and concentrated under vacuo. The crude was purified on a silica gel column chromatography (eluent: 5% of ethanol in dichloromethane) to afford compound A-60 (176 mg, 72%). Molecular Formula: C₂₅H₃₉N₃O₁₀Si. ¹H NMR (200 MHz, DMSO dm₆): δ 10.90 (s, 1H, NH), 8.20 (d, 1H, J=7.5 Hz, H5), 7.20 (d, 1H, J=7.5 Hz, H6), 5.90 (m, 1H, H1′), 5.20 (m, 1H), 5.05 (m, 2H), 4.10 (m, 1H), 3.80-3.60 (m, 2H), 2.00 (m, 14H, H2a′+H2b′+4OAc), 0.80 (s, 9H, C(CH₃)₃), 0.00 (2s, 6H, Si(CH₃)₂). LC/MS: t=16.09 min; MS ES+=570 (M+H)⁺, UV λmax=248 nm, 298 nm.

Compound A-62. To a solution of A-27b (2.19 g, 6.62 mmol) in pyridine (43.8 mL) were added tert-butyldimethylsilylchloride (1.098 g, 7.28 mmol) and imidazole (676 mg, 9.93 mmol). The reaction was stirred at room temperature for one hour, and treated as A-59. The crude material was purified on a silica gel column chromatography (eluent: from 7 to 10% of ethanol in dichloromethane) to afford compound compound A-62 (670 mg, 23%). Molecular Formula: C₁₈H₂₉ClN₄O₅Si. ¹H NMR (300 MHz, DMSO d₆): δ 9.18 (s, 1H, H8), 8.99 (s, 1H, H2), 6.58 (m, 1H. H1′), 5.55 (m, 1H, OH), 5.18-5.26 (m, 2H, 2OH), 3.54-4.26 (m, 6H), 3.12 (m, 1H, H2a′), 2.38 (m, 1H, H2b′), 0.71 (s, 9H, C(CH₃)₃), 0.00 (2s, 6H, Si(CH₃)₂). UV: λmax=265 nm.

Compound A-63. A-62 (670 mg, 1.50 mmol) was dissolved in a saturated solution of NH3 in methanol an heated in a steel bomb at 100° C. for 2 h20. The reaction mixture was then evaporated to dryness and the residue was purified on silica gel chromatography (eluent: from 7 to 10% of ethanol in dichloromethane) to give compound A-63 (250 mg, 39%). Molecular Formula: C₁₈H₃₁N₅O₅Si. ¹H NMR (300 MHz, DMSO d₄₆): δ 8.53 (s, 1H, H8), 8.29 (s, 1H, H2), 7.36 (bs, 2H, NH2), 6.37 (m, 1H, H1′), 5.42 (m, 1H, OH), 5.03-5.10 (m, 2H, 2OH), 3.53-4.22 (m, 6H), 2.96 (m, 1H, H2a′), 2.30 (m, 1H, H2b′), 0.79 (s, 9H, C(CH₃)₃), 0.00 (2s, 6H, Si(CH₃)₂). UV: λmax=260 nm.

### Example 13

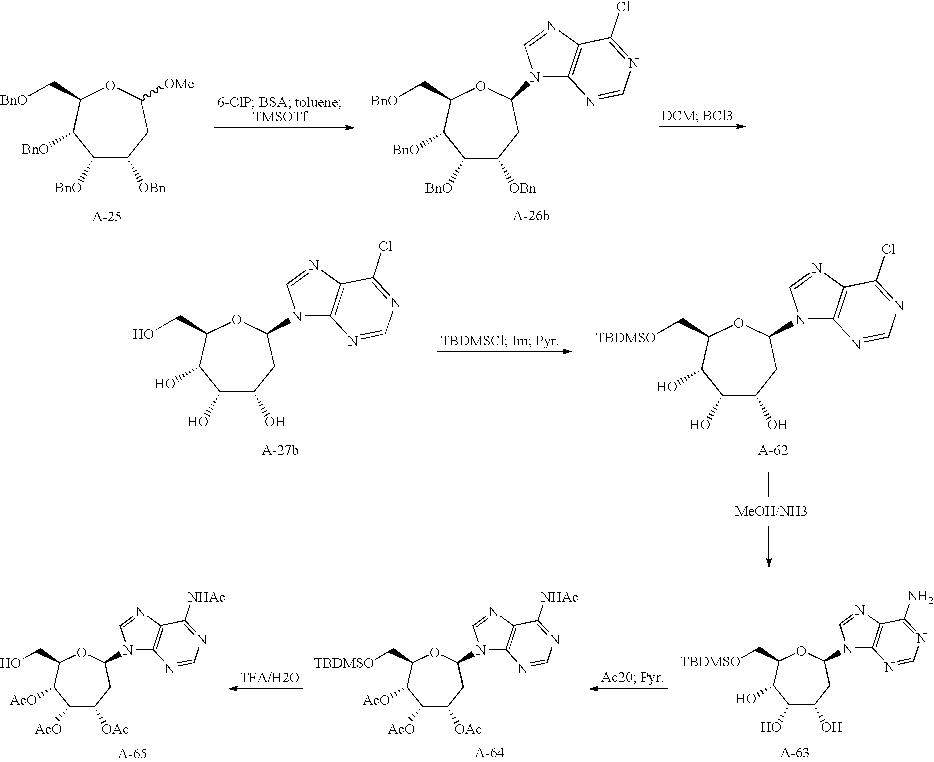

### Example 14

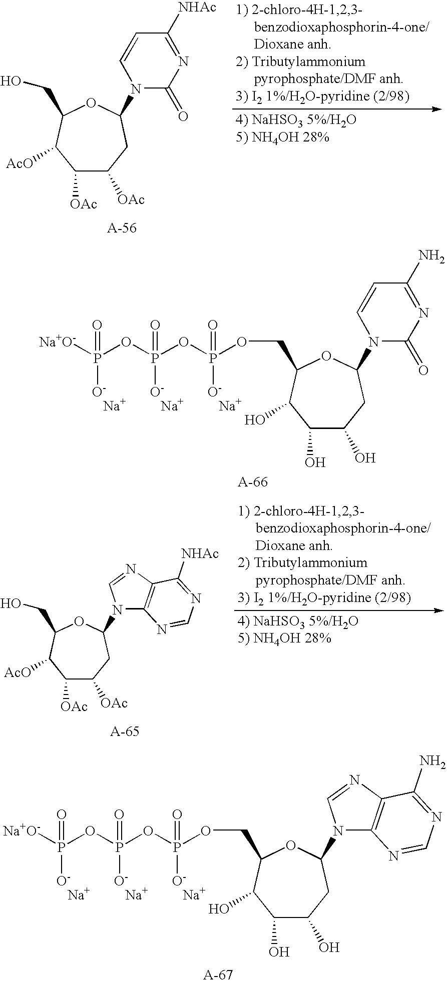

Derivatives were prepared using the methodology described in J. Ludwig and F. Eckstein, Journal of Organic Chemistry, 1989, 54, 631-635.

### Example 15

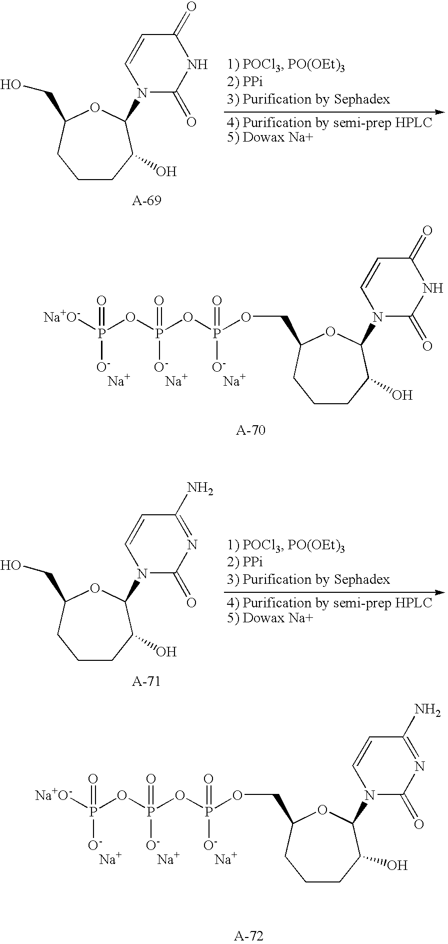

Compounds A-69 and A-71 were prepared according to Approach F′. Triphosphates derivatives were prepared using M. Yoshikawa's procedure.

LC/MS analysis: Compounds were analysed by LC/MS. The LC/MS consists of a Waters Alliance 2790 series binary pump, vacuum degasser, auto sampler, variable wavelength detector (diode array 996) and the Q-TOF Micromass mass spectrometer equipped with an electrospray ionization source. Separation was achieved with a 50×2.1 mm Hypersil BDS C18 column using a gradient mobile phase (gradient sol A 100% H₂O to sol B 80% ACN in 20 min) and a flow rate of 0.2 mL/min. The UV detector (996 PDA) was run from 210 to 400 mn. MS conditions: source block temperature: 100° C., desolvation temperature: 120° C., cone voltage: 20 V, capillary voltage: 3000 V.

HPLC analysis: Chromolith column (100 mm×4.6 C18) was used with the following gradient: from water 100% to acetonitrile 100% in 10 minutes and then acetonitrile for 5 minutes. The flow rate was 2 ml/min. The UV detector was run from 210 to 360 nm.

### Example 16

In the example given below: A, C=H; B, D=OH; BASE=Adenine 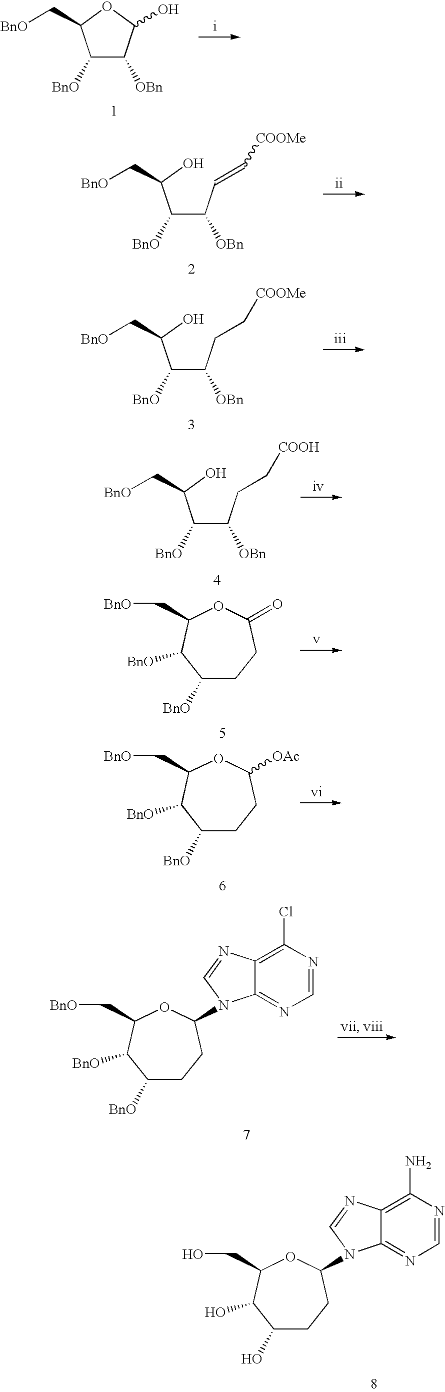
2,3,5-Tri-O-benzyl-(D)-ribose (compound 1) is commercially available.
Step i) The synthesis of compound 2 is described in Ohrui, H.; Jones, G. H.; Moffatt, J. G.; Maddox, M. L.; Christensen, A. T.; Byram, S. K. J. Am. Chem. Soc., 1975, 97, 4602-4613.
Step ii) The synthesis of compound 3 was adapted from Kido, F.; Tsutsumi, K.; Maruta, R.; Yoshikoshi, A. J. Am. Chem. Soc., 1979, 101, 6420-6424: Ester 2 (12.14 g, 25.5 mmol) was dissolved in MeOH (390 mL). To this solution was added NiCl₂.6 H₂O (1.56 g, 6.5 mmol) and the green solution was cooled down to 0° C. NaBH₄ (4.00 g, 102.0 mmol) was added in small portions then, the dark suspension was raised to room temperature and stirred for 4 h. The reaction was quenched with NH₄Cl saturated solution (100 mL) and MeOH was evaporated. The residue was taken up in DCM (300 mL) and extracted with 2 N HCl (2×300 mL), NaHCO₃ saturated solution (300 mL) and brine (300 mL). After evaporation of the organic phase, the crude material was purified by silica gel chromatography (petroleum ether/EtOAc 3:1) to give 3 as a colorless oil (9.47 g, 78%). ¹H NMR (CDCl₃, 300 MHz): 1.96-2.10 (m, 2H), 2.36-2.56 (m, 2H), 2.71 (d, J=4.5 Hz, 1H), 3.60-3.65 (m, 1H), 3.63 (s, 3H), 3.67-3.77 (m, 2H), 3.80-3.95 (m, 2H), 4.50-4.84 (m, 6H), 7.27-7.42 (m, 15H). ES/MS: m/z 501 (M+Na)⁺.
Step iii) The synthesis of compound 4 was adapted from Sasaki, M.; Ishikawa, M.; Fuwa, H.; Tachibana, K. Tetrahedron, 2002, 58, 1889-1911: Ester 3 (9.31 g, 19.5 mmol) was dissolved in THF/MeOH/H₂O 1:1:1 (180 mL). KOH (2.34 g, 41.8 mmol) was added and the yellow solution was stirred at 55° C. for 1 h. The solution was acidified with 1 N HCl (50 mL) and the solvents were evaporated. The residue was taken up in DCM (250 mL) and extracted with 1 N HCl (250 mL) and brine (250 mL). After evaporation of the organic phase, the crude material was purified by silica gel chromatography (DCM/MeOH 97:3) to give 4 as a viscous yellowish oil (8.03 g, 88%). ¹H NMR (CDCl₃, 300 MHz): 1.93-2.14 (m, 2H), 2.39-2.58 (m, 2H), 3.58-3.65 (m, 1H), 3.68-3.79 (m, 2H), 3.82-3.94 (m, 2H), 4.50-4.83 (m, 6H), 7.27-7.42 (m,15H). ES/MS: m/z 487 (M+Na)⁺, m/z 463 (M−H)⁻.
Step iv) The synthesis of compound 5 was adapted from Sasaki, M.; Ishikawa, M.; Fuwa, H.; Tachibana, K. Tetrahedron, 2002, 58, 1889-1911: Acid 3 (7.89 g, 17.0 mmol) and Et₃N (3.32 mL, 23.6 mmol) were dissolved in THF (110 mL) under N₂. 2,4,6-Trichlorobenzoyl chloride (2.95 mL, 18.8 mmol) was added dropwise and the cloudy solution was stirred for 15 h at room temperature. The resulting suspension was diluted with toluene (110 mL) and added dropwise over 8 h to a solution of DMAP (3.17 g, 25.7 mmol) in toluene (850 mL) heated at 90° C. Then, the solvents were evaporated. The residue was taken up in DCM (250 mL) and extracted with 1 N HCl (250 mL), NaHCO₃ saturated solution (250 mL) and brine (250 mL). After evaporation of the organic phase, the crude material was purified by silica gel chromatography (petroleum ether/EtOAc 3:1) to give 5 as a white solid (6.54 g, 86%). ¹H NMR (CDCl₃, 300 MHz): 1.71-1.85 (m, 1H), 2.09-2.21 (m, 1H), 2.38-2.49 (m, 1H), 3.00-3.14 (m, 1H), 3.66-3.73 (dd, J=2.2, 8.0 Hz, 1H), 3.82-3.88 (m, 2H), 3.90-3.96 (m, 1H), 4.53-4.72 (m, 6H), 4.84-4.92 (m, 1H), 7.27-7.41 (m, 15H). ES/MS: m/z 469 (M+Na)⁺.
Step v) The synthesis of compound 6 was adapted from Rychnovsky, S. D.; Dahanukar, V. H. J. Org. Chem., 1996, 61, 7648-7649: To a solution of lactone 5 (3.57 g, 8.0 mmol) in DCM (45 mL) cooled down to −78° C. was added DIBAL-H (1.7 M solution in DCM) (7.05 mL, 12.0 mmol) dropwise. The resulting solution was stirred for 1.5 h at this temperature. Then, pyridine (5.19 mL, 64.0 mmol), DMAP (1.17 g, 9.2 mmol) (dissolved in 6.5 mL of DCM) and Ac₂O (7.62 mL, 80.0 mmol) were slowly added in sequence. The yellowish solution was stirred for 8 h at −78° C. then allowed to warm up to room temperature. The solvents were removed and the crude residue was purified by silica gel chromatography (petroleum ether/EtOAc 4:1) to give the major epimer of 6 as a colorless oil (2.12 g, 55%). ¹H NMR (CDCl₃, 300 MHz): 1.72-1.96 (m, 3H), 2.07 (s, 3H), 2.17-2.33 (m, 1H), 3.46 (dd, J=5.0, 10.1 Hz, 1H), 3.57 (dd, J=4.4, 10.1 Hz, 1H), 3.83-3.90 (m, 1H), 3.98-4.02 (m, 1H), 4.14-4.20 (m, 1H), 4.44-4.73 (m, 6H), 5.89 (dd, J=2.4, 9.3 Hz, 1H), 7.24-7.38 (m, 15H). ES/MS: m/z 513 (M+Na)⁺.
Step vi): Acetate 6 (950 mg, 1.94 mmol) was dissolved in MeCN (9.5 mL). To this solution was added 6-chloropurine (305 mg, 2.22 mmol). The resulting suspension was cooled down to 0° C. then, DBU (850 μL, 5.83 mmol) and TMSOTf (1.48 mL, 7.85 mmol) were added dropwise in sequence. The brown solution was stirred for 24 h at room temperature. MeCN was evaporated and the crude material was purified by silica gel chromatography (petroleum ether/EtOAc 3:1) to give 7 as a yellowish solid (first eluted, major product—350 mg, 31%). ¹H NMR (CDCl₃, 300 MHz): 1.90-2.01 (m, 1H), 2.18-2.54 (m, 3H), 3.45 (dd, J=5.1, 10.1 Hz, 1H), 3.54 (dd, J=4.7, 10.1 Hz, 1H), 3.95-4.03 (m, 1H), 4.05-4.10 (m, 1H), 4.24-4.30 (m, 1H), 4.46-4.51 (m, 2H), 4.55 (s, 2H), 4.66 (d, J=12.0 Hz, 1H), 4.81 (d, J=12.0 Hz, 1H), 6.26 (dd, J=2.6, 10.4 Hz, 1H), 7.20-7.42 (m, 15H), 8.29 (s, 1H), 8.76 (s, 1H). ES/MS: m/z 585 (M+H)⁺.
Step vii): Nucleoside 7 (330 mg, 0.56 mmol) was treated with ammonia-saturated MeOH (50 mL) in a sealed reactor for 15 h at 120° C. The solvent was evaporated and the crude material was rapidly purified by silica gel chromatography (DCM/MeOH 95:5) to give a yellowish solid (265 mg, 82%). ¹H NMR (CDCl₃, 300 MHz): 1.88-1.99 (m, 1H), 2.18-2.54 (m, 3H), 3.44 (dd, J=5.3, 10.3 Hz, 1H), 3.54 (dd, J=4.8, 10.3 Hz, 1H), 3.94-4.01 (m, 1H), 4.06-4.11 (m, 1H), 4.23-4.30 (m, 1H), 4.45-4.51 (m, 2H), 4.53 (s, 2H), 4.67 (d, J=12.0 Hz, 1H), 4.81 (d, J=12.0 Hz, 1H), 5.55 (br, 2H), 6.15 (dd, J=2.7, 9.9 Hz, 1H), 7.19-7.43 (m, 15H), 7.98 (s, 1H), 8.38 (s, 1H). ES/MS: m/z 566 (M+H)⁺.
Step viii): The previous product (240 mg, 0.42 mmol) was dissolved in MeOH (30 mL) and cyclohexene (8 mL). Palladium hydroxide 20% on charcoal (200 mg) was added and the mixture was refluxed for 20 h. The suspension was cooled down, filtered on celite and the solvents were removed. The crude material was purified by reverse phase chromatography (H₂O/MeCN 95:5) to give 8 as a white solid (95 mg, 76%). ¹H NMR (DMSO-D₆, 300 MHz): 1.58-1.74 (m, 1H), 2.03-2.22 (m, 2H), 2.26-2.41 (m, 1H), 3.33-3.53 (m, 2H), 3.67-3.76 (m, 2H), 3.85-3.95 (m, 1H), 4.70 (d, J=4.8 Hz, 1H), 4.79 (t, J=5.4 Hz, 1H), 4.93 (d, J=4.2 Hz, 1H), 5.94 (dd, J=2.7, 10.2 Hz, 1H), 7.26 (br, 2H), 8.13 (s, 1H), 8.30 (s, 1H). ES/MS: m/z 296 (M+H)⁺.

### Example 17

In the example given below: A, C=H; B, D=OH; BASE=Cytosine 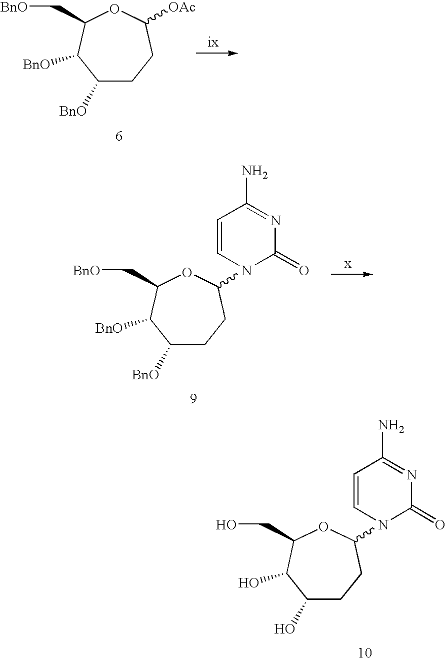
Step ix): The synthesis of compound 9 was adapted from Koshkin, A. A.; Fensholdt, J.; Pfundheller, H. M.; Lomholt, C. J. Org. Chem., 2001, 66, 8504-8512 and Okabe, M.; Sun, R.-C.; Tam, S. Y.-K.; Todaro, L. J.; Coffen, D. L. J. Org. Chem., 1988, 53, 4780-4786: Cytosine (372 mg, 3.32 mmol) and (NH₄)₂SO₄ (2 mg) were suspended in hexamethyldilsilazane (3 mL) and the mixture was refluxed for 1 h. Le solution was evaporated to dryness and coevaporated with toluene (2×10 mL). To the white residue was added acetate 6 (815 mg, 1.66 mmol) dissolved in MeCN (15 mL). The resulting suspension was cooled down to 0° C. and TMSOTf (464 μL, 3.46 mmol) was added. The colorless solution was stirred at room temperature for 15 h. Then, MeCN was evaporated and the crude material was purified by silica gel chromatography (DCM/MeOH 96:4) to give 9 as a yellowish solid (˜7:3 mixture of anomeric epimers—560 mg, 62%). ¹H NMR (DMSO-D₆, 300 MHz): 1.72-1.85 (m, 2H), 2.04-2.30 (m, 2H), 3.12-3.52 (under H₂0 peak, m, 1H), 3.54-3.68 (m, 1H), 3.83 (d, J=8.4 Hz, 0.7H), 3.95-4.14 (m, 2H), 4.17-4.27 (m, 0.3H), 4.34-4.71 (m, 6H), 5.66 (d, J=7.5 Hz, 0.3H), 5.70 (d, J=7.5 Hz, 0.7H), 5.77-5.85 (m, 0.3H), 5.89-5.97 (m, 0.7H), 7.04-7.28 (br, 2H), 7.20-7.42 (m, 15H), 7.59 (d, J=7.5 Hz, 0.7H), 7.77 (d, J=7.5 Hz, 1H). ES/MS: m/z 542 (M+H)⁺.
Step x): Nucleoside 9 (530 mg, 0.98 mmol) was dissolved in MeOH (60 mL) and cyclohexene (16 mL). Palladium hydroxide 20% on charcoal (400 mg) was added and the mixture was refluxed for 20 h. The suspension was cooled down, filtered on celite and the solvents were removed. The crude material was purified by reverse phase chromatography (H₂O/MeCN 98:2) to give 10 as a white solid (˜7:3 mixture of anomeric epimers—207 mg, 77%). ¹H NMR (DMSO-D₆, 300 MHz): 1.47-1.89 (m, 3H), 1.92-2.10 (m, 1H), 3.34-3.50 (under H₂0 peak, m, 2H), 3.52-3.60 (m, 0.7H), 3.62-3.71 (m, 1H), 3.73-3.83 (m, 0.7H), 3.85-3.92 (m, 0.3H), 3.93-4.01 (m, 0.3H), 4.55-4.67 (m, 1.6H), 4.70-4.78 (m, 0.7H), 4.80-4.86 (m, 0.7H), 5.71 (d, J=7.2 Hz, 1H), 5.73-5.81 (m, 1H), 7.00-7.30 (br, 2H), 7.61 (d, J=7.2 Hz, 0.7H), 7.88 (d, J=7.5 Hz, 0.3H). ES/MS: m/z 272 (M+H)⁺, m/z 284 (M+Na)⁺.

### Example 18

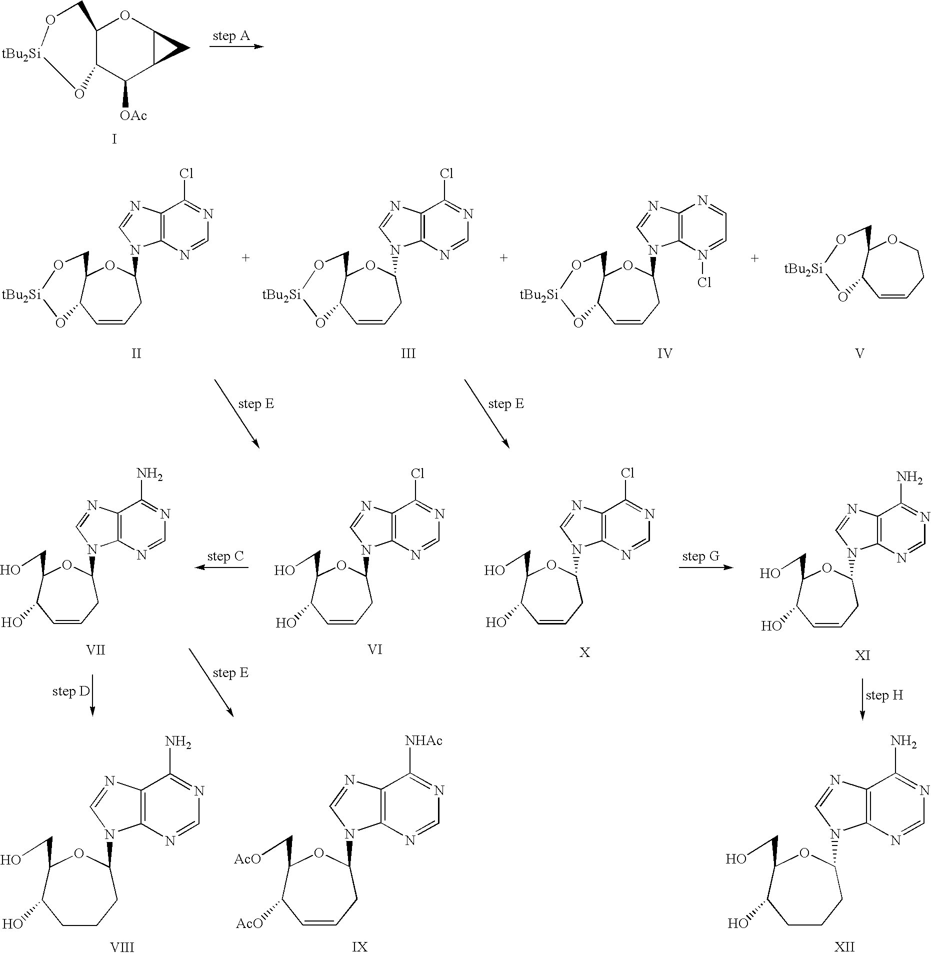 Step A: Compounds II, III, IV and V

The 6-chloropurine (3.3 g, 21.35 mmol) was treated under reflux with an excess of hexamethyldisilazane. The excess of hexamethyldisilazane was removed by distillation under reduced pressure. This residue was stirred in acetonitrile and was added to a solution of the compound I [for preparation see Journal of Organic Chemistry, 1997, Vol 62, No 19, 6615] (7.12 gr, 20.79 mmol) in acetonitrile. Then trimethylsilyl trifluoromethanesulfonate (1 eq.) was added to the mixture and stirred at 20° C. for 6 hours. The reaction mixture was poured into an aqueous solution of sodium hydrogenocarbonate. The aqueous solution was extracted with diethyl ether. The crude product was purified on silica gel to give 1.70 g of the compound II as a beige powder, 1.34 g of the compound III as a white powder, 1.07 g of the compound IV as a yellow powder and 2.76 g of the compound V as a yellow oil.

Compound II:

¹H NMR (CDCl₃) δ ppm: 1.02 (s, 9H), 1.07 (s, 9H), 3.05 (m, 1H, H_{2′}), 2.73 (ddd, 1H, H_{2′}), 3.77 (m, 1H, H_{6′}), 3.89 (t, 1H, J=10.23 Hz, H_{7′}), 4.05 (dd, 1H, J=10.44 Hz, J=4.65 Hz, H_{7′}), 4.72 (m, 1H, H_{5′}), 5.77 (m, 1H, H_{3′}), 5.95 (dd, 1H, J=10.3 Hz, J=1.6 Hz, H_{1′}), 6.03 (m, 1H, H_{4′}), 8.30 (s, 1H, H₈), 8.77 (s, 1H, H₂)

Compound III:

¹H NMR (CDCl₃) δ ppm: 1.01 (s, 9H), 1.04 (s, 9H), 2.69 (ddd, 1H, H_{2′}), 3.75 (m, 2H), 3.91 (m, 1H), 4.71 (m, 2H), 5.79 (m, 1H, H_{3′}), 5.93 (m, 1H, H_{4′}), 6.18 (dd, 1H, J=10.3 Hz, J=3.7 Hz, H_{1′}), 8.24 (s, 1H, H₈), 8.75 (s, 1H, H₂)

compound IV:

¹H NMR (C₆D₆) δ ppm: 1.48 (s, 9H), 1.49 (s, 9H), 2.12 (m, 1H, H_{2′}), 2.49 (m, 1H, H_{2′}), 4.08 (m, 1H, H_{6′}), 4.18 (m, 1H, H_{7′}), 4.52 (dd, 1H, J=10.3 Hz, J=4.4 Hz, H_{7′}), 4.96 (m, 1H, H_{5′}), 5.32 (m, 1H, H_{3′}), 5.83 (m, 1H, H_{1′}), 6.83 (m, 1H, H_{4′}), 8.13 (s, 1H, H₈), 9.24 (s, 1H, H₂)

Step B: Compound VI

Bu₄NF (4.7 mL, 1M in THF) was added to a solution of compound II (970 mg, 2.2 mmol) in THF (7.5 mL) and stirred at 0° C. for 1.5 hours. The reaction mixture was evaporated to dryness to afford a residue which was purified on silica gel using dichloromethane/methanol as eluant and then purified on silica gel reverse-phase (C18) using water/acetonitrile as eluant to give the title compound (180 mg) as a white powder.

¹H NMR (DMSO-d₆) δ ppm: 2.80 (m, 1H, H_{2′}), 3.23 (m, 1H, H_{2′}), 3.46 (m, 1H, H_{7′}), 3.64 (m, 2H), 4.12 (br, 1H, H_{5′}), 4.55 (m, 1H, OH_{7′}), 5.33 (m, 1H, OH_{5′}), 5.66 (m, 1H, H_{3′}), 5.83 (m, 1H, H_{4′}), 6.00 (dd, 1H, J=9.9 Hz, J=1.9 Hz, H_{1′}), 8.82 (s, 1H, H₈), 8.90 (s, 1H, H₂) Mass spectrum: m/z (FAB>0) 297 (M+H)⁺

Step C: Compound VII

The compound from Step B (170 mg, 0.57 mmol) was added to a solution of ammonia in methanol and was stirred in a steel bomb at 100° C. for 2.5 hours. The reaction mixture was evaporated to dryness to afford a residue which was purified on silica gel reverse-phase (C18) using water/acetonitrile (98/2) as eluant to give the title compound (64 mg) as a white powder.

¹H NMR (DMSO-d₆) δ ppm: 2.75 (m, 1H, H_{2′}), 3.14 (m, 1H, H_{2′}), 3.45 (m, 1H, H_{7′}), 3.59 (m, 1H, H_{6′}), 3.68 (m, 1H, H_{7′}), 4.09 (m, 1H, H_{5′}), 4.72 (m, 1H, OH₇′), 5.29 (m, 1H, OH₅′), 5.65 (m, 1H, H_{3′}), 5.81 (m, 2H, H_{4′}and H_{1′}), 7.30 (br, 2H, NH₂), 8.14 (s, 1H, H₈), 8.31 (s, 1H, H₂) Mass spectrum: m/z (FAB>0) 278 (M+H)⁺

Step D: Compound VIII

A mixture of compound from Step C (30 mg, 0.11 mmol), palladium on charcoal (10%) in ethanol/water was stirred under hydrogen until the compound from step C was consumed. The reaction mixture was filtrated through a pad of celite and washed by ethanol/water. The organic layer was evaporated to dryness to afford a slight yellow powder which was crystallized from ethyl acetate to give the title compound (21 mg) as a white powder.

¹H NMR (DMSO-d₆) δ ppm: 1.57-2.34 (m, 6H, 3×CH₂), 3.25-3.70 (m, 3H, H_{6′} and 2×H_{7′}), 4.98 (br, 2H, 2×OH), 5.81 (dd, 1H, H_{1′}), 7.28 (br, 2H, NH₂), 8.14 (s, 1H, H₈), 8.29 (s, 1H, H₂) Mass spectrum: m/z (FAB>0) 280 (M+H)⁺

Step E: Compound IX

The compound VII (900 mg, 3.24 mmol) was dissolved in pyridine (30 mL). An excess of Acetic anhydride was added and the mixture was stirred at 60° C. for 3 hours. The reaction mixture was evaporated to dryness to afford a residue which was purified on silica gel using dichloromethane/ethanol as eluant to give the title compound (590 mg) as a yellow powder.

¹H NMR (DMSO-d₆) δ ppm: 1.87 (s, 3H), 2.08 (s, 3H), 2.25 (s, 3H), 2.86 (m, 2H), 4.03 (m, 1H), 4.13-4.29 (m, 2H), 5.37 (m, 1H), 5.72 (m, 1H), 5.87 (m, 1H), 6.06 (m, 1H), 8.67 (s, 1H), 8.69 (s, 1H), 10.73 (s, 1H, NH) Mass spectrum: m/z (FAB>0) 807 (2M+H)⁺, 404 (M+H)⁺, (FAB<0) 805 (2M−H)⁻, 402 (M−H)

Step F: Compound X

Bu₄NF (2.5 mL, 1M in THF) was added to a solution of compound III (520 mg, 1.1 mmol) in THF (4 mL) and stirred at 0° C. for 1.5 hours. The reaction mixture was evaporated to dryness to afford a residue which was purified on silica gel using dichloromethane/methanol as eluant and then purified on silica gel reverse-phase (C18) using water/acetonitrile as eluant to give the title compound (90 mg) as a white powder.

1H NMR (DMSO-d₆) δ ppm: 2.71 (m, 1H, H_{2′}), 3.37 (m, 1H, H_{2′}), 3.66 (m, 2H), 3.95 (m, 1H), 4.54 (m, 1H, H_{5′}+OH_{7′}), 5.25 (d, 1H, J=6.5 Hz, OH_{5′}), 5.73 (m, 2H, H_{3′}+H_{4′}), 6.21 (dd, 1H, J=11.5 Hz, J=4.4 Hz, H_{1′}), 8.80 (s, 1H, H₈),9.08 (s, 1H, H₂) Mass spectrum: m/z (FAB>0) 297 (M+H)⁺

Step G: Compound XI

The compound from Step F (86 mg, 0.29 mmol) was added to a solution of ammonia in methanol and was stirred in a steel bomb at 100° C. for 2.5 hours. The reaction mixture was evaporated to dryness to afford a residue which was purified on silica gel reverse-phase (C18) using water/acetonitrile (98/2) as eluant to give the title compound (47 mg) as a white powder.

¹H NMR (DMSO-d₆) δ ppm: 2.60 (m, 1H), 3.43-3.67 (m, 3H), 4.06 (m, 1H), 4.43 (m, 1H, H_{5′}), 4.72 (br, 1H, OH₇′), 5.18 (br, 1H, OH₅′), 5.71 (m, 2H, H_{3′} and H_{4′}), 6.07 (dd, 1H, J=11.5 Hz, J=4.1 Hz, H_{1′}), 7.25 (br, 2H, NH₂), 8.14 (s, 1H, H₈), 8.47 (s, 1H, H₂) Mass spectrum: m/z (FAB>0) 278 (M+H)⁺

Step H: Compound XII

A mixture of compound from Step G (30 mg, 0.11 mmol), palladium on charcoal (10%) in ethanol/water was stirred under hydrogen until the compound from step F was consumed. The reaction mixture was filtrated through a pad of celite and washed by ethanol/water. The organic layer was evaporated to dryness to afford a residue which was purified on silica gel reverse-phase (C18) using water as eluant to give the title compound (18 mg) as a white powder.

¹H NMR (DMSO-d₆) δ ppm: 1.45-2.25 (m, 6H, 3×CH₂), 3.49 (m, 3H, H_{6′} and 2×H_{7′}), 4.91 (br, 1H, OH), 5.09 (br, 1H, OH), 6.10 (dd, 1H, H_{1′}), 7.40 (br, 2H, NH₂), 8.29 (s, 1H, H₈), 8.54 (s, 1H, H₂) Mass spectrum: m/z (FAB>0) 559 (2M+H)⁺, 280 (M+H)⁺, (FAB<0) 557 (2M−H)⁻, 278 (M−H)

### Example 19

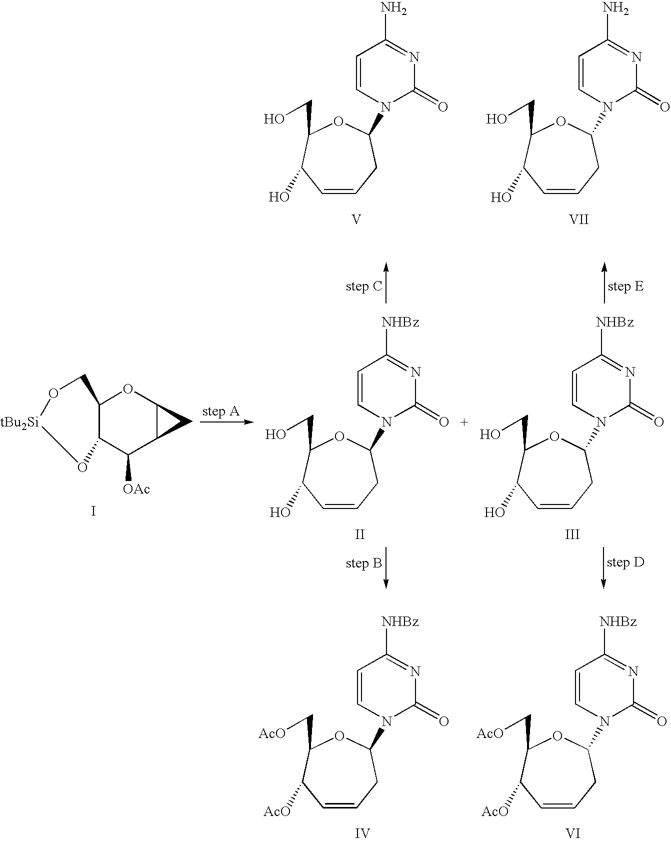 Step A: Compounds II and III

The benzoylcytosine (2.8 g, 13.0 mmol) was treated under reflux with an excess of hexamethyldisilazane. The excess of hexamethyldisilazane was removed by distillation under reduced pressure. This residue was stirred in acetonitrile and was added to a solution of the compound I [for preparation see Journal of Organic Chemistry, 1997, Vol 62, No 19, 6615] (4.5 gr, 13.1 mmol) in acetonitrile. Then trimethylsilyl trifluoromethanesulfonate (1 eq.) was added to the mixture and stirred at 20° C. for 1.5 hours. The reaction mixture was poured into an aqueous solution of 15 sodium hydrogenocarbonate. The aqueous solution was extracted with diethyl ether. The crude product was purified on silica gel using dichloromethane/ethyl acetate as eluant to give a mixture of 2 compounds (2.16 g) which were treated by a solution of Bu₄NF (2.1 eq, 1M in THF) in THF and stirred at 0° C. for 1.5 hours. The reaction mixture was evaporated to dryness to afford a residue which was purified on silica gel using dichloromethane/methanol as eluant and then purified on silica gel reverse-phase (C18) using water/acetonitrile as eluant to give the compound II as a white powder and the compound III as a white powder.

Compound II:

¹H NMR (DMSO-d₆) δ ppm: 2.50 (m, 2H), 3.50 (m, 2H), 3.68 (m, 1H), 4.10 (m, 1H), 4.63 (m, 1H, OH), 5.31 (d, 1H, J=7.9 Hz, OH), 5.52-5.65 (m, 1H), 5.68-5.87 (m, 2H), 7.36 (d, 1H, J=7.0 Hz), 7.55 (m, 2H), 7.63 (m, 1H), 8.01 (d, 2H, J=7.0 Hz), 8.22 (d, 1H, J=8.9 Hz), 11.27 (br, 1H, NH) Mass spectrum: m/z (FAB>0) 358 (M+H)⁺, (FAB<0) 713 (2M−H)⁻, 356 (M−H)⁻

Compound III:

¹H NMR (DMSO-d₆) δ ppm: 2.53-2.60 (m, 1H), 2.87-2.99 (m, 1H), 3.86 (m, 1H, H_{7′}), 3.98 (m, 1H, H_{7′}), 4.41 (m, 1H, H_{6′}), 5.04 (br, 1H, OH_{7′}), 5.33 (br, 1H, OH_{5′}), 5.70 (m, 2H, H_{3′}+H_{4′}), 5.93 (dd, 1H, J=11.2 Hz, J=3.1 Hz, H_{1′}), 7.31 (d, 1H, J=7.4 Hz), 7.51 (m, 2H), 7.64 (m, 1H), 8.01 (d, 2H, J=8.0 Hz), 8.61 (d, 1H, J=7.4 Hz), 11.20 (br, 1H, NH) Mass spectrum: m/z (FAB<0) 356 (M−H)⁻

Step B: Compound IV

The compound II (60 mg, 0.16 mmol) was dissolved in pyridine (2 mL). An excess of Acetic anhydride was added and the mixture was stirred at 20° C. for 5 hours. The reaction mixture was evaporated to dryness. The crude product was purified on silica gel to afford 40 mg of the a slight yellow powder which was crystallized from ethanol to give the title compound (21 mg) as a white powder.

¹H NMR (DMSO-d₆) δ ppm: 1.99 (s, 3H), 2.07 (s, 3H), 2.57-2.85 (m, 2H), 4.03-4.27 (m, 3H), 5.39 (d, 1H, J=7.9 Hz), 5.69 (m, 1H), 5.75-5.92 (m, 2H), 7.37 (d, 1H, J=10.5 Hz), 7.56 (m, 2H), 7.64 (m, 1H), 8.01 (d, 2H, J=8.5 Hz), 8.20 (d, 1H, J=7.7 Hz), 11.27 (br, 1H, NH) Mass spectrum: m/z (FAB>0) 883 (2M+H)⁺, 442 (M+H)⁺, (FAB<0) 881 (2M−H)⁻, 440 (M−H)⁻

Step C: Compound V

The compound II (59 mg, 0.16 mmol) was added to a solution of ammonia in methanol and was stirred at 20° C. for 4.5 hours. The reaction mixture was evaporated to dryness to afford a residue which was purified on silica gel reverse-phase (C18) using water/acetonitrile (99/1) as eluant to give the title compound (23 mg) as a white powder.

¹H NMR (DMSO-d₆) δ ppm: 2.36 (m, 2H), 3.3 (m, 2H), 3.67 (m, 1H), 4.04 (m, 1H), 4.52 (br, 1H, OH), 5.22 (br, 1H, OH), 5.51-5.80 (m, 4H), 7.12 (br, 2H, NH₂), 7.64 (d, 1H, J=7.3 Hz). Mass spectrum: m/z (FAB<0) 252 (M+H)⁻

Step D: Compound VI

The compound III (60 mg, 0.16 mmol) was dissolved in pyridine (2 mL). An excess of Acetic anhydride was added and the mixture was stirred at 20° C. for 5 hours. The reaction mixture was evaporated to dryness. The crude product was purified on silica gel to afford a slight yellow powder which was crystallized from ethanol to give to give the title compound (36 mg) as a white powder.

¹H NMR (DMSO-d₆) δ ppm: 1.97 (s, 3H), 2.05 (s, 3H), 4.11-4.28 (m, 2H), 4.96-5.90 (m, 1H), 5.29 (m, 1H), 5.69 (m, 1H), 5.88-6.08 (m, 2H), 7.37 (m, 1H), 7.53 (m, 2H), 7.64 (m, 1H), 8.01 (d, 2H, J=8.2 Hz), 8.33 (d, 1H, J=7.8 Hz), 11.31 (br, 1H, NH) Mass spectrum: m/z (FAB>0) 442 (M+H)⁺, (FAB<0) 440 (M−H)⁻

Step E: Compound VII

The compound III (58 mg, 0.16 mmol) was added to a solution of ammonia in methanol and was stirred 20° C. for 4.5 hours. The reaction mixture was evaporated to dryness to afford a residue which was purified on silica gel reverse-phase (C18) using water as eluant to give the title compound (33 mg) as a white powder.

¹H NMR (DMSO-d₆) δ ppm: 2.34 (m, 1H), 2.84 (m, 1H), 3.49 (m, 1H), 3.78 (m, 1H), 3.96 (m, 1H), 4.25 (m, 1H), 4.82 (br, 1H, OH), 5.18 (br, 1H, OH), 5.67 (m, 3H), 5.86 (m, 1H), 7.07 (br, 2H, NH₂), 7.96 (d, 1H, J=8.1 Hz). Mass spectrum: m/z (FAB>0) 254 (M+H)⁺

### Example 20

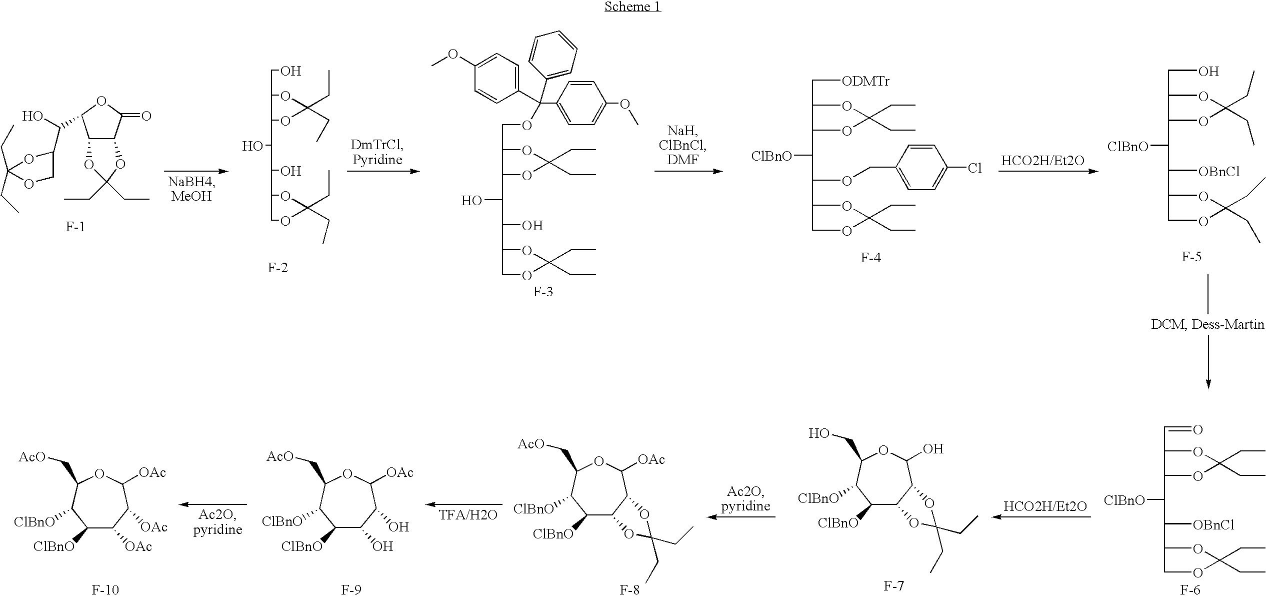

Compound F-1: was prepared according to: Journal of Organic Chemistry, 2000, 65, 4070-87. Molecular Formula: C₁₆H₂₅O₇.

Compound F-2. To a solution of F-1 (60 g, 174 mmol) in methanol (1.21) at 0° C., was added sodium borohydride (NaBH₄) (19.8 g, 523 mmol) per portions. The temperature was allowed to warm to room temperature and the reaction mixture was stirred at the same temperature for 2 hours. A 10% aqueous solution of acetic acid was added until pH=7. The solution was concentrated to dryness, the residue was dissolved in ethyl acetate and washed once with brine. The organic layer was dried over sodium sulfate and concentrated under vacuo to obtain compound F-2 as a crude material directly used in the next step. Molecular Formula: C₁₇H₃₂O₇.

Compound F-3. Dimethoxytrityl chloride (DMTrCl) (66.18 g, 191 mmol) was added to a solution of F-2 (76.02 g, 174 mmol) in anhydrous pyridine (870 ml). The reaction mixture was stirred at room temperature overnight and then evaporated under vacuo. The residue was dissolved in ethyl acetate and washed three times with water. The organic layer was dried over sodium sulfate and concentrated under vacuo. The crude material was purified on a silica gel column chromatography (eluent: from 20 to 30% of diethyl ether in petroleum ether) to obtain compound F-3 (76.02 g, 67%). Molecular Formula: C₃₈H₅₀O₉. ¹H NMR (200 MHz DMSO d₆): δ 7.40-7.20 (m, 9H, DMTr), 7.90 (m, 4H, DMTr), 4.80 (d, 1H, J=7Hz, OH), 4.30 (m, 2H+1OH), 3.95 (m, 2H), 3.70 (s, 6H, 2×OCH₃), 3.65 (m, 1H), 3.30 (m under water, 2H), 3.15-3.00 (m, 2H), 1.60-1.20 (m, 8H, 4×CH₂), 0.90-0.60 (m, 12H, 4×CH₃). MS: FAB−(matrice GT) m/z 649 (M−H)⁻

Compound F4. To a solution of F-3 (76.02 g, 117 mmol) in dimethylformamide (650 ml) was added sodium hydride (NaH 60% dispersion in mineral oil, 15.0 g, 374 mmol) at 0° C. The reaction mixture was stirred at the same temperature for 45 min. Chlorobenzylchloride (60 g, 374 mmol) was added, the temperature was allowed to warm to room temperature and the reaction was stirred overnight. The mixture was diluted in ethyl acetate and washed three times with water. The organic layer was dried over sodium sulfate and concentrated under vacuo to give compound F-4 as a crude material directly used in the next step. Molecular Formula: C₅₂H₆₀Cl₂O₉. ¹H NMR (200 MHz, DMSO d₆): δ 7.40-7.20 (m, 21H, DMTr+ClBn), 4.70-4.10 (m, 6H), 3.95 (m, 1H), 3.85 (m, 2H), 3.70 (d, 6H, 2×OCH₃), 3.40 (m, 1H), 3.35 (m under water, 1H), 3.20 (m, 1H), 3.00 (m, 1H), 1.70-1.20 (m, 8H, 4×CH₂), 0.90-0.70 (m, 12H, 4×CH₃).

Compound F-5. F-4 (117 mmol) was dissolved in a mixture of formic acid (316 ml) and diethyl ether (316 ml) and the reaction mixture was stirred at room temperature for 30 min. The mixture was diluted in ethyl acetate and washed with a saturated aqueous solution of sodium bicarbonate until pH>7. The organic layer was dried over sodium sulfate and concentrated under vacuo. The crude material was purified on a silica gel column chromatography (eluent: from 20 to 50% of diethyl ether in petroleum ether) to obtain compound F-5 (23.55 g, 34%). Molecular Formula: C₃₁H₄₂Cl₂O₇. ¹H NMR (200 MHz, DMSO d₆): δ 7.40-7.20 (m, 8H, ClBn), 4.85 (t, 1H, J=5.8 Hz, OH), 4.70-4.45 (m, 4H, CH₂BnCl), 4.20 (m, 2H), 4.00 (t, 1H, J=3.4 Hz), 3.90 (m, 3H), 3.70 (m, 1H), 3.50 (m, 1H), 3.40 (m, 1H), 1.60-1.40 (m, 8H, 4×CH₂), 0.85-0.70 (m, 12H, 4×CH₃).

Compound F-6. Dess Martin periodinane (18.4 g, 43.45 mmol) was added to a solution of F-5 (23.55 g, 39.5 mmol) in dichloromethane (350 ml). The reaction mixture was stirred at room temperature overnight. Diethyl ether was added and the mixture was filtered through a pad of silica gel/magnesium sulfate/sand and the filtrate was concentrated to dryness. The crude material was purified on a silica gel column chromatography (eluent: 30% of diethyl ether in petroleum ether) to afford compound F-6 (18.21 g, 77%). Molecular Formula: C₃₁H₄₀Cl₂O₇. ¹H NMR (200 MHz, CDCl₃): δ 9.50 (d, 1H, J=2.45 Hz, CHO), 7.40-7.20 (m, 8H, ClBn), 4.80-4.60 (m, 4H, CH₂BnCl), 4.40-3.90 (m, 6H), 3.55 (m, 1H), 1.60 (m, 8H, 4×CH₂), 0.90 (m, 12H, 4×CH₃).

Compound F-7. F-6 (12.97 g, 21.7 mmol) was dissolved in a mixture of formic acid (60 ml) and diethyl ether (60 ml) and the reaction mixture was stirred at room temperature for 5 hours. The mixture was diluted in ethyl acetate and poured into a saturated aqueous solution of sodium bicarbonate, the organic layer washed with a saturated aqueous solution of sodium bicarbonate then with water. The organic layer was dried over sodium sulfate and concentrated under vacuo to obtain compound F-7 as a crude material directly used in the next step. Molecular Formula: C₂₆H₃₂Cl₂O₇.

Compound F-8. To a solution of F-7 (21.7 mmol) in anhydrous pyridine (102 ml) was added acetic anhydride (76.7 ml). The reaction was stirred at room temperature for two days. The mixture was diluted with ethyl acetate and washed successively with chlorhydric acid 1 M, twice with a saturated aqueous solution of sodium bicarbonate and with water. The organic layer was dried over sodium sulfate and concentrated under vacuo. The crude material was purified on a silica gel column chromatography (eluent: from 20 to 50% of diethyl ether in petroleum ether) to afford compound F-8 (10.78 g, 81%). Molecular Formula: C₃₀H₃₆Cl₂O₉. ¹H NMR (400 MHz, CDCl₃): δ 7.40-7.10 (m, 8H), 5.50 (d, 1H, J=8.7 Hz, H1), 4.90 (d, 1H, J=10.7 Hz), 4.80 (d, 1H, J=11.1 Hz), 4.70 (d, 1H, J=10.7 Hz), 4.50-4.20 (m, 5H), 4.00 (m, 1H, H6), 3.80 (t, 1H, H4), 3.40 (t, 1H, H5), 2.20 (s, 3H, CH₃ of OAc), 2.05 (s, 3H, CH₃ of OAc), 1.75 (m, 4H, 2×CH₂), 0.95 (m, 6H, 2×CH₃). MS: FAB+(matrice GT) m/z 671 (M+H)⁺, m/z 551 (M−OAc)⁺.

Compound F-9. F-8 (3.0 g, 4.9 mmol) was dissolved in trifluoroacetic acid (22.5 ml) and water (2.5 ml).The reaction mixture was stirred at room temperature for 2 hours. Evaporation to dryness and two co-evaporations with anhydrous toluene afforded compound F-9 as a crude material which was used directly in the next step. Molecular Formula: C₂₅H₂₈Cl₂O₉.

Compound F-10. To a solution of F-9 (4.9 mmol) in anhydrous pyridine (70 ml) was added acetic anhydride (20 ml). The reaction was stirred at room temperature overnight. The mixture was diluted with ethyl acetate and washed successively with chlorhydric acid 1 M, twice with a saturated aqueous solution of sodium bicarbonate and with water. The organic layer was dried over sodium sulfate and concentrated under vacuo. The crude material was purified on a silica gel column chromatography (eluent: 50% of diethyl ether in petroleum ether) to afford compound F-10 (1.586 g, 51%). Molecular Formula: C₂₉H₃₂Cl₂O₁₁. ¹H NMR (400 MHz, DMSO d₆): δ 7.50-7.20 (m, 8H), 5.80 (m, 1H, H1), 5.35 (m, 1H), 5.25 (m, 1H), 4.70 (dd, 2H, J₁=11.8 Hz, J₂=17.8 Hz), 4.50 (d, 2H, J=11.4 Hz), 4.40 (m, 1H), 4.20-4.10 (m, 2H), 4.00 (m, 1H), 3.60 (m, 1H), 2.10-2.00 (m, 12H, 4×CH₃ from OAc). MS: FAB+(matrice GT) m/z 567 (M−OAc)⁺.

### Example 21

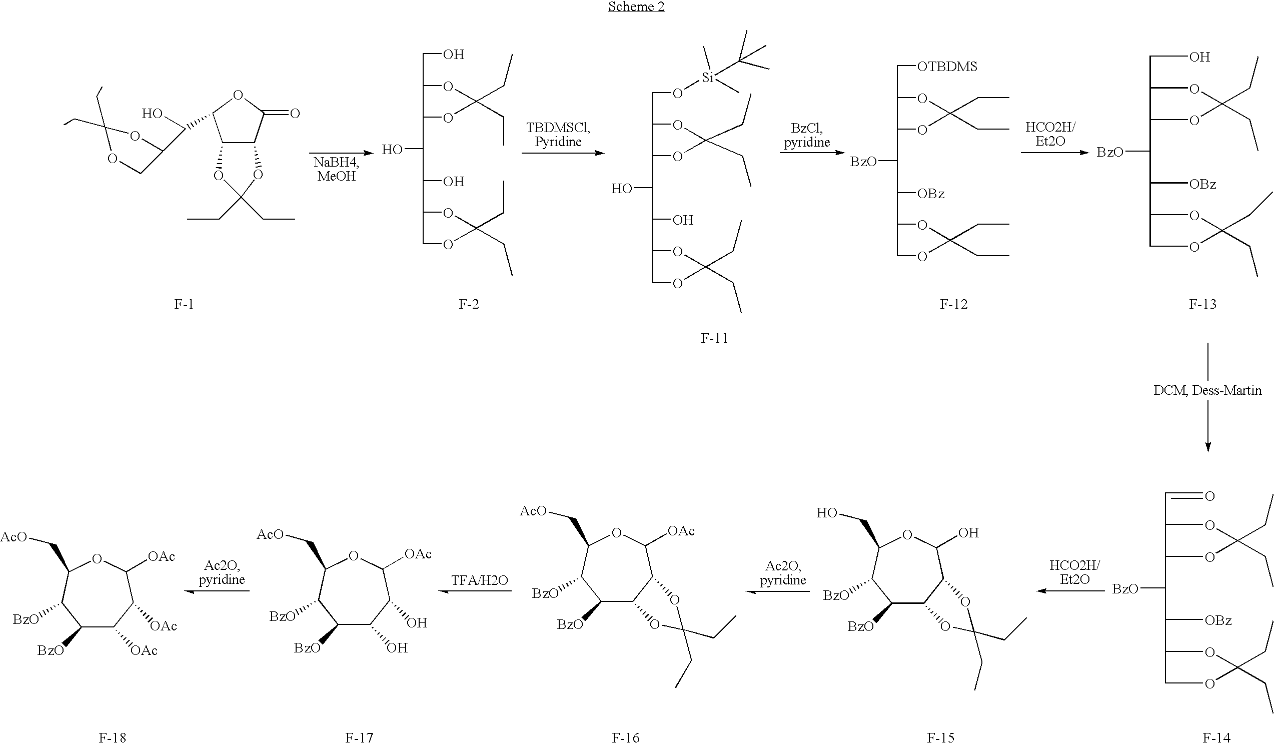

Compound F-11. To a solution of F-2 (28.0 g, 80.4 mmol) in pyridine (400 ml) was added tert-butyldimethylsilylchloride (12.23 g, 80.4 mmol). The reaction was stirred at room temperature for 5 hours, and quenched by addition of ethanol. The reaction was diluted with ethyl acetate and washed three times with a saturated aqueous solution of sodium bicarbonate. The organic layer was dried over sodium sulfate and concentrated under vacuo. The crude material was purified on a silica gel column chromatography (eluent: from 15 to 50% of diethyl ether in petroleum ether) to afford compound F-11 (13.66 g, 37%). Molecular Formula: C₂₃H₄₆O₇Si. ¹H NMR (400 MHz, DMSO d₆): δ 4.75 (d, 1H, OH), 4.35 (d, 1H, OH), 4.20 (m, 1H), 4.10-3.90 (m, 3H), 3.80-3.60 (m, 4H), 3.45 (m, 1H), 1.60-1.40 (m, 8H, 4×CH₂), 0.90-0.80 (m, 27H, 9×CH₃).

Compound F-12. To a solution of F-11 (13.66 g, 29.57 mmol) in pyridine (150 ml) was added dimethylaminopyridine (1.4 g, 11.46 mmol) and benzoyl chloride (17.1 ml, 147.3 mmol) at 0° C. The reaction mixture was stirred at 0° C. for 30 min then at room temperature for 6 hours. The reaction was diluted with ethyl acetate and washed successively twice with a saturated aqueous solution of sodium bicarbonate, twice with chlorhydric acid 1 M and twice with water/brine. The organic layer was dried over sodium sulfate and concentrated under vacuo. The crude material was purified on a silica gel column chromatography (eluent: from 0 to 10% of diethyl ether in petroleum ether) to afford compound F-12 (29 g, >100% presence of pyridine). Molecular Formula: C₃₇H₅₄O₉Si. MS: FAB+(matrice GT) m/z 671 (M+H)⁺.

Compound F-13. F-12 was co-evaporated three times with toluene. Para-Toluenesulfonic acid (1.3 g, 6.91 mmol) was added to a solution of F-12 (29 g, 43.2 mmol) in methanol (260 ml). The reaction mixture was stirred at room temperature for 1 hour. The reaction was quenched by the addition of a saturated aqueous solution of sodium bicarbonate, methanol was evaporated under vacuo, and the mixture was diluted with ethyl acetate. The organic phase was dried over sodium sulfate and evaporated to dryness. The crude material was purified on a silica gel column chromatography (eluent: from 20 to 40% of diethyl ether in petroleum ether) to afford compound F-13 (11.61 g, 50%). Molecular Formula: C₃₁H₄₀O₉. ¹H NMR (400 MHz, DMSO d₆): δ 7.90 (m, 4H, Bz), 7.60-7.40 (m, 6H, Bz), 5.80 (m, 1H), 5.50 (m, 1H), 5.10 (t, 1H, OH), 4.45 (m, 2H), 4.25 (m, 1H), 4.00 (m, 2H), 3.50-3.30 (m, 2H), 1.70-1.30 (m, 8H, 4×CH₂), 0.90-0.70 (m, 12H, 4×CH₃).

Compound F-14. Dess Martin periodinane (8.86 g, 20.88 mmol) was added to a solution of F-13 (11.61 g, 20.87 mmol) in dichloromethane (232 ml). The reaction mixture was stirred at room temperature for 5 hours. Diethyl ether was added and the mixture was filtered through a pad of silica gel/magnesium sulfate/sand and the filtrate was concentrated to dryness. The crude material was purified on a silica gel column chromatography (eluent: 30% of diethyl ether in petroleum ether) to afford compound F-14 (9.58 g, 83%). Molecular Formula: C₃₁H₃₈O₉. ¹H NMR (400 MHz, CDCl₃): δ 9.60 (d, 1H, CHO), 8.00 (m, 4H, Bz), 7.50-7.30 (m, 6H, Bz), 5.80 (m, 1H), 5.60 (m, 1H), 4.85 (m, 1H), 4.50 (m, 2H), 4.00 (m, 2H), 1.70-1.50 (m, 8H, 4×CH₂), 0.90-0.70 (m, 12H, 4×CH₃). MS: FAB+(matrice GT) m/z 555 (M+H)⁺.

Compound F-15. F-14 (9.58 g, 17.3 mmol) was dissolved in a mixture of formic acid (76 ml) and diethyl ether (76 ml) and the reaction mixture was stirred at room temperature for 5 hours. The mixture was diluted in ethyl acetate and poured into a saturated aqueous solution of sodium bicarbonate, the organic layer washed with a saturated aqueous solution of sodium bicarbonate then with water. The organic layer was dried over sodium sulfate and concentrated under vacuo to obtain compound F-15 as a crude material directly used in the next step. Molecular Formula: C₂₆H₃₀O₉.

Compound F-16. To a solution of F-15 (17.3 mmol) in anhydrous pyridine (149 ml) was added acetic anhydride (69 ml). The reaction was stirred at room temperature for two days. The mixture was diluted with ethyl acetate and washed successively with chlorhydric acid 1 M, twice with a saturated aqueous solution of sodium bicarbonate and with water. The organic layer was dried over sodium sulfate and concentrated under vacuo. The crude material was purified on a silica gel column chromatography (eluent: 30% of diethyl ether in petroleum ether) to afford compound F-16 (7.9 g, 79%). Molecular Formula: C₃₀H₃₄O₁₁. ¹H NMR (400 MHz, CDCl₃): δ 7.80 (m, 4H, Bz), 7.50-7.20 (m, 6H, Bz), 5.80 (m, 2H, H4+H1), 5.50 (t, 1H, J=10.2 Hz, H5), 4.7 0(dd, 1H, J₁=7.4 Hz, J₂=9.8 Hz, H3), 4.60 (t, 1H, J=8.4 Hz, H2), 4.40 (m, 1H, H6), 4.25 (dd, 1H, J₁=5.2 Hz, J₂=12.3 Hz, H7a), 4.15 (dd, 1H, J₁=2.5 Hz, J₂=12.3 Hz, H7b), 2.20 (s, 3H, OAc), 2.00 (s, 3H, OAc), 1.70-1.50 (m, 4H, 2×CH₂), 0.90 (m, 6H, 2×CH₃). MS: FAB+(matrice GT) m/z 511 (M−OAc)⁺.

Compound F-17. F-16 (310 mg, 0.54 mmol) was dissolved in trifluoroacetic acid (4.5) and water (0.5 ml).The reaction mixture was stirred at room temperature for 2 hours. Evaporation to dryness and two co-evaporations with anhydrous toluene afforded compound F-17 as a crude material which was used directly in the next step. Molecular Formula: C₂₅H₂₆O₁₁.

Compound F-18. To a solution of F-17 (0.54 mmol) in anhydrous pyridine (5.3 ml) was added acetic anhydride (2.4 ml). The reaction was stirred at room temperature overnight. The mixture was diluted with ethyl acetate and washed successively with chlorhydric acid 1 M, twice with a saturated aqueous solution of sodium bicarbonate and with water. The organic layer was dried over sodium sulfate and concentrated under vacuo. The crude material was purified on a silica gel column chromatography (eluent: 50% of diethyl ether in petroleum ether) to afford compound F-18 (89 mg, 28%). Molecular Formula: C₂₉H₃₀O₁₃. ¹H NMR (400 MHz, DMSO d₆): δ 8.10-7.20 (m, 10H, 2 Bz), 6.10-5.60 (m, 4H), 5.55-5.30 (m, 2H), 4.50-4.10 (m, 2H), 2.20-2.00 (m, 12H, 4×CH₃ of OAc). MS: FAB+(matrice GT) m/z 527 (M−OAc)⁺.

### Example 22

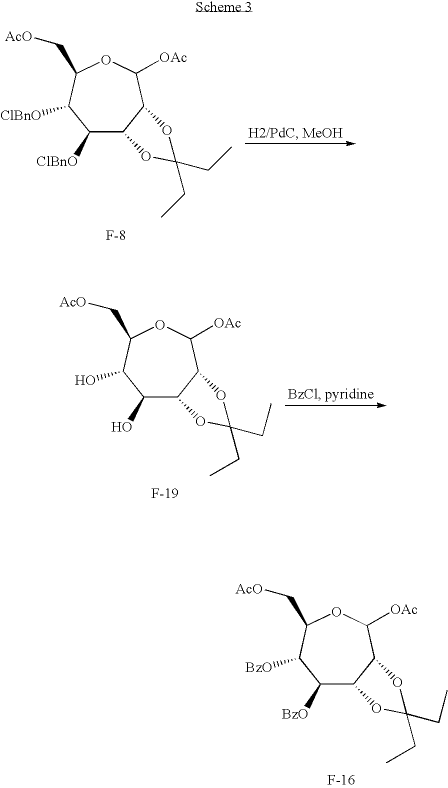

Compound F-19. Palladium on activated charcoal (5%, 150 mg) was added to a solution of F-8 (300 mg, 0.49 mmol) in a mixture of toluene (3.2 ml) and ethanol (1.7 ml). The reaction mixture was hydrogenated at room temperature under atmosphere pressure for 16 hours. The reaction mixture was filtered through a pad of celite. The filtrate was evaporated under vacuo. The residue was purified on a silica gel column chromatography (eluent: from 15 to 20% of diethyl ether in petroleum ether) to give compound F-19 (21 mg, 12%). MS: ESI m/z 363 (M+H)⁺. Molecular Formula: C₁₆H₂₆O₉.

Compound F-16. To a solution of F-19 (21 mg, 0.058 mmol) in pyridine (0.23 ml) was added benzoyl chloride (0.016 ml, 0.14 mmol) at 0° C. The reaction mixture was stirred at room temperature overnight. The reaction was diluted with ethyl acetate and washed successively with a saturated aqueous solution of sodium bicarbonate, chlorhydric acid 1 M and water. The organic layer was dried over sodium sulfate and concentrated under vacuo. The crude material was purified on a silica gel column chromatography (eluent: 30% of diethyl ether in petroleum ether) to afford compound F-16 (13 mg, 40%). Molecular Formula: C₃₀H₃₄O₁₁.

### Example 23

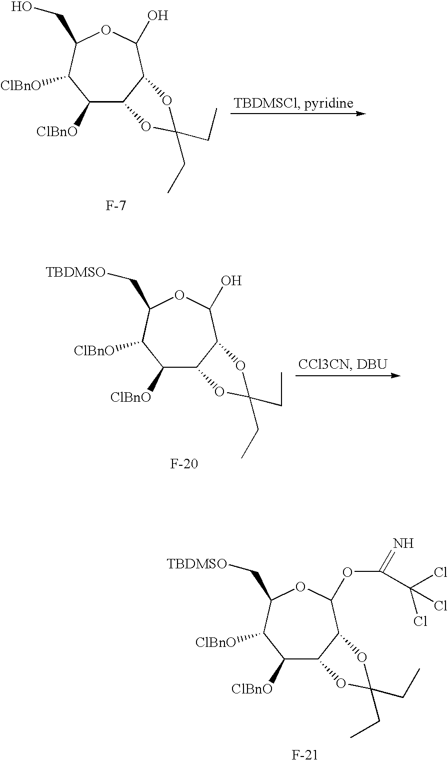

### Scheme 4

Compound F-20. To a solution of F-7 (3.37 mmol) in pyridine (34 ml) was added tert-butyldimethylsilylchloride (609 mg, 4.04 mmol) at 0° C. The reaction was stirred at room temperature overnight, and quenched by addition of ethanol. The reaction was diluted with ethyl acetate and successively washed twice with a saturated aqueous solution of sodium bicarbonate and water. The organic layer was dried over sodium sulfate and concentrated under vacuo. The crude material was purified on a silica gel column chromatography (eluent: from 30% of diethyl ether in petroleum ether) to afford compound F-20 (1.14 g, 53%). Molecular Formula: C₃₂H₄₆Cl₂O₇Si. ¹H NMR (400 MHz, CDCl₃): δ 7.30-7.10 (m, 8H, ClBn), 4.80-4.50 (m, 5H, H1+2CH₂Bn), 4.30 (m, 1H, H3), 4.10 (m, 1H, H2), 3.80 (m, 3H, H4, H7a+H7b), 3.60 (t, 1H, J=9.2 Hz, H6), 3.50 (m, 1H, H5), 1.60 (m, 4H, 2×CH₂), 0.90-0.70 (m, 15H, 5×CH₃), 0.00 (m, 6H, Si(CH₃)₂). MS: ESI m/z 641 (M+H)⁺.

Compound F-21. Trichloroacetonitrile (0.393 ml, 3.92 mmol) and 1,8-diazabicyclo[5,4,0]undec-7-ene (0.053 ml, 0.36 mmol) were added to a solution of F-20 (1.14 g, 1.78 mmol) in dichloromethane (41 ml). The reaction mixture was stirred at room temperature for 3 hours. The reaction was diluted with ethyl acetate and successively washed twice with a saturated aqueous solution of sodium bicarbonate and water. The organic layer was dried over sodium sulfate and concentrated under vacuo. The crude material was purified on a silica gel column chromatography (eluent: from 20% of diethyl ether in petroleum ether) to obtain compound F-21 (1.27 g, 91%). Molecular Formula: C₃₄H₄₆Cl₅NO₇Si. ¹H NMR (400 MHz, CDCl₃): δ 8.55 (s, 1H, NH), 7.30-7.10 (m, 8H, ClBn), 5.60 (d, 1H, J=8.2 Hz, H1), 4.90 (d, 1H, J=10.7 Hz, CH₂Bn), 4.80 (d, 1H, J=11.1 Hz, CH₂Bn), 4.70 (d, 1H, J=10.7 Hz, CH₂Bn), 4.70 (d, 1H, J=11 Hz, CH₂Bn), 4.40 (m, 3H), 3.90 (m, 3H), 3.60 (d, 1H, J=9.7 Hz), 1.70 (m, 4H, 2×CH₂), 0.90 (m, 15H, 5×CH₃), 0.00 (m, 6H, Si(CH₃)₂). MS: ESI m/z 682 (M+H)⁺.

### Example 24

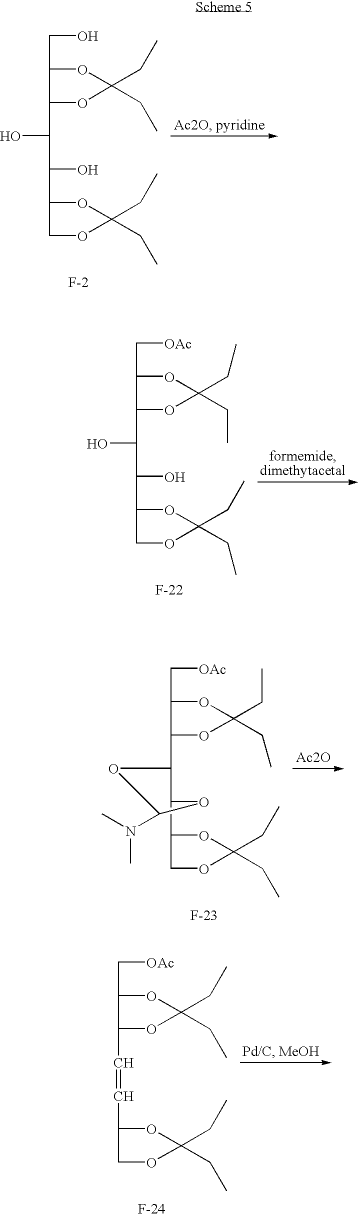

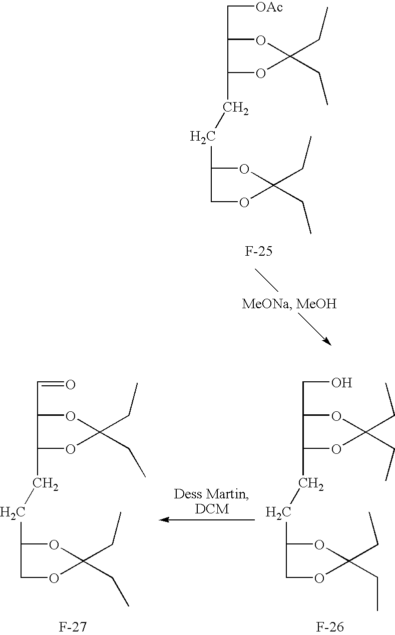

Compound F-24 was prepared according to G. Stork, T. Takashi, I. Kawamoto and T. Suzuki, Journal of the American Chemical Society, 100(26), 8272-8273.

Compound F-22. To a solution of F-2 (40.0 g, 116 mmol) in anhydrous chloroform (800 ml) was added pyridine (12.6 ml, 162.4 mmol) and acetic anhydride (15.3 ml, 162.4 mmol) at 0° C. The reaction was stirred at room temperature overnight. The mixture was diluted with dichloromethane and washed successively with a saturated aqueous solution of sodium bicarbonate and with water. The organic layer was dried over sodium sulfate and concentrated under vacuo. The crude material was purified on a silica gel column chromatography (eluent: 2% of ethanol in dichloromethane) to afford compound F-22 (15.38 g, 34%). Molecular Formula: Cl₉H₃₄O₈. ¹H NMR (200 MHz, DMSO dm₆): δ 4.85 (d, 1H, J=7.4 Hz), 4.60 (d, 1H, J=7.8 Hz), 4.30 (m, 2 H), 4.10-3.90 (m, 5H), 3.75 (m, 1H), 3.60 (m, 1H), 2 (m, 3H, OAc), 1.65-1.45 (m, 8H, 4×CH₂), 0.90 (m, 12H, 4×CH₃). MS: FAB—(matrice GT) m/z 389 (M-H)⁻.

Compound F-23. F-22 (15.38 g, 39.3 mmol) was dissolved with N,N-dimethylformamide dimethylacetal (81 ml) and the reaction mixture was stirred at room temperature for 5 hours. The reaction was diluted in ethyl acetate and washed successively twice with a saturated aqueous solution of sodium bicarbonate and twice with water. The organic layer was dried over sodium sulfate and concentrated under vacuo. The crude material was purified on a silica gel column chromatography (eluent: 30% of diethyl ether in petroleum ether) to give compound F-23 (12.54 g, 72%). Molecular Formula: C₂₂H₃₉NO₈. MS: ESI m/z 446 (M+H)⁺.

Compound F-24. F-23 (12.54 g, 28 mmol) was dissolved with acetic anhydride (63 ml) and the reaction mixture was stirred at reflux during 3 hours. At room temperature, the mixture was dissolved in ethyl acetate and washed successively twice with a saturated aqueous solution of sodium bicarbonate and water. The organic layer was dried over sodium sulfate and concentrated under vacuo. The crude material was purified on a silica gel column chromatography (eluent: 20% of diethyl ether in petroleum ether) to give compound F-24 (6.5 g, 65%). Molecular Formula: C₁₉H₃₂O₆. ¹H NMR (200 MHz, DMSO d₆): δ 5.80 (m, 2H), 4.80 (m, 1H), 4.50 (m, 1 H), 4.36 (m, 1H), 4.10-3.90 (m, 2H), 3.80 (m, 1H), 3.40 (m, 1H), 2.00 (s, 3H, OAc), 1.60 (m, 8H, 4 CH₂), 0.80 (m, 12H, 4 CH₃).

Compound F-25. To a solution of F-24 (5.16 g, 14.47 mmol) in methanol (80 ml) was added 5% palladium on activated charcoal (2.6 g). The reaction mixture was hydrogenated at room temperature under atmosphere pressure for 1 day. The reaction mixture was filtered through a pad of celite. The filtrate was evaporated under vacuo. The residue was purified on a silica gel column chromatography (eluent: from 30% to 50% of diethyl ether in petroleum ether) to give compound F-25 (4.44 g, 86%). Molecular Formula: C₁₉H₃₄O₆. ¹H NMR (200 MHz, DMSO d₆): δ 4.40-4.10 (m, 6H), 3.50 (m, 1H), 2.00 (s, 3H, OAc), 1.60 (m, 12H, 6 CH₂), 0.80 (m, 12H, 4 CH₃).

Compound F-26. Sodium methylate (737 mg, 13.6 mmol) was added to a solution of F-25 (4.44 g, 12.4 mmol) in methanol (62 ml). The reaction mixture was stirred at room temperature during 3 hours. Chlorhydric acid was added until pH=7. The mixture was diluted with ethyl acetate and washed successively with a saturated aqueous solution of sodium bicarbonate and water. The organic layer was dried over sodium sulfate and concentrated under vacuo. The crude material was purified on a silica gel column chromatography (eluent: 30% of diethyl ether in petroleum ether) to give compound F-26 (3.8 g, 97%). Molecular Formula: C₁₇H₃₂O₅. ¹H NMR (200 MHz, CDCl₃): δ 4.20-4.00 (m, 4H), 3.70 (m, 2H), 3.50 (m, 1H), 2.00 (m, 1H, OH), 1.70-1.55 (m, 12H, 6×CH₂), 0.90 (m, 12H, 4×CH₃). MS: ESI m/z 317 (M+H)⁺

Compound F-27. Dess Martin periodinane (5.16 g, 12 mmol) was added to a solution of F-26 (3.5 g, 11 mmol) in dichloromethane (138 ml). The reaction mixture was stirred at room temperature overnight. Diethyl ether was added and the mixture was filtered through a pad of silica gel/magnesium sulfate/sand and the filtrate was concentrated to dryness. The crude material was purified on a silica gel column chromatography (eluent: 30% of diethyl ether in petroleum ether) to afford compound F-27 (3.06 g, 89%). Molecular Formula: C₁₇H₃₀O₆. ¹H NMR (200 MHz, CDCl₃): δ 9.70 (d, 1H, J=3.3 Hz, CHO), 4.35 (m, 2H), 4.05 (m, 2H), 3.35 (m, 1H), 1.60 (m, 12H, 6×CH₂), 0.90 (m, 12H, 4×CH₃).
Illustration 4 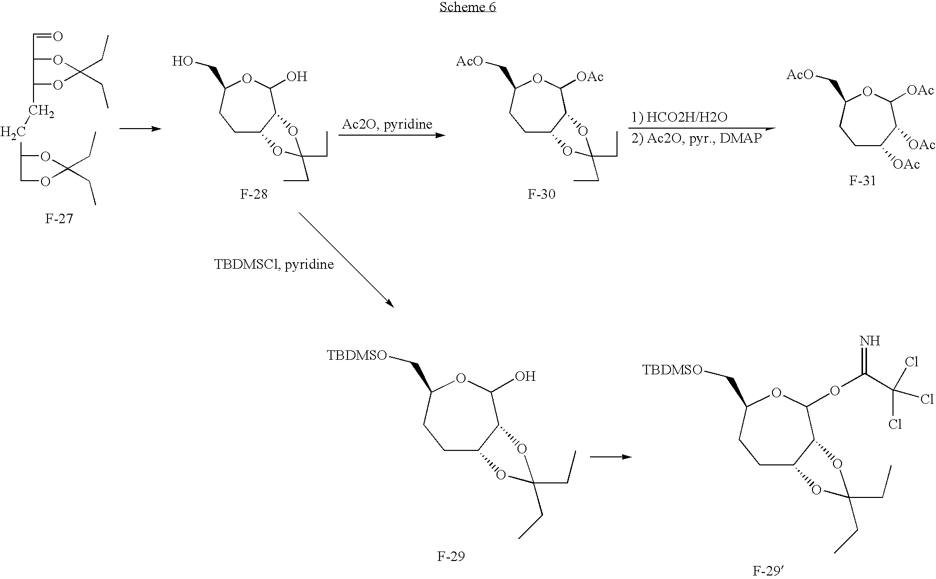
Illustration 5 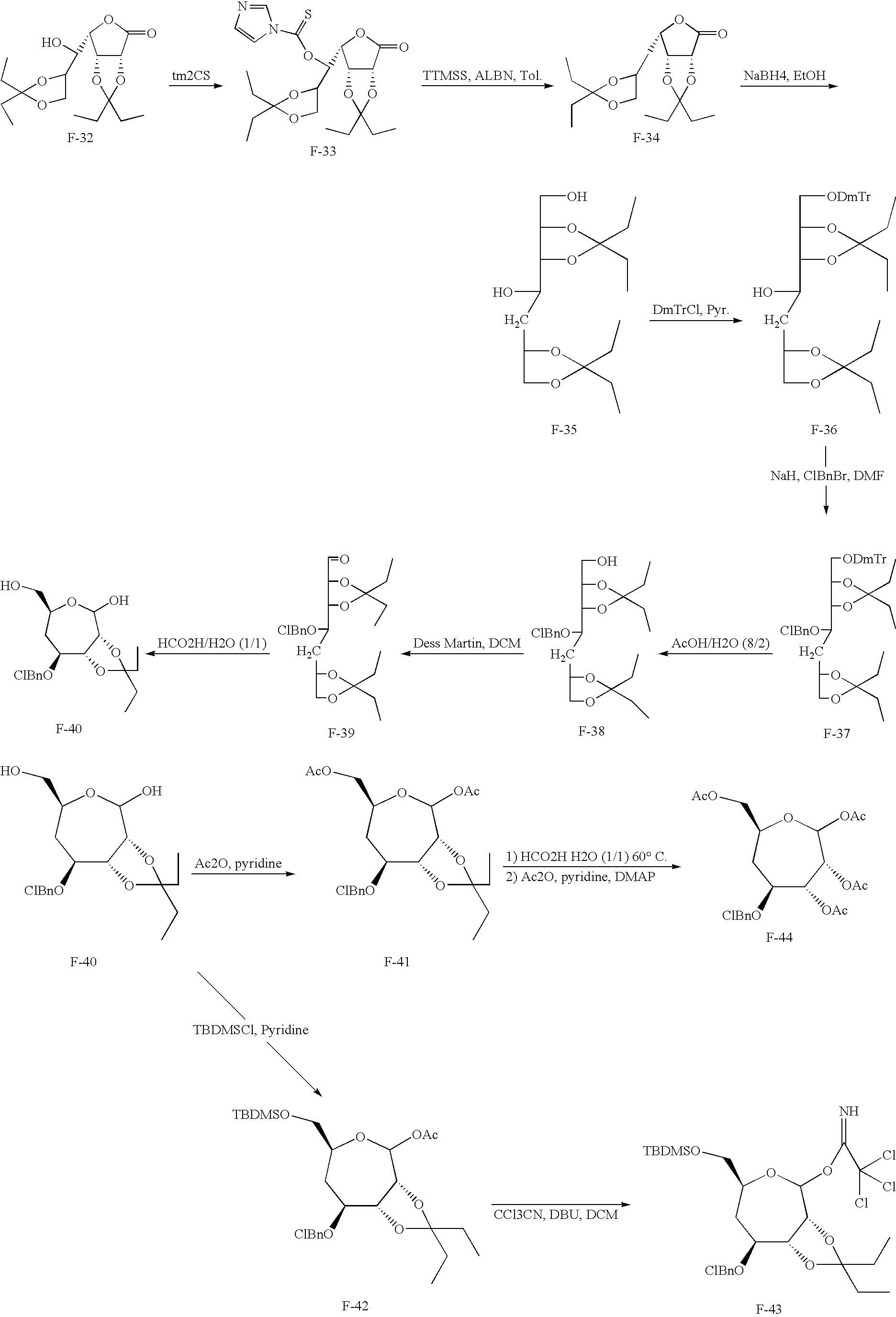
Illustration 6 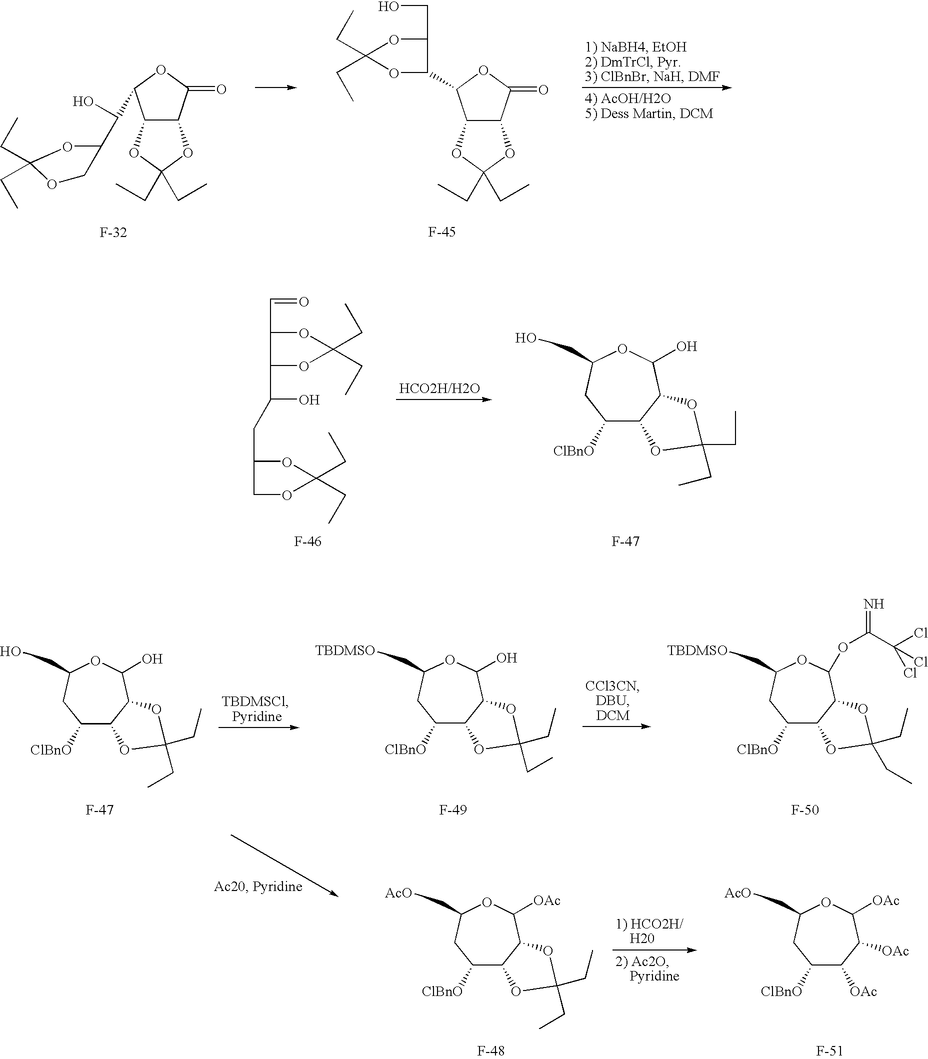
Illustration 7 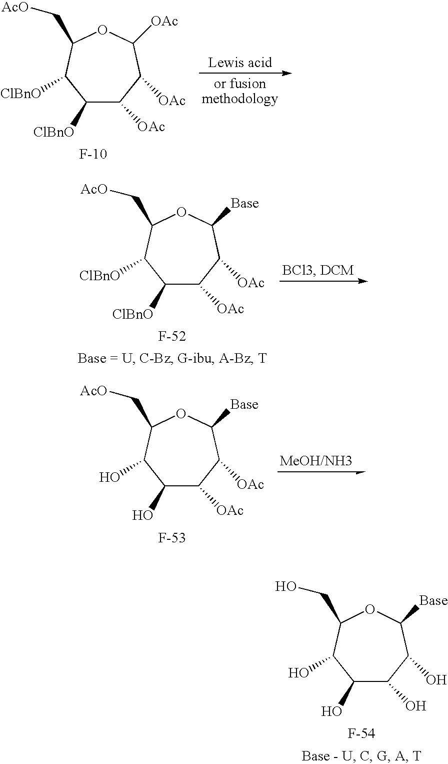
Illustration 8 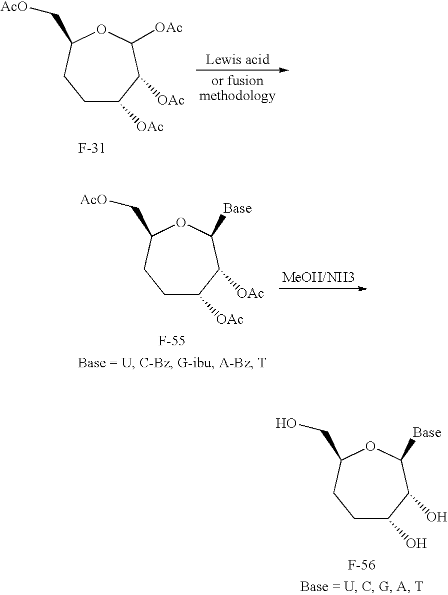
Illustration 9 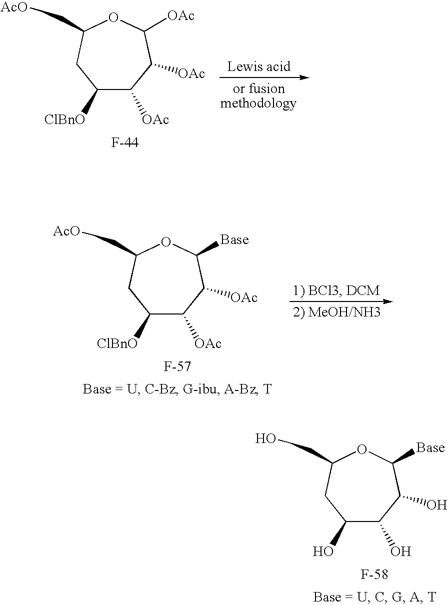
Illustration 10

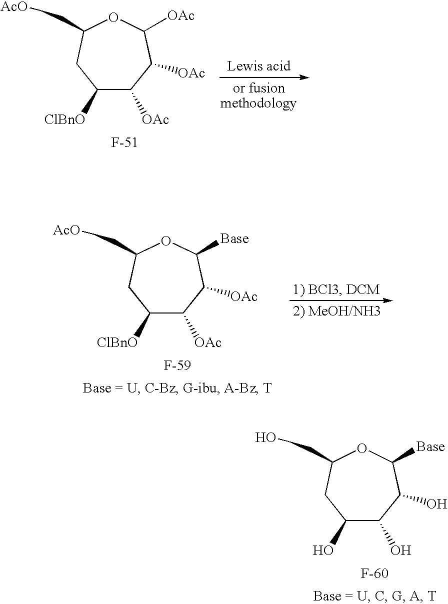

### Example 25

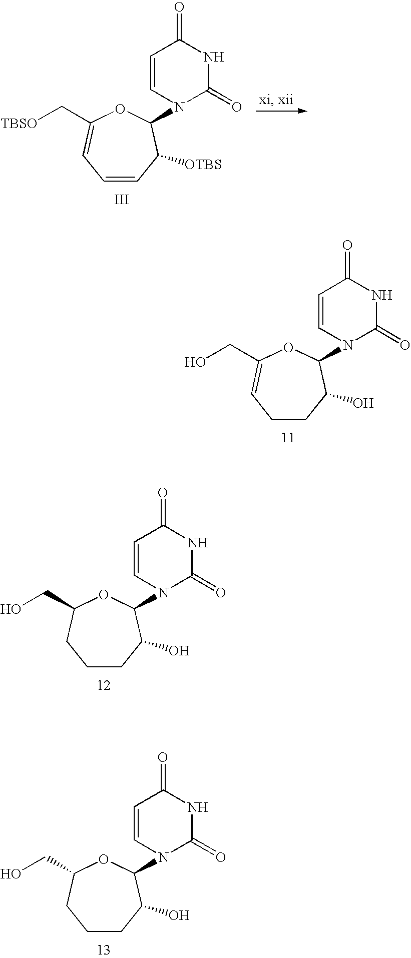

Step xi): Nucleoside III (Nomura, M.; Shuto, S.; Tanaka, M.; Sasaki, T.; Mori, S.; Shigeta, S.; Matsuda, A. J. Med. Chem., 1999, 42, 2901-2908 and Nomura, M.; Endo, K.; Shuto, S.; Matsuda, A. Tetrahedron, 1999, 55, 14847-14854) (390 mg, 0.81 mmol) was dissolved in THF (4 mL). TBAF (1M in THF) (1.78 mL, 1.78 mmol) was added and the solution was stirred for 30 min at room temperature. The solvent was evaporated and the residue was purified by silica gel chromatography (DCM/MeOH 92:8) to give a colorless solid (182 mg, 89%). ¹H NMR (DMSO-D₆, 300 MHz): 3.76-3.94 (m, 2H), 4.52-4.62 (m, 1H), 5.17-5.25 (m, 1H), 5.31 (d, J=7.5 Hz, 1H,5.60 (d, J=7.8 Hz, 1H), 5.64-5.87 (m, 4H), 7.73 (d, J=7.8 Hz, 1H), 11.43 (br, 1H). ES/MS: m/z 253 (M+H)⁺, m/z 251 (M−H)⁻.

Step xii) a) Conditions favoring compound 11: The product from step xi (50 mg, 0.19 mmol) was dissolved in EtOH (7 mL) and treated with palladium (10% on charcoal) (2 mg). The flask was put under an H₂ atmosphere and stirred at room temperature. The reaction was monitored by reverse-phase HPLC. After 1 h, the suspension was filtered on celite and the solvent was evaporated. The crude material was purified by reverse-phase chromatography (H₂O/MeCN gradient 98:2 to 90:10) to give 11 as a white powder (third eluted product, major—15 mg, 30%). ¹H NMR (DMSO-D₆, 300 MHz): 1.52-1.64 (m, 1H), 1.91-2.10 (m, 2H), 2.23-2.39 (m, 1H), 3.63-3.78 (m, 2H), 4.10-4.21 (m, 1H), 4.96 (t, J=5.6 Hz, 1H), 5.14 (t, J=5.6 Hz, 1H), 5.25-5.34 (m, 1H), 5.52 (d, J=9.3 Hz, 1H), 5.62 (d, J=7.8 Hz, 1H), 7.80 (d, J=7.8 Hz, 1H), 11.35 (br, 1H). ES/MS: m/z 255 (M+H)^{+, m/z} 253 (M−H)⁻.

b) Conditions favoring compounds 12/13: The product from step xi (120 mg, 0.45 mmol) was dissolved in EtOH (20 mL) and treated with palladium (10% on charcoal) (10 mg). The flask was put under an H₂ atmosphere and stirred at room temperature. After 2 h, the suspension was filtered on celite and the solvent was evaporated. The crude material was purified by reverse-phase chromatography (H₂O/MeCN gradient 98:2 to 90:10). Two pure fractions were obtained. The first eluted epimer (12 or 13, not established) was obtained as a white powder (23 mg, 20%). ¹H NMR (DMSO-D₆, 300 MHz) (2H from —OH missing): 1.19-1.53 (m, 3H), 1.62-1.88 (m, 2H), 1.95-2.08 (m, 1H), 3.68-3.90 (m, 2H), 4.60-4.86 (m, 1H), 4.90-5.20 (m, 1H), 5.51 (d, J=8.7 Hz, 1H), 5.53 (d, J=7.8 Hz, 1H), 7.65 (d, J=7.8 Hz, 1H), 11.15 (br, 1H). ES/MS: m/z 257 (M+H)⁺, m/z 255 (M−H)⁻. The second eluted epimer (13 or 12, not established) was obtained as a white powder (25 mg, 21%). ¹H NMR (DMSO-D₆, 300 MHz) (2H from —OH missing): 1.44-1.76 (m, 5H), 1.87-2.00 (m, 1H), 3.70-3.82 (m, 2H), 4.59-4.71 (m, 1H), 4.99-5.17 (m, 1H), 5.38 (d, J=8.7 Hz, 1H), 5.57 (d, J=7.8 Hz, 1H), 7.64 (d, J=7.8 Hz, 1H), 11.25 (br, 1H). ES/MS: m/z 257 (M+H)⁺, m/z 255 (M−H)⁻.

### Example 6

### Biological Data

Cell culture systems for determining antiviral activities

A useful cell-based assay to detect HCV and its inhibition assesses the levels of replicon RNA from Huh7 cells harboring the HCV replicon. These cells can be cultivated in standard media, for example DMEM medium (high glucose, no pyruvate), supplemented with 10% fetal bovine serum, 1× non-essential amino acids, Pen-Strep-Glu (100 units/liter, 100 microgram/liter, and 2.92 mg/liter, respectively), and G418 (C₂₀H₄₀N₄O₁₀; 500 to 1000 microgram/milliliter). Antiviral screening assays can be done in the same medium without G418. To keep the cells in the logarithmic growth phase, cells are seeded in 96-well plates at low density (for example, 1000 cells per well). The test compound is then added immediately after seeding the cells and they are incubated for 3 to 7 days at 37° C. in an incubator. The medium is then removed, and the cells prepared for total RNA extraction (replicon RNA+host RNA). Replicon RNA can then be amplified in a real-time RT-PCR (Q-RT-PCR) protocol, and quantified.

The observed differences in quantification of replicon RNA are one way to express the antiviral potency of the test compound. In a typical experiment, a comparable amount of replicon is produced in the negative control and with non-active compounds. This can be concluded if the measured threshold-cycle for HCV RT-PCR in both setting is approximately the same. In such experiments, a way to express the antiviral effectiveness of a compound is to subtract the average threshold RT-PCR cycle of the negative control (Ct_{negative}) from the threshold RT-PCR cycle of the test compound (Ct_{testcompound}). This value is called ΔCt (ΔCt=Ct_{testcompound}−Ct_{negative}). A ΔCt value of 3.3 represents a 1-log reduction in replicon production. As a positive control, recombinant interferon alpha-2a (for example, Roferon-A, Hoffmann-Roche, NJ, USA) can be used alongside the test compound. Furthermore, the compounds can be tested in dilution series (typically at 100, 33, 10, 3 and 1 μM). The ΔCt values for each concentration allow the calculation of the 50% effective concentration (EC₅₀).

The assay described above can be adapted to the other members of the Flaviviridae by changing the cell system and the viral pathogen. Methodologies to determine the efficacy of these antiviral compounds include modifications of the standard techniques as described by Holbrook MR et al. Virus Res. 2000, 69, 31; Markland W et al. Antimicrob. Agents. Chemother. 2000, 44, 859;Diamond MS et al., J. Virol. 2000, 74, 7814;Jordan I et al. J. Infect. Dis. 2000, 182, 1214; Sreenivasan V et al. J. Virol. Methods 1993, 45 (1), 1;or Baginski SG et al. Proc. Natl. Acad. Sci. U.S.A. 2000, 97 (14), 7981 or the real-time RT-PCR technology. As an example, an HCV replicon system in HuH7 cells (Lohmann V et al. Science, 1999, 285 (5424), 110) or modifications thereof (Blight et al. 2000) can be used.

Non-cell Based Assays Adapted for Detecting HCV

Nucleic acid amplification technology is now the method of choice for identification of a large and still growing number of microorganisms such as Mycobacterium tuberculosis, human immunodeficiency virus (HIV), and hepatitis C virus (HCV) in biological samples. Nucleic acid amplification techniques include the polymerase chain reaction (PCR), ligase chain reaction (LCR), nucleic acid sequence-based amplification (NASBA), strand-displacement amplification (SDA), and transcription-mediated amplification (TMA). Several FDA-approved diagnostic products incorporate these molecular diagnostic methods (see Table below). Nucleic acid amplification technology tests involve not only amplification, but detection methodologies as well. The promise of molecular diagnostics lies in the improvement of its specimen-processing, amplification, and target-detection steps, and in the integration of these steps into an automated format.

| | Amplification | Nucleic Acid | |
|---|---|---|---|
| FDA- Approved | Product: Detection | Amplification | Commercial |
| Assays | Method | Method | Source |
| C. trachomatis, | Heterogeneous: | PCR | Roche |
| N. gonorrhoeae, | Colorimetric | | Diagnostics |
| M. tuberculosis, | | | |
| HIV-1 | | | |
| C. trachomatis, | Heterogeneous: | LCR | Abbott |
| N. gonorrhoeae | Chemiluminescence | | Laboratories |
| C. trachomatis, | Homogeneous: | SDA | Becton |
| N. gonorrhoeae | Fluorescence | | Dickinson |
| C. trachomatis, | Homogeneous: | TMA | Gen-Probe |
| M. tuberculosis | Chemiluminescence | | |
| | (HPA) | | |

Amplified-product Detection Schemes

Amplified-product detection schemes are of two basic types: heterogeneous and homogeneous. Heterogeneous detection is characterized by a distinct step, such as washing, designed to remove unhybridized probes from hybridized probes, whereas in homogeneous detection there is no physical separation step to remove free probe from bound probe. Multiple heterogeneous and homogeneous detection methods exist. Any of these heterogeneous or homogeneous assays can be utilized to assess the effectiveness of the compounds of the present invention versus an RNA-dependent RNA polymerase virus, such as HCV.

Heterogeneous Detection: Southern blotting, for example, is a heterogeneous detection technique. In Southern blotting, electrophoresis is used to separate amplification products by size and charge. The size-fractionated products are transferred to a membrane or filter by diffusion, vacuuming, or electroblotting. Labeled detection probes are then hybridized to the membrane-bound targets in solution, the filters are washed to remove any unhybridized probe, and the hybridized probe on the membrane is detected by any of a variety of methods.

Other types of heterogeneous detection are based on specific capture of the amplification products by means of enzyme-linked immunosorbent assays (ELISAs). One method used with PCR involves labeling one primer with a hapten or a ligand, such as biotin, and, after amplification, capturing it with an antibody- or streptavidin-coated microplate. The other primer is labeled with a reporter such as fluorescein, and detection is achieved by adding an antifluorescein antibody, horseradish peroxidase (HRP) conjugate. This type of method is not as specific as using detection probes that hybridize to defined amplification products of interest.

The LCx probe system (Abbott Laboratories; Abbott Park, Ill.) and the Amplicor HIV-1 test (Roche Molecular Systems Inc.; Pleasanton, Calif.) are systems that use heterogeneous detection methods. In the LCx system, hapten-labeled oligonucleotide probes thermocycle in the ligase chain reaction. Either a capture hapten or a detection hapten is covalently attached to each of the four primer oligonucleotides. Upon amplification, each amplified product (amplicon) has one capture hapten and one detection hapten. When amplification is complete, the LCx system instrument transfers the reaction to a new well where antibody-coated microparticles bind the capture haptens. Each microparticle is then irreversibly bound to a glass-fiber matrix. A wash step removes from the microparticle any probe that contains only the detection hapten. The LCx instrument adds an alkaline phosphatase (AP)-antibody conjugate that binds to the detection hapten. A fluorigenic substrate for AP is 4-methylumbelliferyl. Dephosphorylation of 4-methylumbelliferyl by AP converts it to 4-methylumbelliferone, which is fluorescent.

The Amplicor HIV-1 test uses an ELISA format. After amplification by PCR, the amplicon is chemically denatured. Amplicon-specific oligonucleotide probes capture the denatured strands onto a coated microplate. The operator washes away any unincorporated primers and unhybridized material in a wash step and then adds an avidin-HRP conjugate to each well. The conjugate binds to the biotin-labeled amplicon captured on the plate. The operator then adds 3,3′,5,5′-tetramethylbenzidine (TMB), a chromogenic HRP substrate. When hydrogen peroxide is present, HRP oxidizes TMB. The signal is determined calorimetrically.

Homogeneous Detection: Because hybridized and nonhybridized detection probes are not physically separated in homogeneous detection systems, these methods require fewer steps than heterogeneous methods and thus are less prone to contamination. Among the commercially available kits that use homogeneous detection of fluorescent and chemiluminescent labels are the TaqMan system (Applied Biosystems; Foster City, Calif.), BDProbeTecET system (Becton Dickinson; Franklin Lakes, N.J.), QPCR System 5000 (Perkin-Elmer Corp.; Norwalk, Conn.), and Hybridization Protection Assay (Gen-Probe Inc.; San Diego).

The TaqMan system detects amplicon in real time. The detection probe, which hybridizes to a region inside the amplicon, contains a donor fluorophore such as fluoroscein at its 5′ end and a quencher moiety, for example, rhodamine, at its 3′ end. When both quencher and fluorophore are on the same oligonucleotide, donor fluorescence is inhibited. During amplification the probe is bound to the target. Taq polymerase displaces and cleaves the detection probe as it synthesizes the replacement strand. Cleavage of the detection probe results in separation of the fluorophore from the quencher, leading to an increase in the donor fluorescence signal. During each cycle of amplification the process is repeated. The amount of fluorescent signal increases as the amount of amplicon increases.

Molecular beacons use quenchers and fluorophores also. Beacons are probes that are complementary to the target amplicon, but contain short stretches (approximately 5 nucleotides) of complementary oligonucleotides at each end. The 5′ and 3′ ends of the beacons are labeled with a fluorophore and a quencher, respectively. A hairpin structure is formed when the beacon is not hybridized to a target, bringing into contact the fluorophore and the quencher and resulting in fluorescent quenching. The loop region contains the region complementary to the amplicon. Upon hybridization to a target, the hairpin structure opens and the quencher and fluorophore separate, allowing development of a fluorescent signal. A fluorometer measures the signal in real time.

The BDProbeTecET system uses a real-time detection method that combines aspects of TaqMan and molecular beacons. The probe has a hairpin loop structure and contains fluorescein and rhodamine labels. In this system, however, the region complementary to the target molecule is not within the loop but rather in the region 3′ to the rhodamine label. Instead of containing the sequence complementary to the target, the single-stranded loop contains a restriction site for the restriction enzyme BsoBI. The single-stranded sequence is not a substrate for the enzyme. The fluorescein and rhodamine labels are near each other before amplification, which quenches the fluorescein fluorescence. Strand-displacement amplification converts the probe into a double-stranded molecule. The BsoBI restriction enzyme can then cleave the molecule, resulting in separation of the labels and an increase in the fluorescent signal.

The QPCR System 5000 employs electrochemiluminescence with ruthenium labels. A biotinylated primer is used. After amplification, the biotin products are captured on streptavidin-coated paramagnetic beads. The beads are transferred into an electrochemical flow cell by aspiration and magnetically held to the surface of the electrode. Upon electrical stimulation, the ruthenium-labeled probe emits light.

The Hybridization Protection Assay is used in Gen-Probe's nonamplified PACE assays as well as in amplified Mycobacterium tuberculosis and Chlamydia trachomatis assays. The detection oligonucleotide probes in HPA are labeled with chemiluminescent acridinium ester (AE) by means of a linker arm. Hybridization takes place for 15 minutes at 60° C. in the same tube in which the amplification occurred. The selection reagent, a mildly basic buffered solution added after hybridization, hydrolyzes the AE on any unhybridized probe, rendering it nonchemiluminescent. The AE on hybridized probes folds inside the minor groove of the double helix, thereby protecting itself from hydrolysis by the selection reagent. The AE emits a chemiluminescent signal upon hydrolysis by hydrogen peroxide followed by sodium hydroxide. A luminometer records the chemiluminescent signal for 2 seconds (a period termed a light-off) and reports the photons emitted in terms of relative light units (RLU).

Detection-probe design is critical in all methodologies that use probes to detect amplification products. Good detection probes hybridize only to specified amplification product and do not hybridize to nonspecific products. Other key issues in optimizing detection methodologies involve the labeling of probes and the maximization of sample throughput.

Labeling Methods and Reporter Molecules. Detection probes can be labeled several different ways. Enzymatic incorporation of ³²P or ³⁵S into the probes is the most common method for isotopic labeling. Following hybridization and washing, the signal is detected on autoradiographic film.

To perform nonradioactive detection, probes can be enzymatically labeled with a variety of molecules. Biotin can be incorporated enzymatically and then detected with streptavidin-conjugated alkaline phosphatase, using AP substrates like 5-bromo-4-chloro-3-indolyl phosphate (BCIP) and nitroblue tetrazolium (NBT). Chemiluminescent substrates such as Lumi-Phos 530 or Lumi-Phos Plus (Lumigen, Southfield, Mich.) can also be used with AP. In addition, digoxigenin-11-dUTP can be incorporated enzymatically into DNA or RNA, and antidigoxigenin AP conjugates can be used with colorimetric or chemiluminescent detection.

There are numerous other types of reporter molecules, including chemiluminescent moieties such as acridinium esters. Many fluorescent moieties are available as well. Electrochemiluminescent compounds such as tris (2,2′-bipyridine) ruthenium (II) can be used also. Further discussions of these and similar techniques can be found in: Schiff ER, de Medina M, Kahn R S. Semin Liver Dis. 1999;19(Suppl 1:3-15).

Phosphorylation Assay of Nucleoside to Active Triphosphate

To determine the cellular metabolism of the compounds, HepG2 cells are obtained from the American Type Culture Collection (Rockville, Md.), and are grown in 225 cm² tissue culture flasks in minimal essential medium supplemented with non-essential amino acids, 1% penicillin-streptomycin. The medium is renewed every three days, and the cells are subcultured once a week. After detachment of the adherent monolayer with a 10 minute exposure to 30 mL of trypsin-EDTA and three consecutive washes with medium, confluent HepG2 cells are seeded at a density of 2.5×10⁶ cells per well in a 6-well plate and exposed to 10 □M of [³H] labeled active compound (500 dpm/pmol) for the specified time periods. The cells are maintained at 37° C. under a 5% CO₂ atmosphere. At the selected time points, the cells are washed three times with ice-cold phosphate-buffered saline (PBS). Intracellular active compound and its respective metabolites are extracted by incubating the cell pellet overnight at −20° C. with 60% methanol followed by extraction with an additional 20 □L of cold methanol for one hour in an ice bath. The extracts are then combined, dried under gentle filtered air flow and stored at −20° C. until HPLC analysis.

Antiviral nucleosides and nucleoside analogs are generally converted into the active metabolite, the 5′-triphosphate (TP) derivatives by intracellular kinases. The nucleoside-TPs then exert their antiviral effect by inhibiting the viral polymerase during virus replication

The cellular metabolism of the compounds of the invention is examined using MDBK cells, HepG2 cells and human primary hepatocytes exposed to 10 □M [³H]-mCyd. High-pressure liquid chromatography (HPLC) analysis can demonstrate that the compounds are phosphorylated in all three cell types, with the triphosphate form being the predominant metabolite after 24 h.

### Example 7

### Mitochondria Toxicity Assay

HepG2 cells are cultured in 12-well plates as described above and exposed to various concentrations of drugs as taught by Pan-Zhou X-R, Cui L, Zhou X-J, Sommadossi J-P, Darley-Usmer V M. “Differential effects of antiretroviral nucleoside analogs on mitochondrial function in HepG2 cells” Antimicrob. Agents Chemother. 2000; 44:496-503. Lactic acid levels in the culture medium after 4 day drug exposure are measured using a Boehringer lactic acid assay kit. Lactic acid levels are normalized by cell number as measured by hemocytometer count.

Cytotoxicity Assays

Cells are seeded at a rate of between 5×10³ and 5×10⁴/well into 96-well plates in growth medium overnight at 37° C. in a humidified CO₂ (5%) atmosphere. New growth medium containing serial dilutions of the drugs was then added. After incubation for 4 days, cultures were fixed in 50% TCA and stained with sulforhodamineB. The optical density is read at 550 nm. The cytotoxic concentration is expressed as the concentration required to reduce the cell number by 50% (CC₅₀).

Conventional cell proliferation assays are used to assess the cytotoxicity of mCyd and its cellular metabolites in rapidly dividing cells.

### Example 7a

### Effect on Human Bone Marrow Progenitor Cells—Bone Marrow Toxicity Assay

Human bone marrow cells are collected from normal healthy volunteers and the mononuclear population is separated by Ficoll-Hypaque gradient centrifugation as described previously by Sommadossi J-P, Carlisle R. “Toxicity of 3′-azido-3′-deoxythymidine and 9-(1,3-dihydroxy-2-propoxymethyl)guanine for normal human hematopoietic progenitor cells in vitro” Antimicrobial Agents and Chemotherapy 1987; 31:452454;and Sommadossi J-P, Schinazi R F, Chu C K, Xie M-Y. “Comparison of cytotoxicity of the (−)- and (+)-enantiomer of 2′,3′-dideoxy-3′-thiacytidine in normal human bone marrow progenitor cells” Biochemical Pharmacology 1992; 44:1921-1925. The culture assays for CFU-GM and BFU-E are performed using a bilayer soft agar or methylcellulose method. Drugs are diluted in tissue culture medium and filtered. After 14 to 18 days at 37□C in a humidified atmosphere of 5% CO₂ in air, colonies of greater than 50 cells are counted using an inverted microscope. The results are presented as the percent inhibition of colony formation in the presence of drug compared to solvent control cultures.

Cell Protection Assay (CPA)

The assay are performed essentially as described by Baginski, S. G.; Pevear, D. C.; Seipel, M.; Sun, S. C. C.; Benetatos, C. A.; Chunduru, S. K.; Rice, C. M. and M. S. Collett “Mechanism of action of a pestivirus antiviral compound” PNAS USA 2000, 97(14), 7981-7986. MDBK cells (ATCC) are seeded onto 96-well culture plates (4,000 cells per well) 24 hours before use. After infection with BVDV (strain NADL, ATCC) at a multiplicity of infection (MOI) of 0.02 plaque forming units (PFU) per cell, serial dilutions of test compounds are added to both infected and uninfected cells in a final concentration of 0.5% DMSO in growth medium. Each dilution is tested in quadruplicate. Cell densities and virus inocula are adjusted to ensure continuous cell growth throughout the experiment and to achieve more than 90% virus-induced cell destruction in the untreated controls after four days post-infection. After four days, plates are fixed with 50% TCA and stained with sulforhodamine B. The optical density of the wells is read in a microplate reader at 550 nm. The 50% effective concentration (EC₅₀) values are defined as the compound concentration that achieved 50% reduction of cytopathic effect of the virus.

The myelosuppressive effects of certain nucleoside analogs have highlighted the need to test for potential effects of investigational drugs on the growth of human bone marrow progenitor cells in clonogenic assays. In particular, anemia and neutropenia are the most common drug-related clinical toxicities associated with the anti-HIV drug zidovudine (ZDV) or the ribavirin (RBV) component of the standard of care combination therapy used for HCV treatment. These toxicities have been modeled in an in vitro assay that employed bone marrow cells obtained from healthy volunteers (Sommadossi J-P, Carlisle R. Antimicrob. Agents Chemother. 1987;31(3): 452-454). ZDV has been previously shown to directly inhibit human granulocyte-macrophage colony-forming (CFU-GM) and erythroid burst-forming (BFU-E) activity at clinically relevant concentrations of 1-2 μM in this model (Berman E, et al. Blood 1989;74(4):1281-1286;Yoshida Y, Yoshida C. AIDS Res. Hum. Retroviruses 1990;6(7):929-932.;Lerza R, et al. Exp. Hematol. 1997;25(3):252-255;Domsife R E, Averett D R. Antimicrob. Agents Chemother. 1996;40(2):514-519;Kurtzberg J, Carter S G. Exp. Hematol. 1990;18(10):1094-1096; Weinberg R S, et al. Mt. Sinai. J Med. 1998;65(l):5-13).

### Example 7b

### Effect on Mitochondrial Function

Antiviral nucleoside analogs approved for HIV therapy such as ZDV, stavudine (d4T), didanosine (ddl), and zalcitabine (ddC) have been occasionally associated with clinically limiting delayed toxicities such as peripheral neuropathy, myopathy, and pancreatitis (Browne M J, et al. J. Infect. Dis. 1993;167(1):21-29; Fischl M A, et al. Ann. Intern. Med. 1993;18(10):762-769;Richman D D, et al. N. Engl. J. Med. 1987;317(4): 192-197;Yarchoan R, et al. Lancet 1990;336(8714):526-529). These clinical adverse events have been attributed by some experts to inhibition of mitochondrial function due to reduction in mitochondrial DNA (mtDNA) content and nucleoside analog incorporation into mtDNA. In addition, one particular nucleoside analog, fialuridine (1,-2′-deoxy-2′-fluoro-1-β-D-arabinofuranosyl-5-iodo-uracil; FIAU)—caused hepatic failure, pancreatitis, neuropathy, myopathy and lactic acidosis due to direct mitochondrial toxicity (McKenzie R, et al. N Engl J Med 1995;333(17):1099-1105). Drug-associated increases in lactic acid production can be considered a marker of impaired mitochondrial function or oxidative phosphorylation. (Colacino, J. M. Antiviral Res 1996 29(2-3): 125-39).

To assess the potential of the compounds to produce mitochondrial toxicity, several in vitro studies are conducted using the human hepatoma cell lines HepG2 or Huh7. These studies include analysis of lactic acid production, mtDNA content, and determination of changes in morphology (e.g., loss of cristae, matrix dissolution and swelling, and lipid droplet formation) of mitochondrial ultrastructure.

### Example 8

### In Vitro Antiviral Activity Against BVDV

Compounds can exhibit anti-flavivirus or pestivirus activity by inhibiting flavivirus or pestivirus polymerase, by inhibiting other enzymes needed in the replication cycle, or by other pathways.

Plaque Reduction Assay

For each compound the effective concentration is determined in duplicate 24-well plates by plaque reduction assays. Cell monolayers are infected with 100 PFU/well of virus. Then, serial dilutions of test compounds in MEM supplemented with 2% inactivated serum and 0.75% of methyl cellulose are added to the monolayers. Cultures are further incubated at 37° C. for 3 days, then fixed with 50% ethanol and 0.8% Crystal Violet, washed and air-dried. Then plaques are counted to determine the concentration to obtain 90% virus suppression.

Yield Reduction Assay

For each compound the concentration to obtain a 6-log reduction in viral load is determined in duplicate 24-well plates by yield reduction assays. The assay is performed as described by Baginski, S. G.; Pevear, D. C.; Seipel, M.; Sun, S. C. C.; Benetatos, C. A.; Chunduru, S. K.; Rice, C. M. and M. S. Collett “Mechanism of action of a pestivirus antiviral compound” PNAS USA 2000, 97(14), 7981-7986, with minor modifications. Briefly, MDBK cells were seeded onto 24-well plates (2×105 cells per well) 24 hours before infection with BVDV (NADL strain) at a multiplicity of infection (MOI) of 0.1 PFU per cell. Serial dilutions of test compounds are added to cells in a final concentration of 0.5% DMSO in growth medium. Each dilution is tested in triplicate. After three days, cell cultures (cell monolayers and supematants) are lysed by three freeze-thaw cycles, and virus yield was quantified by plaque assay. Briefly, MDBK cells were seeded onto 6-well plates (5×105 cells per well) 24 h before use. Cells are inoculated with 0.2 mL of test lysates for 1 hour, washed and overlaid with 0.5% agarose in growth medium. After 3 days, cell monolayers were fixed with 3.5% formaldehyde and stained with 1% crystal violet (w/v in 50% ethanol) to visualize plaques. The plaques were counted to determine the concentration to obtain a 6-log reduction in viral load.

We claim:

1. A compound of the formula: 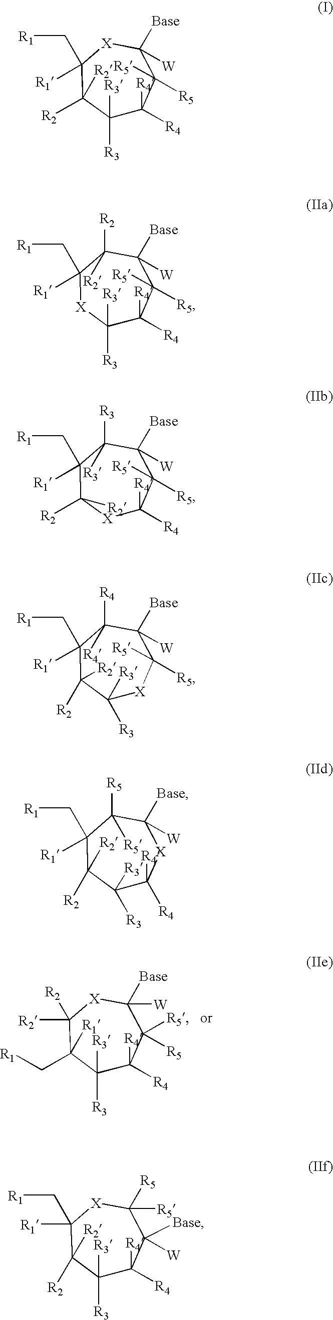
wherein:
X is O, S, SO₂, N—R, CH—R, or C—R—R;
R is H; C₁₋₄ alkyl, C₂₋₄ alkenyl, or C₂₋₄ alkynyl, each of which may be optionally substituted; CN, N₃, halo, OH, CONH₂, NH₂, or amidino;
R₁ is OH, monophosphate, diphosphate, triphosphate, phosphoryl, a phosphate derivative, acyl, alkyl, O-acyl, O-alkyl, O-aryl, O-alkoxyalkyl, O-aryloxyalkyl, O-substituted alkyl, O-substituted alkenyl, O-substituted alkynyl, alkyl sulfonyl, aryl sulfonyl, alkenyl sulfonyl, aralkylsulfonyl, an amino acid residue, or any cleavable substitutent that in vivo provides OH;
R₂, R₃, R₄ and R₅ each independently is H, OH, SH, NH₂, halo, C₁₋₁₀ alkylcarbonyl, monophosphate, diphosphate, triphosphate, phosphoryl, phosphate, phosphonate, phosphinate, phosphonoamidate, carbamate, phosphorothioate, phosphorodithioate, carbonyl, thiocarbonyl, aminoacyl, amidino, NO₂, CN, N₃, sulfonyl, sulfoxido, sulfate, sulfonate, sulfamoyl, sulfonamide, C1-6 alkyl, C₂₋₆ alkenyl, C₂₋₆ alkynyl, acyl, haloalkyl, haloalkenyl, haloalkynyl, cyclopropyl, O-alkyl, O-alkenyl;
O-alkynyl; O-acyl; S-alkyl; S-alkenyl; S-alkynyl; S-acyl; NH-alkyl; N(alkyl)₂; NH(alkenyl); N(alkenyl)₂; NH(alkynyl); N(alkynyl)₂; NH(acyl); N(acyl)₂; CONH₂; COOH; CONH-alkyl; CON(alkyl)₂; COSH₂; COSH-alkyl; COS(alkyl)₂;C₁₋₆ alkyl-O—C₁₋₆ alkyl; C₁₋₆ alkyl-O-alkenyl; C₁₋₆ alkyl-O-alkynyl; C₁₋₆ alkyl-S-alkyl; C₁₋₆ alkyl-S-alkenyl; C₁₋₆ alkyl-S-alkynyl; CH₂CN; or CH₂N₃; and
Each of R_{1′}, R_{2′}, R_{3′}, R_{4′}, R_{5′}and W independently is H, OH, C₁₋₁₀ alkylcarbonyl, phosphoryl, phosphonate, phosphinate, phosphonoamidate, Cl, F, Br, I, CN, NO₂, N₃, NH₂, acylamino, amido, amidino, C₁₋₆ alkyl, C₂₋₆ alkenyl, C₂₋₆ alkynyl, carbonyl, thiocarbonyl, acyl, haloalkyl, haloalkenyl, haloalkynyl, acyl, cyclopropyl, sulfonyl, sulfoxido, sulfate, sulfonate, sulfamoyl, sulfonamido, C₁₋₆ alkyl-O—C₁₋₆ alkyl, C₁₋₆ alkyl-O-alkenyl, C₁₋₆ alkyl-O-alkynyl, C₁₋₆ alkyl-S-alkyl, C₁₋₆ alkyl-S-alkenyl, C₁₋₆ alkyl-S-alkynyl, CONH₂, COOR, CH₂CN, or CH₂N₃; and
Each R₂, R₃, R₄ and R₅ and its corresponding R′ can form a spiro moiety;
Each R₂+R₃, R₃+R₄, or R₄+R₅ independently may join to form a 3-6 membered ring that optionally has 1, 2 or 3 heteroatoms;
Each R_{2′}+R_{3′}, R_{3′}+R_{4′}, or R_{4′}+R_{5′} independently may join to form a 3-6 membered ring that optionally has 1, 2 or 3 heteroatoms;
with the proviso that in general structural Formula (IId), W is OH only when X is CH—R or C—R—R for reasons of chemical stability; and with the further proviso that when any R₁, R₂, R₃, R₄ or R₅ is OH or NH₂, then its corresponding R_{1′}, R_{2′}, R_{3′}, R_{4′} or R_{5′} may not also be OH or NH₁₂;
Base is selected from the group consisting of: 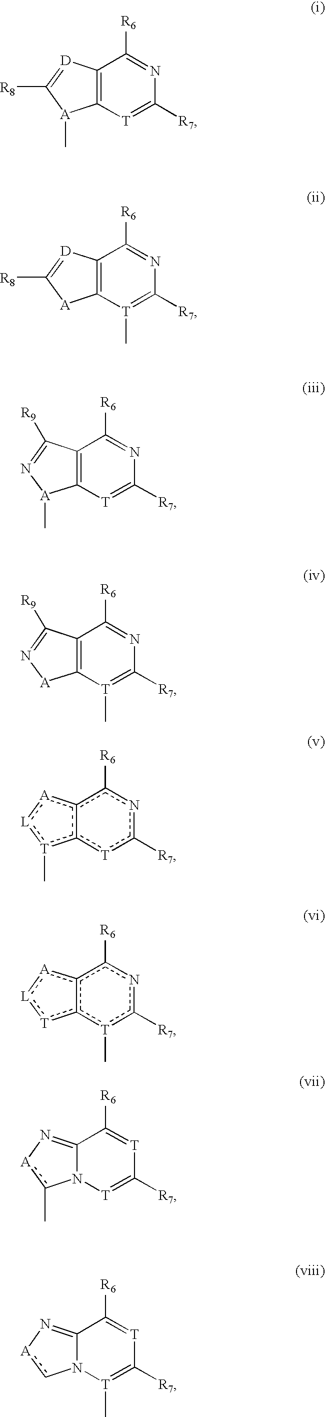 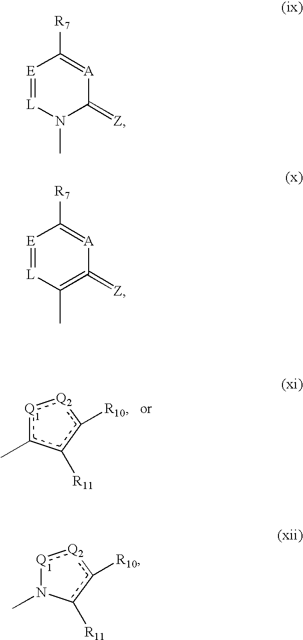
wherein:
Each occurrence of A, L, and T independently is C, CH, CH—R, N, NR, C-alkyl, O or S depending upon correct valence; or C-halo, C—C₁₋₆ alkyl, C—C₂₋₆ alkenyl, C—C₂₋₆ alkynyl, C₁₋₆ alkylamino, C—CF₃, C—OH, C—NH₂, C—NO₂, C—CN, C—N₃, C—COOR, or C—CONH₂;
D is CH, C—CN, C—NO₂, N, C—C₁₋₆ alkyl, C—CONH₂, C—CONH—C₁₋₆alkyl, C—CON(C₁₋₆alkyl)(C₁₋₆alkyl), C—NH₂, C-alkoxy, C—OH, C-alkylamino, C—C(=NH)NH₂,
C—COOH, C—COO-alkyl, C—CSNH₂, C—CSNH-alkyl, C—CSN(alkyl)₂, C-di(C₁₋₆ alkyl)amino, C-halo, C-heterocycle, wherein any alkyl optionally is substituted by from one to three substituents selected from the group consisting of alkoxy, hydroxyl, carboxy, halo and amino, and wherein heterocycle is a 5- or 6-membered ring having one to three heteroatoms;
E is N or C-halo, C—C₁₋6 alkyl, C—C₂₋₆ alkenyl, C—C₂₋₆ alkynyl, C₁₋₆ alkylamino, C—CF₃, C—OH, C—NH₂, C—NO₂, C—CN, C—N₃, C—COOR, or C—CONH₂;
Z is O or S;
R₆, R₇, R₈ and R₉, each independently, is H, OH, SH, NH₂, NO₂, CN, N₃, C₁₋₆ alkyl, C₂₋₆ alkenyl, C₂₋₆ alkynyl, C₁₋₆ alkylamino, di(C₁₋₆ alkyl)amino, C₃₋₆ cycloalkylamino, C₃₋₆ cycloalkyl, halo, C₁₋₆ alkoxy, carboxy, C₁₋₆ alkoxycarbonyl, C₁₋₆ alkylthio, C₁₋₆ alkylsulfonyl, (C₁₋₆ alkyl)₀₋₂ aminomethyl, or CF₃;
R₁₀ and R₁₁ each independently is H, OH, SH, NH₂, halo, C₁₋₁₀ alkylcarbonyl, monophosphate, diphosphate, triphosphate, phosphoryl, phosphate, phosphonate, phosphinate, phosphonoamidate, carbamate, phosphorothioate, phosphorodithioate, carbonyl, thiocarbonyl, aminoacyl, amidino, NO₂, CN, N₃, sulfonyl, sulfoxido, sulfate, sulfonate, sulfamoyl, sulfonamide, C₁₋₆ alkyl, C₂₋₆ alkenyl, C₂₋₆ alkynyl, acyl, haloalkyl, haloalkenyl, haloalkynyl, acyl, cyclopropyl, CONH₂, COOH, CONH-alkyl, CON(alkyl)₂, COSH₂, COSH-alkyl, COS(alkyl)₂, C₁₋₆ alkyl-O—C₁₋₆ alkyl, C₁₋₆ alkyl-O-alkenyl, C₁₋₆ alkyl-O-alkynyl, C₁₋₆ alkyl-S-alkyl, C₁₋₆ alkyl-S-alkenyl, C₁₋₆ alkyl-S-alkynyl, CH₂CN, or CH₂N₃;
Q₁ and Q₂ each independently is N, N—R, O, S, SO, SO₂, CH—R or C—R—R, depending upon the proper valence required; [symbol]
indicates the presence of a single or double bond; or
a pharmaceutically acceptable salt, ester or prodrug, or a or tautomeric form thereof.

2. A compound of the general structural Formula (IV): 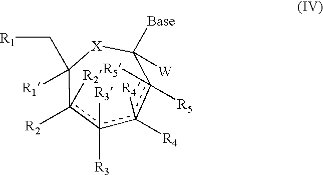
wherein:
X is O, S, SO₂, N—R, CH—R, or C—R—R;
R is H; C₁₋₄ alkyl, C₂₋₄ alkenyl, or C₂₋₄ alkynyl, each of which may be optionally substituted; CN, N₃, halo, OH, CONH₂, NH₂, or amidino;
R₁ is OH, monophosphate, diphosphate, triphosphate, phosphoryl, a phosphate derivative, acyl, alkyl, O-acyl, O-alkyl, O-aryl, O-alkoxyalkyl, O-aryloxyalkyl, O-substituted alkyl, O-substituted alkenyl, O-substituted alkynyl, alkyl sulfonyl, aryl sulfonyl, alkenyl sulfonyl, aralkylsulfonyl, an amino acid residue, or any cleavable substitutent that in vivo provides OH;
R₂, R₃, R₄ and R₅ each independently is H, OH, SH, NH₂, halo, C₁₋₁₀ alkylcarbonyl, monophosphate, diphosphate, triphosphate, phosphoryl, phosphate, phosphonate, phosphinate, phosphonoamidate, carbamate, phosphorothioate, phosphorodithioate, carbonyl, thiocarbonyl, aminoacyl, amidino, NO₂, CN, N₃, sulfonyl, sulfoxido, sulfate, sulfonate, sulfamoyl, sulfonamide, C₁₋₆ alkyl, C₂₋₆ alkenyl, C₂₋₆ alkynyl, acyl, haloalkyl, haloalkenyl, haloalkynyl, cyclopropyl, O-alkyl, O-alkenyl;
O-alkynyl; O-acyl; S-alkyl; S-alkenyl; S-alkynyl; S-acyl; NH-alkyl; N(alkyl)₂; NH(alkenyl); N(alkenyl)₂; NH(alkynyl); N(alkynyl)₂; NH(acyl); N(acyl)₂; CONH₂; COOH; CONH-alkyl; CON(alkyl)₂; COSH₂; COSH-alkyl; COS(alkyl)₂; C₁₋₆ alkyl-O—C₁₋₆ alkyl; C₁₋₆ alkyl-O-alkenyl; C₁₋₆ alkyl-O-alkynyl; C₁₋₆ alkyl-S-alkyl; C₁₋₆ alkyl-S-alkenyl; C₁₋₆ alkyl-S-alkynyl; CH₂CN; or CH₂N₃; and
Each of R_{1′}, R_{2′}, R_{3′}, R_{4′}, R_{5′} and W independently is H, OH, C₁₋₁₀ alkylcarbonyl, phosphoryl, phosphonate, phosphinate, phosphonoamidate, Cl, F, Br, I, CN, NO₂, N₃, NH₂, acylamino, amido, amidino, C₁₋₆ alkyl, C₂₋₆ alkenyl, C₂₋₆ alkynyl, carbonyl, thiocarbonyl, acyl, haloalkyl, haloalkenyl, haloalkynyl, acyl, cyclopropyl, sulfonyl, sulfoxido, sulfate, sulfonate, sulfamoyl, sulfonamido, C₁₋₆ alkyl-O—C₁₋₆ alkyl, C₁₋₆ alkyl-O-alkenyl, C₁₋₆ alkyl-O-alkynyl, C₁₋₆ alkyl-S-alkyl, C₁₋₆ alkyl-S-alkenyl, C₁₋₆ alkyl-S-alkynyl, CONH₂, COOR, CH₂CN, or CH₂N₃; and
Each R₂, R₃, R₄ and R₅ and its corresponding R′ can form a spiro moiety;
Each R₂+R₃, R₃+R₄, or R₄+R₅ independently may join to form a 3-6 membered ring that optionally has 1, 2 or 3 heteroatoms;
Each R_{2′}+R_{3′}, R_{3′}+R_{4′}, or R_{4′}+R_{5′} independently may join to form a 3-6 membered ring that optionally has 1, 2 or 3 heteroatoms;
with the proviso that at least two of R₂, R₃, R₄, R₅ and W must be OH;
with the additional proviso that W is OH only when X is CH—R or C—R—R for reasons of chemical stability; and
with the further proviso that when any R₁, R₂, R₃, R₄ or R₅ is OH or NH₂, then its corresponding R_{1′}, R_{2′}, R_{3′}, R_{4′} or R_{5′} may not also be OH or NH₂;
Base is selected from the group consisting of: 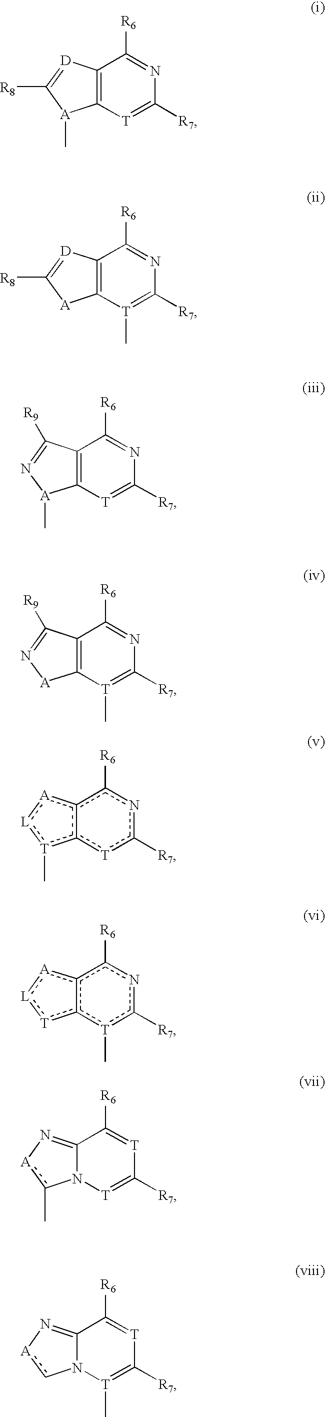 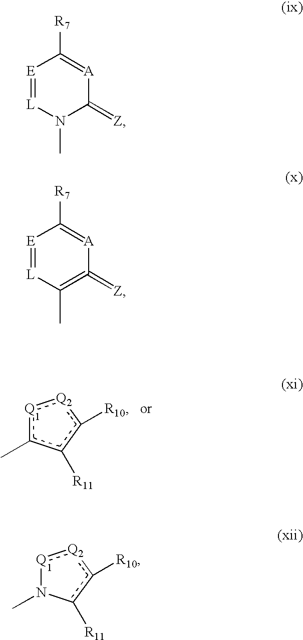
wherein:
Each occurrence of A, L, and T independently is C, CH, CH—R, N, NR, C-alkyl, O or S depending upon correct valence; or C-halo, C—C₁₋₆ alkyl, C—C₂₋₆ alkenyl, C—C₂₋₆ alkynyl, C₁₋₆ alkylamino, C—CF₃, C—OH, C—NH₂, C—NO₂, C—CN, C—N₃, C—COOR, or C—CONH₂;
D is CH, C—CN, C—NO₂, N, C—C₁₋₆ alkyl, C—CONH₂, C—CONH—C₁₋₆alkyl, C—CON(C₁₋₆alkyl)(C₁₋₆alkyl), C—NH₂, C-alkoxy, C—OH, C-alkylamino, C—C(=NH)NH₂,
C—COOH, C—COO-alkyl, C—CSNH₂, C—CSNH-alkyl, C—CSN(alkyl)₂, C-di(C₁₋₆ alkyl)amino, C-halo, C-heterocycle, wherein any alkyl optionally is substituted by from one to three substituents selected from the group consisting of alkoxy, hydroxyl, carboxy, halo and amino, and wherein heterocycle is a 5- or 6-membered ring having one to three heteroatoms;
E is N or C-halo, C—C₁₋₆ alkyl, C—C₂₋₆ alkenyl, C—C₂₋₆ alkynyl, C₁₋₆ alkylamino, C—CF₃, C—OH, C—NH₂, C—NO₂, C—CN, C—N₃, C—COOR, or C—CONH₂;
Z is O or S;
R₆, R₇, R₈ and R₉, each independently, is H, OH, SH, NH₂, NO₂, CN, N₃, C₁₋₆ alkyl, C₂₋₆ alkenyl, C₂₋₆ alkynyl, C₁₋₆ alkylamino, di(C₁₋₆ alkyl)amino, C₃₋₆ cycloalkylamino, C₃₋₆ cycloalkyl, halo, C₁₋₆ alkoxy, carboxy, C₁₋₆ alkoxycarbonyl, C₁₋₆ alkylthio, C₁₁₋₆ alkylsulfonyl, (C₁₋₆ alkyl)₀₋₂ aminomethyl, or CF₃;
R₁₀ and R₁₁ each independently is H, OH, SH, NH₂, halo, C₁₋₁₀ alkylcarbonyl, monophosphate, diphosphate, triphosphate, phosphoryl, phosphate, phosphonate, phosphinate, phosphonoamidate, carbamate, phosphorothioate, phosphorodithioate, carbonyl, thiocarbonyl, aminoacyl, amidino, NO₂, CN, N₃, sulfonyl, sulfoxido, sulfate, sulfonate, sulfamoyl, sulfonamide, C₁₋₆ alkyl, C₂₋₆ alkenyl, C₂₋₆ alkynyl, acyl, haloalkyl, haloalkenyl, haloalkynyl, acyl, cyclopropyl, CONH₂, COOH, CONH-alkyl, CON(alkyl)₂, COSH₂, COSH-alkyl, COS(alkyl)₂, C₁₋₆ alkyl-O—C₁₋₆ alkyl, C₁₋₆ alkyl-O-alkenyl, C₁₋₆ alkyl-O-alkynyl, C₁₋₆ alkyl-S-alkyl, C₁₋₆ alkyl-S-alkenyl, C₁₋₆ alkyl-S-alkynyl, CH₂CN, or CH₂N₃;
Q₁ and Q₂ each independently is N, N—R, O, S, SO, SO₂, CH—R or C—R—R, depending upon the proper valence required; [symbol]
indicates the presence of a single or double bond; or
a pharmaceutically acceptable salt, ester or prodrug, or a or tautomeric form thereof.

3. The compound of claim 1, wherein Base is:

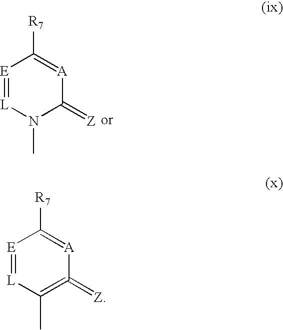

4. The compound of claim 1, wherein Base is:

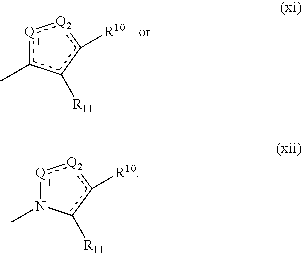

5. The compound of claim 1, wherein Base is:

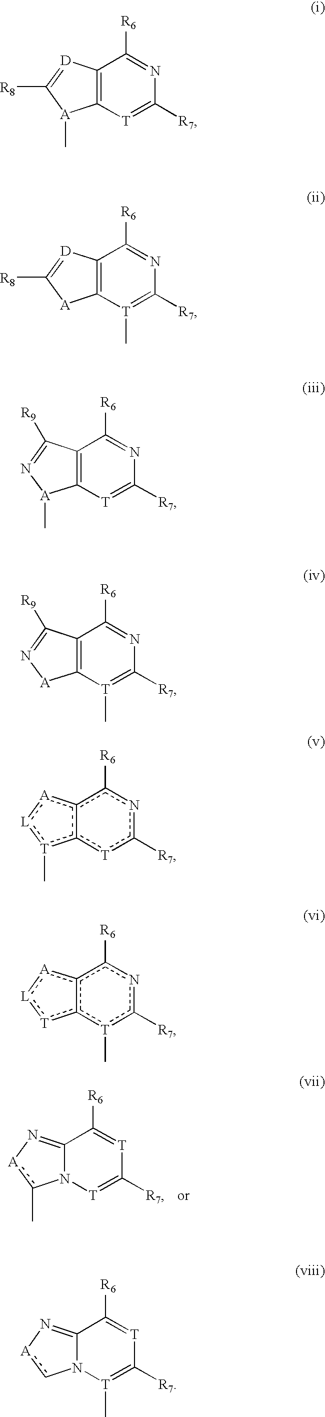

6. The compound of claim 2, wherein Base is:

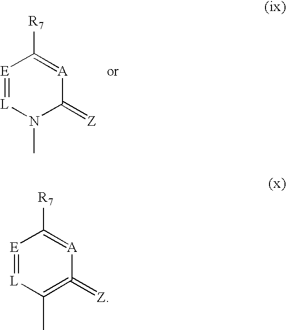

7. The compound of claim 2, wherein Base is:

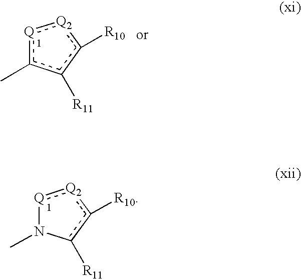

8. The compound of claim 2, wherein Base is:

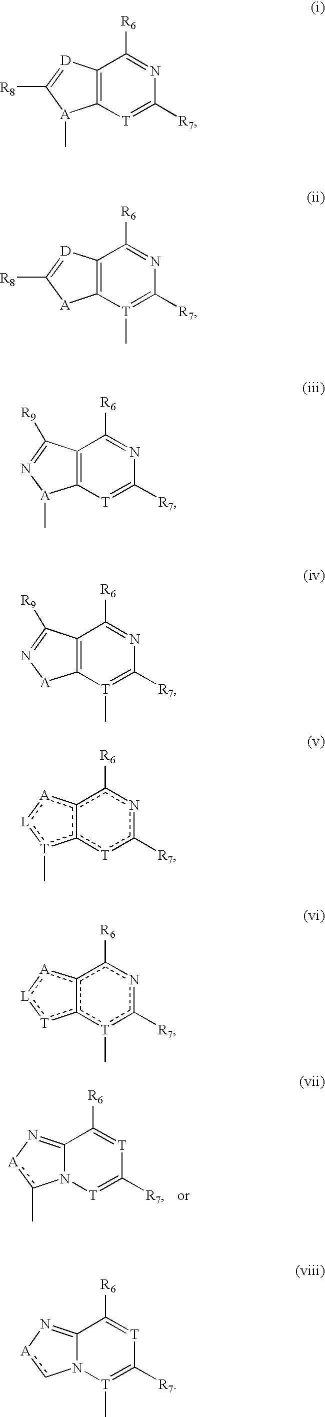

9. The compound of claim 1, wherein X is O.

10. The compound of claim 1, wherein at least three of R₁, R₂, R₃, R₄ and R₅ are OH.

11. The compound of claim 1, wherein R₁ is OH or mono, di or triphosphate.

12. The compound of claim 1, wherein at least two of R₁, R₂, R₃, R₄ and R₅ are OH.

13. The compound of claim 1, wherein R₅ is F or H; and R₅ is F or H.

14. The compound of claim 1, wherein R₅ is alkyl, NH₂ or H; and R_{5′} is alkyl, NH₂ or H.

15. The compound of claim 1, wherein R₅ is methyl or OH; and R_{5′} is methyl or OH.

16. The compound of claim 1, wherein:
X is O;
R₁, R₂, R₃, R₄ and R₅ are independently H, OH, mono, di or triphosphate, O-alkyl or O-acyl;
R_{1′}, R_{2′}, R_{3′}, R_{4′} and R_{5′} are independently H, OH, NH₂ or fluoro;
W is H; and
Base is adenine, guanine, 6-chloropurine or a pyrrolopyrimidine.

17. The compound of claim 1, wherein:
X is O, S or NR;
R₁, R₂, R₃, and R₄ are independently H, OH or O-acyl; R₅ and R_{5′} are independently F, NH₂ or H;
W is H; and
Base is a purine or pyrimidine.

18. The compound of claim 1, wherein:
X is O;
R₁, R₂, R₃, and R₄ are independently H, OH or O-acyl;
R₅ and R_{5′} are independently F, methyl or NH₂;
W is H; and
Base is a purine or pyrimidine.

19. The compound of claims 1, wherein the pharmaceutically acceptable salt is selected from the group consisting of tosylate, methanesulfonate, acetate, citrate, malonate, tartarate, succinate, benzoate, ascorbate, α-ketoglutarate, α-glycerophosphate, formate, fumarate, propionate, glycolate, lactate, pyruvate, oxalate, maleate, salicylate, sulfate, nitrate, hydrobromate, phosphate, hydrochloride and dihydrochloride.

20. The compound of claim 19, wherein the salt is a hydrochloride or dihydrochloride salt.

21. A pharmaceutical composition comprising an effective amount to treat a host with a Flaviviridae virus infection of a compound of claim 1 or 2 together with a pharmaceutically acceptable carrier or diluent.

22. The pharmaceutical composition of claim 21, wherein the virus is hepatitis C.

23. The pharmaceutical composition of claim 21, wherein the virus is a flavivirus.

24. The pharmaceutical composition of claim 21, wherein the virus is a pestivirus.

25. The pharmaceutical composition of claim 21, wherein the carrier is suitable for oral or intravenous delivery.

26. The pharmaceutical composition of claim 21, wherein the compound is in the form of a dosage unit.

27. The pharmaceutical composition of claim 26, wherein the dosage unit contains 50-1000 mg of the compound.

28. The pharmaceutical composition of claim 26, wherein the dosage unit is a tablet or capsule.

29. A process for the synthesis of a seven-membered ring nucleoside analogue:
a. reacting a 2,3,5-tri-O-protected pentose to provide a 6-hydroxy-4, 5, 7-tri-O-protected methyl hept-2-enoate derivative;
b. reducing the alkene from step a. to produce a 6-hydroxy-4,5,7-tri-O-protected methyl heptanoate;
c. hydrolyzing the product from step b. to provide a 6-hydroxy-4,5, 7-tri-O-protected heptanoic acid derivative;
d. cyclizing the product from step c. to provide a seven-membered lactone;
e. reducing and acylating the lactone to provide a 2-O-acyl seven-membered ring ether;
f. reacting the product of step e. with a nitrogen base to form a protected seven-membered ring nucleosise analogue.

30. A process for the synthesis of a seven-membered ring nucleoside analogue comprising:
a. reacting a 4,5,6,8-tetra-O-protected seven-membered ring glycal with an epoxidizing agent to form an epoxide;
b. reacting the epoxide from step a. with a nitrogen base to provide a protected seven-membered ring nucleoside analogue;
c. removing the protecting groups from the product of step b. to provide a seven-membered ring nucleoside analogue.

31. A process for the synthesis of a seven-membered ring nucleoside analogue comprising:
a. reacting a protected seven-membered ring glycoside with a nitrogen base in the presence of a silylating agent and a Lewis acid to form a protected seven-membered ring nucleoside analogue;
b. removing the protecting groups from the product of step a. to provide a seven-membered ring nucleoside.

32. A process for the synthesis of a seven-membered ring nucleoside analogue comprising:
a. reacting a nitrogen base with a substituted 2,3-cyclopropyl-4-acetoxy tetrahydropyran derivative to produce an unsaturated seven-membered ring nucleoside analogue; and
b. optionally reducing the unsaturated nucleoside to produce a seven membered ring nucleoside analogue.

33. A method of treating a host infected with a Flaviviridae virus by administering to said host an antivirally-effective amount of a compound of claim 1 or 2, optionally in a pharmaceutically acceptable carrier.

34. The method of claim 33, wherein Base is a pyrimidine base.

35. The method of claim 33, wherein Base is a purine base.

36. The method of claim 33, wherein X is O.

37. The method of claim 33, wherein R₁ is mono, di or triphosphate.

38. The method of claim 33, wherein R₅ is alkyl or H; and R_{5′} is alkyl or H.

39. The method of claim 33, wherein the host is infected with hepatitis C.

40. The method of claim 33, wherein the host is a human.

41. The method of claim 33, wherein the carrier is suitable for oral or intravenous delivery.

42. The method of claim 33, wherein the compound or pharmaceutically acceptable salt, ester or prodrug thereof, is administered in combination or alternation with at least one additional antiviral drug.

43. The method of claim 42, wherein the additional antiviral drug is selected from the group consisting of a protease inhibitor, thiazolidine derivative, helicase inhibitor, benzanilide, polymerase inhibitor, gliotoxin, antisense phosphorothioate oligodeoxynucleotide, inhibitors of IRES-dependent translation, ribozyme, nucleoside analogue, 2′-fluoronucleoside analogue, 1-amino-alkylcyclohexanes, squalene, amantadine, bile acid, N-(phosphonoacetyl)-L-aspartic acid, benzenedicarboxamide, a polyadenylic acid derivative, benzimidazoles, 2′,3′-dideoxyinosine, piperidines, ribavirin and interferon.

44. The method of claim 43, wherein the antiviral agent is an interferon.
